SEQUENCE LISTING (1 sequence)

SEQ ID NO: 1 - protein, 290 aa, Unknown
  Description of Unknown Tyrosine-protein kinase sequence
MGSSHHHHHHSSGLVPRGSHMASGSWEIDPKDLTFLKELGTGQFGVVKYGKWRGQYDVAIKMIKEGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASEKVYTIMYSCWHEKADERPTFKILLSNILDVMDEES

(19) United States
(12) Patent Application Publication
(10) Pub. No.: US 2024/0018153 A1
(43) Pub. Date: Jan. 18, 2024

(54) BENZENESULFONAMIDE DERIVATIVES AND USES THEREOF

(71) Applicants: 2692372 ONTARIO, INC., Mississauga (CA); DUNAD THERAPEUTICS LTD., Great Cambourne, Cambridge (GB)

(72) Inventors: Patrick T. GUNNING, Mississauga (CA); Jeff OMEARA, Halton Hills (CA); Siawash AHMAR, North York (CA); Graham L. SIMPSON, Great Wymondley (GB); Peter HUNT, Storrington (GB); David Alexander ROSA, Toronto (CA); Ji Sung PARK, Etobicoke (CA)

(21) Appl. No.: 18/253,730

(22) PCT Filed: Nov. 18, 2021
(86) PCT No.: PCT/IB2021/000813

§ 371 Date: May 19, 2023

Related U.S. Application Data
(60) Provisional application No. 63/116,731, filed on Nov. 20, 2020.

(51) Int. Cl. C07D 487/04; C07D 403/04; C07D 239/48; C07D 401/12; C07D 213/76; C07D 213/74; C07C 317/32; C07D 405/12; C07K 1/10

(52) U.S. Cl. CPC C07D 487/04; C07D 403/04; C07D 239/48; C07D 401/12; C07D 213/76; C07D 213/74; C07C 317/32; C07D 405/12; C07K 1/10

(57) ABSTRACT

Provided herein are benzenesulfonamide compounds, pharmaceutical compositions comprising said compounds, and methods for using said compounds for the treatment of diseases.

### CROSS-REFERENCES

This application claims the benefit of U.S. Provisional Application No. 63/116,731, filed Nov. 20, 2020, which is hereby incorporated by reference in its entirety herein.

### SEQUENCE LISTINGS

The instant application contains a Sequence Listing which has been submitted electronically in ASCII format and is hereby incorporated by reference in its entirety. Said ASCII copy, created on Nov. 17, 2021, is named 59091-702_601_SL.txt and is 2,904 bytes in size.

### BACKGROUND

Chemical modification is an important tool to alter structure and function of proteins. One way to achieve chemical modification of proteins is to use protein binders, such as covalent small molecule binder (e.g., inhibitors). As a result, protein binders (e.g., covalent small molecule binders (e.g., inhibitors of proteins)) are considered to be useful in multiple applications, including therapeutics.

### BRIEF SUMMARY OF THE DISCLOSURE

Provided herein are protein binders (e.g., covalent small molecule protein binders (e.g., inhibitors). Also provided herein are protein binders (e.g., covalent small molecule protein binders (e.g., inhibitors)) of tubulin polymerization. Also provided herein are pharmaceutical compositions comprising said compounds, and methods for using said compounds for the treatment of diseases.

One embodiment provides a compound, or a salt, solvate, tautomer, or regioisomer thereof, having the structure of Formula (I):

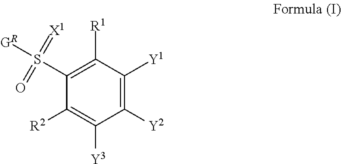

wherein,
G^{R} is alkyl, haloalkyl, heteroalkyl, —N(R⁵)₂, or G;
G is or comprises a protein-binding ligand, is or comprises (e.g., unsaturated) carbocycle, is or comprises (e.g., unsaturated) heterocycle, or is -L²-G¹, wherein L² is a linker (e.g., —O— or —NR⁵—), and G¹ is hydrogen or an organic residue (e.g., is or comprises a protein-binding ligand, is or comprises (e.g., unsaturated) carbocycle, or is or comprises (e.g., unsaturated) heterocycle);
X¹ is absent, O or NR;
each Y¹, Y², and Y³ is independently hydrogen, halo, alkyl, haloalkyl (e.g., with at least two Y being halo or haloalkyl, such as fluoroalkyl, e.g., at least one Y (e.g., Y²) being halo) (e.g., Y¹, Y², Y³ all being F), or G;
R is hydrogen or R⁷;
R¹ is R⁷;
R² is hydrogen, halogen, or R⁷;
each R is independently hydrogen, -L¹R⁴, —C(═O)L¹R⁴, —C(═O)OL¹R⁴, or —C(═O)NR⁴L¹R⁴, wherein each L is independently alkyl, heteroalkyl, aryl, or heteroaryl; and each R⁴ is independently hydrogen, cycloalkyl, heterocycloalkyl, aryl, or heteroaryl;
each R⁵ is independently hydrogen, —CN, —C(═O)R⁶, —C(═O)OR⁶, —C(═O)NR³R⁶, alkyl, heteroalkyl, aryl, or heteroaryl;
each R⁶ is independently hydrogen, alkyl, or heteroalkyl;
each R⁷ is independently G, —CN, —OR³, —S(═O)ₓR³, —S(═O)(═NR³)R³, —S(═O)₂N(R³)₂, —OS(═O)₂R³, —N(R³)₂, —NR³C(═O)R³, —NR³C(═O)N(R³)₂, —NR³C(═NR³)N(R³)₂, —C(═O)R³, —OC(═O)R³, —C(═O)OR³, —OC(═O)OR³, —OC(═O)N(R³)₂, —C(═O)N(R³)₂, alkyl, heteroalkyl, cycloalkyl, heterocycloalkyl, aryl, or heteroaryl; and
x is 0, 1, or 2.

In some embodiments, the compound (e.g., of Formula (I)) comprises only one G.

In some embodiments, when X¹ is O, G^{R} is G, G is L²G¹ and L² is amino or —NR⁵, then Y¹, Y², and Y³ are not all F.

In some embodiments, when —S(═O)(═X¹)G^{R} is —S(═O)(═X¹)G, X¹ is O, then G is not:
(R)-3-(4-phenoxyphenyl)-1-(1λ²-piperidin-3-yl)-1H-pyrazolo[3,4-d]pyrimidin-4-amine;
1-(2-(λ²-azaneyl)ethyl)-3-(4-phenoxyphenyl)-1H-pyrazolo[3,4-d]pyrimidin-4-amine;
(R)-3-(4-phenoxyphenyl)-1-(1λ²-pyrrolidin-3-yl)-1H-pyrazolo[3,4-d]pyrimidin-4-amine;
4-(λ²-azaneyl)-7H-pyrrolo[2,3-d]pyrimidine;
N4-(3-(λ²-azaneyl)phenyl)-5-fluoro-N2-(4-(2-methoxyethoxy)phenyl)pyrimidine-2,4-diamine;
4-(λ²-azaneyl)-5-fluoro-N-(4-(2-methoxyethoxy)phenyl)pyrimidin-2-amine; or
3-(4-phenoxyphenyl)-1λ²-pyrazolo[5,4-d]pyrimidin-4-amine.

In some embodiments, when —S(═O)(═X¹)G^{R} is —S(═O)(═X¹)N(R⁵)G, X¹ is O, then one or more of G and R⁵ is not or does not comprise: substituted or unsubstituted phenyl; substituted or unsubstituted benzyl; 1-naphthyl; pyridin-3-yl; pyridin-4-yl; 2-fluoropyridin-4-yl; or 2,6-difluoropyridin-3-yl.

In some embodiments, G is -L²-G¹, wherein L² is a linker, and G¹ is an organic residue (e.g., is or comprises a protein-binding ligand, is or comprises (e.g., unsaturated) carbocycle, or is or comprises (e.g., unsaturated) heterocycle). In some embodiments, L² is a substituted or unsubstituted unsaturated alkylene (e.g., alkenylene or alkynylene), substituted or unsubstituted arylene, or substituted or unsubstituted heteroarylene, and G¹ is an organic residue (e.g., is or comprises a protein-binding ligand). In some embodiments, L² is a bond, —O—, —NR⁸—, —N(R⁸)₂⁺—, —S—, —S(═O)—, —S(═O)₂—, —CH═CH—, ═CH—, —C≡C—, —C(═O)—, —C(═O)O—, —OC(═O)—, —OC(═O)O—, —C(═O)NR⁸—, —NR⁸C(═O)—, —OC(═O)NR⁸—, —NR⁸C(═O)O—, —NR⁸C(═O)NR⁸—, —NR⁸S(═O)₂—, —NR⁸S(═O)(═NR⁸)—, —S(═O)₂NR⁸—, —S(═O)(═NR⁸)NR⁸—, —C(═O)NR⁸S(═O)₂—, —S(═O)₂NR⁸C(═O)—, substituted or unsubstituted C₁-C₄ alkylene, substituted or unsubstituted C₁-C₈ heteroalkylene, —(C₁-C₄ alkylene)-O—, —O—(C₁-C₄ alkylene)-, —(C₁-C₄ alkylene)-NR⁸—, —NR⁸—(C₁-C₄ alkylene)-, —(C₁-C₄ alkylene)-N(R⁸)₂⁺—, or —N(R⁸)₂⁺—(C₁-C₄ alkylene)-; each R⁸ is independently hydrogen, substituted or unsubstituted C₁-C₄ alkyl, substituted or unsubstituted C₁-C₄ haloalkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and G¹ is an organic residue (e.g., is or comprises a protein-binding ligand).

In some embodiments, G is substituted or unsubstituted unsaturated carbocycle or substituted or unsubstituted unsaturated heterocycle, wherein G and R⁵ on a single N, if present, are optionally taken together to form a substituted or unsubstituted N-containing heterocycloalkyl. In some embodiments, G comprises one or more cyclic ring systems selected from substituted or unsubstituted unsaturated carbocycles and substituted or unsubstituted unsaturated heterocycles. In some embodiments, G comprises two or more cyclic ring systems selected from substituted or unsubstituted unsaturated carbocycles and substituted or unsubstituted unsaturated heterocycles.

In some embodiments, G¹ comprises one or more cyclic ring systems selected from substituted or unsubstituted carbocycles and substituted or unsubstituted heterocycles. In some embodiments, G¹ comprises two or more cyclic ring systems selected from substituted or unsubstituted carbocycles and substituted or unsubstituted heterocycles.

In some embodiments, the two or more cyclic ring systems are connected via a bond. In some embodiments, the two or more cyclic ring systems are connected via one or more linker and/or bond. In some embodiments, the linker is —O—, —NR⁸—, —N(R⁸)₂⁺—, —S—, —S(═O)—, —S(═O)₂—, —CH═CH—, ═CH—, —C≡C—, —C(═O)—, —C(═O)O—, —OC(═O)—, —OC(═O)O—, —C(═O)NR⁸—, —NR⁸C(═O)—, —OC(═O)NR⁸—, —NR⁸C(═O)O—, —NR⁸C(═O)NR⁸—, —NR⁸S(═O)₂—, —S(═O)₂NR⁸—, —C(═O)NR⁸S(═O)₂—, —S(═O)₂NR⁸C(═O)—, substituted or unsubstituted C₁-C₄ alkylene, substituted or unsubstituted C₁-C₈ heteroalkylene, —(C₁-C₄ alkylene)-O—, —O—(C₁-C₄ alkylene)-, —(C₁-C₄ alkylene)-NR⁸—, —NR⁸—(C₁-C₄ alkylene)-, —(C₁-C₄ alkylene)-N(R⁸)₂⁺—, or —N(R⁸)₂⁺—(C₁-C₄ alkylene)-; and each R⁸ is independently hydrogen, substituted or unsubstituted C₁-C₄ alkyl, substituted or unsubstituted C₁-C₄ haloalkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl.

In some embodiments, the cyclic ring system comprises substituted or unsubstituted monocyclic aryl or substituted or unsubstituted monocyclic heteroaryl. In some embodiments, the cyclic ring system comprises substituted or unsubstituted bicyclic aryl or substituted or unsubstituted bicyclic heteroaryl.

In some embodiments, G or G¹ is or comprises a protein-binding ligand selected from a BTK, EGFR, EGFR T790M, JAK3, or tubulin binding ligand.

In some embodiments, G or G¹ is or comprises a protein-binding ligand selected from:

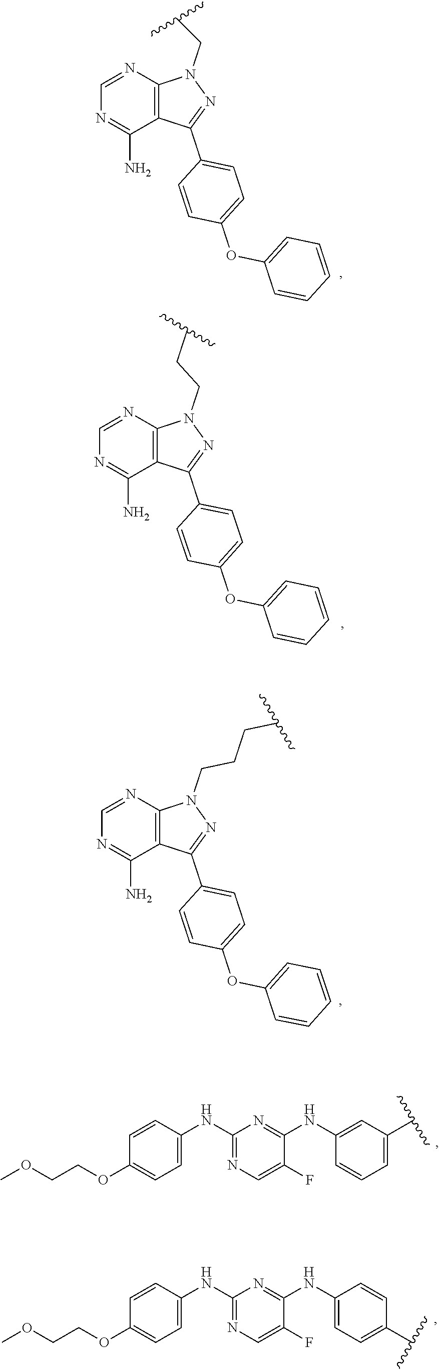 [SMILES: *Cn1nc(-c2ccc(Oc3ccccc3)cc2)c2c(N)ncnc21 ; *CCn1nc(-c2ccc(Oc3ccccc3)cc2)c2c(N)ncnc21 ; *CCCn1nc(-c2ccc(Oc3ccccc3)cc2)c2c(N)ncnc21 ; *c1cccc(Nc2nc(Nc3ccc(OCCOC)cc3)ncc2F)c1 ; *c1ccc(Nc2nc(Nc3ccc(OCCOC)cc3)ncc2F)cc1]

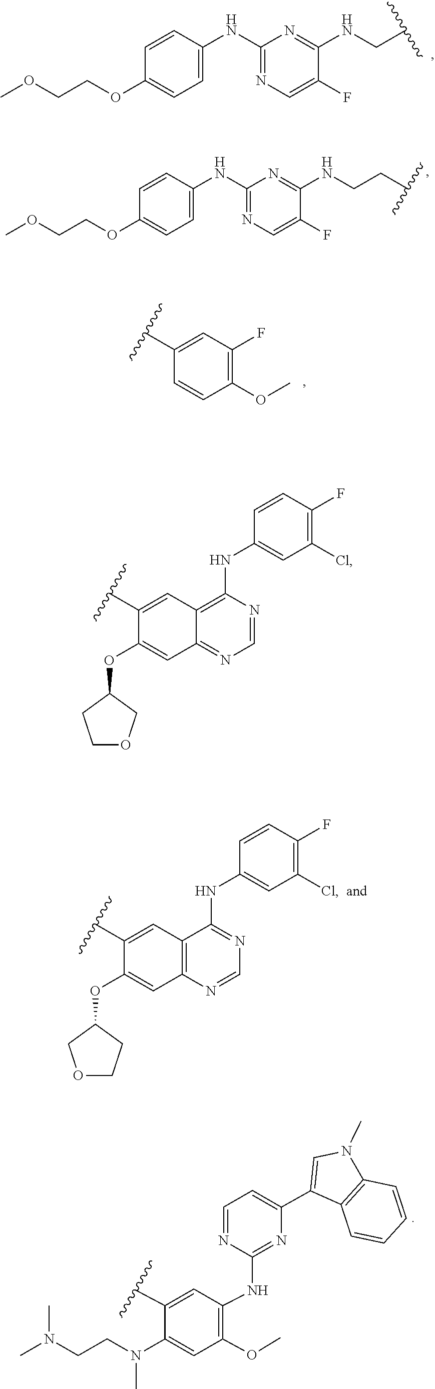

In some embodiments, G or G¹ is or comprises a protein-binding ligand selected from:

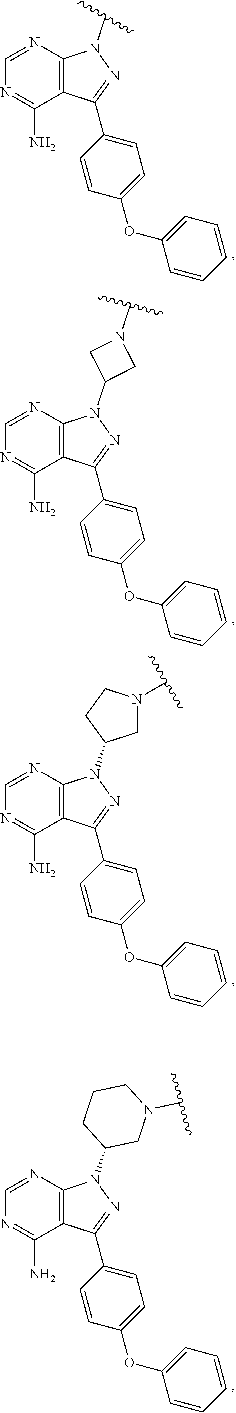

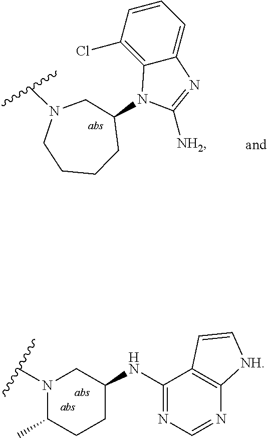 [SMILES: *N1CCCC[C@H](n2c(N)nc3cccc(Cl)c32)C1 ; *N1C[C@@H](Nc2ncnc3[nH]ccc23)CC[C@@H]1C]

In some embodiments, G or G¹ is or comprises a protein-binding ligand that is:

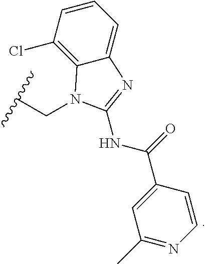 [SMILES: *Cn1c(NC(=O)c2ccnc(C)c2)nc2cccc(Cl)c21]

In some embodiments, G or G¹ is or comprises a protein-binding ligand that is:

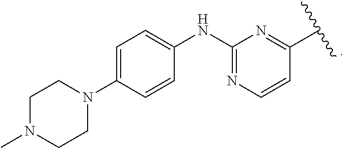 [SMILES: *c1ccnc(Nc2ccc(N3CCN(C)CC3)cc2)n1]

In some embodiments, G or G¹ is or comprises a protein-binding ligand that is:

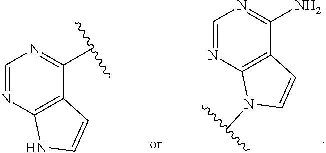 [SMILES: *c1ncnc2[nH]ccc12 ; *n1ccc2c(N)ncnc21]

In some embodiments, each R⁵ is independently hydrogen, —CN, —CH₃, —CH₂CH₃, —CH₂NH₂, —CH₂NHCH₃, —CH₂N(CH₃)₂, —CH₂F, —CHF₂, —CF₃, cyclopropyl, cyclobutyl, or cyclopentyl. In some embodiments, each R⁵ is independently hydrogen, —CN, —CH₃, —CF₃, or cyclopropyl. In some embodiments, each R⁵ is hydrogen.

In some embodiments, each R⁸ is independently hydrogen, substituted or unsubstituted C₁-C₄ alkyl, or substituted or unsubstituted C₁-C₄ heteroalkyl. In some embodiments, each R⁸ is independently hydrogen, —OCH₂F, —OCHF₂, —OCF₃, —OCH₂CH₂F, —OCH₂CHF₂, —OCH₂CF₃, —NHCF₃, or —NHCH₂CF₃. In some embodiments, each R⁸ is independently hydrogen, —OCH₃, —OCH₂CH₃, —OCH₂F, —OCHF₂, —OCF₃, —OCH₂CH₂F, —OCH₂CHF₂, —OCH₂CF₃, cyclopropyloxy, or cyclobutyloxy. In some embodiments, each R⁸ is independently hydrogen, —CH₃, or —OCH₃.

In some embodiments, X¹ is O, NH, or N(substituted or unsubstituted alkyl). In some embodiments, X¹ is O, NH, or N(alkyl). In some embodiments, X¹ is O, NH, or N(CH₃). In some embodiments, X¹ is O. In some embodiments, X¹ is NH or N(CH₃).

In some embodiments, each Y¹, Y², and Y³ is independently halo or alkyl. In some embodiments, Y² is fluoro.

In some embodiments, R² is fluoro.

In some embodiments, Y¹ and Y³ are fluoro.

In some embodiments, R², Y¹, and Y³ are fluoro.

In some embodiments, R², Y¹, and Y³ are fluoro and G^{R} is G.

In some embodiments, R², Y¹, and Y³ are fluoro, R¹ is R⁷ (e.g., sulfone (e.g., —SO₂CH₃), sulfoxide (e.g., —S(═O)CH₃), sulfonamide (e.g., —SO₂NH₂ or —SO₂N(CH₃)₂), —OR³ (e.g., R³ being hydrogen, substituted or unsubstituted alkyl (e.g., haloalkyl), or substituted or unsubstituted aryl (e.g., phenyl)), or substituted or unsubstituted alkyl (e.g., haloalkyl)), and G^{R} is G.

In some embodiments, R², Y¹, Y², and Y³ are fluoro and R¹ is G.

In some embodiments, X is absent or O; R², Y¹, Y², and Y³ are fluoro; G^{R} is —NH₂, —N(CH₃)₂, or substituted or unsubstituted alkyl; and R¹ is G.

Provided in some embodiments herein is a compound having a structure represented by Formula (I-A):

D1-L-D2 Formula (I-A)
wherein:
   D1 is a radical of a protein-binding ligand;
   D2 is a warhead radical (e.g., an aromatic (e.g., substituted phenyl) warhead radical); and
   L is a linker,
or a pharmaceutically acceptable salt or solvate thereof.

In some embodiments, D2 covalently modifies a target protein (e.g., tubulin (e.g., β-tubulin), Janus kinase 3 (JAK3), epidermal growth factor receptor (EGFR), Bruton's tyrosine kinase (BTK), Fibroblast Growth Factor Receptor 4 (FGFR4), receptor-interacting serine/threonine-protein kinase 2 (RIPK2), or cytoplasmic tyrosine-protein kinase (BMX)).

In some embodiments, D2 binds to, disrupts, and/or modifies a target protein (e.g., tubulin, JAK3, EGFR, BTK, FGFR4, RIPK2, or BMX).

In some embodiments, D2 comprises one or more warhead group, each warhead group being independently selected from the group consisting of substituted or unsubstituted sulfonamide (e.g., unsubstituted sulfonamide or sulfonamide substituted with alkyl (e.g., methyl)), sulfone, sulfoxide, substituted or unsubstituted amino (e.g., a secondary amine (e.g., —NH— or —NCH₃—) or a tertiary amine (e.g., >N—)), or substituted aryl (e.g., aryl substituted with one or more substituent, each substituent being independently selected from sulfone, sulfoxide, sulfonamide, halogen (e.g., fluoro), hydroxy, substituted or unsubstituted alkoxy (e.g., unsubstituted alkoxy (e.g., methoxy), alkoxy substituted with halogen (e.g., fluoro) (e.g., —OCH₂F, —OCHF₂, or —OCF₃), or alkoxy substituted with substituted or unsubstituted aryl (e.g., phenyl)), substituted or unsubstituted alkyl (e.g., alkyl substituted with halogen (e.g., fluoro) (e.g., —CH₂F, —CHF₂, or —CF₃)))).

In some embodiments, D2 comprises an aryl substituted with one or more substituent, each substituent being independently selected from halogen (e.g., fluoro), hydroxy, substituted or unsubstituted alkoxy (e.g., unsubstituted alkoxy (e.g., methoxy), alkoxy substituted with halogen (e.g., fluoro) (e.g., —OCH₂F, —OCHF₂, or —OCF₃), or alkoxy substituted with substituted or unsubstituted aryl (e.g., phenyl)), substituted or unsubstituted alkyl (e.g., alkyl substituted with halogen (e.g., fluoro) (e.g., —CH₂F, —CHF₂, or —CF₃))).

In some embodiments, D2 comprises a sulfone, a sulfoxide, or a sulfonamide.

In some embodiments, D2 comprises a sulfone and an aryl substituted with one or more substituent, each substituent being independently selected from halogen (e.g., fluoro), hydroxy, substituted or unsubstituted alkoxy (e.g., unsubstituted alkoxy (e.g., methoxy), alkoxy substituted with halogen (e.g., fluoro) (e.g., —OCH₂F, —OCHF₂, or —OCF₃), or alkoxy substituted with substituted or unsubstituted aryl (e.g., phenyl)), substituted or unsubstituted alkyl (e.g., alkyl substituted with halogen (e.g., fluoro) (e.g., —CH₂F, —CHF₂, or —CF₃))).

In some embodiments, D2 comprises a sulfoxide and an aryl substituted with one or more substituent, each substituent being independently selected from halogen (e.g., fluoro), hydroxy, substituted or unsubstituted alkoxy (e.g., unsubstituted alkoxy (e.g., methoxy), alkoxy substituted with halogen (e.g., fluoro) (e.g., —OCH₂F, —OCHF₂, or —OCF₃), or alkoxy substituted with substituted or unsubstituted aryl (e.g., phenyl)), substituted or unsubstituted alkyl (e.g., alkyl substituted with halogen (e.g., fluoro) (e.g., —CH₂F, —CHF₂, or —CF₃))).

In some embodiments, D2 comprises a sulfonamide and an aryl substituted with one or more substituent, each substituent being independently selected from halogen (e.g., fluoro), hydroxy, substituted or unsubstituted alkoxy (e.g., unsubstituted alkoxy (e.g., methoxy), alkoxy substituted with halogen (e.g., fluoro) (e.g., —OCH₂F, —OCHF₂, or —OCF₃), or alkoxy substituted with substituted or unsubstituted aryl (e.g., phenyl)), substituted or unsubstituted alkyl (e.g., alkyl substituted with halogen (e.g., fluoro) (e.g., —CH₂F, —CHF₂, or —CF₃))).

In some embodiments, D2 is or comprises an aryl substituted with halogen (e.g., fluoro).

In some embodiments, D2 is or comprises an aryl substituted with halogen (e.g., fluoro) and alkyl substituted with halogen (e.g., fluoro) (e.g., —CH₂F, —CHF₂, or —CF₃).

In some embodiments, D2 is or comprises an aryl substituted with halogen (e.g., fluoro) and hydroxy.

In some embodiments, D2 is or comprises an aryl substituted with halogen (e.g., fluoro) and unsubstituted alkoxy (e.g., methoxy).

In some embodiments, D2 is or comprises an aryl substituted with halogen (e.g., fluoro) and alkoxy substituted with substituted or unsubstituted an aryl (e.g., phenyl).

In some embodiments, D2 is or comprises an aryl substituted with halogen (e.g., fluoro) and alkoxy substituted with halogen (e.g., fluoro) (e.g., —OCH₂F, —OCHF₂, or —OCF₃).

In some embodiments, D2 is or comprises an aryl substituted with halogen (e.g., fluoro) and sulfone.

In some embodiments, D2 is or comprises an aryl substituted with halogen (e.g., fluoro) and sulfoxide.

In some embodiments, D2 is or comprises an aryl substituted with halogen (e.g., fluoro) and unsubstituted sulfonamide.

In some embodiments, D2 is or comprises an aryl substituted with halogen (e.g., fluoro) and substituted sulfonamide (e.g., sulfonamide substituted with alkyl (e.g., methyl)).

In some embodiments, D2 comprises a sulfone.

In some embodiments, D2 comprises a sulfonamide.

In some embodiments, D2 comprises a sulfoxide.

In some embodiments, the linker is a non-releasable linker (e.g., the linker does not decompose (e.g., hydrolyze) or release the warhead radical (or a free form thereof), the radical of the protein-binding ligand (or a free form thereof), or any other portion of the compound (e.g., a radical of any Formula provided herein) (or a free form thereof)).

In some embodiments, the linker comprises one or more linker group, each linker group being independently selected from the group consisting of —O—, (substituted or unsubstituted) amino, substituted or unsubstituted (e.g., acyclic (e.g., straight or branched) or cyclic) alkyl(ene), substituted or unsubstituted (e.g., acyclic (e.g., straight or branched) or cyclic) heteroalkyl(ene), and substituted or unsubstituted alkoxy.

In some embodiments, the linker comprises one or more linker group, each linker group being independently selected from the group consisting of (substituted or unsubstituted) amino and substituted or unsubstituted (e.g., acyclic (e.g., straight or branched) or cyclic) heteroalkyl(ene).

In some embodiments, the linker is —O—, (substituted or unsubstituted) amino or substituted or unsubstituted (e.g., acyclic (e.g., straight or branched) or cyclic) heteroalkyl(ene).

In some embodiments, L is a bond, substituted or unsubstituted alkylene (e.g., C(═O), methylene, ethylene, or alkyl substituted with oxo and/or heterocyclyl (e.g., azetidinyl, pyrrolidinyl, or piperidinyl)), substituted or unsubstituted heteroalkylene (e.g., —N═CH—, —CH₂NH—, —CH₂NCH₃—, —CH₂CH₂NH—, —NHCH₂CH₂NH—, —CH₂CH₂NCH₃—, —CH₂CH₂NHCH₂—, —NHCH₂CH₂NHCH₂—, —NHCH₂CH₂CH₂NH—, —CH₂CH₂CH₂NH—, or heteroalkyl substituted with oxo (e.g., —C(═O)NH—, —CH₂CH₂N(CH₃)C(═O)—, —CH₂CH₂NHC(═O)—, —NHCH₂CH₂N(CH₃)C(═O)—, —NHCH₂CH₂NHC(═O)—, —CH₂CH₂CH₂N(CH₃)C(═O)—, or —CH₂CH₂CH₂NHC(═O)—)), substituted or unsubstituted alkoxy (e.g., methoxy), substituted or unsubstituted piperidinyl, substituted or unsubstituted pyrrolidinyl, substituted or unsubstituted azetidinyl, or substituted or unsubstituted amino (e.g., —NH—, amino substituted with substituted or unsubstituted aryl (e.g., —NH-phenyl-, aryl substituted with amino (e.g., —NH-phenyl-NH—) or aryl substituted with alkoxy (e.g., —NH-phenyl-OCH₂—))).

In some embodiments, L is a bond.

In some embodiments, D1 has a structure represented in Table 2 or Table 3 (e.g., and L is a bond).

In some embodiments, disclosed herein is a compound or a salt, solvate, tautomer, or regioisomer thereof, wherein the compound is a compound from Table 1, Table 2, or Table 3.

Provided in some embodiments herein is a compound selected from Table 4, Table 5, Table 6, Table 7, or Table 8.

A pharmaceutically acceptable composition comprising a compound disclosed herein, or a salt, solvate, tautomer, or regioisomer thereof, and one or more of pharmaceutically acceptable excipients.

In some embodiments, disclosed herein is a protein modified with a compound disclosed herein, or a salt, solvate, tautomer, or regioisomer thereof, wherein the compound forms a covalent bond with a sulfur atom of a cysteine residue of the protein.

In some embodiments, disclosed herein is a method of modifying (e.g., attaching to and/or degrading) a polypeptide with a compound, comprising contacting the polypeptide with a compound disclosed herein, or a salt, solvate, tautomer, or regioisomer thereof, to form a covalent bond with a sulfur atom of a cysteine residue of the polypeptide.

In some embodiments, disclosed herein is a method of binding a compound to a polypeptide, comprising contacting the polypeptide with a compound disclosed herein, or a salt, solvate, tautomer, or regioisomer thereof.

In some embodiments, disclosed herein is a method of disrupting a polypeptide (e.g. the function thereof), comprising contacting the polypeptide with a compound disclosed herein, or a salt, solvate, tautomer, or regioisomer thereof.

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A shows time-dependent inhibition of BTK by ibrutinib. Product formation versus pre-incubation time data were fitted to a mono-exponential decay model, showing concentration-dependent first order loss of enzyme activity.

FIG. 1B shows time-dependent inhibition of BTK by ibrutinib. IC₅₀ values are shown to decrease as pre-incubation times increase.

FIG. 2A shows time-dependent inhibition of BTK by Compound I-40. Product formation versus pre-incubation time data were fitted to a mono-exponential decay model, showing concentration-dependent first order loss of enzyme activity.

FIG. 2B shows time-dependent inhibition of BTK by Compound I-40. IC₅₀ values are shown to decrease as pre-incubation times increase.

FIG. 3A shows time-dependent inhibition of BTK by Compound I-37. Product formation versus pre-incubation time data were fitted to a mono-exponential decay model, showing concentration-dependent first order loss of enzyme activity.

FIG. 3B shows time-dependent inhibition of BTK by Compound I-37. IC₅₀ values are shown to decrease as pre-incubation times increase.

FIG. 4A shows time-dependent inhibition of BTK by Compound I-32. Product formation versus pre-incubation time data were fitted to a mono-exponential decay model, showing concentration-dependent first order loss of enzyme activity.

FIG. 4B shows time-dependent inhibition of BTK by Compound I-32. IC₅₀ values are shown to decrease as pre-incubation times increase.

FIG. 5A shows time-dependent inhibition of BTK by Compound I-38. Product formation versus pre-incubation time data were fitted to a mono-exponential decay model, showing concentration-dependent first order loss of enzyme activity.

FIG. 5B shows time-dependent inhibition of BTK by Compound I-38. IC₅₀ values are shown to decrease as pre-incubation times increase.

FIG. 6A shows time-dependent inhibition of BTK by Compound I-34. Product formation versus pre-incubation time data were fitted to a mono-exponential decay model, showing concentration-dependent first order loss of enzyme activity.

FIG. 6B shows time-dependent inhibition of BTK by Compound I-34. IC₅₀ values are shown to decrease as pre-incubation times increase.

FIG. 7A shows time-dependent inhibition of BTK by Compound I-33. Product formation versus pre-incubation time data were fitted to a mono-exponential decay model, showing concentration-dependent first order loss of enzyme activity.

FIG. 7B shows time-dependent inhibition of BTK by Compound I-33. IC₅₀ values are shown to decrease as pre-incubation times increase.

FIG. 8A shows time-dependent inhibition of EGFR by ibrutinib. Product formation versus pre-incubation time data were fitted to a mono-exponential decay model, showing concentration-dependent first order loss of enzyme activity.

FIG. 8B shows time-dependent inhibition of EGFR by ibrutinib. IC₅₀ values are shown to decrease as pre-incubation times increase.

FIG. 9A shows time-dependent inhibition of EGFR by Compound I-37. Product formation versus pre-incubation time data were fitted to a mono-exponential decay model, showing concentration-dependent first order loss of enzyme activity.

FIG. 9B shows time-dependent inhibition of EGFR by Compound I-37. IC₅₀ values are shown to decrease as pre-incubation times increase.

FIG. 10A shows time-dependent inhibition of EGFR by Compound I-2. Product formation versus pre-incubation time data were fitted to a mono-exponential decay model, showing concentration-dependent first order loss of enzyme activity.

FIG. 10B shows time-dependent inhibition of EGFR by Compound I-2. IC₅₀ values are shown to decrease as pre-incubation times increase.

FIG. 11A shows time-dependent inhibition of EGFR by Compound I-3. Product formation versus pre-incubation time data were fitted to a mono-exponential decay model, showing concentration-dependent first order loss of enzyme activity.

FIG. 11B shows time-dependent inhibition of EGFR by Compound I-3. IC₅₀ values are shown to decrease as pre-incubation times increase.

FIG. 12A shows time-dependent inhibition of EGFR by Compound I-4. Product formation versus pre-incubation time data were fitted to a mono-exponential decay model, showing concentration-dependent first order loss of enzyme activity.

FIG. 12B shows time-dependent inhibition of EGFR by Compound I-4. IC₅₀ values are shown to decrease as pre-incubation times increase.

FIG. 13 shows time-dependent product formation (activity) of EGFR after incubation with various compounds provided herein, followed by jump dilution, relative to DMSO control.

FIG. 14 shows time-dependent product formation (activity) of BTK after incubation with various compounds provided herein, followed by jump dilution, relative to DMSO control.

FIG. 15A shows the mass spectroscopy trace for the intact mass analysis of His-tagged BTK protein (SEQ ID NO: 1) showing the mass (32,650 Da).

FIG. 15B shows the mass spectroscopy trace for the intact mass analysis of His-tagged BTK (SEQ ID NO: 1) incubated with Compound I-56 showing the covalent adduct mass (33,209 Da) for modification by one molecule of Compound I-56.

FIG. 16A shows the mass spectroscopy trace for the intact mass analysis with His-tagged BTK protein (SEQ ID NO: 1) incubated with Compound I-55 showing the covalent adduct mass (33,243 Da) for modification by one molecule of Compound I-55.

FIG. 16B shows the mass spectroscopy trace for the intact mass analysis with His-tagged BTK protein (SEQ ID NO: 1) incubated with Compound I-24 showing the covalent adduct mass (33,263 Da) for modification by one molecule of Compound I-24.

FIG. 17A shows the peptide fragment coverage of His-BTK KD (SEQ ID NO: 1) after trypsin digestion showing coverage of 92% of the sequence and confirming covalent modification of peptide 467QRPIFIITEYMANGCLLNYLR487 at C481 by example Compound I-24.

FIG. 17B shows the MSMS spectrum of peptide 467QRPIFIITEYMANGCLLNYLR487 from Compound I-24 treated His-BTK KD (SEQ ID NO: 1) digest where the Cys is modified by one Compound I-24 (observed mass 3140.4 Da). The alignment of b and y ions confirms that Cys-481 is the amino acid that is modified by Compound I-24.

FIG. 18A shows the peptide fragment coverage of His-BTK KD (SEQ ID NO: 1) after trypsin digestion showing coverage of 88% of the sequence and confirming covalent modification of two peptides—467QRPIFIITEYMANGCLLNYLR487 at C481, and 457LVQLYGVCTK466 at C464 by example Compound I-55.

FIG. 18B shows the MSMS spectrum of peptide 467QRPIFIITEYMANGCLLNYLR487 from example Compound I-55 treated His-BTK KD (SEQ ID NO: 1) digest where C481 is modified by one Compound I-55 (observed mass 3121.4 Da). The alignment of b and y ions confirms that C481 is the amino acid that is modified by Compound I-55.

FIG. 18C shows the MSMS spectrum of peptide 457LVQLYGVCTK466 from example Compound I-55 treated His-BTK KD (SEQ ID NO: 1) digest where C464 is modified by one Compound I-55 (observed mass 1716.7 Da). The alignment of b and y ions confirms that C464 is the amino acid that is modified by Compound I-55.

FIG. 19A shows the peptide fragment coverage of His-BTK KD (SEQ ID NO: 1) after trypsin digestion showing coverage of 88% of the sequence and confirming covalent modification of peptide 467QRPIFIITEYMANGCLLNYLR487 at C481 by Compound I-56.

FIG. 19B shows the MSMS spectrum of peptide 467QRPIFIITEYMANGCLLNYLR487 from Compound I-56 treated His-BTK KD (SEQ ID NO: 1) digest where the Cys is modified by one Compound I-56 (observed mass 3087.4 Da). The alignment of b and y ions confirms that Cys-481 is the amino acid that is modified by Compound I-56.

FIG. 20 illustrates an example of a warhead portion, a linker portion, and a protein-binding ligand portion of a compound provided herein.

### DETAILED DESCRIPTION OF THE DISCLOSURE

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. In some embodiments, about is within 10% of the stated number or numerical range. In some embodiments, about is within 5% of the stated number or numerical range. In some embodiments, about is within 1% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ moiety.

“Hydroxy” or “hydroxyl” refers to the —OH moiety.

“Alkyl” generally refers to a non-aromatic straight or branched hydrocarbon chain radical consisting solely of carbon and hydrogen atoms, partially or fully saturated, cyclic or acyclic, having from one to fifteen carbon atoms (e.g., C₁-C₁₄ alkyl). Unless otherwise state, alkyl is saturated or unsaturated (e.g., an alkenyl, which comprises at least one carbon-carbon double bond). Disclosures provided herein of an “alkyl” are intended to include independent recitations of a saturated “alkyl,” unless otherwise stated. Alkyl groups described herein are generally monovalent, but may also be divalent (which may also be described herein as “alkylene” or “alkylenyl” groups). In certain embodiments, an alkyl comprises one to thirteen carbon atoms (e.g., C₁-C₁₂ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. In general, alkyl groups are each independently substituted or unsubstituted. Each recitation of “alkyl” provided herein, unless otherwise stated, includes a specific and explicit recitation of an unsaturated “alkyl” group. Similarly, unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl). In certain embodiments, an alkyl includes alkenyl, alkynyl, cycloalkyl, carbocycloalkyl, cycloalkylalkyl, haloalkyl, and fluoroalkyl, as defined herein.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, ortrifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is as defined above. Unless stated otherwise specifically in the specification, an alkoxy group is optionally substituted, as defined above for an alkyl group.

“Alkoxyalkyl” refers to an alkyl moiety comprising at least one alkoxy substituent, where alkyl is as defined above. Unless stated otherwise specifically in the specification, an alkoxyalkyl group is optionally substituted, as defined above for an alkyl group.

“Alkylamino” refers to a moiety of the formula —NHRₐ or —NRₐR_{b} where Rₐ and R_{b} are each independently an alkyl group as defined above. Unless stated otherwise specifically in the specification, an alkylamino group is optionally substituted, as defined above for an alkyl group.

“Alkylaminoalkyl” refers to an alkyl moiety comprising at least one alkylamino substituent. The alkylamino substituent can be on a tertiary, secondary or primary carbon. Unless stated otherwise specifically in the specification, an alkylaminoalkyl group is optionally substituted, as defined above for an alkyl group.

“Aminoalkyl” refers to an alkyl moiety comprising at least one amino substituent. The amino substituent can be on a tertiary, secondary or primary carbon. Unless stated otherwise specifically in the specification, an aminoalkyl group is optionally substituted, as defined above for an alkyl group.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Arylene” refers to a divalent aryl group which links one part of the molecule to another part of the molecule. Unless stated specifically otherwise, an arylene is optionally substituted, as defined above for an aryl group.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

The term “carbocycle” or “carbocyclic” refers to a ring or ring system where the atoms forming the backbone of the ring are all carbon atoms. The term thus distinguishes carbocyclic group from a “heterocycle” or “heterocyclic” in which the ring backbone contains at least one atom which is different from carbon. In some embodiments, carbocycles are monocyclic, bicyclic, polycyclic, spirocyclic or bridged compounds. Carbocycle includes aromatic and partially or fully saturated ring systems. Heterocycle includes aromatic and partially or fully saturated ring systems. In some embodiments, carbocycle comprises cycloalkyl and aryl. In some embodiments, a carbocycle provided herein is optionally substituted (e.g., carbocycle substituted with one or more carbocycle substitutent, each carbocycle substituent being independently selected from the group consisting of alkyl, oxo, halo, hydroxyl, heteroalkyl, alkoxy, aryl, and heteroaryl). In some embodiments, a heterocycle provided herein is optionally substituted (e.g., heterocycle substituted with one or more heterocycle substitutent, each heterocycle substituent being independently selected from the group consisting of alkyl, oxo, halo, hydroxyl, heteroalkyl, alkoxy, aryl, and heteroaryl).

“Cyclic ring” refers to a carbocycle or heterocycle, including aromatic, non-saturated, and saturated carbocycle and heterocycle. A “cyclic ring” is optionally monocyclic or polycyclic (e.g., bicyclic).

“Cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a cycloalkyl comprises three to ten carbon atoms. In other embodiments, a cycloalkyl comprises five to seven carbon atoms. The cycloalkyl is attached to the rest of the molecule by a single bond. Cycloalkyl is saturated (i.e., containing single C—C bonds only) or unsaturated (i.e., containing one or more double bonds or triple bonds). Examples of monocyclic cycloalkyls include, e.g., cyclopropyl, cyclobutyl, cyclopentyl, cyclohexyl, cycloheptyl, and cyclooctyl. An unsaturated cycloalkyl is also referred to as “cycloalkenyl.” Examples of monocyclic cycloalkenyls include, e.g., cyclopentenyl, cyclohexenyl, cycloheptenyl, and cyclooctenyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “cycloalkyl” is meant to include cycloalkyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Cycloalkylalkyl” refers to a radical of the formula —R^{c}-cycloalkyl where R^{c} is an alkylene chain as defined above. The alkylene chain and the cycloalkyl radical is optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

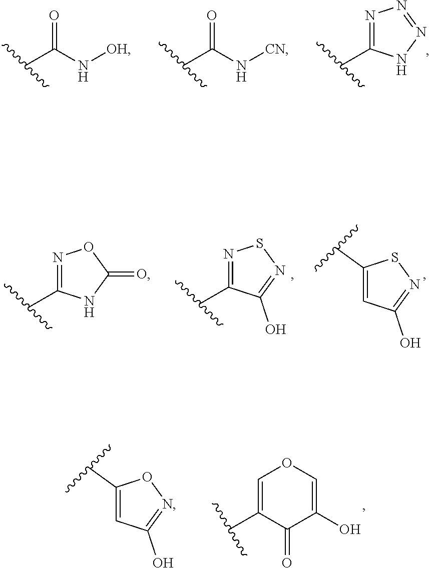

and the like.

“Halo” or “halogen” refers to bromo, chloro, fluoro or iodo substituents. A “haloalkyl” refers to an alkyl radical, as described herein, that is substituted with one or more halo radical, such as described above.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

The term “heteroalkyl” refers to an alkyl group as defined above in which one or more skeletal atoms of the alkyl are selected from an atom other than carbon, e.g., oxygen, nitrogen (e.g. —NH—, —N(alkyl)-, or —N(aryl)-), sulfur (e.g. —S—, —S(═O)—, or —S(═O)₂—), phosphorous (e.g. >P—, >P(═O)—, or —P(═O)₂), or combinations thereof. In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₁₈ heteroalkyl. In some embodiments, a heteroalkyl is a C₁-C₁₂ heteroalkyl. In some embodiments, a heteroalkyl is a C₁-C₆ heteroalkyl. In some embodiments, a heteroalkyl is a C₁-C₄ heteroalkyl. Representative heteroalkyl groups include, but are not limited to —OCH₂OMe, —OCH₂CH₂OH, —CH₂CH₂OMe, or —OCH₂CH₂OCH₂CH₂NH₂. In some embodiments, heteroalkyl includes alkoxy, alkoxyalkyl, alkylamino, alkylaminoalkyl, aminoalkyl, heterocycloalkyl, heterocycloalkyl, and heterocycloalkylalkyl, as defined herein. Unless stated otherwise specifically in the specification, a heteroalkyl group is optionally substituted, as defined above for an alkyl group.

“Heteroalkylene” refers to a divalent heteroalkyl group defined above which links one part of the molecule to another part of the molecule. Unless stated specifically otherwise, a heteroalkylene is optionally substituted, as defined above for an alkyl group.

The term “heterocycle” or “heterocyclic” refers to heteroaromatic rings (also known as heteroaryls) and heterocycloalkyl rings (also known as heteroalicyclic groups) that includes at least one heteroatom selected from nitrogen, oxygen and sulfur, wherein each heterocyclic group has from 3 to 12 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 12 atoms in its ring system and aromatic heterocyclic groups include rings having 5 to 12 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3 h-indolyl, indolin-2-onyl, isoindolin-1-onyl, isoindoline-1,3-dionyl, 3,4-dihydroisoquinolin-1 (2H)-onyl, 3,4-dihydroquinolin-2 (1H)-onyl, isoindoline-1,3-dithionyl, benzo[d]oxazol-2 (3H)-onyl, 1H-benzo[d]imidazol-2 (3H)-onyl, benzo[d]thiazol-2 (3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

“Heterocycloalkyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocycloalkyl radical is a monocyclic, bicyclic, tricyclic or tetracyclic ring system, which optionally includes fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the ring(s). In some embodiments, heterocycloalkyl comprises 2-12 C atoms, 0-6 N atoms, 0-4 O atoms, and 0-4 S atoms. In some embodiments, heterocycloalkyl comprises 2-10 C atoms, 0-4 N atoms, 0-2 O atoms, and 0-2 S atoms. In some embodiments, heterocycloalkyl comprises 2-8 C atoms, 0-3 N atoms, 0-1 O atoms, and 0-1 S atoms. In some embodiments, heterocycloalkyl is a saturated or partially unsaturated 3-7 membered monocyclic, 6-10 membered bicyclic, or 13-16 membered polycyclic (e.g., tricyclic or tetracyclic) ring system having 1, 2, 3, or 4 heteroatom ring members each independently selected from N, O, and S. In some embodiments, heterocycloalkyl comprises 1 or 2 heteroatom ring members each independently selected from N, O, and S. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocycloalkyl” is meant to include heterocycloalkyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocycloalkyl” or “N-attached heterocycloalkyl” refers to a heterocycloalkyl radical as defined above containing at least one nitrogen and where the point of attachment of the heterocycloalkyl radical to the rest of the molecule is through a nitrogen atom in the heterocycloalkyl radical. An N-heterocycloalkyl radical is optionally substituted as described above for heterocycloalkyl radicals. Examples of such N-heterocycloalkyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocycloalkyl” or “C-attached heterocycloalkyl” refers to a heterocycloalkyl radical as defined above containing at least one heteroatom and where the point of attachment of the heterocycloalkyl radical to the rest of the molecule is through a carbon atom in the heterocycloalkyl radical. A C-heterocycloalkyl radical is optionally substituted as described above for heterocycloalkyl radicals. Examples of such C-heterocycloalkyl radicals include, but are not limited to, 2-morpholinyl, 2- or 3- or 4-piperidinyl, 2-piperazinyl, 2- or 3-pyrrolidinyl, and the like.

“Heterocycloalkylalkyl” refers to a radical of the formula —R^{c}-heterocycloalkyl where R^{c} is an alkylene chain as defined above. If the heterocycloalkyl is a nitrogen-containing heterocycloalkyl, the heterocycloalkyl is optionally attached to the alkyl radical at the nitrogen atom. The alkylene chain of the heterocycloalkylalkyl radical is optionally substituted as defined above for an alkylene chain. The heterocycloalkyl part of the heterocycloalkylalkyl radical is optionally substituted as defined above for a heterocycloalkyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted cycloalkyl, optionally substituted cycloalkylalkyl, optionally substituted heterocycloalkyl, optionally substituted heterocycloalkylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocycloalkyl (optionally substituted with halogen, hydroxy, methoxy, or trifluoromethyl), heterocycloalkylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Heteroarylene” refers to a divalent heteroaryl group which links one part of the molecule to another part of the molecule. Unless stated specifically otherwise, a heteroarylene is optionally substituted, as defined above for a heteroaryl group.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

In general, optionally substituted groups are each independently substituted or unsubstituted. Each recitation of an optionally substituted group provided herein, unless otherwise stated, includes an independent and explicit recitation of both an unsubstituted group and a substituted group (e.g., substituted in certain embodiments, and unsubstituted in certain other embodiments). Unless otherwise stated, a substituted group provided herein (e.g., substituted alkyl) is substituted by one or more substituent, each substituent being independently selected from the group consisting of halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2), where each R^{a} is independently hydrogen, alkyl (e.g., optionally substituted with halogen, hydroxy, methoxy, or trifluoromethyl), fluoroalkyl, carbocyclyl (e.g., optionally substituted with halogen, hydroxy, methoxy, or trifluoromethyl), carbocyclylalkyl (e.g., optionally substituted with halogen, hydroxy, methoxy, or trifluoromethyl), aryl (e.g., optionally substituted with halogen, hydroxy, methoxy, or trifluoromethyl), aralkyl (e.g., optionally substituted with halogen, hydroxy, methoxy, or trifluoromethyl), heterocyclyl (e.g., optionally substituted with halogen, hydroxy, methoxy, or trifluoromethyl), heterocyclylalkyl (e.g., optionally substituted with halogen, hydroxy, methoxy, or trifluoromethyl), heteroaryl (e.g., optionally substituted with halogen, hydroxy, methoxy, or trifluoromethyl), or heteroarylalkyl (e.g., optionally substituted with halogen, hydroxy, methoxy, or trifluoromethyl).

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

Recitations of structures described herein also include recitations of tautomers thereof, e.g., a switch of a single bond and adjacent double bond, for example

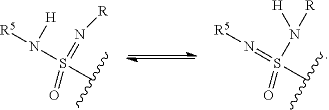

In some embodiments, the present disclosure provides a tautomer of a compound or fragment described herein or an equilibrium of tautomers. A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

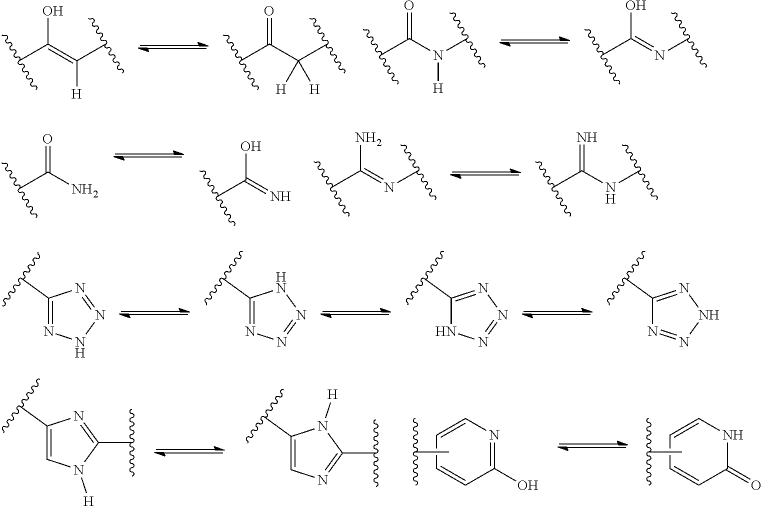

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (2H), tritium (H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. In some embodiments, isotopic substitution with ¹⁸F is contemplated. All isotopic variations of the compounds of the present disclosure, whether radioactive or not, are encompassed within the scope of the present disclosure.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [Curr., Pharm. Des., 2000; 6 (10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64 (1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

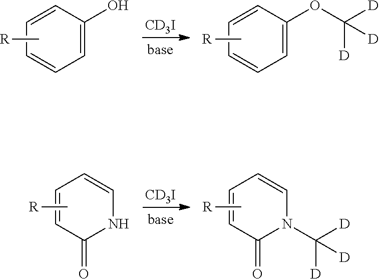

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

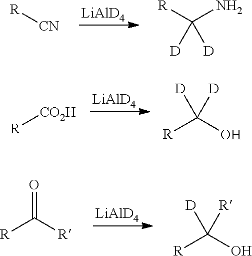

Deuterium gas and palladium catalyst are employed to reduce unsaturated carbon-carbon linkages and to perform a reductive substitution of aryl carbon-halogen bonds as illustrated, by way of example only, in the reaction schemes below.

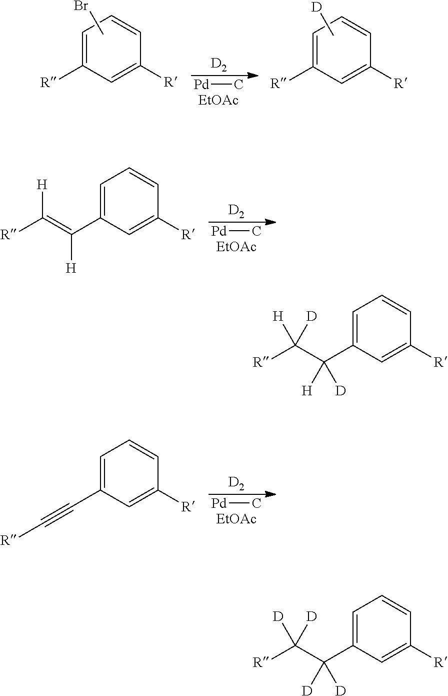

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the inhibitor of cyclin-dependent kinases (CDKs) compounds described herein is intended to encompass any and all pharmaceutically suitable salt forms. Exemplary pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. The compounds provided herein optionally exist in either unsolvated as well as solvated forms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

Chemical modification is an important tool to alter structure and function of proteins. One way to achieve chemical modification of proteins is to use protein binders (e.g., a (e.g., covalent) small molecule inhibitor). As a result, binders (e.g., covalent small molecule binders (e.g., inhibitors) of proteins) are considered to be useful in multiple applications, including therapeutics. Covalent binding (e.g., inhibition) of a target protein may minimize the required systemic drug exposure. In some embodiments, protein (e.g., functional) activity can only be restored by de novo protein synthesis, resulting in a prolonged therapeutic effect long after the compound is cleared from the blood. Strategically placing an electrophilic moiety on the protein binder (e.g., inhibitor) will allow it to undergo attack by a nucleophilic amino acid residue upon binding to the target protein, forming a reversible or irreversible bond that is much stronger than typical noncovalent interactions. However, the ability to form a covalent bond with the target enzyme has raised concerns about indiscriminate reactivity with off-target proteins, even though some of the most prescribed drugs are covalent irreversible binders. This led to the disfavor of covalent modifiers as drug candidates until the recent successful development of irreversible covalent kinase inhibitors ibrutinib and afatinib, which form an irreversible covalent bond between an acrylamide warhead and a nonconserved cysteine residue on the ATP-binding site but also with nontargeted cellular thiols. The ability to form covalent adducts with off-target proteins has been linked to an increased risk of unpredictable idiosyncratic toxicity along with the daily drug dose administered to patients. Accordingly, there is a need to reduce the risk of non-target covalent interactions by incorporating less reactive electrophilic moieties into binders (e.g., to form covalent small molecule binders (e.g., inhibitors). In some embodiments, described herein is a protein binder, such as a covalent small molecule binder (e.g., inhibitor). In some embodiments, described herein is a covalent small molecule protein binder which acts functionally as a protein. In some embodiments, described herein is a covalent small molecule binder which acts functionally as an inhibitor. In some embodiments, described herein is a pharmaceutical composition comprising a protein binder (e.g., a covalent small molecule binder (e.g., inhibitor) and one or more of pharmaceutically acceptable excipients. In other embodiments, a protein binder (e.g., covalent small molecule binder (e.g., inhibitor)) is used to treat or prevent a disease or condition in a subject in need thereof.

In some embodiments, a protein binder provided herein, such as a covalent small molecule binder (e.g., inhibitor) is a benzenesulfonamide derivative compound. In some embodiments, a benzenesulfonamide derivative compound as described herein is used to treat or prevent a disease or condition in a subject in need thereof.

In some instances, a protein binder provided herein, such as any compound provided herein, such as a compound of any one of Tables 4-8, binds to, (e.g., covalently) interacts with, modulates (e.g., inhibits), destabilizes, imparts a conformational change, (functionally) disrupts a protein described herein, such as, for example, tubulin, JAK3, EGFR, BTK, FGFR4, RIPK2, and/or BMX. In some instances, a protein binder provided herein binds to tubulin, JAK3, EGFR, BTK, FGFR4, RIPK2, and/or BMX. In some instances, a protein binder provided herein interacts with tubulin, JAK3, EGFR, BTK, FGFR4, RIPK2, and/or BMX. In some instances, a protein binder provided herein covalently interacts with tubulin, JAK3, EGFR, BTK, FGFR4, RIPK2, and/or BMX. In some instances, a protein binder provided herein modulates tubulin, JAK3, EGFR, BTK, FGFR4, RIPK2, and/or BMX. In some instances, a protein binder provided herein inhibits tubulin, JAK3, EGFR, BTK, FGFR4, RIPK2, and/or BMX. In some instances, a protein binder provided herein destabilizes tubulin, JAK3, EGFR, BTK, FGFR4, RIPK2, and/or BMX. In some instances, a protein binder provided herein imparts a conformational change to KRAS (e.g., upon binding). In some instances, a protein binder provided herein disrupts tubulin, JAK3, EGFR, BTK, FGFR4, RIPK2, and/or BMX. In some instances, a protein binder provided herein functionally disrupts tubulin, JAK3, EGFR, BTK, FGFR4, RIPK2, and/or BMX.

In some instances, an inhibitor is a protein binder that degrades and/or disrupts the functionality of a protein described herein, such as tubulin, JAK3, EGFR, BTK, FGFR4, RIPK2, and/or BMX.

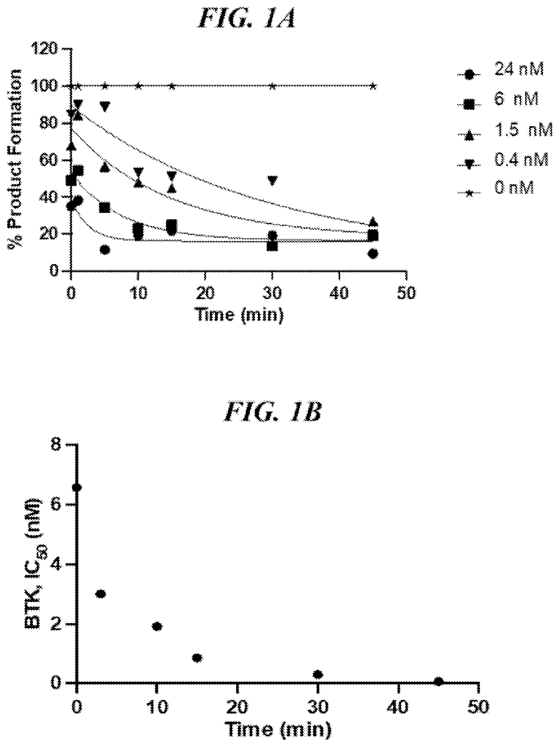

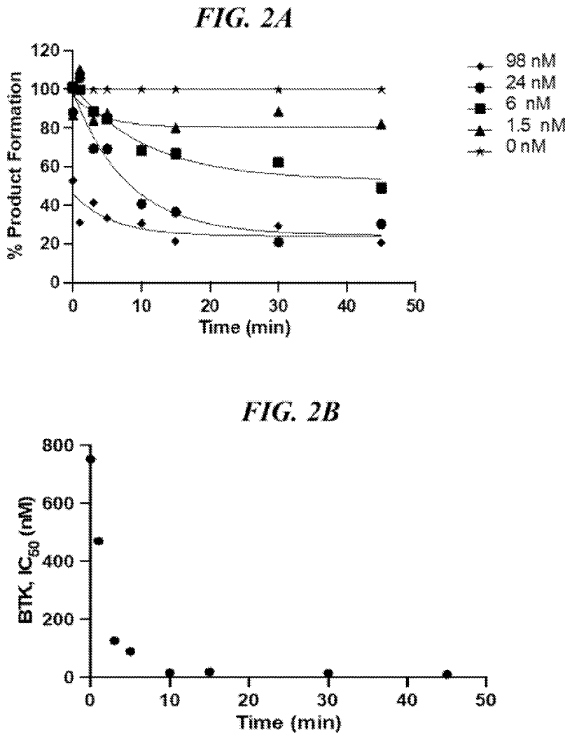

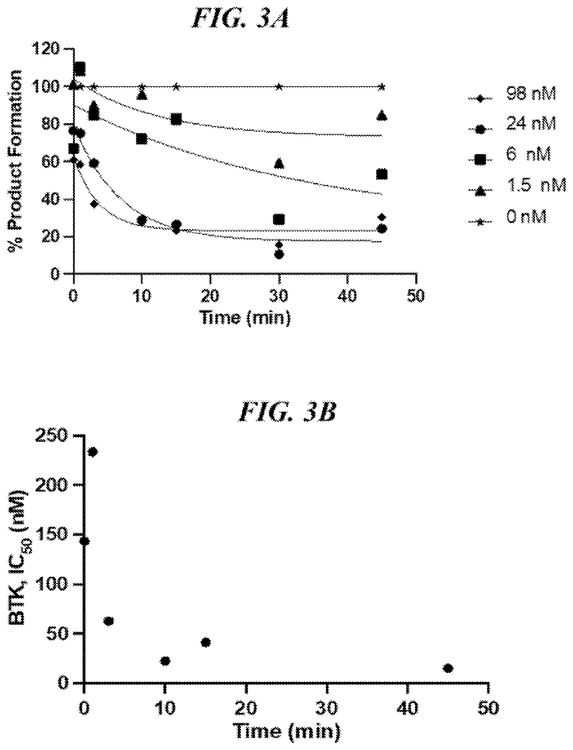

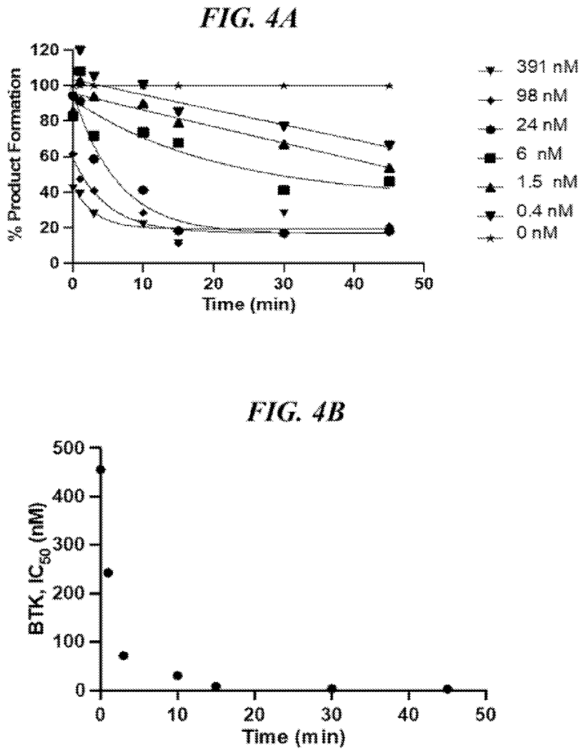

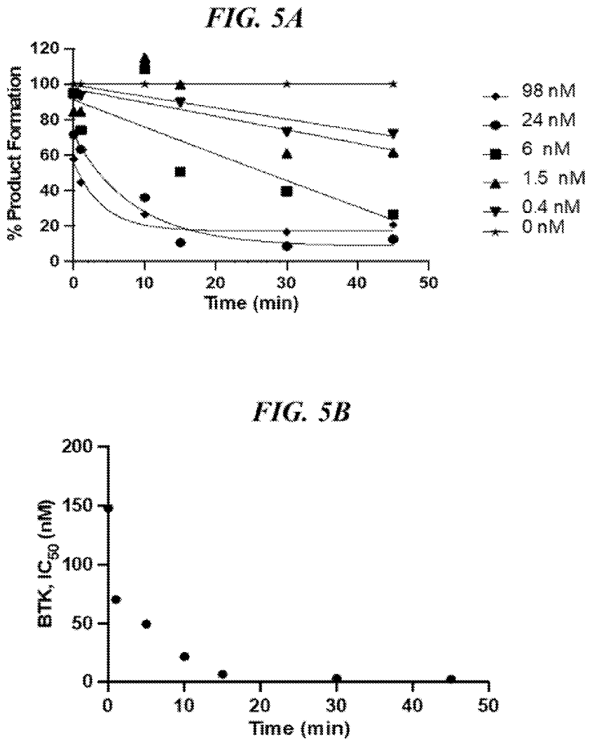

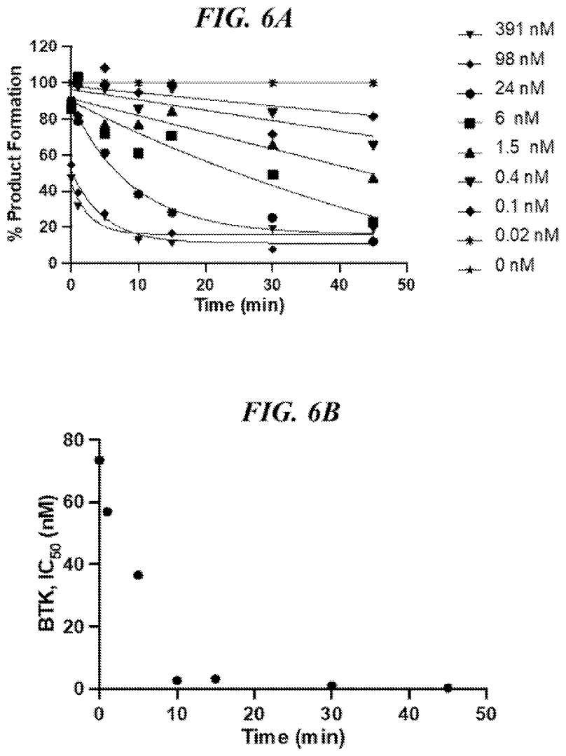

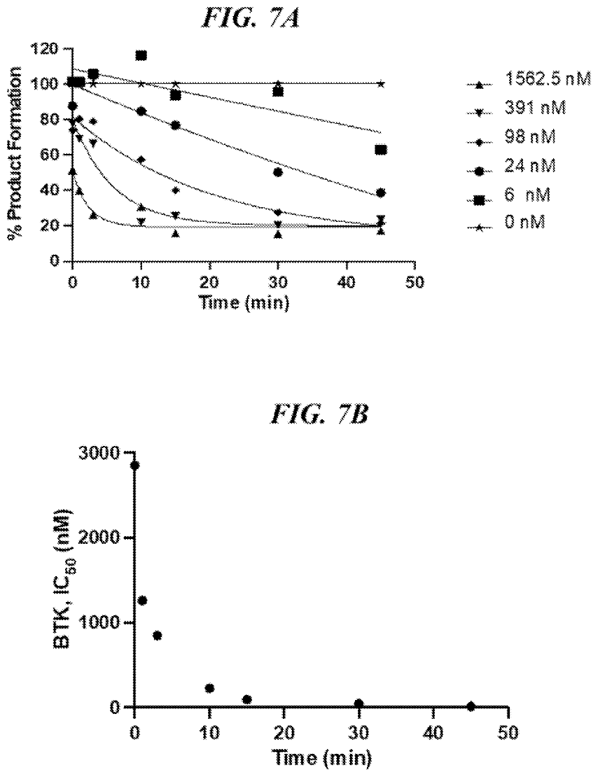

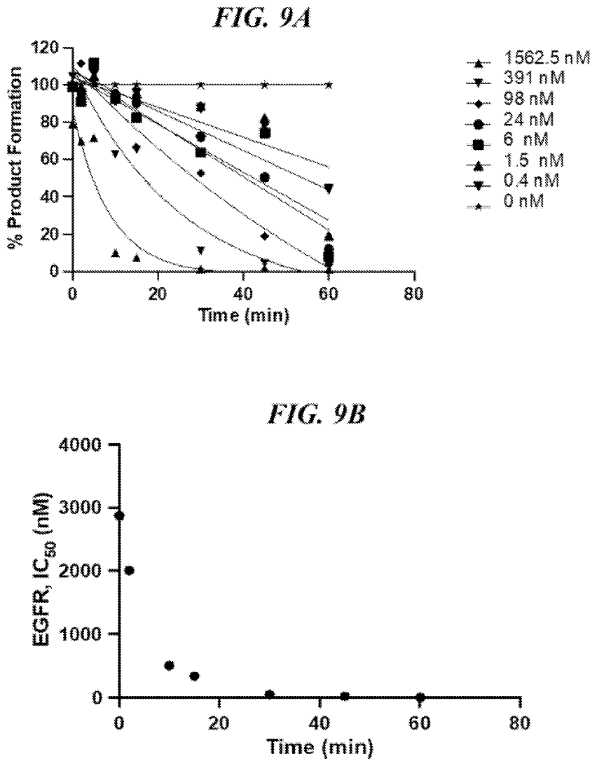

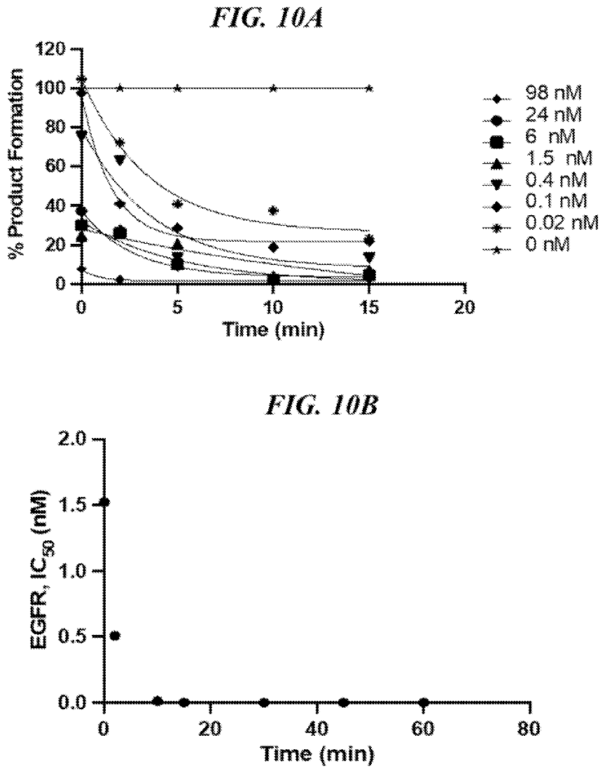

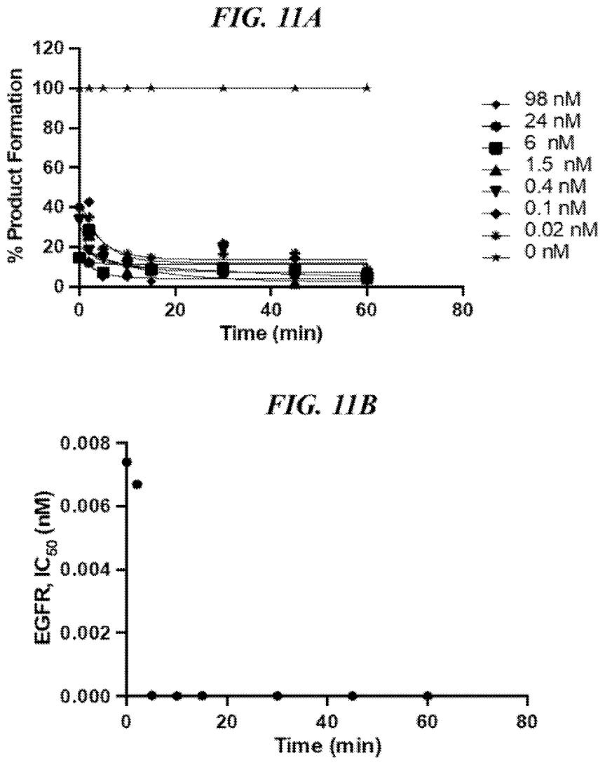

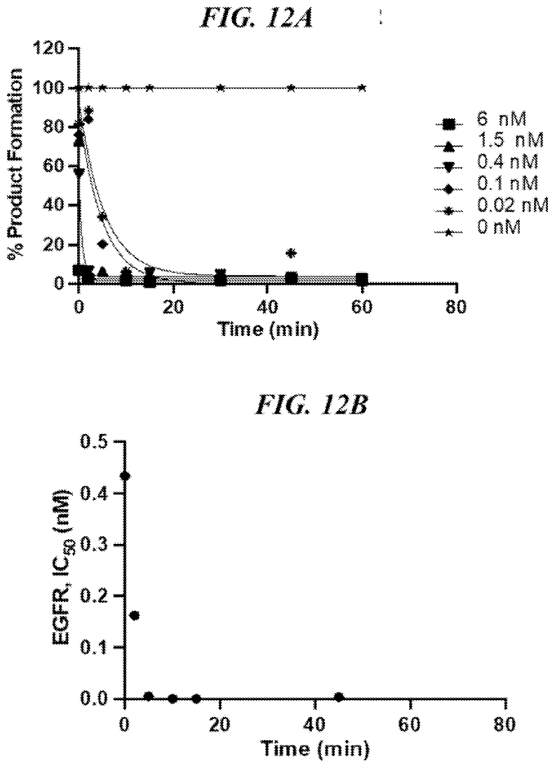

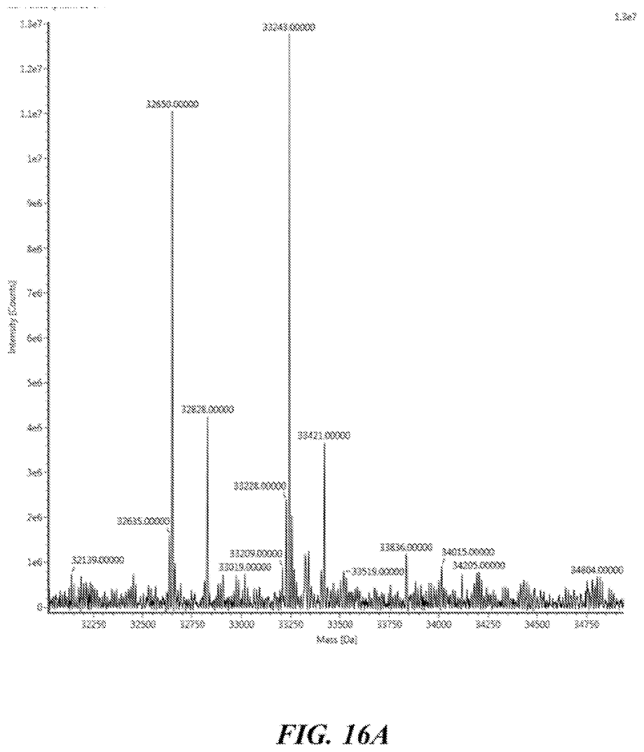

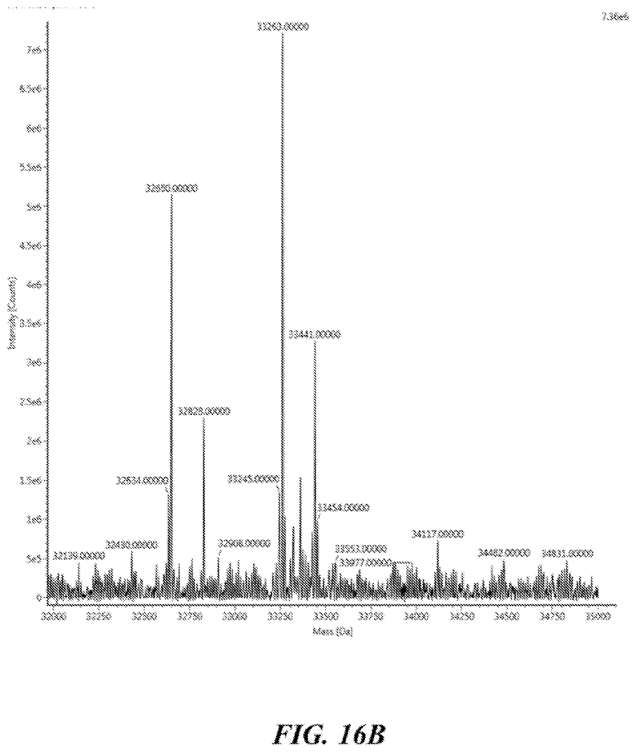

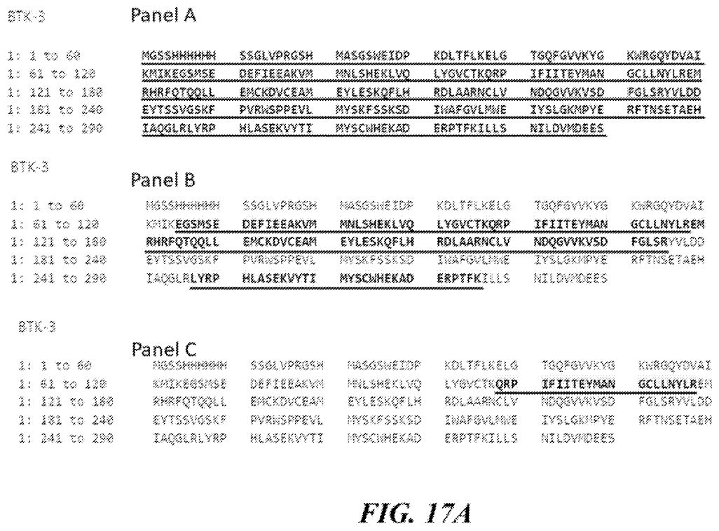

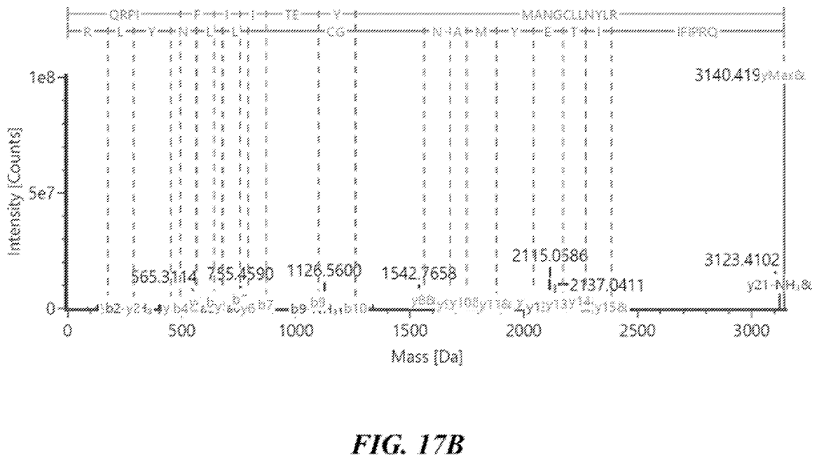

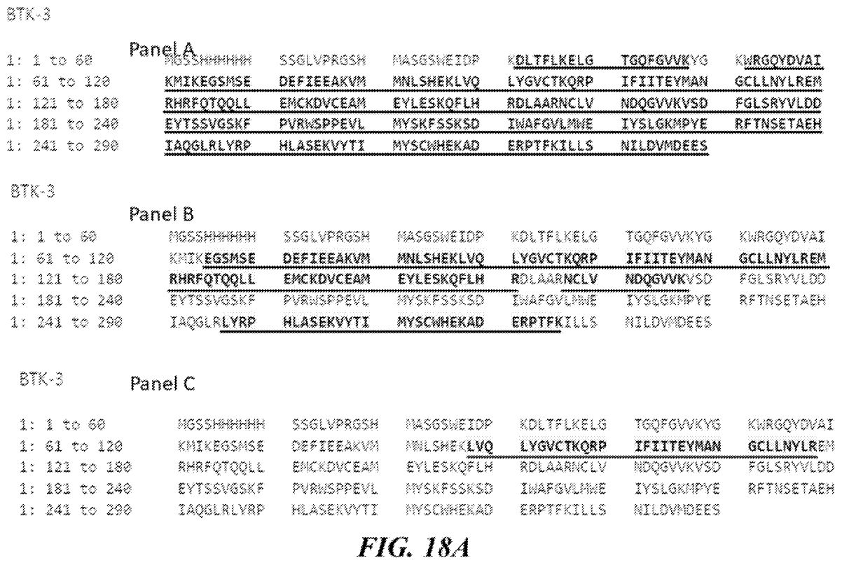

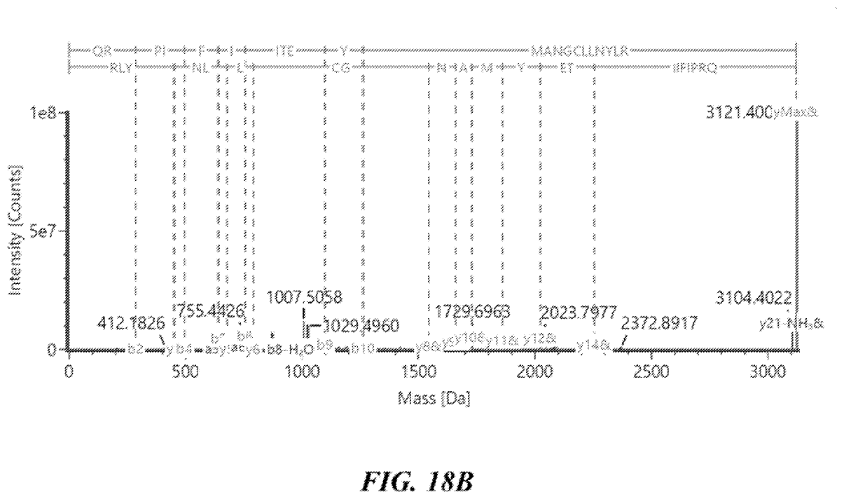

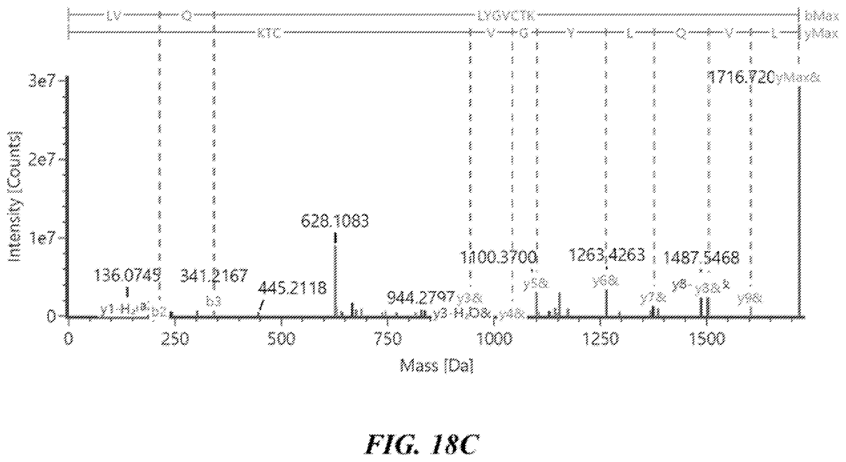

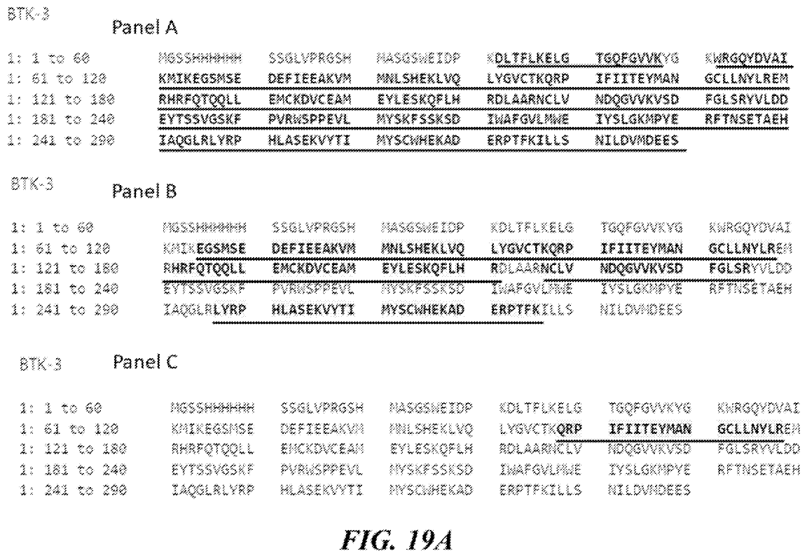

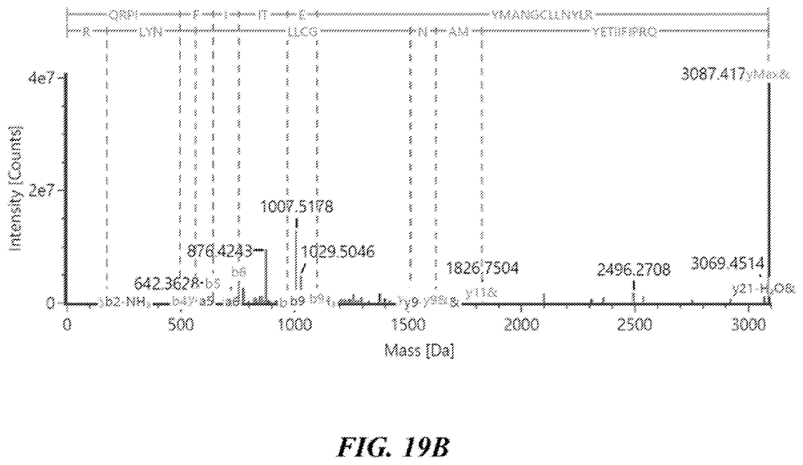

In some instances, a compound provided herein is an irreversible binder (e.g., inhibitor). In some instances, mass spectrometry, enzyme kinetics, discontinuous exposure (e.g., jump dilution), or any combination thereof are used to determine the amount a compound modifies a target protein. In some instances, mass spectrometry (e.g., of the protein drug target modified (e.g., tubulin, JAK3, EGFR, BTK, FGFR4, RIPK2, or BMX) in the presence of a compound provided herein) is used to determine if a compound is an irreversible binder (e.g., inhibitor), such as shown in FIGS. 1A-19B. In some instances, a protein (e.g., tubulin, JAK3, EGFR, BTK, FGFR4, RIPK2, or BMX) modulated (e.g., inhibited) by a compound provided herein is subjected to mass spectral analysis (e.g., to assess the formation of permanent, irreversible covalent adducts). In some instances, analytical methods to examine peptide fragments (e.g., generated upon tryptic cleavage of a protein (e.g., tubulin, JAK3, EGFR, BTK, FGFR4, RIPK2, or BMX)) include, but are not limited to mass spectroscopy. In some instances, such methods identify permanent, irreversible covalent protein adducts (e.g., by observing a mass peak that corresponds to the mass of a control sample plus the mass of an irreversible adduct).

In some instances, such as when a protein described herein interacts (e.g., is bound (e.g., covalently and/or irreversibly bound)) with a compound provided herein, binding of a protein described herein leads to functional inhibition of the protein target (e.g., in a cellular environment).

In some embodiments, a compound provided herein comprises a group (e.g., a warhead) that irreversibly or covalently binds to a protein (e.g., tubulin, JAK3, EGFR, BTK, FGFR4, RIPK2, or BMX). In some instances, a warhead provided herein is a functional group that covalently binds to an amino acid residue (such as cysteine, lysine, histidine, or other residues capable of being covalently modified), present in or near the binding pocket of a target protein (e.g., tubulin, JAK3, EGFR, BTK, FGFR4, RIPK2, or BMX). In some instances, a warhead provided herein irreversibly inhibits tubulin, JAK3, EGFR, BTK, FGFR4, RIPK2, or BMX. In some instances, a warhead provided herein covalently and irreversibly inhibits tubulin, JAK3, EGFR, BTK, FGFR4, RIPK2, or BMX either alone or in combination with L (e.g., warhead-L-).

Sequence of His Tagged BTK:
(SEQ ID NO: 1)
MGSSHHHHHH SSGLVPRGSH MASGSWEIDP KDLTFLKELG
TGQFGVVKYG KWRGQYDVAI KMIKEGSMSEDEFIEEAKVM
MNLSHEKLVQ LYGVCTKQRP IFIITEYMAN GCLLNYLREM
RHRFQTQQLL EMCKDVCEAMEYLESKQFLH RDLAARNCLV
NDQGVVKVSD FGLSRYVLDD EYTSSVGSKF PVRWSPPEVL
RFTNSETAEHIAQGLRLYRP MYSKFSSKSDIWAFGVLMWE
IYSLGKMPYE HLASEKVYTI MYSCWHEKAD ERPTFKILLSN
ILDVMDEESNILDVMDEES

Provided in some embodiments herein is a compound having a structure represented by Formula (I-A): D1-L-D2. In some embodiments, D1 is a radical of a protein-binding ligand. In some embodiments, D2 is a warhead radical. In some embodiments, L is a linker. In some embodiments, the compound is a pharmaceutically acceptable salt or solvate.

In some embodiments, D2 is a warhead radical, such as an aromatic warhead radical, such as a substituted phenyl warhead radical, such as a phenyl warhead radical substituted with halogen (e.g., fluorine).

In some embodiments, D2 covalently modifies a target protein. In some embodiments, D2 covalently modifies tubulin, JAK3, EGFR, BTK (SEQ ID NO: 1), FGFR4, RIPK2, or BMX. In some embodiments, D2 covalently modifies tubulin. In some embodiments, D2 covalently modifies JAK3. In some embodiments, D2 covalently modifies EGFR. In some embodiments, D2 covalently modifies BTK (SEQ ID NO:1). In some embodiments, D2 covalently modifies FGFR4. In some embodiments, D2 covalently modifies RIPK2. In some embodiments, D2 covalently modifies BMX.

In some embodiments, D2 binds to, disrupts, and/or modifies a target protein, such as in vitro, such as using differential scanning fluorimetry (DSF), such as described in the Examples. In some embodiments, D2 binds to, disrupts, and/or modifies tubulin, JAK3, EGFR, BTK (SEQ ID NO:1), FGFR4, RIPK2, or BMX, such as in vitro, such as using differential scanning fluorimetry (DSF), such as described in the Examples.

In some instances, an enzyme (e.g., BTK (SEQ ID NO: 1)) is inhibited (e.g., time-dependent inhibition of BTK) with a compound provided herein (e.g., Compound I-40, Compound I-37, Compound I-32, Compound I-38, Compound I-34, and Compound I-33). In some instances, the inhibition is demonstrated by enzyme kinetic analysis of their pre-incubation time dependence. In some instances, BTK (SEQ ID NO:1) is pre-incubated with varying concentrations of a compound provided herein for varying times, followed by measurement of residual activity. In some instances, residual activity decreases as a function of pre-incubation time (e.g., in a mono-exponential fashion) in a concentration dependent manner (e.g., see FIGS. 1A-7A). In some instances, the IC₅₀ value of a compound provided herein decreases as pre-incubation time was increased (e.g., see FIGS. 1B-7B). In some instances, such trends are consistent with the irreversible inhibition of BTK (SEQ ID NO:1).

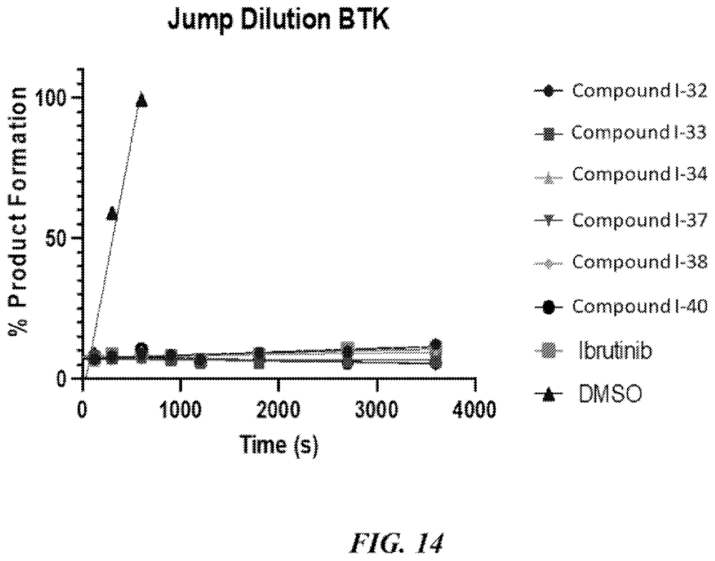

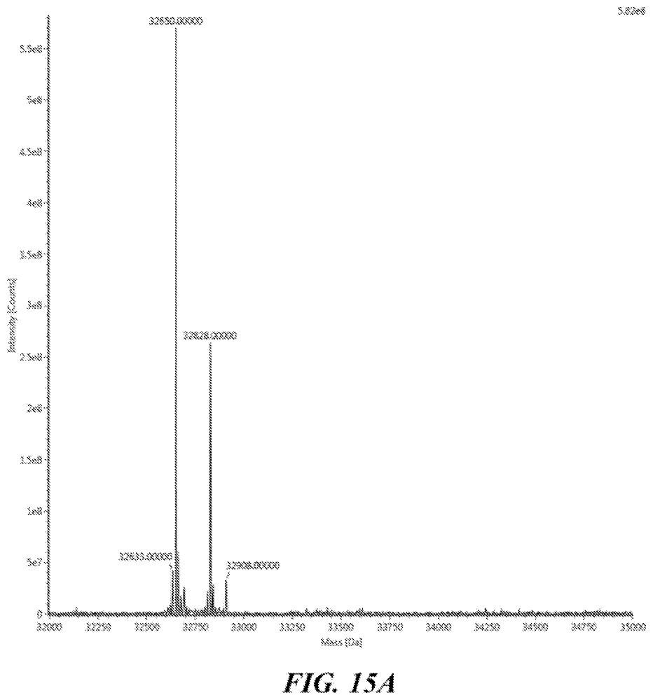

In some instances, an enzyme (e.g., BTK (SEQ ID NO:1)) is covalently modified by a compound provided herein (e.g., Compound I-32, Compound I-33, Compound I-34, Compound 1-37, Compound I-38, and Compound I-40). In some instances, covalent modification is demonstrated by “jump dilution”. For example, after a (e.g., 1.5-h) incubation of (e.g., 142 nM) BTK in the presence of (e.g., 10×IC₅₀ concentration of) a compound provided herein, a (e.g., 100-fold) dilution is performed (e.g., to remove excess compound). In some instances, such as shown in FIG. 14, the residual activity of BTK (SEQ ID NO:1) is substantially reduced (e.g., to less than 1% of that of the enzyme incubated with DMSO alone FIG. 14)). In some instances, such trends are consistent with >99% of BTK being inhibited irreversibly (e.g., during the incubation period).

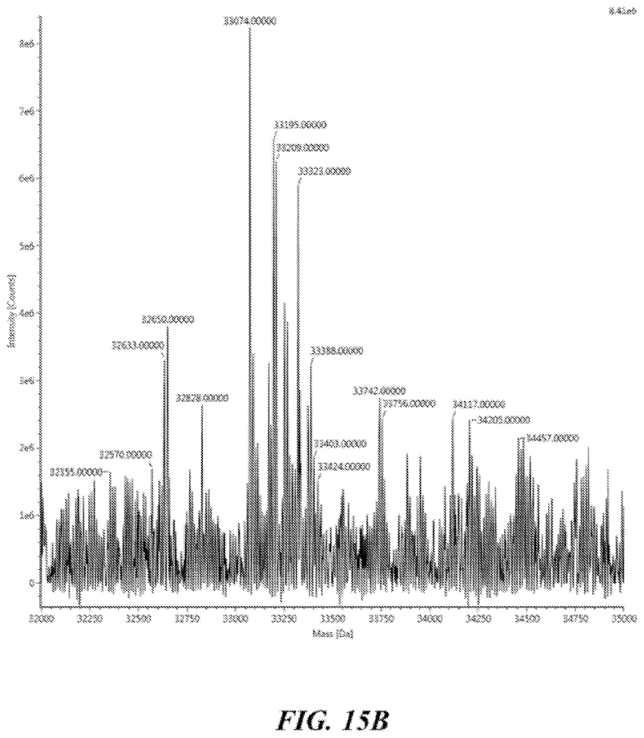

In some instances, an enzyme (e.g., BTK (SEQ ID NO:1)) is covalently modified with a compound provided herein (e.g., Compound I-55, Compound I-24 and Compound I-56). In some instances, covalent modification is demonstrated by intact mass analysis. For example, after incubation with BTK and analysis by LCMS/MS, single adducts of BTK (SEQ ID NO:1) with a compound provided herein is identified (e.g., see FIGS. 15A-19B).

In some instances, an enzyme (e.g., BTK (SEQ ID NO:1)) is covalently modified with a compound provided herein (e.g., Compound I-55, Compound I-24 and Compound I-56). In some instances, covalent modification is demonstrated by intact mass analysis. In some instances, a protein (e.g., BTK (SEQ ID NO: 1)) inhibited by compound and/or pharmaceutically acceptable salt provided herein is subjected to mass spectral analysis (e.g., to assess the formation of permanent, irreversible covalent adducts). Analytical methods to examine peptide fragments generated upon tryptic cleavage of a protein can be performed. Using Such methods can provide identification of permanent, irreversible covalent protein adducts (e.g., by observing a mass peak that corresponds to the mass of a control sample plus the mass of an irreversible adduct) (e.g., see FIGS. 15A-19B).

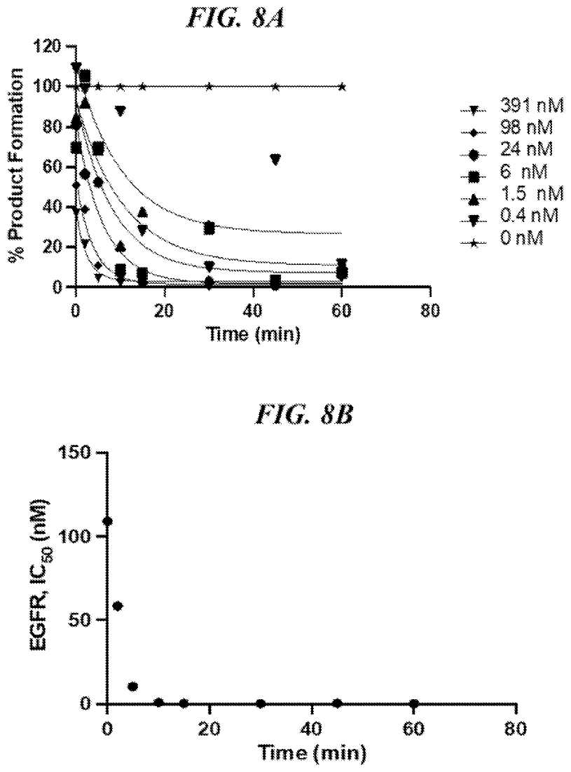

In some instances, an enzyme (e.g., EGFR) is inhibited (e.g., time-dependent inhibition of EGFR) with a compound provided herein (e.g., Compound I-37, Compound I-2, Compound I-3, and Compound I-4). In some instances, the inhibition is demonstrated by enzyme kinetic analysis of their pre-incubation time dependence. In some instances, EGFR is pre-incubated with varying concentrations of a compound provided herein for varying times, followed by measurement of residual activity. In some instances, residual activity decreases as a function of pre-incubation time (e.g., in a mono-exponential fashion) in a concentration dependent manner (e.g., see FIGS. 8A-12A). In some instances, the IC₅₀ value of a compound provided herein decreases as pre-incubation time was increased (e.g., see FIGS. 8B-12B). In some instances, such trends are consistent with the irreversible inhibition of EGFR.

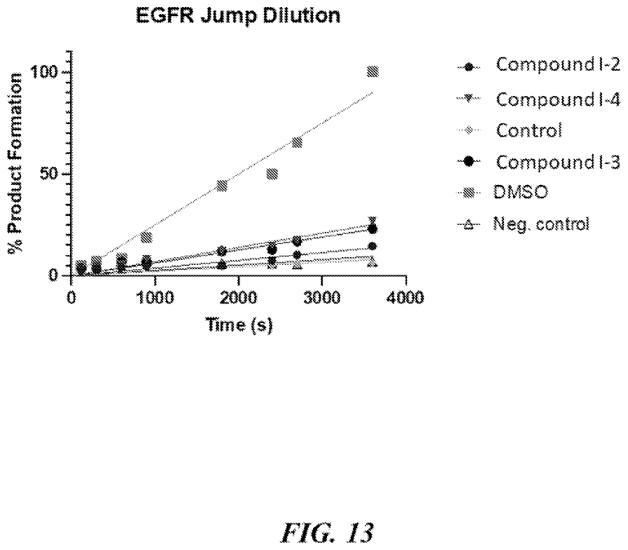

In some instances, an enzyme (e.g., EGFR) is covalently modified by a compound provided herein (e.g., Compound I-2, Compound I-4, and Compound I-3). In some instances, covalent modification is demonstrated by “jump dilution”. For example, after a (e.g., 1.5-h) incubation of (e.g., 46 nM) EGFR in the presence of (e.g., 10×IC₅₀ concentration of) a compound provided herein, a (e.g., 100-fold) dilution is performed (e.g., to remove excess compound). In some instances, such as shown in FIG. 13, the residual activity of EGFR is substantially reduced (e.g., to less than 20% of that of the enzyme incubated with DMSO alone (FIG. 13)). In some instances, such trends are consistent with >80% of EGFR being inhibited irreversibly (e.g., during the incubation period).

In some embodiments, a compound provided herein irreversibly and covalent modifies a protein described herein, such as, for example BTK, such as at cysteine-464, cysteine 481, and/or cysteine-527 in the full-length protein (for example, see FIGS. 15A-19B).

In some embodiments, D2 comprises one or more warhead group. In some embodiments, D2 comprises one or more warhead group, each warhead group being independently selected from the group consisting of substituted or unsubstituted sulfonamide, sulfone, sulfoxide, substituted or unsubstituted amino, or substituted aryl. In some embodiments, D2 comprises one or more warhead group. In some embodiments, D2 comprises one or more warhead group, each warhead group being independently selected from the group consisting of substituted or unsubstituted sulfonamide, sulfone, sulfoxide, or substituted aryl.

In some embodiments, D2 comprises a sulfone, a sulfoxide, or a sulfonamide.

In some embodiments, D2 comprises substituted or unsubstituted sulfonamide. In some embodiments, D2 comprises unsubstituted sulfonamide. In some embodiments, D2 comprises sulfonamide substituted with alkyl. In some embodiments, D2 comprises sulfonamide substituted with methyl.

In some embodiments, D2 comprises substituted or unsubstituted sulfone.

In some embodiments, D2 comprises substituted or unsubstituted sulfoxide.

In some embodiments, D2 comprises substituted or unsubstituted amino. In some embodiments, D2 comprises a secondary amine (e.g., —NH— or —NCH₃—) or a tertiary amine (e.g., >N—)).

In some embodiments, D2 comprises substituted aryl.

In some embodiments, D2 comprises an aryl substituted with one or more substituent, each substituent being independently selected from halogen (e.g., fluoro), hydroxy, substituted or unsubstituted alkoxy (e.g., unsubstituted alkoxy (e.g., methoxy), alkoxy substituted with halogen (e.g., fluoro) (e.g., —OCH₂F, —OCHF₂, or —OCF₃), or alkoxy substituted with substituted or unsubstituted aryl (e.g., phenyl)), substituted or unsubstituted alkyl (e.g., alkyl substituted with halogen (e.g., fluoro) (e.g., —CH₂F, —CHF₂, or —CF₃))).

In some embodiments, D2 comprises a sulfone and an aryl substituted with one or more substituent, each substituent being independently selected from halogen (e.g., fluoro), hydroxy, substituted or unsubstituted alkoxy (e.g., unsubstituted alkoxy (e.g., methoxy), alkoxy substituted with halogen (e.g., fluoro) (e.g., —OCH₂F, —OCHF₂, or —OCF₃), or alkoxy substituted with substituted or unsubstituted aryl (e.g., phenyl)), substituted or unsubstituted alkyl (e.g., alkyl substituted with halogen (e.g., fluoro) (e.g., —CH₂F, —CHF₂, or —CF₃))).

In some embodiments, D2 comprises a sulfoxide and an aryl substituted with one or more substituent, each substituent being independently selected from halogen (e.g., fluoro), hydroxy, substituted or unsubstituted alkoxy (e.g., unsubstituted alkoxy (e.g., methoxy), alkoxy substituted with halogen (e.g., fluoro) (e.g., —OCH₂F, —OCHF₂, or —OCF₃), or alkoxy substituted with substituted or unsubstituted aryl (e.g., phenyl)), substituted or unsubstituted alkyl (e.g., alkyl substituted with halogen (e.g., fluoro) (e.g., —CH₂F, —CHF₂, or —CF₃))).

In some embodiments, D2 comprises a sulfonamide and an aryl substituted with one or more substituent, each substituent being independently selected from halogen (e.g., fluoro), hydroxy, substituted or unsubstituted alkoxy (e.g., unsubstituted alkoxy (e.g., methoxy), alkoxy substituted with halogen (e.g., fluoro) (e.g., —OCH₂F, —OCHF₂, or —OCF₃), or alkoxy substituted with substituted or unsubstituted aryl (e.g., phenyl)), substituted or unsubstituted alkyl (e.g., alkyl substituted with halogen (e.g., fluoro) (e.g., —CH₂F, —CHF₂, or —CF₃))).

In some embodiments, D2 is or comprises an aryl substituted with halogen (e.g., fluoro). In some embodiments, D2 is or comprises an aryl substituted with fluoro.

In some embodiments, D2 comprises an aryl substituted with halogen (e.g., fluoro) and alkyl substituted with halogen (e.g., fluoro) (e.g., —CH₂F, —CHF₂, or —CF₃). In some embodiments, D2 comprises an aryl substituted with fluoro and alkyl substituted with halogen fluoro. In some embodiments, D2 comprises an aryl substituted with fluoro and —CH₂F, —CHF₂, or —CF₃.

In some embodiments, D2 comprises an aryl substituted with halogen (e.g., fluoro) and hydroxy. In some embodiments, D2 is an aryl substituted with fluoro and hydroxy.

In some embodiments, D2 comprises an aryl substituted with halogen (e.g., fluoro) and unsubstituted alkoxy (e.g., methoxy). In some embodiments, D2 is an aryl substituted with fluoro and methoxy.

In some embodiments, D2 comprises an aryl substituted with halogen and alkoxy substituted with substituted or unsubstituted an aryl. In some embodiments, D2 is an aryl substituted with halogen and alkoxy substituted with substituted or unsubstituted an aryl. In some embodiments, D2 is an aryl substituted with fluoro and alkoxy substituted with substituted or unsubstituted phenyl.

In some embodiments, D2 comprises an aryl substituted with halogen (e.g., fluoro) and alkoxy substituted with halogen (e.g., fluoro) (e.g., —OCH₂F, —OCHF₂, or —OCF₃). In some embodiments, D2 is an aryl substituted with fluoro and alkoxy substituted with fluoro. In some embodiments, D2 is an aryl substituted with fluoro and —OCH₂F, —OCHF₂, or —OCF₃.

In some embodiments, D2 comprises an aryl substituted with halogen (e.g., fluoro) and sulfone. In some embodiments, D2 is an aryl substituted with fluoro and sulfone.

In some embodiments, D2 comprises an aryl substituted with halogen (e.g., fluoro) and sulfoxide. In some embodiments, D2 is an aryl substituted with fluoro and sulfoxide.

In some embodiments, D2 comprises an aryl substituted with halogen (e.g., fluoro) and unsubstituted sulfonamide. In some embodiments, D2 is an aryl substituted with fluoro and unsubstituted sulfonamide.

In some embodiments, D2 comprises an aryl substituted with halogen and substituted sulfonamide. In some embodiments, D2 is an aryl substituted with fluoro and sulfonamide substituted with alkyl. In some embodiments, D2 is an aryl substituted with fluoro and sulfonamide substituted with methyl.

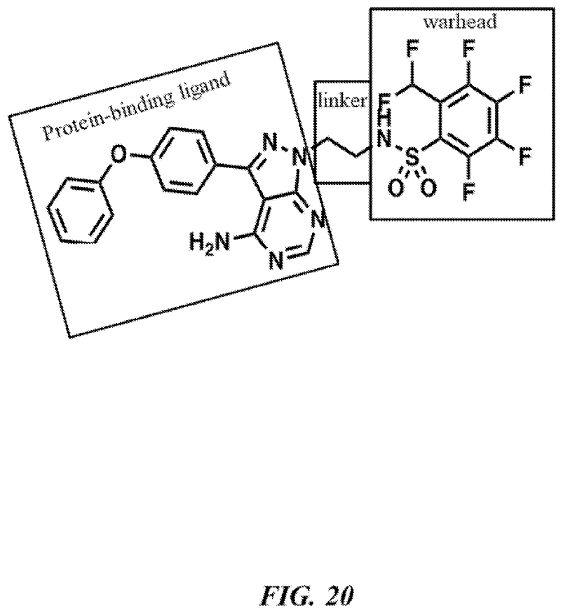

In some embodiments, provided herein is a compound (e.g., of Formula (I-A)), wherein the compound (e.g., of Formula (I-A)) has a warhead (e.g., D2) of any one of the compounds of Table 4, Table 5, Table 6, Table 7, or Table 8, such as wherein the warhead (e.g., D2) is the part of the compound identified with a box around it in FIG. 20.

In some embodiments, D2 comprises one or more activating group, such as an activating group that binds to, disrupts, and/or modifies a protein (e.g., tubulin, JAK3, EGFR, BTK, FGFR4, RIPK2, or BMX) either alone or in combination with L (e.g., when D2 is amino (e.g., tertiary amine (e.g., >N—)) and L is substituted or unsubstituted pipirizinyl or substituted or unsubstituted azetidinyl).

In some embodiments, D1 is a radical of a protein-binding ligand, such as a protein-binding ligand provided elsewhere herein (e.g., G or G¹).

In some embodiments, D1 is a radical of a tubulin-binding ligand. In some embodiments, D1 is a radical of a JAK3-binding ligand. In some embodiments, D1 is a radical of a EGFR-binding ligand. In some embodiments, D1 is a radical of a BTK-binding ligand. In some embodiments, D1 is a radical of a FGFR4-binding ligand. In some embodiments, D1 is a radical of a RIPK2-binding ligand. In some embodiments, D1 is a radical of a BMX-binding ligand.

In some embodiments, D1 has a structure represented in Table 2 or Table 3. In some embodiments, D1 has a structure represented in Table 2 or Table 3 and L is a bond.

Unless stated specifically otherwise herein, each instance of radical indicates that a hydrogen (i.e., a hydrogen radical (H·)) is removed from a free form of a compound provided herein, such as any protein-binding ligand (e.g., D1) or warhead (e.g., D2) described herein. In some instances, the removal of the hydrogen radical from the compound provided herein, such as any protein-binding ligand (e.g., D1) or warhead (e.g., D2) described herein, provides a radical of a protein-binding ligand or a warhead that is taken together with any point of a linker provided herein (e.g., L, L¹, or L²) to form a bond (e.g., between the linker and the radical of the protein-binding ligand or the warhead). In some instances, a carbon atom (e.g., of any protein-binding ligand (e.g., a substituted heterocycle or a substituted carbocycle) or warhead described herein) loses an H· to become a point of attachment to L. In some instances, >NH loses an H· to become >N-(point of attachment), such as >N-L-D1, >N-L-D2, >N-D1, or >N-D2. In some instances, —OH loses an H· to become —O-(point of attachment), such as —O-L-D1, —O-L-D2, —O-D1, or —O-D2. In some instances, —S(═O)_{g}H (where g is 1 or 2) loses an H· to become —S(═O)_{g}-(point of attachment), such as —S(═O)_{g}-L-D1, —S(═O)_{g}-L-D2, —S(═O)_{g}-D1, or —S(═O)_{g}-D2. In some instances, the linker is a bond. In some instances, D1-L- is a protein-binding ligand.

In some embodiments, provided herein is a compound (e.g., of Formula (I-A)), wherein the compound (e.g., of Formula (I-A)) comprises a protein-binding ligand (e.g., D1) of any one of the compounds of Table 4, Table 5, Table 6, Table 7, or Table 8, such as wherein the protein-binding ligand (e.g., D1) is the part of the compound identified with a box around it in FIG. 20.

In some embodiments, D1 (a protein-binding ligand provided herein) binds to, disrupts, and/or modifies a protein (e.g., tubulin, JAK3, EGFR, BTK, FGFR4, RIPK2, and/or BMX) either alone or in combination with D2 (a warhead radical provided herein) and/or L (a linker provided herein). In some instances, D1 has activity such that a compound provided herein binds to, disrupts, and/or modifies a protein (e.g., tubulin, JAK3, EGFR, BTK, FGFR4, RIPK2, and/or BMX) at a concentration of about 10 mM or less (e.g., 500 uM or less, 100 uM or less, or 10 uM or less). In some instances, D1 has activity such that a compound provided herein has Ki to a protein (e.g., tubulin, JAK3, EGFR, BTK, FGFR4, RIPK2, and/or BMX) of about 250 uM or less (e.g., about 50 uM or less or about 1 uM or less).

In some embodiments, L is a linker.

In some embodiments, the linker is a non-releasable linker.

In some instances, the linker does not decompose (e.g., hydrolyze) or release the warhead radical (or a free form thereof), the radical of the protein-binding ligand (or a free form thereof), or any other portion of the compound (e.g., a radical of any Formula provided herein) (or a free form thereof)).

In some embodiments, the linker comprises one or more linker group, each linker group being independently selected from the group consisting of a bond, —O—, (substituted or unsubstituted) amino (e.g., —NH—, —NCH₃—, methylamine, or dimethylamine), substituted or unsubstituted (e.g., acyclic (e.g., straight or branched) or cyclic) alkyl(ene) (e.g., straight unsubstituted alkyl (e.g., methylene, ethylene, or the like) or straight alkylene substituted with oxo, amino (e.g., —NH—, —NCH₃—, or methylamine), heterocyclyl (e.g., (methylene) piperidinyl or piperazinyl), and/or aryl (e.g., (methylene) phenyl)), substituted or unsubstituted (e.g., acyclic (e.g., straight or branched) or cyclic) heteroalkyl(ene) (e.g., cyclic heteroalkylene (e.g., piperazinyl or 1,4-diazepanyl) substituted with alkyl (e.g., methyl) and/or oxo, or straight heteroalkylene substituted with oxo, heterocyclyl (e.g., azetidinyl, pyrrolidinyl, piperidinyl, or piperazinyl), aryl (e.g., phenyl), and/or heteroaryl (e.g., substituted or unsubstituted oxazolyl, pyridinyl, imidazolyl, or pyrazolyl)), substituted or unsubstituted alkoxy (e.g., unsubstituted alkoxy (e.g., methoxy, ethoxy, or the like) or alkoxy substituted with oxo, amino (e.g., —NH—, —NCH₃—, substituted (e.g., methylamine) or —NH-azetidinyl-), cycloalkyl (e.g., cyclobutyl substituted with amino (e.g., —NH—, —NCH₃—, or methylamine)), and/or heterocyclyl (e.g., azetidinyl or pyrrolidinyl)), and substituted or unsubstituted aryl (e.g., aryl substituted with alkyl (e.g., methyl)).

In some embodiments, the linker comprises one or more linker group, each linker group being independently selected from the group consisting of —O—, substituted or unsubstituted amino, substituted or unsubstituted alkylene, substituted or unsubstituted heteroalkylene, and substituted or unsubstituted alkoxy.

In some embodiments, the linker comprises one or more linker group, each linker group being independently selected from the group consisting of —O—, substituted or unsubstituted amino and substituted or unsubstituted heteroalkylene. In some embodiments, the linker comprises one or more linker group, each linker group being independently selected from the group consisting of —O—, substituted or unsubstituted amino and substituted or unsubstituted acyclic (e.g., straight or branched) heteroalkylene. In some embodiments, the linker comprises one or more linker group, each linker group being independently selected from the group consisting of —O—, substituted or unsubstituted amino and substituted or unsubstituted cyclic heteroalkylene. In some embodiments, the linker comprises one or more linker group, each linker group being independently selected from the group consisting of —O—, substituted or unsubstituted amino and substituted or unsubstituted heterocyclyl.

In some embodiments, the linker comprises one or more linker group, each linker group being independently selected from the group consisting of substituted or unsubstituted amino and substituted or unsubstituted heteroalkylene. In some embodiments, the linker comprises one or more linker group, each linker group being independently selected from the group consisting of substituted or unsubstituted amino and substituted or unsubstituted acyclic (e.g., straight or branched) heteroalkylene. In some embodiments, the linker comprises one or more linker group, each linker group being independently selected from the group consisting of substituted or unsubstituted amino and substituted or unsubstituted cyclic heteroalkylene. In some embodiments, the linker comprises one or more linker group, each linker group being independently selected from the group consisting of substituted or unsubstituted amino and substituted or unsubstituted heterocyclyl.

In some embodiments, the linker comprises —O—.

In some embodiments, the linker comprises substituted or unsubstituted amino.

In some embodiments, the linker comprises substituted or unsubstituted alkylene. In some embodiments, the linker comprises substituted or unsubstituted acyclic (e.g., straight or branched) alkylene. In some embodiments, the linker comprises substituted or unsubstituted cyclic alkylene.

In some embodiments, the linker comprises substituted or unsubstituted heteroalkylene. In some embodiments, the linker comprises substituted or unsubstituted acyclic (e.g., straight or branched) heteroalkylene. In some embodiments, the linker comprises substituted or unsubstituted cyclic heteroalkylene (e.g., heterocycyl).

In some embodiments, the linker comprises substituted or unsubstituted heterocycyl.

In some embodiments, the linker comprises substituted or unsubstituted alkoxy.

In some embodiments, L is a bond, substituted or unsubstituted alkylene, substituted or unsubstituted heteroalkylene, substituted or unsubstituted alkoxy, or substituted or unsubstituted amino.

In some embodiments, L is a bond, substituted or unsubstituted alkylene, substituted or unsubstituted heteroalkylene, or substituted or unsubstituted amino.

In some embodiments, L is a bond, substituted or unsubstituted alkylene, substituted or unsubstituted piperizinyl, substituted or unsubstituted azetidinyl, substituted or unsubstituted pyrrolidinyl, or substituted or unsubstituted amino.

In some embodiments, L is a bond.

In some embodiments, L is substituted or unsubstituted alkylene. In some embodiments, L is C(═O), methylene, ethylene, or alkyl substituted with oxo and/or heterocyclyl (e.g., azetidinyl, pyrrolidinyl, or piperidinyl). In some embodiments, L is C(═O), methylene, ethylene, or alkyl substituted with oxo and/or azetidinyl, pyrrolidinyl, or piperidinyl.

In some embodiments, L is substituted or unsubstituted heteroalkylene. In some embodiments, L is —N═CH—, —CH₂NH—, —CH₂NCH₃—, —CH₂CH₂NH—, —NHCH₂CH₂NH—, —CH₂CH₂NCH₃—, —CH₂CH₂NHCH₂—, —NHCH₂CH₂NHCH₂—, —NHCH₂CH₂CH₂NH—, —CH₂CH₂CH₂NH—, or heteroalkyl substituted with oxo. In some embodiments, L is substituted or unsubstituted heteroalkylene. In some embodiments, L is —N═CH—, —CH₂NH—, —CH₂NCH₃—, —CH₂CH₂NH—, —NHCH₂CH₂NH—, —CH₂CH₂NCH₃—, —CH₂CH₂NHCH₂—, —NHCH₂CH₂NHCH₂—, —NHCH₂CH₂CH₂NH—, —CH₂CH₂CH₂NH—, —C(═O)NH—, —CH₂CH₂N(CH₃)C(═O)—, —CH₂CH₂NHC(═O)—, —NHCH₂CH₂N(CH₃)C(═O)—, —NHCH₂CH₂NHC(═O)—, —CH₂CH₂CH₂N(CH₃)C(═O)—, or —CH₂CH₂CH₂NHC(═O)—.

In some embodiments, L is substituted or unsubstituted amino. In some embodiments, L is —NH— or amino substituted with substituted or unsubstituted aryl. In some embodiments, L is —NH—, —NH-phenyl-, aryl substituted with amino (e.g., —NH-phenyl-NH—) or aryl substituted with alkoxy (e.g., —NH-phenyl-OCH₂—)).

In some embodiments, provided herein is a compound (e.g., of Formula (I-A)), wherein the compound (e.g., of Formula (I-A)) comprises a linker (e.g., L) of any one of the compounds of Table 4, Table 5, Table 6, Table 7, or Table 8, such as wherein the linker (e.g., L) is the part of the compound identified with a box around it in FIG. 20.

In some instances, such as when L is substituted or unsubstituted heterocyclyl, L is part of D1 and/or D2.

Microtubules are composed of alpha/beta-tubulin heterodimers and constitute a crucial component of the cell cytoskeleton. In addition, microtubules play a pivotal role during cell division, in particular when the replicated chromosomes are separated during mitosis. Interference with the ability to form microtubules from alpha/beta-tubulin heterodimeric subunits generally leads to cell cycle arrest. This event can, in certain cases, induce programmed cell death.

Microtubules are subcellular organelles located in most eukaryotic cells and are involved in a variety of cell functions including mitosis, intracellular movement, cell movement and maintenance of cell shape. Microtubule assembly involves polymerization of tubulin and additional construction with other components of the microtubule (referred to as “microtubule-associated proteins” or MAPs).

Tubulin itself consists of two 50 kDa subunits (alpha- and beta-tubulin) which combine in a heterodimer. The heterodimer binds two molecules of guanosine triphosphate (GTP). One of the GTP molecules is tightly bound and cannot be removed without denaturing the heterodimer, while the other GTP molecule is freely exchangeable with other GTPs. This exchangeable GTP is believed to be involved in tubulin function. In particular, the tubulin heterodimer can combine in a head-to-tail arrangement in the presence of GTP to form a long protein fiber, known as a protofilament. These protofilaments can then group together to form a protein sheet which then curls into a tube-like structure known as a microtubule. Interference with this process of microtubule construction affects the downstream processes of mitosis and maintenance of cell shape. Most of the naturally-occurring antimitotic agents have been shown to exert their effect by binding to tubulin, rather than MAPs or other proteins involved in mitosis. For example, tubulin is the biochemical target for several clinically useful anticancer drugs, including vincristine, vinblastine and paclitaxel. Another natural product, colchicine, was instrumental in the purification of tubulin as a result of its potent binding, with beta-tubulin being the target for colchicine. Colchicine and other colchicine site agents bind at a site on beta-tubulin that results in inhibition of a cross-link between cys-239 and cys-354 (wherein the numbering refers to the (2 isotype) by such non-specific divalent sulfhydryl reactive agents as N,N′-ethylenebis-iodoacetamide. However, simple alkylation of cys-239 does not appear to inhibit colchicine binding to tubulin. In addition to colchicine, other natural products are known that bind at the colchicine site and inhibit microtubule assembly, for example, podophyllotoxin, steganacin and combretastatin. Still other agents bind to sites on tubulin referred to as the Vinca alkaloid site and the Rhizoxin/Maytansine site. However, none of the noted natural products are thought to operate by covalent modification of tubulin.

Based on the essential role of tubulin in the processes of cell transport and cell division, compounds which alter the tubulin activity are considered to be useful in treating or preventing various disorders. Accordingly, in some embodiments, also described herein is a covalent small molecule binder (e.g., inhibitor) of tubulin. In some embodiments also described herein is a pharmaceutical composition comprising a covalent small molecule binder (e.g., inhibitor) of tubulin and one or more of pharmaceutically acceptable excipients. In other embodiments, a covalent small molecule binder (e.g., inhibitor) of tubulin is used to treat or prevent a disease or condition in a subject in need thereof.

In some embodiments, a covalent small molecule protein binder (e.g., inhibitor) of tubulin is a benzenesulfonamide derivative compound. In some embodiments, a benzenesulfonamide derivative compound as described herein is used to treat or prevent a disease or condition in a subject in need thereof.

In other embodiments, a pharmaceutical composition comprising a benzenesulfonamide derivative compound as described herein and one or more of pharmaceutically acceptable excipients is used to treat or prevent a disease or condition in a subject in need thereof.

In some embodiments, disclosed herein is a method of treating a disease comprising administering to a subject in need thereof a therapeutically effective amount of a benzenesulfonamide derivative compound as described herein.

In other embodiments, disclosed herein is a method of treating a disease comprising administering to a subject in need thereof a therapeutically effective amount of a pharmaceutical composition comprising a benzenesulfonamide derivative compound as described herein and one or more of pharmaceutically acceptable excipients.

In some embodiments, disclosed herein is a protein modified with a benzenesulfonamide derivative compound as described herein, wherein the compound forms a covalent bond with a sulfur atom of a cysteine residue of the protein. In some embodiments, disclosed herein is a method of modifying (e.g., attaching to and/or degrading) a polypeptide with a benzenesulfonamide derivative compound as described herein, comprising contacting the polypeptide with the compound to form a covalent bond with a sulfur atom of a cysteine residue of the polypeptide. In some embodiments, disclosed herein is a method of binding a compound to a polypeptide, comprising contacting the polypeptide with a benzenesulfonamide derivative compound as described herein. In some embodiments, the protein or polypeptide described herein is tubulin.

In one aspect, provided herein is a benzenesulfonamide derivative compound. In some embodiments, a benzenesulfonamide derivative compound is a protein-binding compound. In some embodiments, a benzenesulfonamide derivative compound is a protein-binding ligand inhibitory compound.

One embodiment provides a compound, or pharmaceutically acceptable salt, solvate, tautomer, or regioisomer thereof, having the structure of Formula (I):

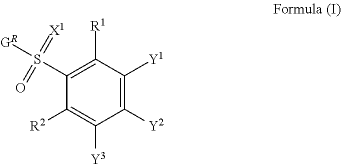

wherein,
G^{R} is alkyl, haloalkyl, heteroalkyl, —N(R⁵)₂, or G;
G is or comprises a protein-binding ligand, is or comprises (e.g., unsaturated) carbocycle, is or comprises (e.g., unsaturated) heterocycle, or is -L²-G¹, wherein L² is a linker (e.g., —O— or —NR⁵—), and G¹ is hydrogen or an organic residue (e.g., is or comprises a protein-binding ligand, is or comprises (e.g., unsaturated) carbocycle, or is or comprises (e.g., unsaturated) heterocycle);
X¹ is absent, O or NR;
each Y¹, Y², and Y is independently hydrogen, halo, alkyl, haloalkyl (e.g., with at least two Y being halo or haloalkyl, such as fluoroalkyl, e.g., at least one Y (e.g., Y²) being halo) (e.g., Y¹, Y², Y³ all being F), or G;
R is hydrogen or R⁷;
R¹ is R⁷;
R² is hydrogen, halogen, or R⁷;
each R³ is independently hydrogen, -L¹R⁴, —C(═O)L¹R⁴, —C(═O)OL¹R⁴, or —C(═O)NR⁴L¹R⁴, wherein each L is independently alkyl, heteroalkyl, aryl, or heteroaryl; and each R⁴ is independently hydrogen, cycloalkyl, heterocycloalkyl, aryl, or heteroaryl;
each R⁵ is independently hydrogen, —CN, —C(═O)R⁶, —C(═O)OR⁶, —C(═O)NR³R⁶, alkyl, heteroalkyl, aryl, or heteroaryl;
each R⁶ is independently hydrogen, alkyl, or heteroalkyl;
each R⁷ is independently G, —CN, —OR³, —S(═O)ₓR³, —S(═O)(═NR³)R³, —S(═O)₂N(R³)₂, —OS(═O)₂R³, —N(R³)₂, —NR³C(═O)R³, —NR³C(═O)N(R³)₂, —NR³C(═NR³)N(R³)₂, —C(═O)R³, —OC(═O)R³, —C(═O)OR³, —OC(═O)OR³, —OC(═O)N(R³)₂, —C(═O)N(R³)₂, alkyl, heteroalkyl, cycloalkyl, heterocycloalkyl, aryl, or heteroaryl; and
x is 0, 1, or 2.

In some embodiments, the compound comprises only one G.

In some embodiments, when X¹ is O, G^{R} is G, G is L²G¹ and L² is amino or —NR⁵, then Y¹, Y², and Y³ are not all F.

In some embodiments, when —S(═O)(═X¹)G^{R} is —S(═O)(═X¹)G, X¹ is O, then G is not:
(R)-3-(4-phenoxyphenyl)-1-(1λ²-piperidin-3-yl)-1H-pyrazolo[3,4-d]pyrimidin-4-amine;
1-(2-(λ²-azaneyl)ethyl)-3-(4-phenoxyphenyl)-1H-pyrazolo[3,4-d]pyrimidin-4-amine;
(R)-3-(4-phenoxyphenyl)-1-(12-pyrrolidin-3-yl)-1H-pyrazolo[3,4-d]pyrimidin-4-amine;
4-(λ²-azaneyl)-7H-pyrrolo[2,3-d]pyrimidine;
N4-(3-(λ²-azaneyl)phenyl)-5-fluoro-N2-(4-(2-methoxyethoxy)phenyl)pyrimidine-2,4-diamine;
4-(λ²-azaneyl)-5-fluoro-N-(4-(2-methoxyethoxy)phenyl)pyrimidin-2-amine; or
3-(4-phenoxyphenyl)-1λ²-pyrazolo[5,4-d]pyrimidin-4-amine.

In some embodiments, when —S(═O)(═X¹)G^{R} is —S(═O)(═X¹)N(R⁵)G, X¹ is O, then one or more of G and R⁵ is not or does not comprise: substituted or unsubstituted phenyl; substituted or unsubstituted benzyl; 1-naphthyl; pyridin-3-yl; pyridin-4-yl; 2-fluoropyridin-4-yl; or 2,6-difluoropyridin-3-yl.

In some embodiments, G is -L²-G¹, wherein L² is a linker, and G¹ is an organic residue (e.g., is or comprises a protein-binding ligand, is or comprises (e.g., unsaturated) carbocycle, or is or comprises (e.g., unsaturated) heterocycle). In some embodiments, L² is a substituted or unsubstituted unsaturated alkylene (e.g., alkenylene or alkynylene), substituted or unsubstituted arylene, or substituted or unsubstituted heteroarylene, and G¹ is an organic residue (e.g., is or comprises a protein-binding ligand). In some embodiments, L² is a bond, —O—, —NR⁸—, —N(R⁸)₂⁺—, —S—, —S(═O)—, —S(═O)₂—, —CH═CH—, ═CH—, —C≡C—, —C(═O)—, —C(═O)O—, —OC(═O)—, —OC(═O)O—, —C(═O)NR⁸—, —NR⁸C(═O)—, —OC(═O)NR⁸—, —NR⁸C(═O)O—, —NR⁸C(═O)NR⁸—, —NR⁸S(═O)₂—, —NR⁸S(═O)(═NR⁸)—, —S(═O)₂NR⁸—, —S(═O)(═NR⁸)NR⁸—, —C(═O)NR⁸S(═O)₂—, —S(═O)₂NR⁸C(═O)—, substituted or unsubstituted C₁-C₄ alkylene, substituted or unsubstituted C₁-C₈ heteroalkylene, —(C₁-C₄ alkylene)-O—, —O—(C₁-C₄ alkylene)-, —(C₁-C₄ alkylene)-NR⁸—, —NR⁸—(C₁-C₄ alkylene)-, —(C₁-C₄ alkylene)-N(R⁸)₂⁺—, or —N(R⁸)₂⁺—(C₁-C₄ alkylene)-; each R⁸ is independently hydrogen, substituted or unsubstituted C₁-C₄ alkyl, substituted or unsubstituted C₁-C₄ haloalkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and G¹ is an organic residue (e.g., is or comprises a protein-binding ligand).

In some embodiments, G is substituted or unsubstituted unsaturated carbocycle or substituted or unsubstituted unsaturated heterocycle, wherein G and R⁵ on a single N, if present, are optionally taken together to form a substituted or unsubstituted N-containing heterocycloalkyl. In some embodiments, G comprises one or more cyclic ring systems selected from substituted or unsubstituted unsaturated carbocycles and substituted or unsubstituted unsaturated heterocycles. In some embodiments, G comprises two or more cyclic ring systems selected from substituted or unsubstituted unsaturated carbocycles and substituted or unsubstituted unsaturated heterocycles.

In some embodiments, G¹ comprises one or more cyclic ring systems selected from substituted or unsubstituted carbocycles and substituted or unsubstituted heterocycles. In some embodiments, G¹ comprises two or more cyclic ring systems selected from substituted or unsubstituted carbocycles and substituted or unsubstituted heterocycles.

In some embodiments, the two or more cyclic ring systems are connected via a bond. In some embodiments, the two or more cyclic ring systems are connected via one or more linker and/or bond. In some embodiments, the linker is —O—, —NR⁸—, —N(R⁸)₂⁺—, —S—, —S(═O)—, —S(═O)₂—, —CH═CH—, ═CH—, —C≡C—, —C(═O)—, —C(═O)O—, —OC(═O)—, —OC(═O)O—, —C(═O)NR⁸—, —NR⁸C(═O)—, —OC(═O)NR⁸—, —NR⁸C(═O)O—, —NR⁸C(═O)NR⁸—, —NR⁸S(═O)₂—, —S(═O)₂NR⁸—, —C(═O)NR⁸S(═O)₂—, —S(═O)₂NR⁸C(═O)—, substituted or unsubstituted C₁-C₄ alkylene, substituted or unsubstituted C₁-C₈ heteroalkylene, —(C₁-C₄ alkylene)-O—, —O—(C₁-C₄ alkylene)-, —(C₁-C₄ alkylene)-NR⁸—, —NR⁸—(C₁-C₄ alkylene)-, —(C₁-C₄ alkylene)-N(R)₂—, or —N(R⁸)₂⁺—(C₁-C₄ alkylene)-; and each R⁸ is independently hydrogen, substituted or unsubstituted C₁-C₄ alkyl, substituted or unsubstituted C₁-C₄ haloalkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl.

In some embodiments, the cyclic ring system comprises substituted or unsubstituted monocyclic aryl or substituted or unsubstituted monocyclic heteroaryl. In some embodiments, the cyclic ring system comprises substituted or unsubstituted bicyclic aryl or substituted or unsubstituted bicyclic heteroaryl.

In some embodiments, G or G¹ is or comprises a protein-binding ligand selected from a BTK, EGFR, EGFR T790M, JAK3, RIPK2, or tubulin binding ligand. In some embodiments, G or G¹ is or comprises a protein-binding ligand selected from a BTK, EGFR, JAK3, RIPK2, or tubulin binding ligand.

In some embodiments, G or G¹ is or comprises a protein-binding ligand selected from:

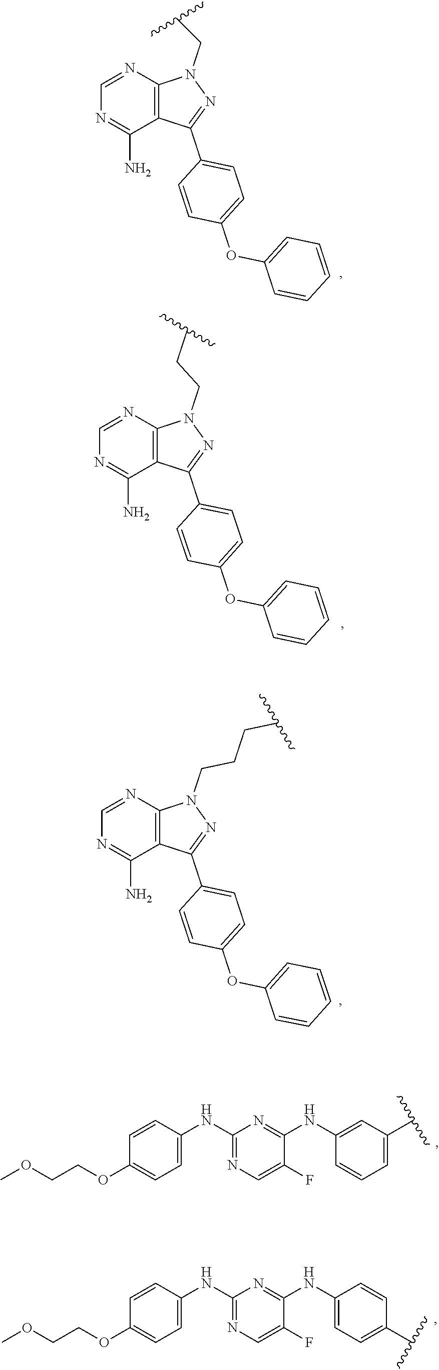 [SMILES: *Cn1nc(-c2ccc(Oc3ccccc3)cc2)c2c(N)ncnc21 ; *CCn1nc(-c2ccc(Oc3ccccc3)cc2)c2c(N)ncnc21 ; *CCCn1nc(-c2ccc(Oc3ccccc3)cc2)c2c(N)ncnc21 ; *c1cccc(Nc2nc(Nc3ccc(OCCOC)cc3)ncc2F)c1 ; *c1ccc(Nc2nc(Nc3ccc(OCCOC)cc3)ncc2F)cc1]

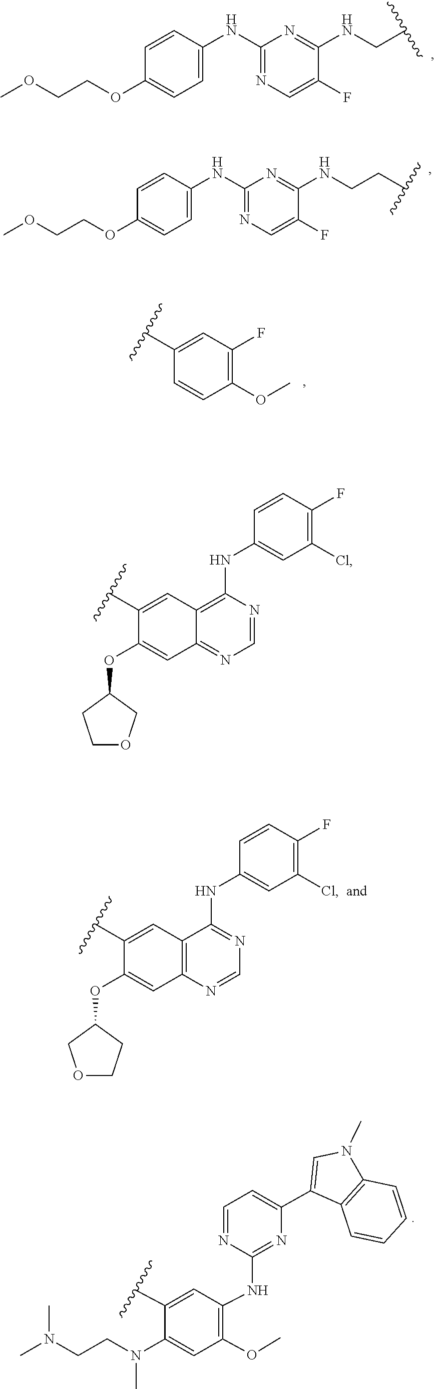

In some embodiments, G or G¹ is or comprises a protein-binding ligand selected from:

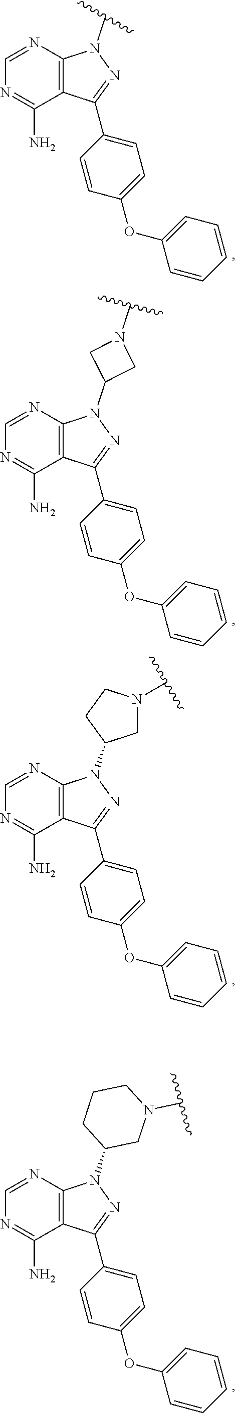

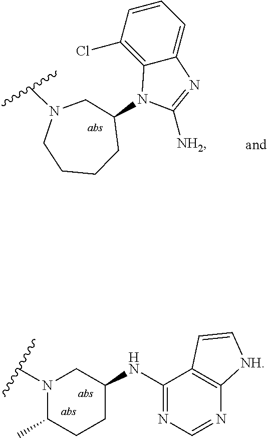 [SMILES: *N1CCCC[C@H](n2c(N)nc3cccc(Cl)c32)C1 ; *N1C[C@@H](Nc2ncnc3[nH]ccc23)CC[C@@H]1C]

In some embodiments, G or G¹ is or comprises a protein-binding ligand that is:

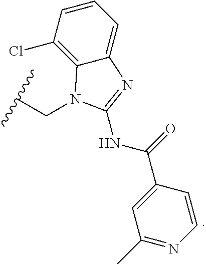 [SMILES: *Cn1c(NC(=O)c2ccnc(C)c2)nc2cccc(Cl)c21]

In some embodiments, G or G¹ is or comprises a protein-binding ligand that is:

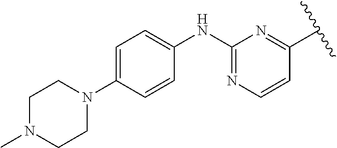 [SMILES: *c1ccnc(Nc2ccc(N3CCN(C)CC3)cc2)n1]

In some embodiments, G or G¹ is or comprises a protein-binding ligand that is:

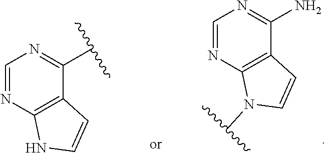 [SMILES: *c1ncnc2[nH]ccc12 ; *n1ccc2c(N)ncnc21]

In some embodiments, each R⁵ is independently hydrogen, —CN, substituted or unsubstituted alkyl, or substituted or unsubstituted heteroalkyl. In some embodiments, each R⁵ is independently hydrogen, —CN, —CH₃, —CH₂CH₃, —CH₂NH₂, —CH₂NHCH₃, —CH₂N(CH₃)₂, —CH₂F, —CHF₂, —CF₃, cyclopropyl, cyclobutyl, or cyclopentyl. In some embodiments, each R⁵ is independently hydrogen, —CN, —CH₃, —CF₃, or cyclopropyl. In some embodiments, each R⁵ is hydrogen. In some embodiments, each R⁵ is independently hydrogen, substituted or unsubstituted alkyl, or substituted or unsubstituted heteroalkyl. In some embodiments, each R⁵ is independently hydrogen, —OCH₂F, —OCHF₂, —OCF₃, —OCH₂CH₂F, —OCH₂CHF₂, —OCH₂CF₃, —NHCF₃, or —NHCH₂CF₃. In some embodiments, each R⁵ is independently hydrogen, —OCH₃, —OCH₂CH₃, —OCH₂F, —OCHF₂, —OCF₃, —OCH₂CH₂F, —OCH₂CHF₂, —OCH₂CF₃, cyclopropyloxy, or cyclobutyloxy. In some embodiments, each R⁵ is independently hydrogen, —CH₃, or —OCH₃. In some embodiments, each R⁵ is independently hydrogen or —CH₃. In some embodiments, each R⁵ is —CH₃.

In some embodiments, each R⁵ is independently hydrogen, —CN, —CH₃, —CH₂CH₃, —CH₂NH₂, —CH₂NHCH₃, —CH₂N(CH₃)₂, —CH₂F, —CHF₂, —CF₃, cyclopropyl, cyclobutyl, or cyclopentyl. In some embodiments, each R⁵ is independently hydrogen, —CN, —CH₃, —CF₃, or cyclopropyl. In some embodiments, each R⁵ is hydrogen.

In some embodiments, each R⁸ is independently hydrogen, substituted or unsubstituted C₁-C₄ alkyl, or substituted or unsubstituted C₁-C₄ heteroalkyl. In some embodiments, each R⁸ is independently hydrogen, —OCH₂F, —OCHF₂, —OCF₃, —OCH₂CH₂F, —OCH₂CHF₂, —OCH₂CF₃, —NHCF₃, or —NHCH₂CF₃. In some embodiments, each R⁸ is independently hydrogen, —OCH₃, —OCH₂CH₃, —OCH₂F, —OCHF₂, —OCF₃, —OCH₂CH₂F, —OCH₂CHF₂, —OCH₂CF₃, cyclopropyloxy, or cyclobutyloxy. In some embodiments, each R⁸ is independently hydrogen, —CH₃, or —OCH₃.

In some embodiments, X¹ is O, NH, or N(substituted or unsubstituted alkyl). In some embodiments, X¹ is O, NH, or N(alkyl). In some embodiments, X¹ is O, NH, or N(CH₃). In some embodiments, X¹ is O. In some embodiments, X¹ is NH or N(CH₃).

In some embodiments, each Y¹, Y², and Y³ is independently halo or alkyl. In some embodiments, Y¹ is fluoro. In some embodiments, Y² is fluoro. In some embodiments, Y³ is fluoro. In some embodiments, each Y¹, Y², and Y³ is independently halo or haloalkyl. In some embodiments, each Y¹, Y², and Y³ is independently halo. In some embodiments, each Y¹, Y², and Y³ is independently F or Cl.

In some embodiments, X¹ is O, NH, or N(substituted or unsubstituted alkyl). In some embodiments, X¹ is O, NH, or N(unsubstituted alkyl). In some embodiments, X¹ is O, NH, or N(CH₃). In some embodiments, X¹ is O. In some embodiments, X¹ is NH or N(CH₃).

In some embodiments X¹ and the O of O═S<=X¹ are absent.

In some embodiments, R² is fluoro.

In some embodiments, Y¹ and Y³ are fluoro.

In some embodiments, R², Y¹, and Y³ are fluoro.

In some embodiments, R², Y¹, and Y³ are fluoro and G^{R} is G.

In some embodiments, R², Y¹, and Y³ are fluoro, R¹ is R⁷, and G^{R} is G. In some embodiments, R², Y¹, and Y³ are fluoro, R¹ is sulfone (e.g., —SO₂CH₃), sulfoxide (e.g., —S(═O)CH₃), sulfonamide (e.g., —SO₂NH₂ or —SO₂N(CH₃)₂), —OR³ (e.g., R³ being hydrogen, substituted or unsubstituted alkyl (e.g., haloalkyl), or substituted or unsubstituted aryl (e.g., phenyl)), or substituted or unsubstituted alkyl (e.g., haloalkyl)), and G^{R} is G. In some embodiments, R², Y¹, and Y³ are fluoro, R¹ is —SO₂CH₃, —S(═O)CH₃, —SO₂NH₂ or —SO₂N(CH₃)₂, —OR³ (e.g., R³ being hydrogen, haloalkyl, or substituted or unsubstituted phenyl), or haloalkyl, and G^{R} is G.

In some embodiments, R², Y¹, Y², and Y³ are fluoro and R¹ is G.

In some embodiments, X is absent or O; R², Y¹, Y², and Y³ are fluoro; G^{R} is —NH₂, —N(CH₃)₂, or substituted or unsubstituted alkyl; and R¹ is G. In some embodiments, X is absent; R², Y¹, Y², and Y³ are fluoro; G^{R} is —NH₂, —N(CH₃)₂, or substituted or unsubstituted alkyl; and R¹ is G. In some embodiments, X is O; R², Y¹, Y², and Y³ are fluoro; G^{R} is —NH₂, —N(CH₃)₂, or substituted or unsubstituted alkyl; and R¹ is G.

In some embodiments, D2 is selected from

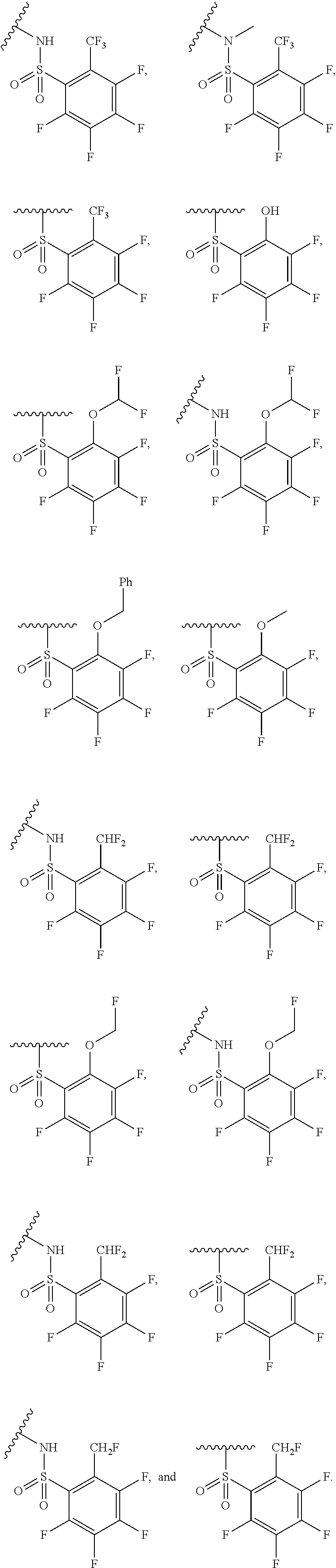

In some embodiments, D2 is selected from

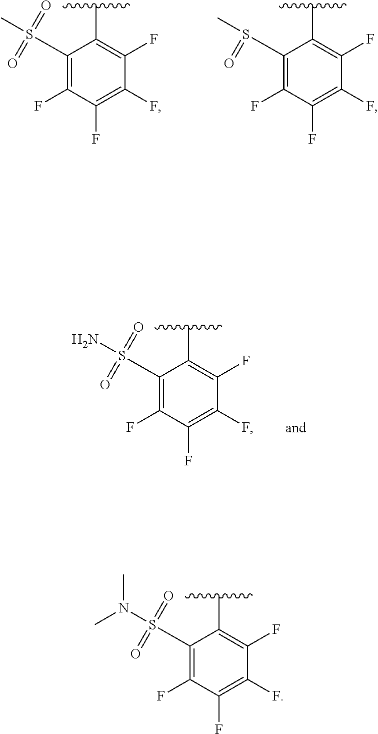 [SMILES: *c1c(F)c(F)c(F)c(F)c1S(C)(=O)=O ; *c1c(F)c(F)c(F)c(F)c1S(C)=O ; *c1c(F)c(F)c(F)c(F)c1S(N)(=O)=O ; *c1c(F)c(F)c(F)c(F)c1S(=O)(=O)N(C)C]

In some embodiments D2 or

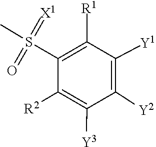

is selected from

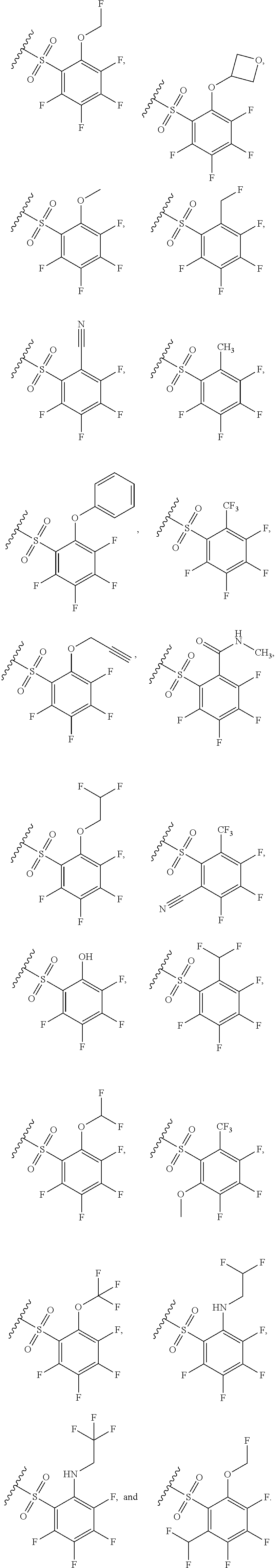

In some embodiments,

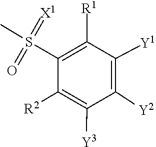

is selected from

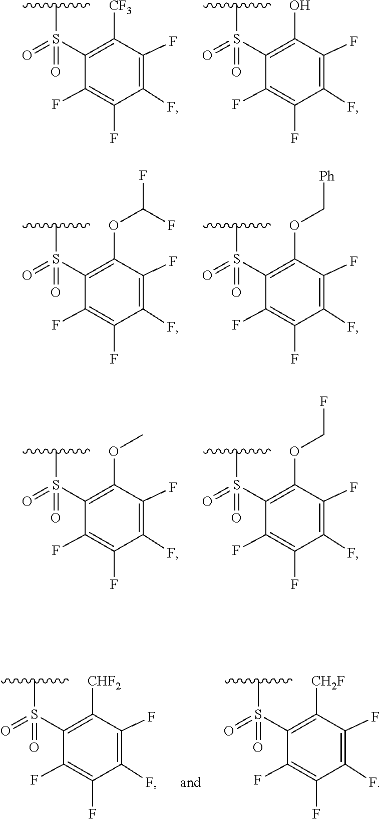

In some embodiments

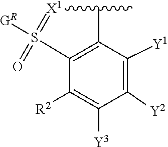

is selected from

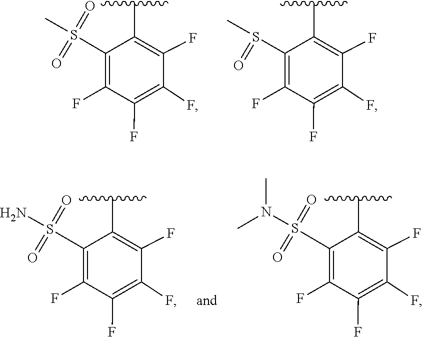

In some embodiments, the benzenesulfonamide derivative compound described herein has a structure provided in Table 1. In Table 1 and in other tables herein, when stating R¹=R², it is to be understood that R¹ and R² are the same as R¹ in the previous recitation. For example, in compound 7aaa, R¹=R², means that R¹ and R² are both H because R¹ is H in the previously recited compound 7aa. Thus, in compound 7aaa, R¹/R²/Y is H/H/F.

TABLE 1

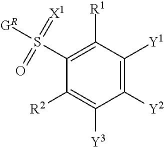

| # | G^{R} | R⁵ | X¹ | R¹/ R²/Y* |
|---|---|---|---|---|
| 7a | 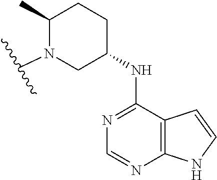 [SMILES: *N1C[C@@H](Nc2ncnc3[nH]ccc23)CC[C@@H]1C] | N/A | O | H/F/F (7aa); R¹ = R²/F (7aaa) OH/F/F (7ab); R¹ = R²/F (7aba) OMe/F/F (7ac); R¹ = R²/F (7aca) OCFH₂/F/F (7ad); R¹ = R²/F (7ada) OCF₂H/F/F (7ae); R¹ = R²/F (7aea) OCF₃/F/F (7af); R¹ = R²/F (7afa) OCH₂CF₃/F/F (7ag); R¹ = R²/F (7aga) O-cyclopropyl/F/F (7ah); R¹ = R²/F (7aha) OCH₂Ph/F/F (7ai); R¹ = R²/F (7aia) NH₂/F/F (7aj); R¹ = R²/F (7aja) NHMe/F/F (7ak); R¹ = R²/F (7aka) |
| | | | | NH-cyclopropyl/F/F (7al); R¹ = R²/F (7ala) |
| | | | | NHCH₂Ph/F/F (7am); R¹ = R²/F (7ama) |
| | | | | SH/F/F (7an); R¹ = R²/F (7ana) |
| | | | | SMe/F/F (7ao); R¹ = R²/F (7aoa) |
| | | | | SCH₂Ph/F/F (7ap); R¹ = R²/F (7apa) |
| | | | | Me/F/F (7aq); R¹ = R²/F (7aqa) |
| | | | | CF₃/F/F (7ar); R¹ = R²/F (7ara) |
| | | | | Ph/F/F (7as); R¹ = R²/F (7asa) |
| | | | | CO₂H/F/F (7at); R¹ = R²/F (7ata) |
| | | | | CO₂Me/F/F (7au); R¹ = R²/F (7aua) |
| | | | | CO₂NHMe/F/F (7av); R¹ = R²/F (7ava) |
| | | | | CN/F/F (7aw); R¹ = R²/F (7awa) |
| 7b | 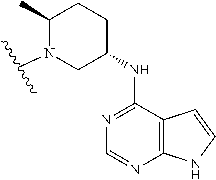 [SMILES: *N1C[C@@H](Nc2ncnc3[nH]ccc23)CC[C@@H]1C] | N/A | NH | H/F/F (7ba); R¹ = R²/F (7baa) OH/F/F (7bb); R¹ = R²/F (7bba) OMe/F/F (7bc); R¹ = R²/F (7bca) OCFH₂/F/F (7bd); R¹ = R²/F (7bda) OCF₂H/F/F (7be); R¹ = R²/F (7bea) OCF₃/F/F (7bf); R¹ = R²/F (7bfa) OCH₂CF₃/F/F (7bg); R¹ = R²/F (7bga) O-cyclopropyl/F/F (7bh); R¹ = R²/F (7bha) OCH₂Ph/F/F (7bi); R¹ = R²/F (7bia) NH₂/F/F (7bj); R¹ = R²/F (7bja) NHMe/F/F (7bk); R¹ = R²/F (7bka) |
| | | | | NH-cyclopropyl/F/F (7bl); R¹ = R²/F (7bla) |
| | | | | NHCH₂Ph/F/F (7bm); R¹ = R²/F (7bma) |
| | | | | SH/F/F (7bn); R¹ = R²/F (7bna) |
| | | | | SMe/F/F (7bo); R¹ = R²/F (7boa) |
| | | | | SCH₂Ph/F/F (7bp); R¹ = R²/F (7bpa) |
| | | | | Me/F/F (7bq); R¹ = R²/F (7bqa) |
| | | | | CF₃/F/F (7br); R¹ = R²/F (7bra) |
| | | | | Ph/F/F (7bs); R¹ = R²/F (7bsa) |
| | | | | CO₂H/F/F (7bt); R¹ = R²/F (7bta) |
| | | | | CO₂Me/F/F (7bu); R¹ = R²/F (7bua) |
| | | | | CO₂NHMe/F/F (7bv); R¹ = R²/F (7bva) |
| | | | | CN/F/F (7bw); R¹ = R²/F (7bwa) |
| 7c | 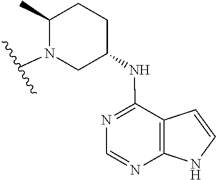 [SMILES: *N1C[C@@H](Nc2ncnc3[nH]ccc23)CC[C@@H]1C] | N/A | NMe | H/F/F (7ca); R¹ = R²/F (7caa) OH/F/F (7cb); R¹ = R²/F (7cba) OMe/F/F (7cc); R¹ = R²/F (7cca) OCFH₂/F/F (7cd); R¹ = R²/F (7cda) OCF₂H/F/F (7ce); R¹ = R²/F (7cea) OCF₃/F/F (7cf); R¹ = R²/F (7cfa) OCH₂CF₃/F/F (7cg); R¹ = R²/F (7cga) O-cyclopropyl/F/F (7ch); R¹ = R²/F (7cha) OCH₂Ph/F/F (7ci); R¹ = R²/F (7cia) NH₂/F/F (7cj); R¹ = R²/F (7cja) NHMe/F/F (7ck); R¹ = R²/F (7cka) |
| | | | | NH-cyclopropyl/F/F (7cl); R¹ = R²/F (7cla) |
| | | | | NHCH₂Ph/F/F (7cm); R¹ = R²/F (7cma) |
| | | | | SH/F/F (7cn); R¹ = R²/F (7cna) |
| | | | | SMe/F/F (7co); R¹ = R²/F (7coa) |
| | | | | SCH₂Ph/F/F (7cp); R¹ = R²/F (7cpa) |
| | | | | Me/F/F (7cq); R¹ = R²/F (7cqa) |
| | | | | CF₃/F/F (7cr); R¹ = R²/F (7cra) |
| | | | | Ph/F/F (7cs); R¹ = R²/F (7csa) |
| | | | | CO₂H/F/F (7ct); R¹ = R²/F (7cta) |
| | | | | CO₂Me/F/F (7cu); R¹ = R²/F (7cua) |
| | | | | CO₂NHMe/F/F (7cv); R¹ = R²/F (7cva) |
| | | | | CN/F/F (7cw); R¹ = R²/F (7cwa) |
| 26a | 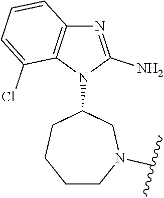 [SMILES: *N1CCCC[C@H](n2c(N)nc3cccc(Cl)c32)C1] | N/A | O | H/F/F (26aa); R¹ = R²/F (26aaa) OH/F/F (26ab); R¹ = R²/F (26aba) OMe/F/F (26ac); R¹ = R²/F (26aca) OCFH₂/F/F (26ad); R¹ = R²/F (26ada) OCF₂H/F/F (26ae); R¹ = R²/F (26aea) OCF₃/F/F (26af); R¹ = R²/F (26afa) OCH₂CF₃/F/F (26ag); R¹ = R²/F (26aga) O-cyclopropyl/F/F (26ah); R¹ = R²/F (26aha) OCH₂Ph/F/F (26ai); R¹ = R²/F (26aia) NH₂/F/F (26aj); R¹ = R²/F (26aja) NHMe/F/F (26ak); R¹ = R²/F (26aka) NH-cyclopropyl/F/F (26al); R¹ = R²/F (26ala) |
| | | | | NHCH₂Ph/F/F (26am); R¹ = R²/F (26ama) |
| | | | | SH/F/F (26an); R¹ = R²/F (26ana) |
| | | | | SMe/F/F (26ao); R¹ = R²/F (26aoa) |
| | | | | SCH₂Ph/F/F (26ap); R¹ = R²/F (26apa) |
| | | | | Me/F/F (26aq); R¹ = R²/F (26aqa) |
| | | | | CF₃/F/F (26ar); R¹ = R²/F (26ara) |
| | | | | Ph/F/F (26as); R¹ = R²/F (26asa) |
| | | | | CO₂H/F/F (26at); R¹ = R²/F (26ata) |
| | | | | CO₂Me/F/F (26au); R¹ = R²/F (26aua) |
| | | | | CO₂NHMe/F/F (26av); R¹ = R²/F (26ava) |
| | | | | CN/F/F (26aw); R¹ = R²/F (26awa) |
| 26b | 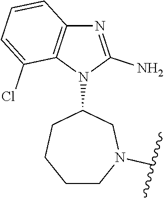 [SMILES: *N1CCCC[C@H](n2c(N)nc3cccc(Cl)c32)C1] | N/A | NH | H/F/F (26ba); R¹ = R²/F (26baa) OH/F/F (26bb); R¹ = R²/F (26bba) OMe/F/F (26bc); R¹ = R²/F (26bca) OCFH₂/F/F (26bd); R¹ = R²/F (26bda) OCF₂H/F/F (26be); R¹ = R²/F (26bea) OCF₃/F/F (26bf); R¹ = R²/F (26bfa) OCH₂CF₃/F/F (26bg); R¹ = R²/F (26bga) O-cyclopropyl/F/F (26bh); R¹ = R²/F (26bha) OCH₂Ph/F/F (26bi); R¹ = R²/F (26bia) NH₂/F/F (26bj); R¹ = R²/F (26bja) NHMe/F/F (26bk); R¹ = R²/F (26bka) NH-cyclopropyl/F/F (26bl); R¹ = R²/F (26bla) |
| | | | | NHCH₂Ph/F/F (26bm); R¹ = R²/F (26bma) |
| | | | | SH/F/F (26bn); R¹ = R²/F (26bna) |
| | | | | SMe/F/F (26bo); R¹ = R²/F (26boa) |
| | | | | SCH₂Ph/F/F (26bp); R¹ = R²/F (26bpa) |
| | | | | Me/F/F (26bq); R¹ = R²/F (26bqa) |
| | | | | CF₃/F/F (26br); R¹ = R²/F (26bra) |
| | | | | Ph/F/F (26bs); R¹ = R²/F (26bsa) |
| | | | | CO₂H/F/F (26bt); R¹ = R²/F (26bta) |
| | | | | CO₂Me/F/F (26bu); R¹ = R²/F (26bua) |
| | | | | CO₂NHMe/F/F (26bv); R¹ = R²/F (26bva) |
| | | | | CN/F/F (26bw); R¹ = R²/F (26bwa) |
| 26c | 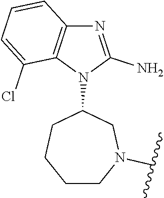 [SMILES: *N1CCCC[C@H](n2c(N)nc3cccc(Cl)c32)C1] | N/A | NMe | H/F/F (26ca); R¹ = R²/F (26caa) OH/F/F (26cb); R¹ = R²/F (26cba) OMe/F/F (26cc); R¹ = R²/F (26cca) OCFH₂/F/F (26cd); R¹ = R²/F (26cda) OCF₂H/F/F (26ce); R¹ = R²/F (26cea) OCF₃/F/F (26cf); R¹ = R²/F (26cfa) OCH₂CF₃/F/F (26cg); R¹ = R²/F (26cga) O-cyclopropyl/F/F (26ch); R¹ = R²/F (26cha) OCH₂Ph/F/F (26ci); R¹ = R²/F (26cia) NH₂/F/F (26cj); R¹ = R²/F (26cja) NHMe/F/F (26ck); R¹ = R²/F (26cka) NH-cyclopropyl/F/F (26cl); R¹ = R²/F (26cla) |
| | | | | NHCH₂Ph/F/F (26cm); R¹ = R²/F (26cma) |
| | | | | SH/F/F (26cn); R¹ = R²/F (26cna) |
| | | | | SMe/F/F (26co); R¹ = R²/F (26coa) |
| | | | | SCH₂Ph/F/F (26cp); R¹ = R²/F (26cpa) |
| | | | | Me/F/F (26cq); R¹ = R²/F (26cqa) |
| | | | | CF₃/F/F (26cr); R¹ = R²/F (26cra) |
| | | | | Ph/F/F (26cs); R¹ = R²/F (26csa) |
| | | | | CO₂H/F/F (26ct); R¹ = R²/F (26cta) |
| | | | | CO₂Me/F/F (26cu); R¹ = R²/F (26cua) |
| | | | | CO₂NHMe/F/F (26cv); R¹ = R²/F (26cva) |
| | | | | CN/F/F (26cw); R¹ = R²/F (26cwa) |
| 85a | 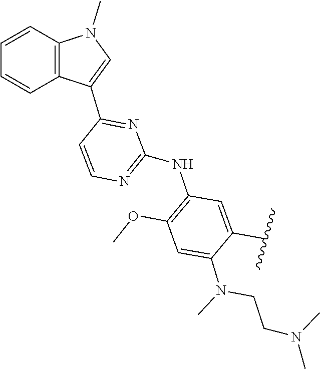 [SMILES: *c1cc(Nc2nccc(-c3cn(C)c4ccccc34)n2)c(OC)cc1N(C)CCN(C)C] | H | O | H/F/F (85aa); R¹ = R²/F (85aaa) OH/F/F (85ab); R¹ = R²/F (85aba) OMe/F/F (85ac); R¹ = R²/F (85aca) OCFH₂/F/F (85ad); R¹ = R²/F (85ada) OCF₂H/F/F (85ae); R¹ = R²/F (85aea) OCF₃/F/F (85af); R¹ = R²/F (85afa) OCH₂CF₃/F/F (85ag); R¹ = R²/F (85aga) O-cyclopropyl/F/F (85ah); R¹ = R²/F (85aha) OCH₂Ph/F/F (85ai); R¹ = R²/F (85aia) NH₂/F/F (85aj); R¹ = R²/F (85aja) NHMe/F/F (85ak); R¹ = R²/F (85aka) NH-cyclopropyl/F/F (85al); R¹ = R²/F (85ala) NHCH₂Ph/F/F (85am); R¹ = R²/F (85ama) SH/F/F (85an); R¹ = R²/F (85ana) SMe/F/F (85ao); R¹ = R²/F (85aoa) SCH₂Ph/F/F (85ap); R¹ = R²/F (85apa) Me/F/F (85aq); R¹ = R²/F (85aqa) CF₃/F/F (85ar); R¹ = R²/F (85ara) Ph/F/F (85as); R¹ = R²/F (85asa) CO₂H/F/F (85at); R¹ = R²/F (85ata) CO₂Me/F/F (85au); R¹ = R²/F (85aua) CO₂NHMe/F/F (85av); R¹ = R²/F (85ava) CN/F/F (85aw); R1 = R2/F (85awa) |
| 85b | 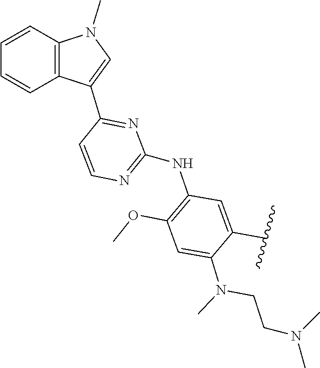 [SMILES: *c1cc(Nc2nccc(-c3cn(C)c4ccccc34)n2)c(OC)cc1N(C)CCN(C)C] | Me | O | H/F/F (85ba); R¹ = R²/F (85baa) OH/F/F (85bb); R¹ = R²/F (85bba) OMe/F/F (85bc); R¹ = R²/F (85bca) OCFH₂/F/F (85bd); R¹ = R²/F (85bda) OCF₂H/F/F (85be); R¹ = R²/F (85bea) OCF₃/F/F (85bf); R¹ = R²/F (85bfa) OCH₂CF₃/F/F (85bg); R¹ = R²/F (85bga) O-cyclopropyl/F/F (85bh); R¹ = R²/F (85bha) OCH₂Ph/F/F (85bi); R¹ = R²/F (85bia) NH₂/F/F (85bj); R¹ = R²/F (85bja) NHMe/F/F (85bk); R¹ = R²/F (85bka) NH-cyclopropyl/F/F (85bl); R¹ = R²/F (85bla) NHCH₂Ph/F/F (85bm); R¹ = R²/F (85bma) SH/F/F (85bn); R¹ = R²/F (85bna) SMe/F/F (85bo); R¹ = R²/F (85boa) SCH₂Ph/F/F (85bp); R¹ = R²/F (85bpa) Me/F/F (85bq); R¹ = R²/F (85bqa) CF₃/F/F (85br); R¹ = R²/F (85bra) Ph/F/F (85bs); R¹ = R²/F (85bsa) CO₂H/F/F (85bt); R¹ = R²/F (85bta) CO₂Me/F/F (85bu); R¹ = R²/F (85bua) CO₂NHMe/F/F (85bv); R¹ = R²/F (85bva) CN/F/F (85bw); R1 = R2/F (85bwa) |
| 85c | 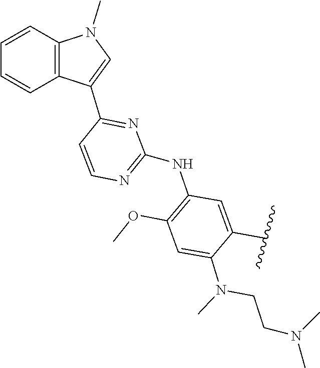 [SMILES: *c1cc(Nc2nccc(-c3cn(C)c4ccccc34)n2)c(OC)cc1N(C)CCN(C)C] | H | NH | H/F/F (85ca); R¹ = R²/F (85caa) OH/F/F (85cb); R¹ = R²/F (85cba) OMe/F/F (85cc); R¹ = R²/F (85cca) OCFH₂/F/F (85cd); R¹ = R²/F (85cda) OCF₂H/F/F (85ce); R¹ = R²/F (85cea) OCF₃/F/F (85cf); R¹ = R²/F (85cfa) OCH₂CF₃/F/F (85cg); R¹ = R²/F (85cga) O-cyclopropyl/F/F (85ch); R¹ = R²/F (85cha) OCH₂Ph/F/F (85ci); R¹ = R²/F (85cia) NH₂/F/F (85cj); R¹ = R²/F (85cja) NHMe/F/F (85ck); R¹ = R²/F (85cka) NH-cyclopropyl/F/F (85cl); R¹ = R²/F (85cla) NHCH₂Ph/F/F (85cm); R¹ = R²/F (85cma) SH/F/F (85cn); R¹ = R²/F (85cna) SMe/F/F (85co); R¹ = R²/F (85coa) SCH₂Ph/F/F (85cp); R¹ = R²/F (85cpa) Me/F/F (85cq); R¹ = R²/F (85cqa) CF₃/F/F (85cr); R¹ = R²/F (85cra) Ph/F/F (85cs); R¹ = R²/F (85csa) CO₂H/F/F (85ct); R¹ = R²/F (85cta) CO₂Me/F/F (85cu); R¹ = R²/F (85cua) CO₂NHMe/F/F (85cv); R¹ = R²/F (85cva) CN/F/F (85cw); R1 = R2/F (85cwa) |
| 85d | 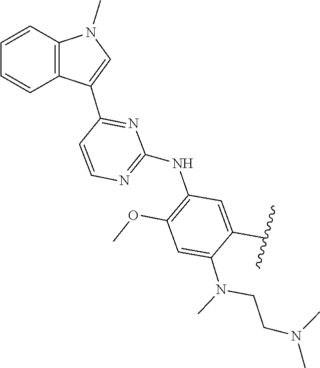 [SMILES: *c1cc(Nc2nccc(-c3cn(C)c4ccccc34)n2)c(OC)cc1N(C)CCN(C)C] | Me | NH | H/F/F (85da); R¹ = R²/F (85daa) OH/F/F (85db); R¹ = R²/F (85dba) OMe/F/F (85dc); R¹ = R²/F (85dca) OCFH₂/F/F (85dd); R¹ = R²/F (85dda) OCF₂H/F/F (85de); R¹ = R²/F (85dea) OCF₃/F/F (85df); R¹ = R²/F (85dfa) OCH₂CF₃/F/F (85dg); R¹ = R²/F (85dga) O-cyclopropyl/F/F (85dh); R¹ = R²/F (85dha) OCH₂Ph/F/F (85di); R¹ = R²/F (85dia) NH₂/F/F (85dj); R¹ = R²/F (85dja) NHMe/F/F (85dk); R¹ = R²/F (85dka) NH-cyclopropyl/F/F (85dl); R¹ = R²/F (85dla) NHCH₂Ph/F/F (85dm); R¹ = R²/F (85dma) SH/F/F (85dn); R¹ = R²/F (85dna) SMe/F/F (85do); R¹ = R²/F (85doa) SCH₂Ph/F/F (85dp); R¹ = R²/F (85dpa) Me/F/F (85dq); R¹ = R²/F (85dqa) CF₃/F/F (85dr); R¹ = R²/F (85dra) Ph/F/F (85ds); R¹ = R²/F (85dsa) CO₂H/F/F (85dt); R¹ = R²/F (85dta) CO₂Me/F/F (85du); R¹ = R²/F (85dua) CO₂NHMe/F/F (85dv); R¹ = R²/F (85dva) CN/F/F (85dw); R1 = R2/F (85dwa) |
| 85e | 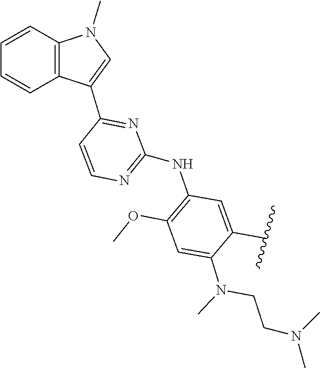 [SMILES: *c1cc(Nc2nccc(-c3cn(C)c4ccccc34)n2)c(OC)cc1N(C)CCN(C)C] | H | NMe | H/F/F (85ea); R¹ = R²/F (85eaa) OH/F/F (85eb); R¹ = R²/F (85eba) OMe/F/F (85ec); R¹ = R²/F (85eca) OCFH₂/F/F (85ed); R¹ = R²/F (85eda) OCF₂H/F/F (85ee); R¹ = R²/F (85eea) OCF₃/F/F (85ef); R¹ = R²/F (85efa) OCH₂CF₃/F/F (85eg); R¹ = R²/F (85ega) O-cyclopropyl/F/F (85eh); R¹ = R²/F (85eha) OCH₂Ph/F/F (85ei); R¹ = R²/F (85eia) NH₂/F/F (85ej); R¹ = R²/F (85eja) NHMe/F/F (85ek); R¹ = R²/F (85eka) NH-cyclopropyl/F/F (85el); R¹ = R²/F (85ela) NHCH₂Ph/F/F (85em); R¹ = R²/F (85ema) SH/F/F (85en); R¹ = R²/F (85ena) SMe/F/F (85e0); R¹ = R²/F (85eoa) SCH₂Ph/F/F (85ep); R¹ = R²/F (85epa) Me/F/F (85eq); R¹ = R²/F (85eqa) CF₃/F/F (85er); R¹ = R²/F (85era) Ph/F/F (85es); R¹ = R²/F (85esa) CO₂H/F/F (85et); R¹ = R²/F (85eta) CO₂Me/F/F (85eu); R¹ = R²/F (85eua) CO₂NHMe/F/F (85ev); R¹ = R²/F (85eva) |
| | | | | CN/F/F (85ew); R¹ = R²/F (85ewa) |
| 85f | 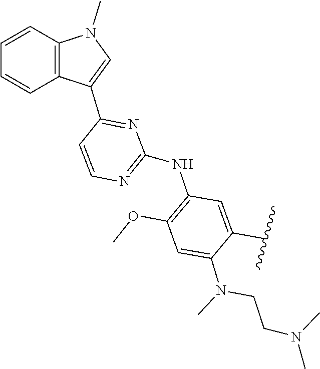 [SMILES: *c1cc(Nc2nccc(-c3cn(C)c4ccccc34)n2)c(OC)cc1N(C)CCN(C)C] | Me | NMe | H/F/F (85fa); R¹ = R²/F (85faa) OH/F/F (85fb); R¹ = R²/F (85fba) OMe/F/F (85fc); R¹ = R²/F (85fca) OCFH₂/F/F (85fd); R¹ = R²/F (85fda) OCF₂H/F/F (85fe); R¹ = R²/F (85fea) OCF₃/F/F (85ff); R¹ = R²/F (85ffa) OCH₂CF₃/F/F (85fg); R¹ = R²/F (85fga) O-cyclopropyl/F/F (85fh); R¹ = R²/F (85fha) OCH₂Ph/F/F (85fi); R¹ = R²/F (85fia) NH₂/F/F (85fj); R¹ = R²/F (85fja) NHMe/F/F (85fk); R¹ = R²/F (85fka) NH-cyclopropyl/F/F (85fl); R¹ = R²/F (85fla) NHCH₂Ph/F/F (85fm); R¹ = R²/F (85fma) SH/F/F (85fn); R¹ = R²/F (85fna) SMe/F/F (85fo); R¹ = R²/F (85foa) SCH₂Ph/F/F (85fp); R¹ = R²/F (85fpa) Me/F/F (85fq); R¹ = R²/F (85fqa) CF₃/F/F (85fr); R¹ = R²/F (85fra) Ph/F/F (85fs); R¹ = R²/F (85fsa) CO₂H/F/F (85ft); R¹ = R²/F (85fta) CO₂Me/F/F (85fu); R¹ = R²/F (85fua) CO₂NHMe/F/F (85fv); R¹ = R²/F (85fva) CN/F/F (85fw); R¹ = R²/F (85fwa) |

*each of Y¹, Y², and Y³ is Y

In some embodiments, disclosed herein is a pharmaceutically acceptable salt, solvate, tautomer, regioisomer, or stereoisomer of a compound of Table 1.

In some embodiments, the benzenesulfonamide derivative compound described herein has a structure provided in Table 2. For the benzenesulfonamide derivative compound in Table 2, R¹, R², Y¹, Y², and Y³ are as described in Table 1.

TABLE 2

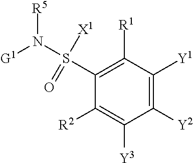

| # | 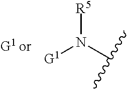 | R⁵ | X¹ |
|---|---|---|---|
| 4a | 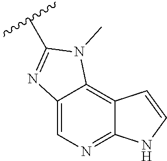 [SMILES: *c1nc2cnc3[nH]ccc3c2n1C] | H | O |
| 4b | 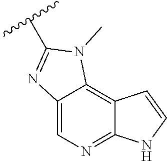 [SMILES: *c1nc2cnc3[nH]ccc3c2n1C] | Me | O |
| 4c | 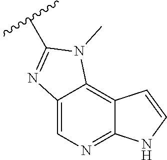 [SMILES: *c1nc2cnc3[nH]ccc3c2n1C] | H | NH |
| 4d | 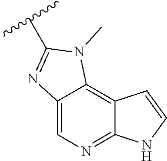 [SMILES: *c1nc2cnc3[nH]ccc3c2n1C] | Me | NH |
| 4e | 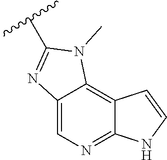 [SMILES: *c1nc2cnc3[nH]ccc3c2n1C] | H | NMe |
| 4f | 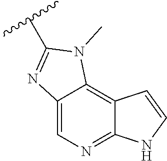 [SMILES: *c1nc2cnc3[nH]ccc3c2n1C] | Me | NMe |
| 5a | 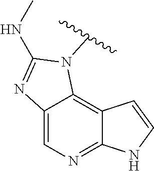 [SMILES: *n1c(NC)nc2cnc3[nH]ccc3c21] | N/A | O |
| 5b | 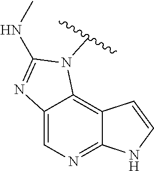 [SMILES: *n1c(NC)nc2cnc3[nH]ccc3c21] | N/A | NH |
| 5c | 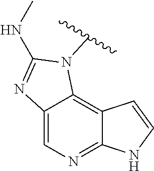 [SMILES: *n1c(NC)nc2cnc3[nH]ccc3c21] | N/A | NMe |
| 6a | 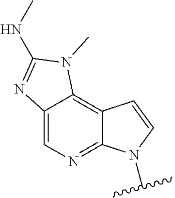 [SMILES: *n1ccc2c1ncc1nc(NC)n(C)c12] | N/A | O |
| 6b | 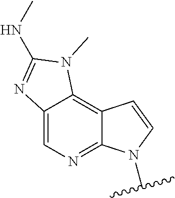 [SMILES: *n1ccc2c1ncc1nc(NC)n(C)c12] | N/A | NH |
| 6c | 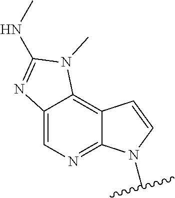 [SMILES: *n1ccc2c1ncc1nc(NC)n(C)c12] | N/A | NMe |
| 7a | 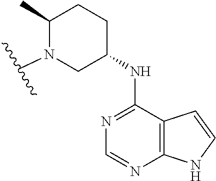 [SMILES: *N1C[C@@H](Nc2ncnc3[nH]ccc23)CC[C@@H]1C] | N/A | O |
| 7b | 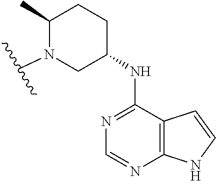 [SMILES: *N1C[C@@H](Nc2ncnc3[nH]ccc23)CC[C@@H]1C] | N/A | NH |
| 7c | 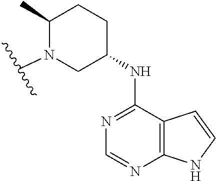 [SMILES: *N1C[C@@H](Nc2ncnc3[nH]ccc23)CC[C@@H]1C] | N/A | NMe |
| 8a | 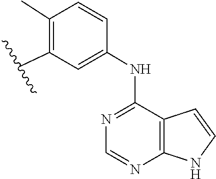 [SMILES: *c1cc(Nc2ncnc3[nH]ccc23)ccc1C] | H | O |
| 8b | 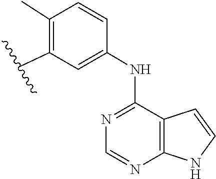 [SMILES: *c1cc(Nc2ncnc3[nH]ccc23)ccc1C] | Me | O |
| 8c | 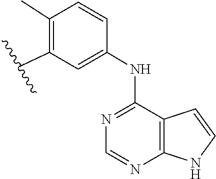 [SMILES: *c1cc(Nc2ncnc3[nH]ccc23)ccc1C] | H | NH |
| 8d | 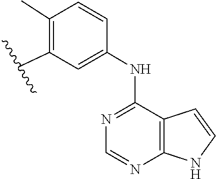 [SMILES: *c1cc(Nc2ncnc3[nH]ccc23)ccc1C] | Me | NH |
| 8e | 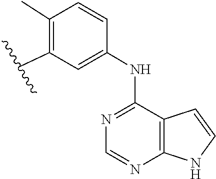 [SMILES: *c1cc(Nc2ncnc3[nH]ccc23)ccc1C] | H | NMe |
| 8f | 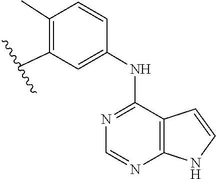 [SMILES: *c1cc(Nc2ncnc3[nH]ccc23)ccc1C] | Me | NMe |
| 9a | 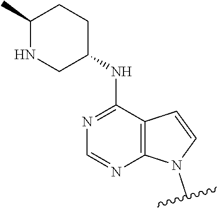 [SMILES: *n1ccc2c(N[C@H]3CC[C@H](C)NC3)ncnc21] | N/A | O |
| 9b | 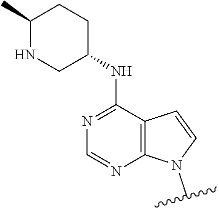 [SMILES: *n1ccc2c(N[C@H]3CC[C@H](C)NC3)ncnc21] | N/A | NH |
| 9c | 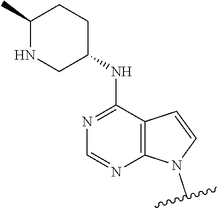 [SMILES: *n1ccc2c(N[C@H]3CC[C@H](C)NC3)ncnc21] | N/A | NMe |
| 10a | 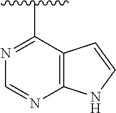 [SMILES: *c1ncnc2[nH]ccc12] | H | O |
| 10b | 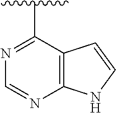 [SMILES: *c1ncnc2[nH]ccc12] | Me | O |
| 10c | 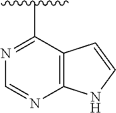 [SMILES: *c1ncnc2[nH]ccc12] | H | NH |
| 10d | 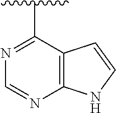 [SMILES: *c1ncnc2[nH]ccc12] | Me | NH |
| 10e | 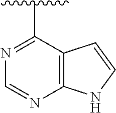 [SMILES: *c1ncnc2[nH]ccc12] | H | NMe |
| 10f | 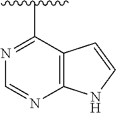 [SMILES: *c1ncnc2[nH]ccc12] | Me | NMe |
| 21a | 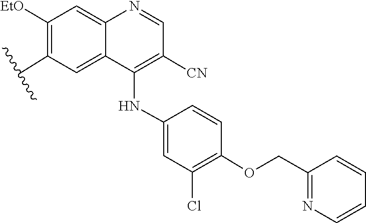 [SMILES: *c1cc2c(Nc3ccc(OCc4ccccn4)c(Cl)c3)c(C#N)cnc2cc1OCC] | H | O |
| 21b | 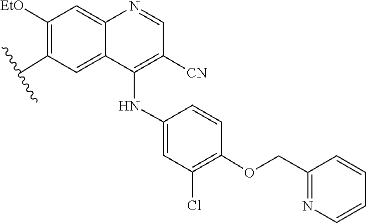 [SMILES: *c1cc2c(Nc3ccc(OCc4ccccn4)c(Cl)c3)c(C#N)cnc2cc1OCC] | Me | O |
| 21c | 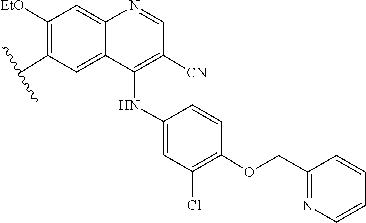 [SMILES: *c1cc2c(Nc3ccc(OCc4ccccn4)c(Cl)c3)c(C#N)cnc2cc1OCC] | H | NH |
| 21d | 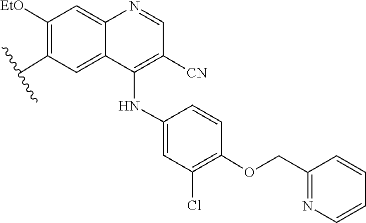 [SMILES: *c1cc2c(Nc3ccc(OCc4ccccn4)c(Cl)c3)c(C#N)cnc2cc1OCC] | Me | NH |
| 21e | 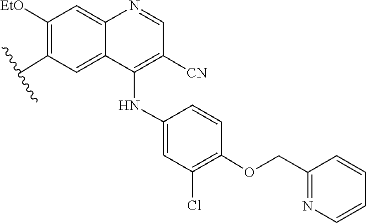 [SMILES: *c1cc2c(Nc3ccc(OCc4ccccn4)c(Cl)c3)c(C#N)cnc2cc1OCC] | H | NMe |
| 21f | 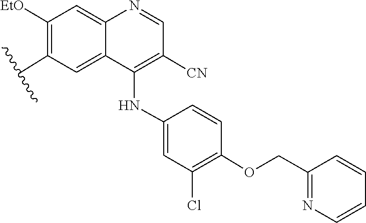 [SMILES: *c1cc2c(Nc3ccc(OCc4ccccn4)c(Cl)c3)c(C#N)cnc2cc1OCC] | Me | NMe |
| 22a | 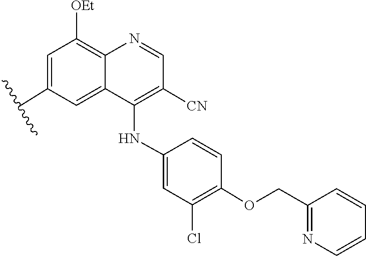 [SMILES: *c1cc(OCC)c2ncc(C#N)c(Nc3ccc(OCc4ccccn4)c(Cl)c3)c2c1] | H | O |
| 22b | 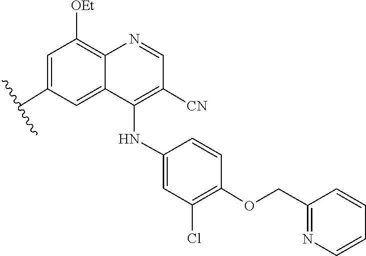 [SMILES: *c1cc(OCC)c2ncc(C#N)c(Nc3ccc(OCc4ccccn4)c(Cl)c3)c2c1] | Me | O |
| 22c | 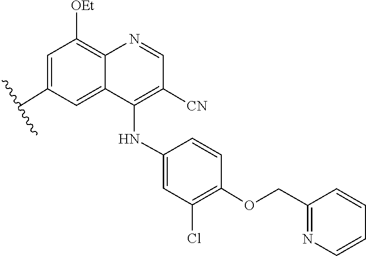 [SMILES: *c1cc(OCC)c2ncc(C#N)c(Nc3ccc(OCc4ccccn4)c(Cl)c3)c2c1] | H | NH |
| 22d | 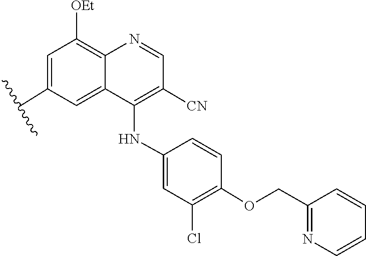 [SMILES: *c1cc(OCC)c2ncc(C#N)c(Nc3ccc(OCc4ccccn4)c(Cl)c3)c2c1] | Me | NH |
| 22e | 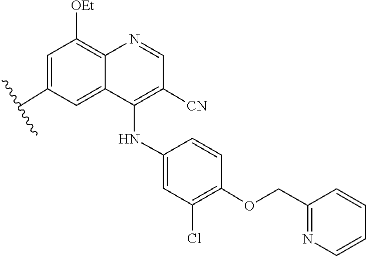 [SMILES: *c1cc(OCC)c2ncc(C#N)c(Nc3ccc(OCc4ccccn4)c(Cl)c3)c2c1] | H | NMe |
| 22f | 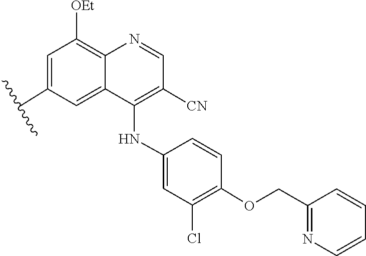 [SMILES: *c1cc(OCC)c2ncc(C#N)c(Nc3ccc(OCc4ccccn4)c(Cl)c3)c2c1] | Me | NMe |
| 23a | 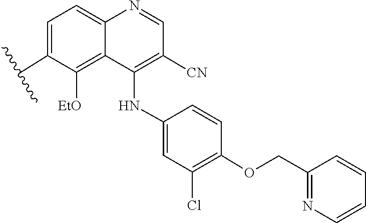 [SMILES: *c1ccc2ncc(C#N)c(Nc3ccc(OCc4ccccn4)c(Cl)c3)c2c1OCC] | H | O |
| 23b | 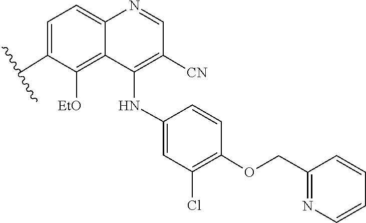 [SMILES: *c1ccc2ncc(C#N)c(Nc3ccc(OCc4ccccn4)c(Cl)c3)c2c1OCC] | Me | O |
| 23c | 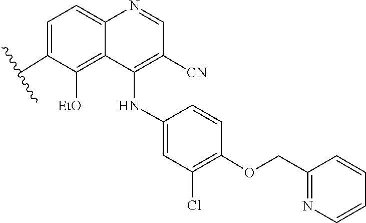 [SMILES: *c1ccc2ncc(C#N)c(Nc3ccc(OCc4ccccn4)c(Cl)c3)c2c1OCC] | H | NH |
| 23d | 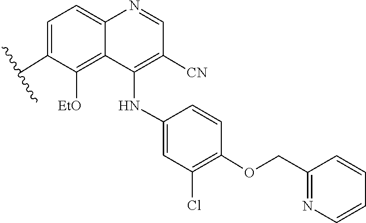 [SMILES: *c1ccc2ncc(C#N)c(Nc3ccc(OCc4ccccn4)c(Cl)c3)c2c1OCC] | Me | NH |
| 23e | 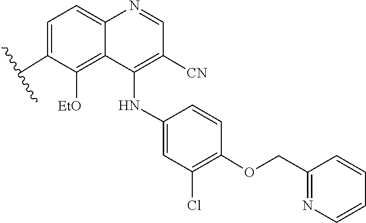 [SMILES: *c1ccc2ncc(C#N)c(Nc3ccc(OCc4ccccn4)c(Cl)c3)c2c1OCC] | H | NMe |
| 23f | 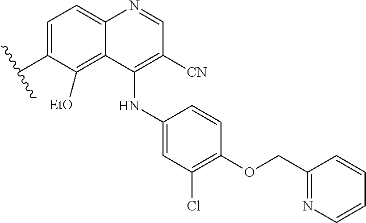 [SMILES: *c1ccc2ncc(C#N)c(Nc3ccc(OCc4ccccn4)c(Cl)c3)c2c1OCC] | Me | NMe |
| 24a | 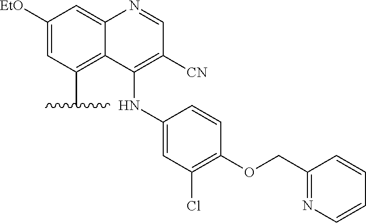 [SMILES: *c1cc(OCC)cc2ncc(C#N)c(Nc3ccc(OCc4ccccn4)c(Cl)c3)c12] | H | O |
| 24b | 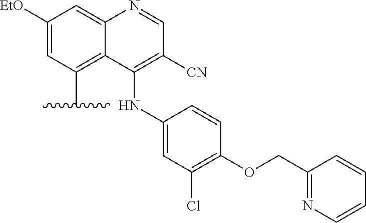 [SMILES: *c1cc(OCC)cc2ncc(C#N)c(Nc3ccc(OCc4ccccn4)c(Cl)c3)c12] | Me | O |
| 24c | 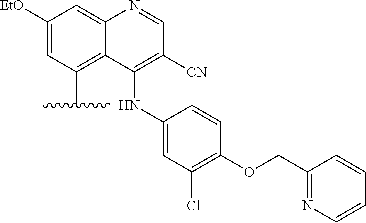 [SMILES: *c1cc(OCC)cc2ncc(C#N)c(Nc3ccc(OCc4ccccn4)c(Cl)c3)c12] | H | NH |
| 24d | 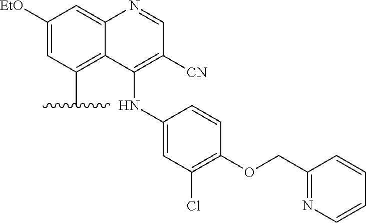 [SMILES: *c1cc(OCC)cc2ncc(C#N)c(Nc3ccc(OCc4ccccn4)c(Cl)c3)c12] | Me | NH |
| 24e | 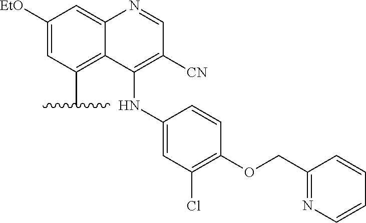 [SMILES: *c1cc(OCC)cc2ncc(C#N)c(Nc3ccc(OCc4ccccn4)c(Cl)c3)c12] | H | NMe |
| 24f | 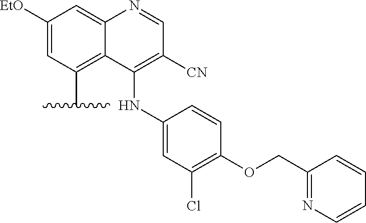 [SMILES: *c1cc(OCC)cc2ncc(C#N)c(Nc3ccc(OCc4ccccn4)c(Cl)c3)c12] | Me | NMe |
| 25a | 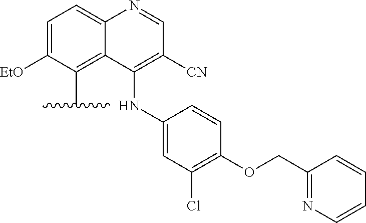 [SMILES: *c1c(OCC)ccc2ncc(C#N)c(Nc3ccc(OCc4ccccn4)c(Cl)c3)c12] | H | O |
| 25b | 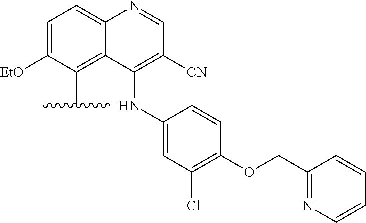 [SMILES: *c1c(OCC)ccc2ncc(C#N)c(Nc3ccc(OCc4ccccn4)c(Cl)c3)c12] | Me | O |
| 25c | 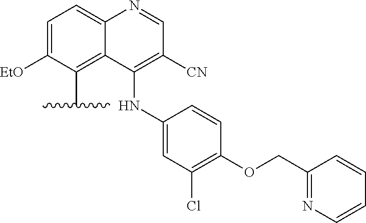 [SMILES: *c1c(OCC)ccc2ncc(C#N)c(Nc3ccc(OCc4ccccn4)c(Cl)c3)c12] | H | NH |
| 25d | 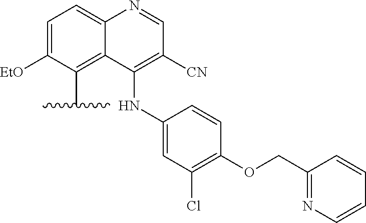 [SMILES: *c1c(OCC)ccc2ncc(C#N)c(Nc3ccc(OCc4ccccn4)c(Cl)c3)c12] | Me | NH |
| 25e | 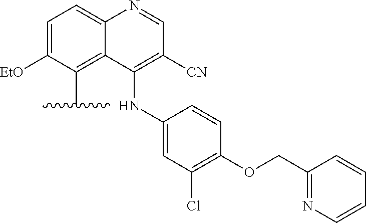 [SMILES: *c1c(OCC)ccc2ncc(C#N)c(Nc3ccc(OCc4ccccn4)c(Cl)c3)c12] | H | NMe |
| 25f | 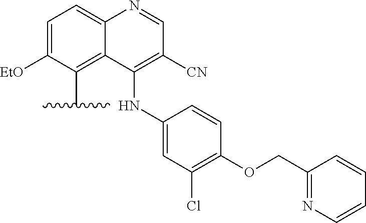 [SMILES: *c1c(OCC)ccc2ncc(C#N)c(Nc3ccc(OCc4ccccn4)c(Cl)c3)c12] | Me | NMe |
| 26a | 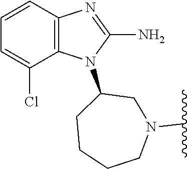 [SMILES: *N1CCCC[C@@H](n2c(N)nc3cccc(Cl)c32)C1] | N/A | O |
| 26b | 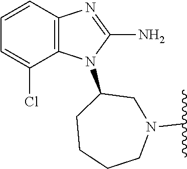 [SMILES: *N1CCCC[C@@H](n2c(N)nc3cccc(Cl)c32)C1] | N/A | NH |
| 26c | 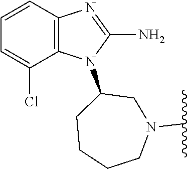 [SMILES: *N1CCCC[C@@H](n2c(N)nc3cccc(Cl)c32)C1] | N/A | NMe |
| 27a | 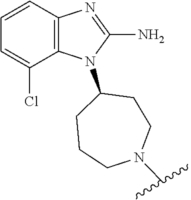 [SMILES: *N1CCC[C@@H](n2c(N)nc3cccc(Cl)c32)CC1] | N/A | O |
| 27b | 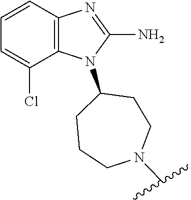 [SMILES: *N1CCC[C@@H](n2c(N)nc3cccc(Cl)c32)CC1] | N/A | NH |
| 27c | 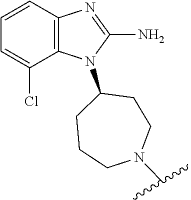 [SMILES: *N1CCC[C@@H](n2c(N)nc3cccc(Cl)c32)CC1] | N/A | NMe |
| 28a | 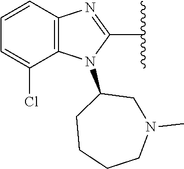 [SMILES: *c1nc2cccc(Cl)c2n1[C@@H]1CCCCN(C)C1] | H | O |
| 28b | 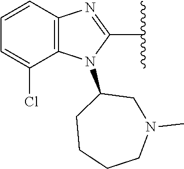 [SMILES: *c1nc2cccc(Cl)c2n1[C@@H]1CCCCN(C)C1] | Me | O |
| 28c | 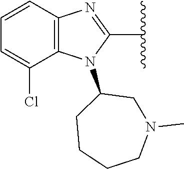 [SMILES: *c1nc2cccc(Cl)c2n1[C@@H]1CCCCN(C)C1] | H | NH |
| 28d | 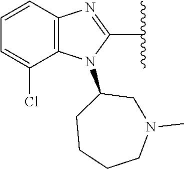 [SMILES: *c1nc2cccc(Cl)c2n1[C@@H]1CCCCN(C)C1] | Me | NH |
| 28e | 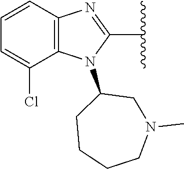 [SMILES: *c1nc2cccc(Cl)c2n1[C@@H]1CCCCN(C)C1] | H | NMe |
| 28f | 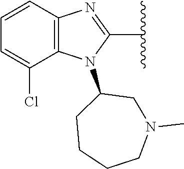 [SMILES: *c1nc2cccc(Cl)c2n1[C@@H]1CCCCN(C)C1] | Me | NMe |
| 29a | 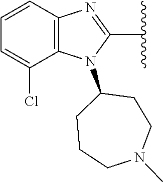 [SMILES: *c1nc2cccc(Cl)c2n1[C@@H]1CCCN(C)CC1] | H | O |
| 29b | 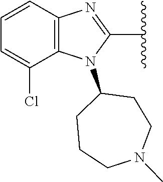 [SMILES: *c1nc2cccc(Cl)c2n1[C@@H]1CCCN(C)CC1] | Me | O |
| 29c | 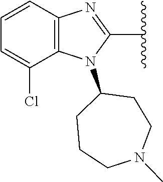 [SMILES: *c1nc2cccc(Cl)c2n1[C@@H]1CCCN(C)CC1] | H | NH |
| 29d | 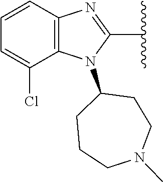 [SMILES: *c1nc2cccc(Cl)c2n1[C@@H]1CCCN(C)CC1] | Me | NH |
| 29e | 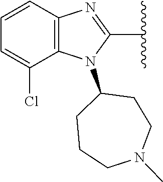 [SMILES: *c1nc2cccc(Cl)c2n1[C@@H]1CCCN(C)CC1] | H | NMe |
| 29f | 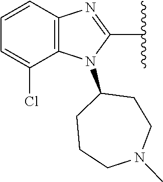 [SMILES: *c1nc2cccc(Cl)c2n1[C@@H]1CCCN(C)CC1] | Me | NMe |
| 45a | 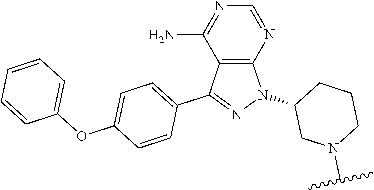 [SMILES: *N1CCC[C@@H](n2nc(-c3ccc(Oc4ccccc4)cc3)c3c(N)ncnc32)C1] | N/A | O |
| 45b | 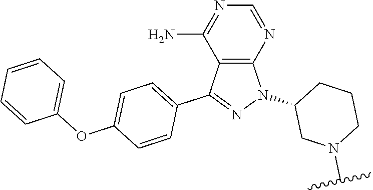 [SMILES: *N1CCC[C@@H](n2nc(-c3ccc(Oc4ccccc4)cc3)c3c(N)ncnc32)C1] | N/A | NH |
| 45c | 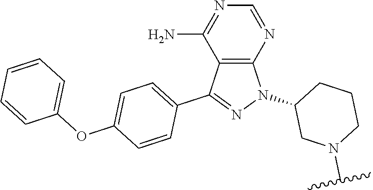 [SMILES: *N1CCC[C@@H](n2nc(-c3ccc(Oc4ccccc4)cc3)c3c(N)ncnc32)C1] | N/A | NMe |
| 46a | 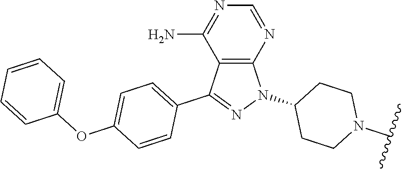 | N/A | O |
| 46b | 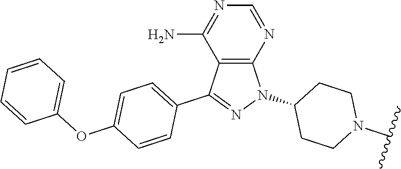 | N/A | NH |
| 46c | 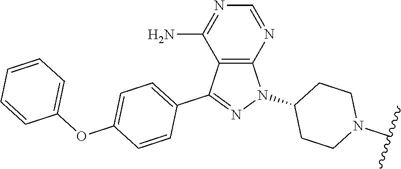 | N/A | NMe |
| 47a | 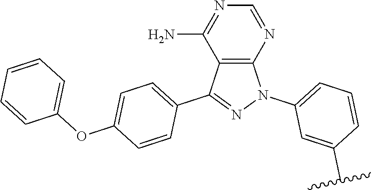 [SMILES: *c1cccc(-n2nc(-c3ccc(Oc4ccccc4)cc3)c3c(N)ncnc32)c1] | H | O |
| 47b | 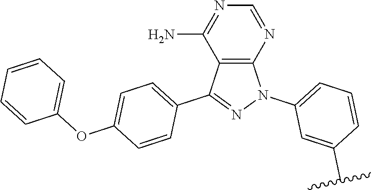 [SMILES: *c1cccc(-n2nc(-c3ccc(Oc4ccccc4)cc3)c3c(N)ncnc32)c1] | Me | O |
| 47c | 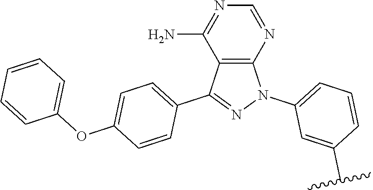 [SMILES: *c1cccc(-n2nc(-c3ccc(Oc4ccccc4)cc3)c3c(N)ncnc32)c1] | H | NH |
| 47d | 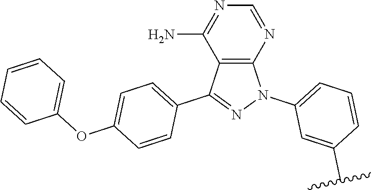 [SMILES: *c1cccc(-n2nc(-c3ccc(Oc4ccccc4)cc3)c3c(N)ncnc32)c1] | Me | NH |
| 47e | 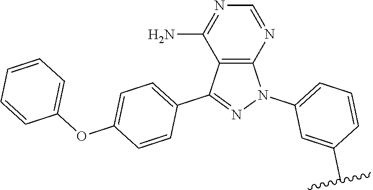 [SMILES: *c1cccc(-n2nc(-c3ccc(Oc4ccccc4)cc3)c3c(N)ncnc32)c1] | H | NMe |
| 47f | 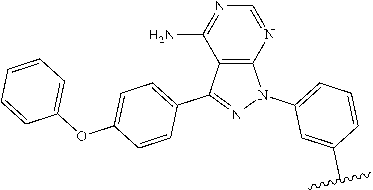 [SMILES: *c1cccc(-n2nc(-c3ccc(Oc4ccccc4)cc3)c3c(N)ncnc32)c1] | Me | NMe |
| 48a | 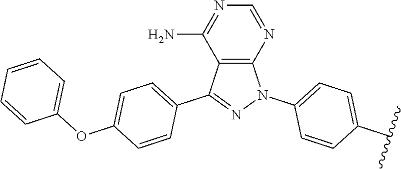 [SMILES: *c1ccc(-n2nc(-c3ccc(Oc4ccccc4)cc3)c3c(N)ncnc32)cc1] | H | O |
| 48b | 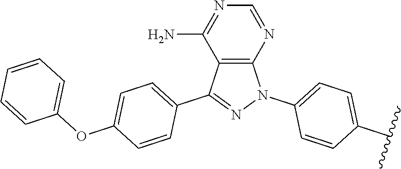 [SMILES: *c1ccc(-n2nc(-c3ccc(Oc4ccccc4)cc3)c3c(N)ncnc32)cc1] | Me | O |
| 48c | 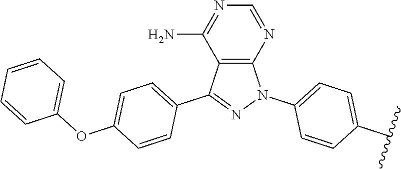 [SMILES: *c1ccc(-n2nc(-c3ccc(Oc4ccccc4)cc3)c3c(N)ncnc32)cc1] | H | NH |
| 48d | 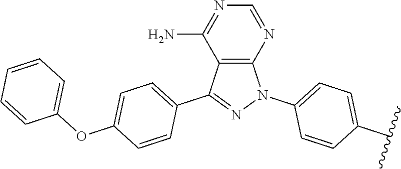 [SMILES: *c1ccc(-n2nc(-c3ccc(Oc4ccccc4)cc3)c3c(N)ncnc32)cc1] | Me | NH |
| 48e | 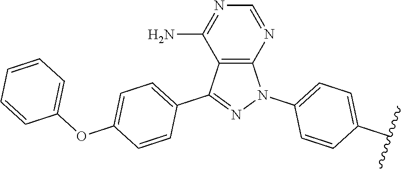 [SMILES: *c1ccc(-n2nc(-c3ccc(Oc4ccccc4)cc3)c3c(N)ncnc32)cc1] | H | NMe |
| 48f | 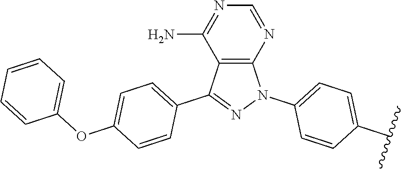 [SMILES: *c1ccc(-n2nc(-c3ccc(Oc4ccccc4)cc3)c3c(N)ncnc32)cc1] | Me | NMe |
| 49a | 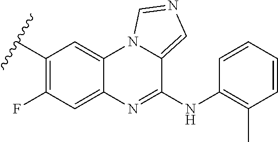 [SMILES: *c1cc2c(cc1F)nc(Nc1ccccc1C)c1cncn12] | H | O |
| 49b | 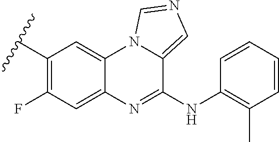 [SMILES: *c1cc2c(cc1F)nc(Nc1ccccc1C)c1cncn12] | Me | O |
| 49c | 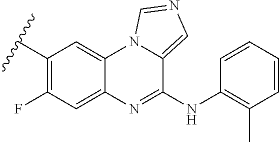 [SMILES: *c1cc2c(cc1F)nc(Nc1ccccc1C)c1cncn12] | H | NH |
| 49d | 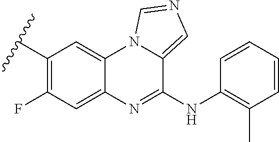 [SMILES: *c1cc2c(cc1F)nc(Nc1ccccc1C)c1cncn12] | Me | NH |
| 49e | 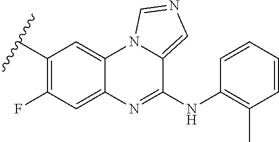 [SMILES: *c1cc2c(cc1F)nc(Nc1ccccc1C)c1cncn12] | H | NMe |
| 49f | 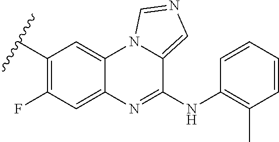 [SMILES: *c1cc2c(cc1F)nc(Nc1ccccc1C)c1cncn12] | Me | NMe |
| 50a | 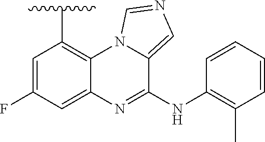 [SMILES: *c1cc(F)cc2nc(Nc3ccccc3C)c3cncn3c12] | H | O |
| 50b | 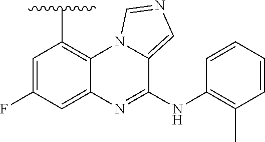 [SMILES: *c1cc(F)cc2nc(Nc3ccccc3C)c3cncn3c12] | Me | O |
| 50c | 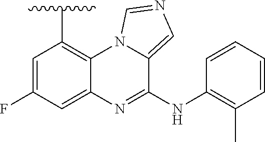 [SMILES: *c1cc(F)cc2nc(Nc3ccccc3C)c3cncn3c12] | H | NH |
| 50d | 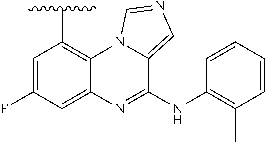 [SMILES: *c1cc(F)cc2nc(Nc3ccccc3C)c3cncn3c12] | Me | NH |
| 50e | 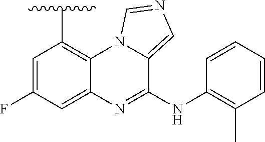 [SMILES: *c1cc(F)cc2nc(Nc3ccccc3C)c3cncn3c12] | H | NMe |
| 50f | 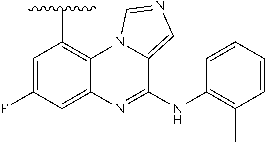 [SMILES: *c1cc(F)cc2nc(Nc3ccccc3C)c3cncn3c12] | Me | NMe |
| 66a | 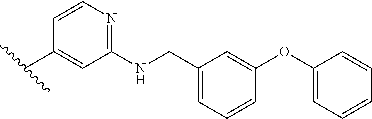 [SMILES: *c1ccnc(NCc2cccc(Oc3ccccc3)c2)c1] | H | O |
| 66b | 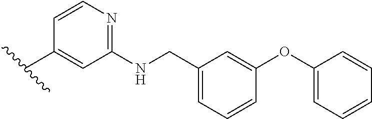 [SMILES: *c1ccnc(NCc2cccc(Oc3ccccc3)c2)c1] | Me | O |
| 66c | 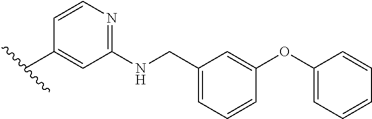 [SMILES: *c1ccnc(NCc2cccc(Oc3ccccc3)c2)c1] | H | NH |
| 66d | 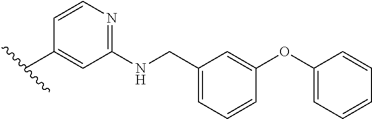 [SMILES: *c1ccnc(NCc2cccc(Oc3ccccc3)c2)c1] | Me | NH |
| 66e | 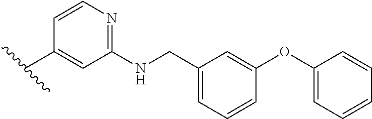 [SMILES: *c1ccnc(NCc2cccc(Oc3ccccc3)c2)c1] | H | NMe |
| 66f | 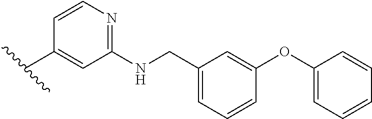 [SMILES: *c1ccnc(NCc2cccc(Oc3ccccc3)c2)c1] | Me | NMe |
| 67a | 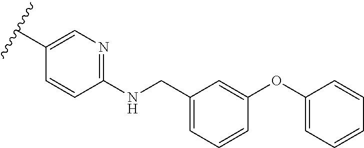 [SMILES: *c1ccc(NCc2cccc(Oc3ccccc3)c2)nc1] | H | O |
| 67b | 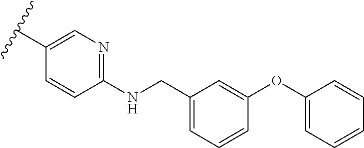 [SMILES: *c1ccc(NCc2cccc(Oc3ccccc3)c2)nc1] | Me | O |
| 670 | 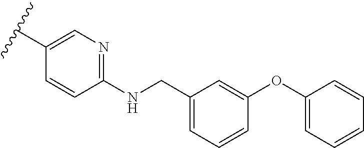 [SMILES: *c1ccc(NCc2cccc(Oc3ccccc3)c2)nc1] | H | NH |
| 67d | 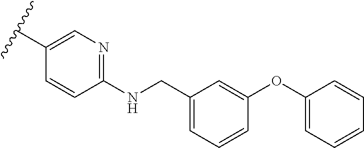 [SMILES: *c1ccc(NCc2cccc(Oc3ccccc3)c2)nc1] | Me | NH |
| 67e | 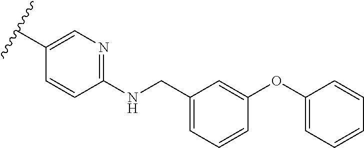 [SMILES: *c1ccc(NCc2cccc(Oc3ccccc3)c2)nc1] | H | NMe |
| 67f | 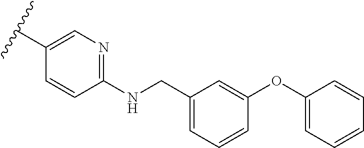 [SMILES: *c1ccc(NCc2cccc(Oc3ccccc3)c2)nc1] | Me | NMe |
| 68a | 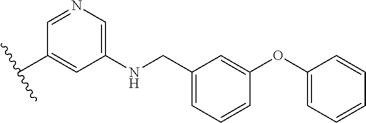 [SMILES: *c1cncc(NCc2cccc(Oc3ccccc3)c2)c1] | H | O |
| 68b | 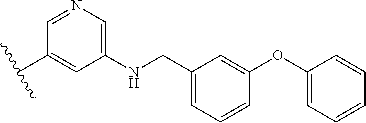 [SMILES: *c1cncc(NCc2cccc(Oc3ccccc3)c2)c1] | Me | O |
| 680 | 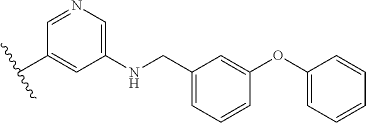 [SMILES: *c1cncc(NCc2cccc(Oc3ccccc3)c2)c1] | H | NH |
| 68d | 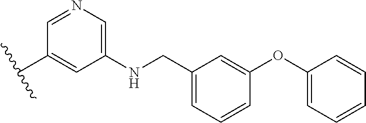 [SMILES: *c1cncc(NCc2cccc(Oc3ccccc3)c2)c1] | Me | NH |
| 68e | 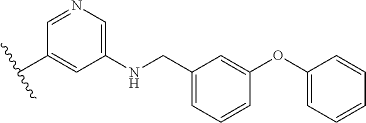 [SMILES: *c1cncc(NCc2cccc(Oc3ccccc3)c2)c1] | H | NMe |
| 68f | 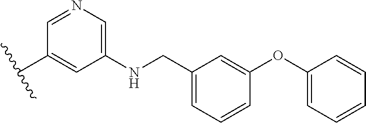 [SMILES: *c1cncc(NCc2cccc(Oc3ccccc3)c2)c1] | Me | NMe |
| 69a | 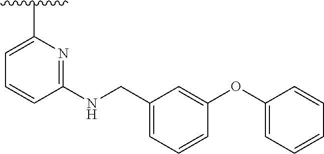 [SMILES: *c1cccc(NCc2cccc(Oc3ccccc3)c2)n1] | H | O |
| 69b | 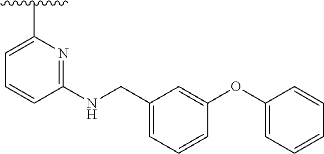 [SMILES: *c1cccc(NCc2cccc(Oc3ccccc3)c2)n1] | Me | O |
| 69c | 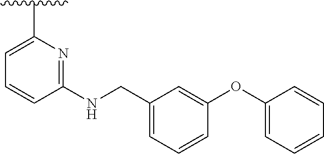 [SMILES: *c1cccc(NCc2cccc(Oc3ccccc3)c2)n1] | H | NH |
| 69d | 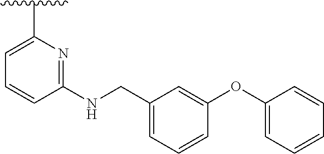 [SMILES: *c1cccc(NCc2cccc(Oc3ccccc3)c2)n1] | Me | NH |
| 69e | 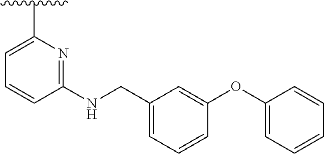 [SMILES: *c1cccc(NCc2cccc(Oc3ccccc3)c2)n1] | H | NMe |
| 69f | 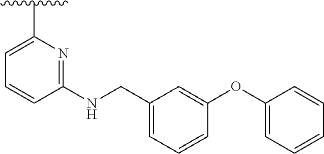 [SMILES: *c1cccc(NCc2cccc(Oc3ccccc3)c2)n1] | Me | NMe |
| 70a | 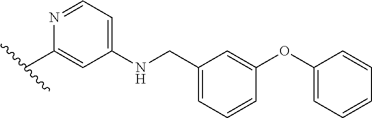 [SMILES: *c1cc(NCc2cccc(Oc3ccccc3)c2)ccn1] | H | O |
| 70b | 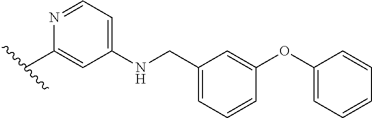 [SMILES: *c1cc(NCc2cccc(Oc3ccccc3)c2)ccn1] | Me | O |
| 70c | 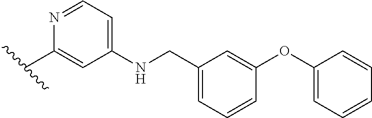 [SMILES: *c1cc(NCc2cccc(Oc3ccccc3)c2)ccn1] | H | NH |
| 70d | 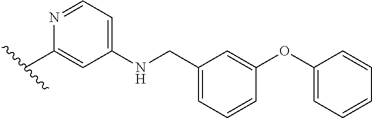 [SMILES: *c1cc(NCc2cccc(Oc3ccccc3)c2)ccn1] | Me | NH |
| 70e | 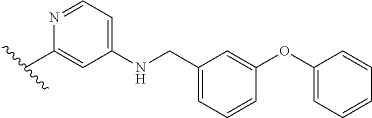 [SMILES: *c1cc(NCc2cccc(Oc3ccccc3)c2)ccn1] | H | NMe |
| 70f | 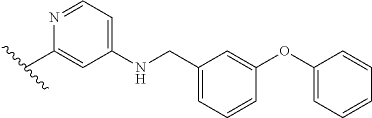 [SMILES: *c1cc(NCc2cccc(Oc3ccccc3)c2)ccn1] | Me | NMe |
| 71a | 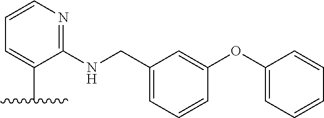 [SMILES: *c1cccnc1NCc1cccc(Oc2ccccc2)c1] | H | O |
| 71b | 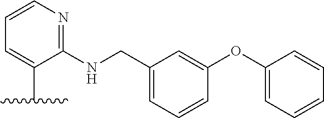 [SMILES: *c1cccnc1NCc1cccc(Oc2ccccc2)c1] | Me | O |
| 71c | 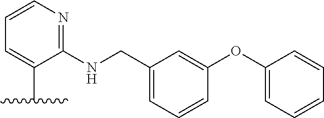 [SMILES: *c1cccnc1NCc1cccc(Oc2ccccc2)c1] | H | NH |
| 71d | 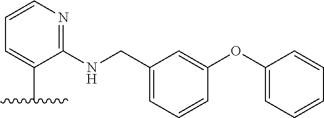 [SMILES: *c1cccnc1NCc1cccc(Oc2ccccc2)c1] | Me | NH |
| 71e | 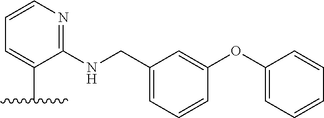 [SMILES: *c1cccnc1NCc1cccc(Oc2ccccc2)c1] | H | NMe |
| 71f | 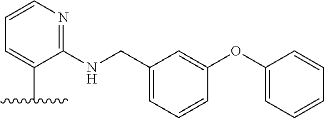 [SMILES: *c1cccnc1NCc1cccc(Oc2ccccc2)c1] | Me | NMe |
| 72a | 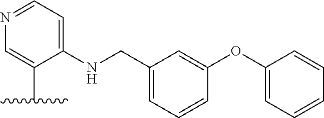 [SMILES: *c1cnccc1NCc1cccc(Oc2ccccc2)c1] | H | O |
| 72b | 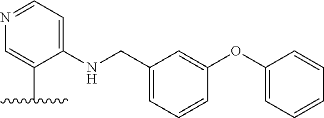 [SMILES: *c1cnccc1NCc1cccc(Oc2ccccc2)c1] | Me | O |
| 72c | 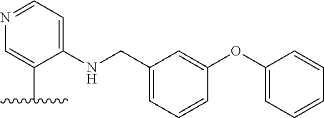 [SMILES: *c1cnccc1NCc1cccc(Oc2ccccc2)c1] | H | NH |
| 72d | 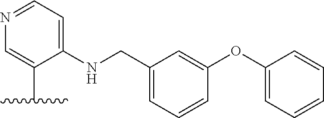 [SMILES: *c1cnccc1NCc1cccc(Oc2ccccc2)c1] | Me | NH |
| 72e | 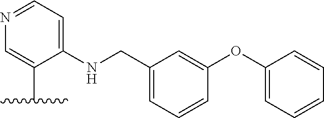 [SMILES: *c1cnccc1NCc1cccc(Oc2ccccc2)c1] | H | NMe |
| 72f | 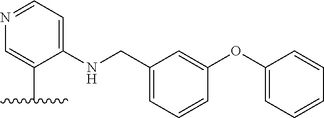 [SMILES: *c1cnccc1NCc1cccc(Oc2ccccc2)c1] | Me | NMe |
| 73a | 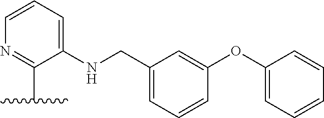 [SMILES: *c1ncccc1NCc1cccc(Oc2ccccc2)c1] | H | O |
| 73b | 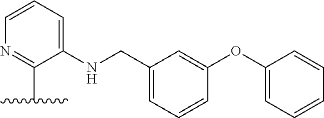 [SMILES: *c1ncccc1NCc1cccc(Oc2ccccc2)c1] | Me | O |
| 73c | 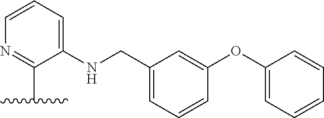 [SMILES: *c1ncccc1NCc1cccc(Oc2ccccc2)c1] | H | NH |
| 73d | 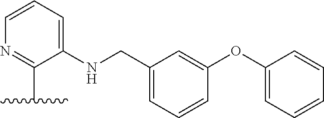 [SMILES: *c1ncccc1NCc1cccc(Oc2ccccc2)c1] | Me | NH |
| 73e | 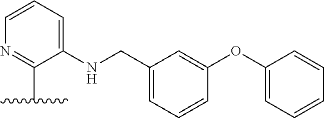 [SMILES: *c1ncccc1NCc1cccc(Oc2ccccc2)c1] | H | NMe |
| 73f | 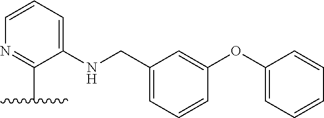 [SMILES: *c1ncccc1NCc1cccc(Oc2ccccc2)c1] | Me | NMe |
| 74a | 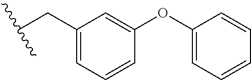 [SMILES: *Cc1cccc(Oc2ccccc2)c1] | H | O |
| 74b | 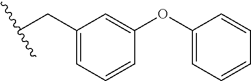 [SMILES: *Cc1cccc(Oc2ccccc2)c1] | Me | O |
| 74c | 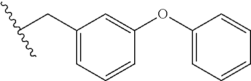 [SMILES: *Cc1cccc(Oc2ccccc2)c1] | H | NH |
| 74d | 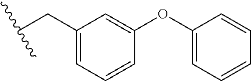 [SMILES: *Cc1cccc(Oc2ccccc2)c1] | Me | NH |
| 74e | 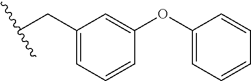 [SMILES: *Cc1cccc(Oc2ccccc2)c1] | H | NMe |
| 74f | 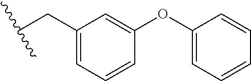 [SMILES: *Cc1cccc(Oc2ccccc2)c1] | Me | NMe |
| 75a | 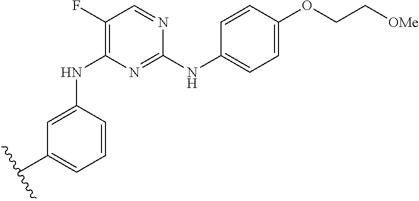 [SMILES: *c1cccc(Nc2nc(Nc3ccc(OCCOC)cc3)ncc2F)c1] | H | O |
| 75b | 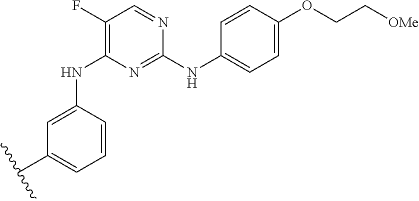 [SMILES: *c1cccc(Nc2nc(Nc3ccc(OCCOC)cc3)ncc2F)c1] | Me | O |
| 75c | 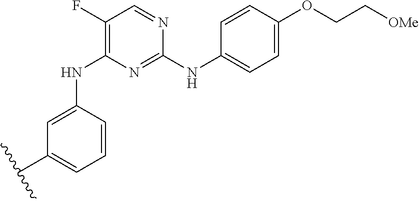 [SMILES: *c1cccc(Nc2nc(Nc3ccc(OCCOC)cc3)ncc2F)c1] | H | NH |
| 75d | 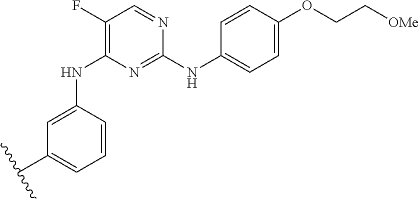 [SMILES: *c1cccc(Nc2nc(Nc3ccc(OCCOC)cc3)ncc2F)c1] | Me | NH |
| 75e | 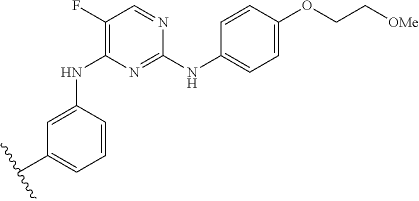 [SMILES: *c1cccc(Nc2nc(Nc3ccc(OCCOC)cc3)ncc2F)c1] | H | NMe |
| 75f | 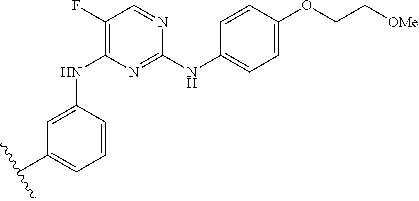 [SMILES: *c1cccc(Nc2nc(Nc3ccc(OCCOC)cc3)ncc2F)c1] | Me | NMe |
| 76a | 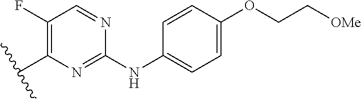 [SMILES: *c1nc(Nc2ccc(OCCOC)cc2)ncc1F] | H | O |
| 76b | 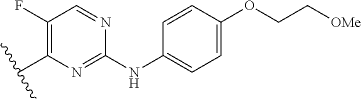 [SMILES: *c1nc(Nc2ccc(OCCOC)cc2)ncc1F] | Me | O |
| 76c | 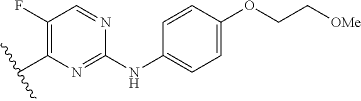 [SMILES: *c1nc(Nc2ccc(OCCOC)cc2)ncc1F] | H | NH |
| 76d | 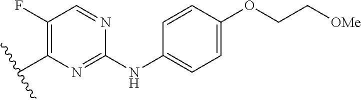 [SMILES: *c1nc(Nc2ccc(OCCOC)cc2)ncc1F] | Me | NH |
| 76e | 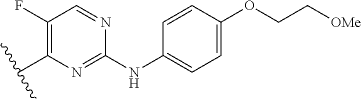 [SMILES: *c1nc(Nc2ccc(OCCOC)cc2)ncc1F] | H | NMe |
| 76f | 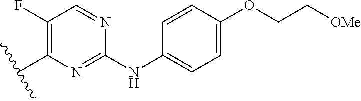 [SMILES: *c1nc(Nc2ccc(OCCOC)cc2)ncc1F] | Me | NMe |
| 77a | 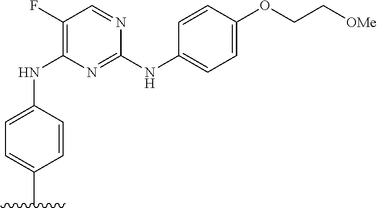 [SMILES: *c1ccc(Nc2nc(Nc3ccc(OCCOC)cc3)ncc2F)cc1] | H | O |
| 77b | 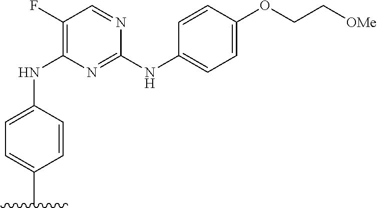 [SMILES: *c1ccc(Nc2nc(Nc3ccc(OCCOC)cc3)ncc2F)cc1] | Me | O |
| 77c | 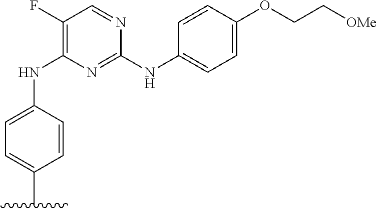 [SMILES: *c1ccc(Nc2nc(Nc3ccc(OCCOC)cc3)ncc2F)cc1] | H | NH |
| 77d | 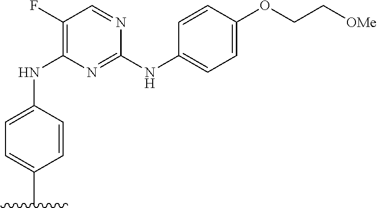 [SMILES: *c1ccc(Nc2nc(Nc3ccc(OCCOC)cc3)ncc2F)cc1] | Me | NH |
| 77e | 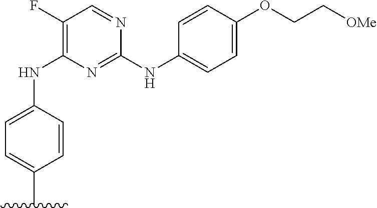 [SMILES: *c1ccc(Nc2nc(Nc3ccc(OCCOC)cc3)ncc2F)cc1] | H | NMe |
| 77f | 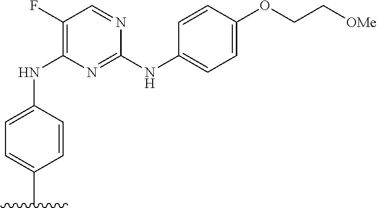 [SMILES: *c1ccc(Nc2nc(Nc3ccc(OCCOC)cc3)ncc2F)cc1] | Me | NMe |
| 78a | 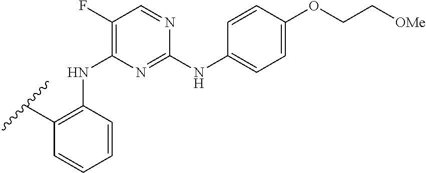 [SMILES: *c1ccccc1Nc1nc(Nc2ccc(OCCOC)cc2)ncc1F] | H | O |
| 78b | 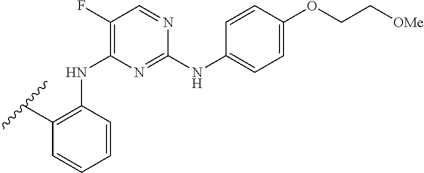 [SMILES: *c1ccccc1Nc1nc(Nc2ccc(OCCOC)cc2)ncc1F] | Me | O |
| 78c | 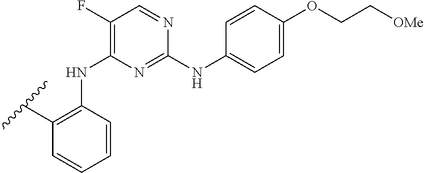 [SMILES: *c1ccccc1Nc1nc(Nc2ccc(OCCOC)cc2)ncc1F] | H | NH |
| 78d | 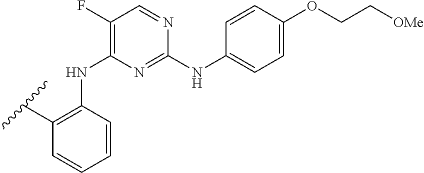 [SMILES: *c1ccccc1Nc1nc(Nc2ccc(OCCOC)cc2)ncc1F] | Me | NH |
| 78e | 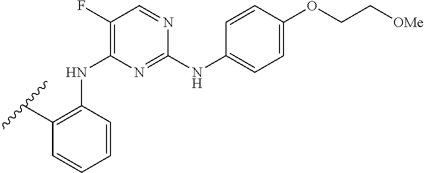 [SMILES: *c1ccccc1Nc1nc(Nc2ccc(OCCOC)cc2)ncc1F] | H | NMe |
| 78f | 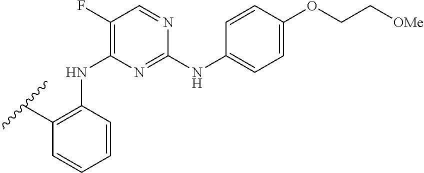 [SMILES: *c1ccccc1Nc1nc(Nc2ccc(OCCOC)cc2)ncc1F] | Me | NMe |

In some embodiments, disclosed herein is a pharmaceutically acceptable salt, solvate, tautomer, regioisomer, or stereoisomer of a compound of Table 2.

In some embodiments, the benzenesulfonamide derivative compound described herein has a structure provided in Table 3. For the benzenesulfonamide derivative compound in Table 3, R¹, R², Y¹, Y², and Y³ are as described in Table 1.

TABLE 3

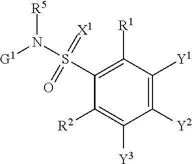

| # | 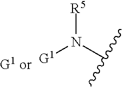 | R⁵ | X¹ |
|---|---|---|---|
| 4a | 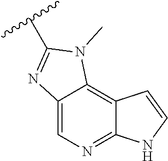 [SMILES: *c1nc2cnc3[nH]ccc3c2n1C] | H | O |
| 5a | 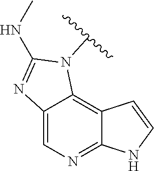 [SMILES: *n1c(NC)nc2cnc3[nH]ccc3c21] | N/A | O |
| 6a | 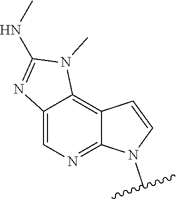 [SMILES: *n1ccc2c1ncc1nc(NC)n(C)c12] | N/A | O |
| 7a | 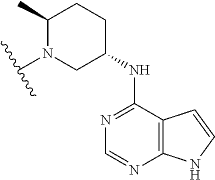 [SMILES: *N1C[C@@H](Nc2ncnc3[nH]ccc23)CC[C@@H]1C] | N/A | O |
| 8a | 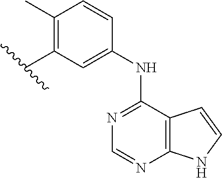 [SMILES: *c1cc(Nc2ncnc3[nH]ccc23)ccc1C] | H | O |
| 21a | 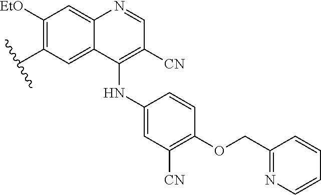 [SMILES: *c1cc2c(Nc3ccc(OCc4ccccn4)c(C#N)c3)c(C#N)cnc2cc1OCC] | H | O |
| 26a | 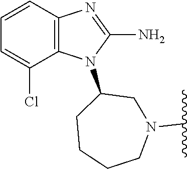 [SMILES: *N1CCCC[C@@H](n2c(N)nc3cccc(Cl)c32)C1] | N/A | O |
| 49a | 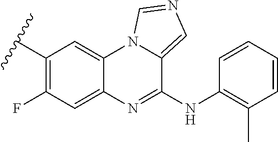 [SMILES: *c1cc2c(cc1F)nc(Nc1ccccc1C)c1cncn12] | H | O |

In some embodiments, disclosed herein is a pharmaceutically acceptable salt, solvate, tautomer, regioisomer, or stereoisomer of a compound of Table 3.

In some embodiments, the compound described herein has a structure provided in Table 4.

TABLE 4

| Compound | |
|---|---|
| Number | Structure |
| I-1 | 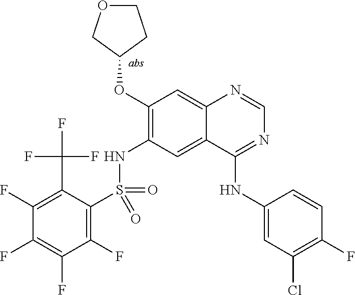 [SMILES: O=S(=O)(Nc1cc2c(Nc3ccc(F)c(Cl)c3)ncnc2cc1O[C@H]1CCOC1)c1c(F)c(F)c(F)c(F)c1C(F)(F)F] |
| I-2 | 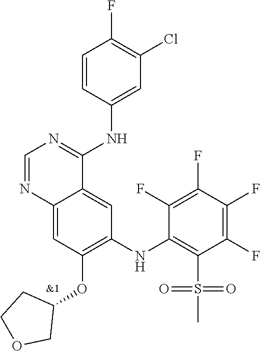 [SMILES: CS(=O)(=O)c1c(F)c(F)c(F)c(F)c1Nc1cc2c(Nc3ccc(F)c(Cl)c3)ncnc2cc1O[C@H]1CCOC1] |
| I-3 | 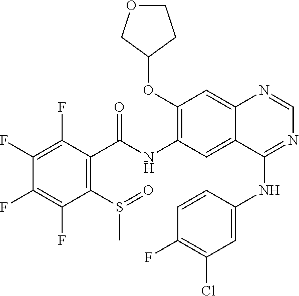 [SMILES: CS(=O)c1c(F)c(F)c(F)c(F)c1C(=O)Nc1cc2c(Nc3ccc(F)c(Cl)c3)ncnc2cc1OC1CCOC1] |
| I-4 | 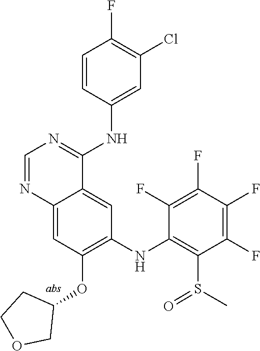 [SMILES: CS(=O)c1c(F)c(F)c(F)c(F)c1Nc1cc2c(Nc3ccc(F)c(Cl)c3)ncnc2cc1O[C@H]1CCOC1] |
| I-5 | 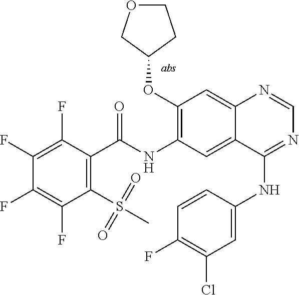 [SMILES: CS(=O)(=O)c1c(F)c(F)c(F)c(F)c1C(=O)Nc1cc2c(Nc3ccc(F)c(Cl)c3)ncnc2cc1O[C@H]1CCOC1] |

In some embodiments, the compound described herein has a structure provided in Table 5.

TABLE 5

| Compound | |
|---|---|
| Number | Structure |
| I-6 | 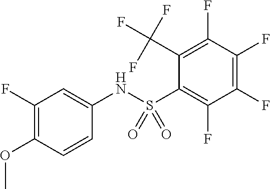 [SMILES: COc1ccc(NS(=O)(=O)c2c(F)c(F)c(F)c(F)c2C(F)(F)F)cc1F] |
| I-7 | 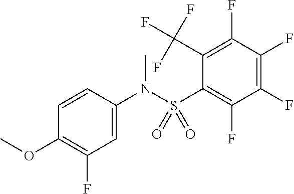 [SMILES: COc1ccc(N(C)S(=O)(=O)c2c(F)c(F)c(F)c(F)c2C(F)(F)F)cc1F] |
| I-8 | 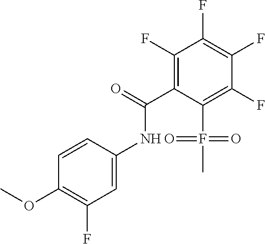 |
| I-9 | 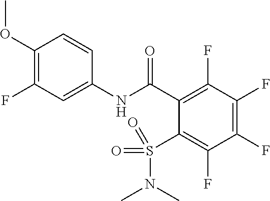 [SMILES: COc1ccc(NC(=O)c2c(F)c(F)c(F)c(F)c2S(=O)(=O)N(C)C)cc1F] |
| I-10 | 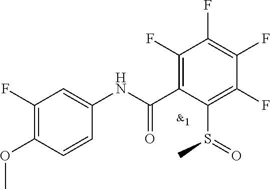 [SMILES: COc1ccc(NC(=O)c2c(F)c(F)c(F)c(F)c2[S@](C)=O)cc1F] |
| I-11 | 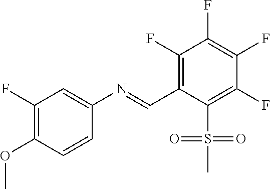 [SMILES: COc1ccc(/N=C/c2c(F)c(F)c(F)c(F)c2S(C)(=O)=O)cc1F] |
| I-12 | 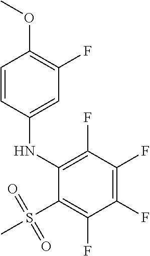 [SMILES: COc1ccc(Nc2c(F)c(F)c(F)c(F)c2S(C)(=O)=O)cc1F] |
| I-13 | 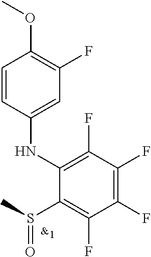 [SMILES: COc1ccc(Nc2c(F)c(F)c(F)c(F)c2[S@](C)=O)cc1F] |
| I-14 | 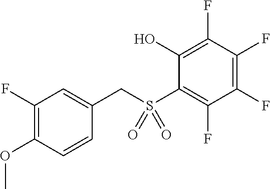 [SMILES: COc1ccc(CS(=O)(=O)c2c(O)c(F)c(F)c(F)c2F)cc1F] |
| I-15 | 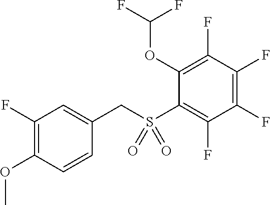 [SMILES: COc1ccc(CS(=O)(=O)c2c(F)c(F)c(F)c(F)c2OC(F)F)cc1F] |
| I-16 | 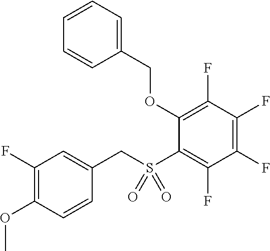 [SMILES: COc1ccc(CS(=O)(=O)c2c(F)c(F)c(F)c(F)c2OCc2ccccc2)cc1F] |
| I-17 | 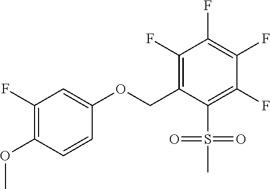 [SMILES: COc1ccc(OCc2c(F)c(F)c(F)c(F)c2S(C)(=O)=O)cc1F] |
| I-18 | 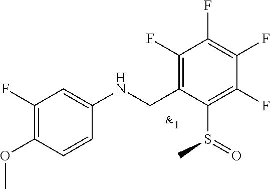 [SMILES: COc1ccc(NCc2c(F)c(F)c(F)c(F)c2[S@](C)=O)cc1F] |
| I-19 | 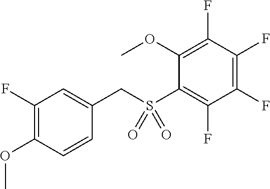 [SMILES: COc1ccc(CS(=O)(=O)c2c(F)c(F)c(F)c(F)c2OC)cc1F] |
| I-20 | 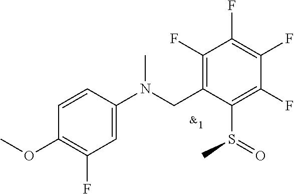 [SMILES: COc1ccc(N(C)Cc2c(F)c(F)c(F)c(F)c2[S@](C)=O)cc1F] |
| I-21 | 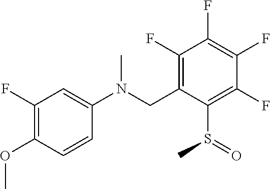 [SMILES: COc1ccc(N(C)Cc2c(F)c(F)c(F)c(F)c2[S@](C)=O)cc1F] |

In some embodiments, the compound described herein has a structure provided in Table 6.

TABLE 6

| Compound | |
|---|---|
| Number | Structure |
| I-22 | 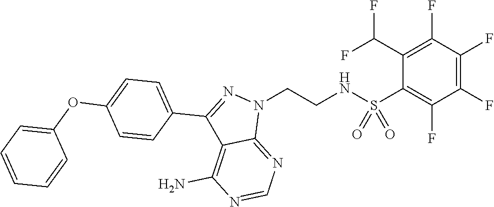 [SMILES: Nc1ncnc2c1c(-c1ccc(Oc3ccccc3)cc1)nn2CCNS(=O)(=O)c1c(F)c(F)c(F)c(F)c1C(F)F] |
| I-23 | 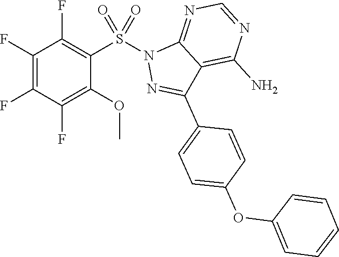 [SMILES: COc1c(F)c(F)c(F)c(F)c1S(=O)(=O)n1nc(-c2ccc(Oc3ccccc3)cc2)c2c(N)ncnc21] |
| I-24 | 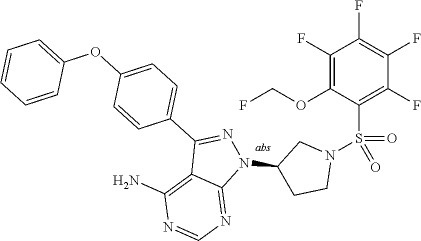 [SMILES: Nc1ncnc2c1c(-c1ccc(Oc3ccccc3)cc1)nn2[C@@H]1CCN(S(=O)(=O)c2c(F)c(F)c(F)c(F)c2OCF)C1] |
| I-25 | 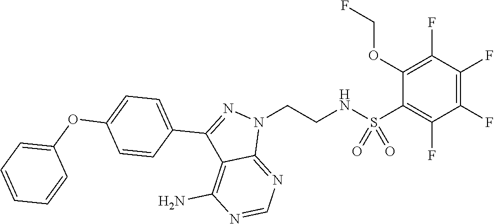 [SMILES: Nc1ncnc2c1c(-c1ccc(Oc3ccccc3)cc1)nn2CCNS(=O)(=O)c1c(F)c(F)c(F)c(F)c1OCF] |
| I-26 | 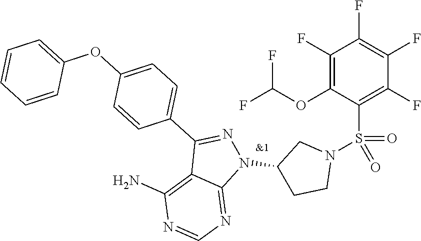 [SMILES: Nc1ncnc2c1c(-c1ccc(Oc3ccccc3)cc1)nn2[C@H]1CCN(S(=O)(=O)c2c(F)c(F)c(F)c(F)c2OC(F)F)C1] |
| I-27 | 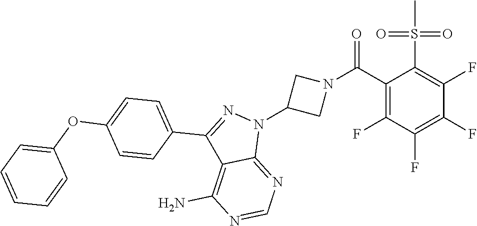 [SMILES: CS(=O)(=O)c1c(F)c(F)c(F)c(F)c1C(=O)N1CC(n2nc(-c3ccc(Oc4ccccc4)cc3)c3c(N)ncnc32)C1] |
| I-28 | 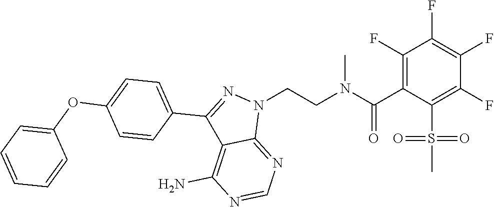 [SMILES: CN(CCn1nc(-c2ccc(Oc3ccccc3)cc2)c2c(N)ncnc21)C(=O)c1c(F)c(F)c(F)c(F)c1S(C)(=O)=O] |
| I-29 | 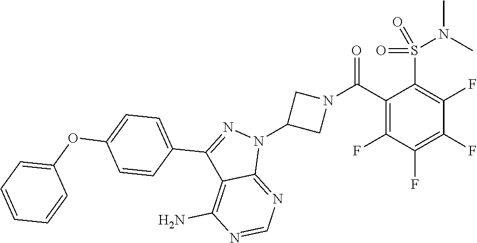 [SMILES: CN(C)S(=O)(=O)c1c(F)c(F)c(F)c(F)c1C(=O)N1CC(n2nc(-c3ccc(Oc4ccccc4)cc3)c3c(N)ncnc32)C1] |
| I-30 | 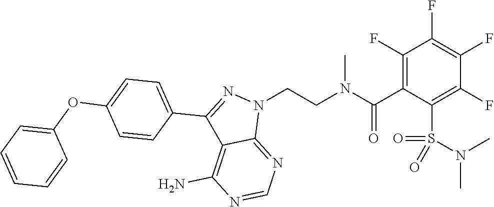 [SMILES: CN(CCn1nc(-c2ccc(Oc3ccccc3)cc2)c2c(N)ncnc21)C(=O)c1c(F)c(F)c(F)c(F)c1S(=O)(=O)N(C)C] |
| I-31 | 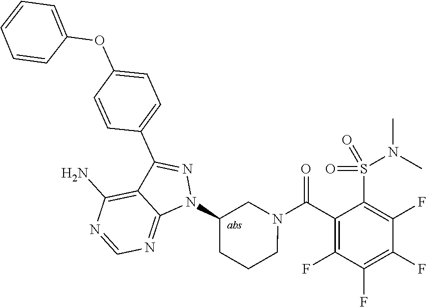 [SMILES: CN(C)S(=O)(=O)c1c(F)c(F)c(F)c(F)c1C(=O)N1CCC[C@@H](n2nc(-c3ccc(Oc4ccccc4)cc3)c3c(N)ncnc32)C1] |
| I-32 | 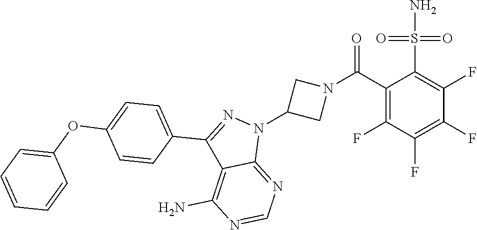 [SMILES: Nc1ncnc2c1c(-c1ccc(Oc3ccccc3)cc1)nn2C1CN(C(=O)c2c(F)c(F)c(F)c(F)c2S(N)(=O)=O)C1] |
| I-33 | 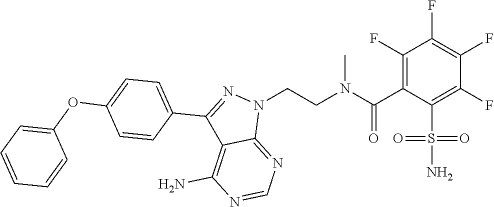 [SMILES: CN(CCn1nc(-c2ccc(Oc3ccccc3)cc2)c2c(N)ncnc21)C(=O)c1c(F)c(F)c(F)c(F)c1S(N)(=O)=O] |
| I-34 | 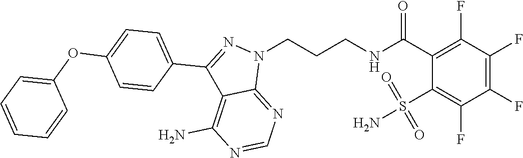 [SMILES: Nc1ncnc2c1c(-c1ccc(Oc3ccccc3)cc1)nn2CCCNC(=O)c1c(F)c(F)c(F)c(F)c1S(N)(=O)=O] |
| I-35 | 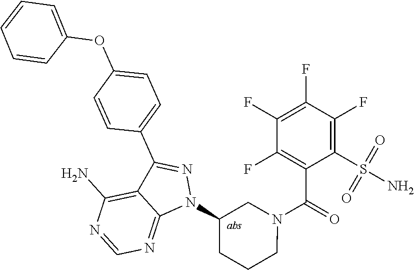 [SMILES: Nc1ncnc2c1c(-c1ccc(Oc3ccccc3)cc1)nn2[C@@H]1CCCN(C(=O)c2c(F)c(F)c(F)c(F)c2S(N)(=O)=O)C1] |
| I-36 | 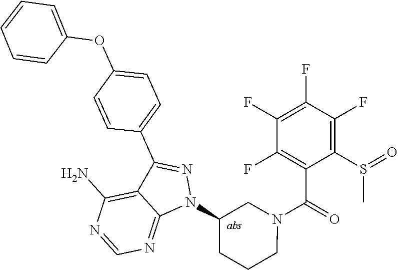 [SMILES: CS(=O)c1c(F)c(F)c(F)c(F)c1C(=O)N1CCC[C@@H](n2nc(-c3ccc(Oc4ccccc4)cc3)c3c(N)ncnc32)C1] |
| I-37 | 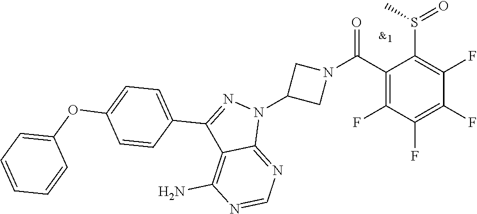 [SMILES: C[S@](=O)c1c(F)c(F)c(F)c(F)c1C(=O)N1CC(n2nc(-c3ccc(Oc4ccccc4)cc3)c3c(N)ncnc32)C1] |
| I-38 | 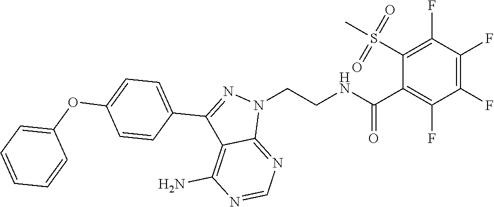 [SMILES: CS(=O)(=O)c1c(F)c(F)c(F)c(F)c1C(=O)NCCn1nc(-c2ccc(Oc3ccccc3)cc2)c2c(N)ncnc21] |
| I-39 | 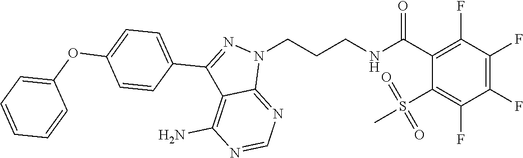 [SMILES: CS(=O)(=O)c1c(F)c(F)c(F)c(F)c1C(=O)NCCCn1nc(-c2ccc(Oc3ccccc3)cc2)c2c(N)ncnc21] |
| I-40 | 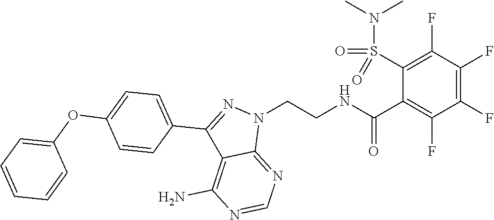 [SMILES: CN(C)S(=O)(=O)c1c(F)c(F)c(F)c(F)c1C(=O)NCCn1nc(-c2ccc(Oc3ccccc3)cc2)c2c(N)ncnc21] |
| I-41 | 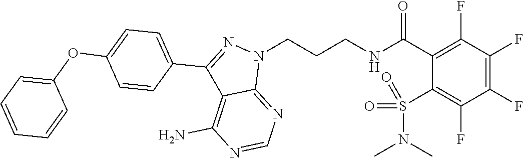 [SMILES: CN(C)S(=O)(=O)c1c(F)c(F)c(F)c(F)c1C(=O)NCCCn1nc(-c2ccc(Oc3ccccc3)cc2)c2c(N)ncnc21] |
| I-42 | 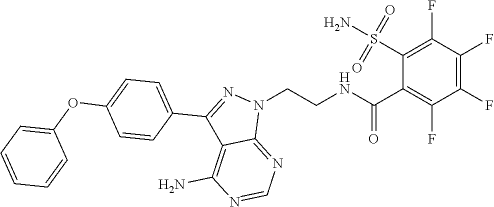 [SMILES: Nc1ncnc2c1c(-c1ccc(Oc3ccccc3)cc1)nn2CCNC(=O)c1c(F)c(F)c(F)c(F)c1S(N)(=O)=O] |
| I-43 | 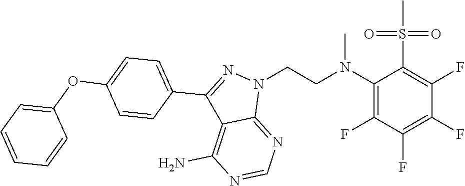 [SMILES: CN(CCn1nc(-c2ccc(Oc3ccccc3)cc2)c2c(N)ncnc21)c1c(F)c(F)c(F)c(F)c1S(C)(=O)=O] |
| I-44 | 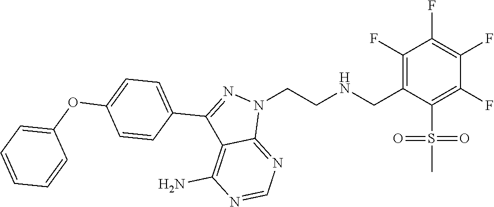 [SMILES: CS(=O)(=O)c1c(F)c(F)c(F)c(F)c1CNCCn1nc(-c2ccc(Oc3ccccc3)cc2)c2c(N)ncnc21] |

In some embodiments, the compound described herein has a structure provided in Table 7.

TABLE 7

| Compound | |
|---|---|
| Number | Structure |
| I-45 | 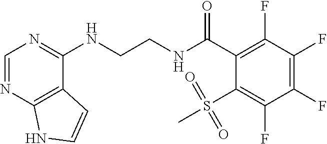 [SMILES: CS(=O)(=O)c1c(F)c(F)c(F)c(F)c1C(=O)NCCNc1ncnc2[nH]ccc12] |
| I-46 | 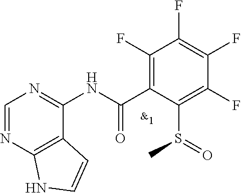 [SMILES: C[S@](=O)c1c(F)c(F)c(F)c(F)c1C(=O)Nc1ncnc2[nH]ccc12] |
| I-47 | 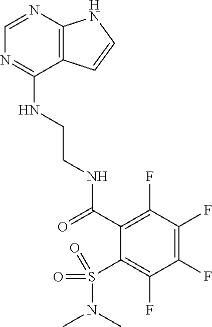 [SMILES: CN(C)S(=O)(=O)c1c(F)c(F)c(F)c(F)c1C(=O)NCCNc1ncnc2[nH]ccc12] |
| I-48 | 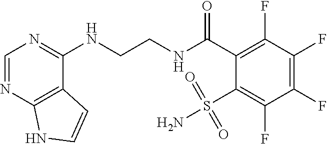 [SMILES: NS(=O)(=O)c1c(F)c(F)c(F)c(F)c1C(=O)NCCNc1ncnc2[nH]ccc12] |
| I-49 | 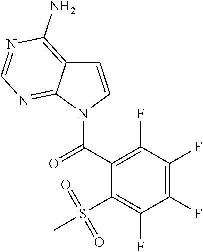 [SMILES: CS(=O)(=O)c1c(F)c(F)c(F)c(F)c1C(=O)n1ccc2c(N)ncnc21] |
| I-50 | 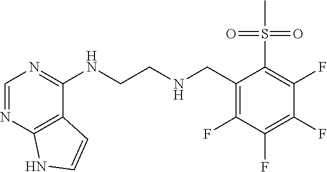 [SMILES: CS(=O)(=O)c1c(F)c(F)c(F)c(F)c1CNCCNc1ncnc2[nH]ccc12] |

In some embodiments, the compound described herein has a structure provided in Table 8.

TABLE 8

| Compound Number | Structure |
|---|---|
| I-51 | 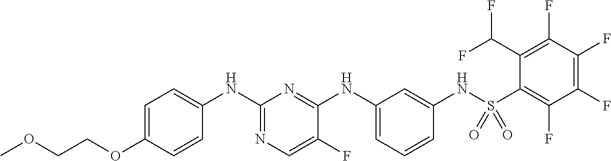 [SMILES: COCCOc1ccc(Nc2ncc(F)c(Nc3cccc(NS(=O)(=O)c4c(F)c(F)c(F)c(F)c4C(F)F)c3)n2)cc1] |
| I-52 | 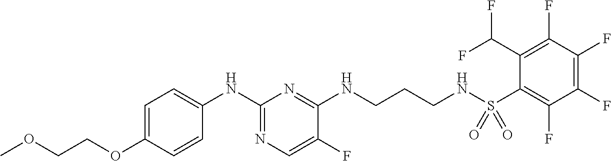 [SMILES: COCCOc1ccc(Nc2ncc(F)c(NCCCNS(=O)(=O)c3c(F)c(F)c(F)c(F)c3C(F)F)n2)cc1] |
| I-53 | 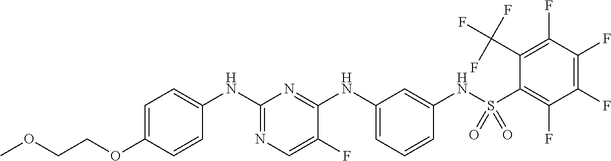 [SMILES: COCCOc1ccc(Nc2ncc(F)c(Nc3cccc(NS(=O)(=O)c4c(F)c(F)c(F)c(F)c4C(F)(F)F)c3)n2)cc1] |
| I-54 | 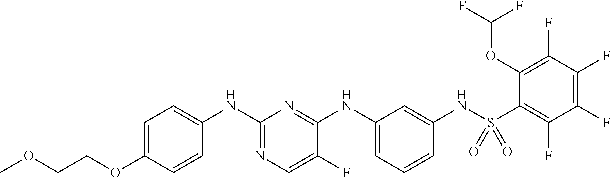 [SMILES: COCCOc1ccc(Nc2ncc(F)c(Nc3cccc(NS(=O)(=O)c4c(F)c(F)c(F)c(F)c4OC(F)F)c3)n2)cc1] |
| I-55 | 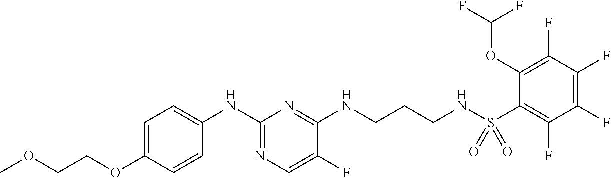 [SMILES: COCCOc1ccc(Nc2ncc(F)c(NCCCNS(=O)(=O)c3c(F)c(F)c(F)c(F)c3OC(F)F)n2)cc1] |
| I-56 | 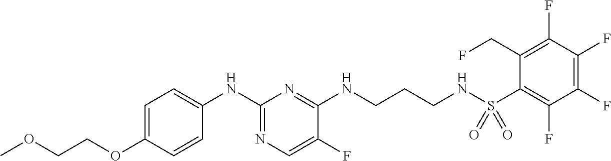 [SMILES: COCCOc1ccc(Nc2ncc(F)c(NCCCNS(=O)(=O)c3c(F)c(F)c(F)c(F)c3CF)n2)cc1] |
| I-57 | 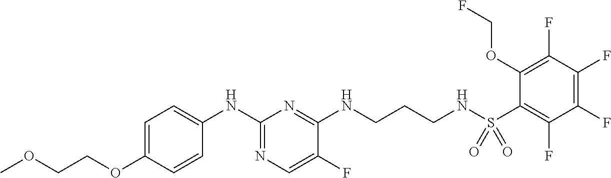 [SMILES: COCCOc1ccc(Nc2ncc(F)c(NCCCNS(=O)(=O)c3c(F)c(F)c(F)c(F)c3OCF)n2)cc1] |
| I-58 | 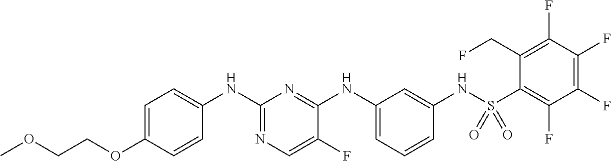 [SMILES: COCCOc1ccc(Nc2ncc(F)c(Nc3cccc(NS(=O)(=O)c4c(F)c(F)c(F)c(F)c4CF)c3)n2)cc1] |
| I-59 | 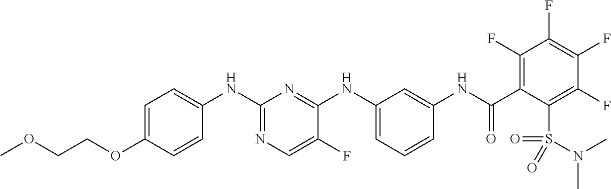 |
| I-60 | 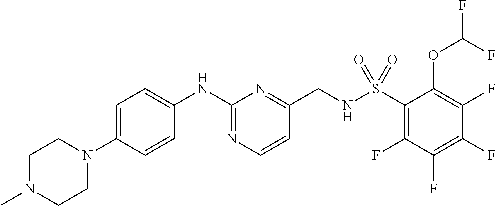 [SMILES: CN1CCN(c2ccc(Nc3nccc(CNS(=O)(=O)c4c(F)c(F)c(F)c(F)c4OC(F)F)n3)cc2)CC1] |
| I-61 | 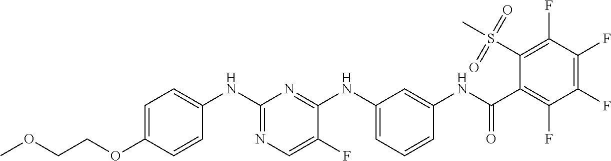 [SMILES: COCCOc1ccc(Nc2ncc(F)c(Nc3cccc(NC(=O)c4c(F)c(F)c(F)c(F)c4S(C)(=O)=O)c3)n2)cc1] |
| I-62 | 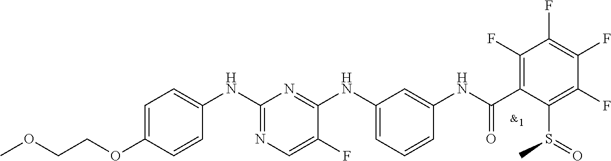 [SMILES: COCCOc1ccc(Nc2ncc(F)c(Nc3cccc(NC(=O)c4c(F)c(F)c(F)c(F)c4[S@](C)=O)c3)n2)cc1] |
| I-63 | 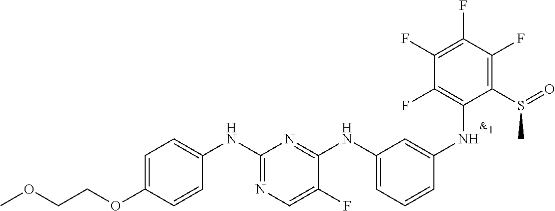 [SMILES: COCCOc1ccc(Nc2ncc(F)c(Nc3cccc(Nc4c(F)c(F)c(F)c(F)c4[S@](C)=O)c3)n2)cc1] |
| I-64 | 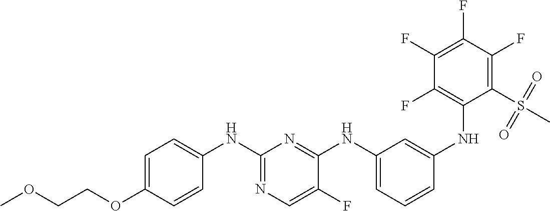 |
| I-65 | 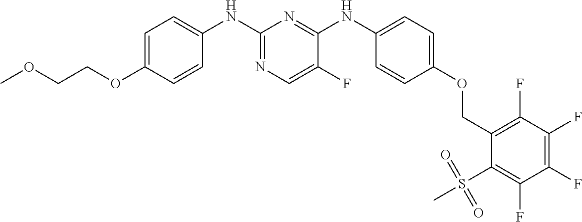 [SMILES: COCCOc1ccc(Nc2ncc(F)c(Nc3ccc(OCc4c(F)c(F)c(F)c(F)c4S(C)(=O)=O)cc3)n2)cc1] |

### Preparation of Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference useful for the preparation and selection of pharmaceutical salts of the benzenesulfonamide derivative compounds described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the benzenesulfonamide derivative compound described herein is administered as a pure chemical. In other embodiments, the benzenesulfonamide derivative compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one benzenesulfonamide derivative compound as described herein, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or the patient) of the composition.

One embodiment provides a pharmaceutical composition comprising a pharmaceutically acceptable excipient and a compound of Formula (I), or a compound disclosed in Table 1, Table 2, or Table 3, or a pharmaceutically acceptable salt, solvate, tautomer, or regioisomer thereof.

One embodiment provides a method of preparing a pharmaceutical composition comprising mixing a compound of Formula (I), or a compound disclosed in Table 1, Table 2, or Table 3, or a pharmaceutically acceptable salt, solvate, tautomer, or regioisomer thereof, and a pharmaceutically acceptable carrier.

In certain embodiments, the benzenesulfonamide derivative compound as described by Formula (I), or a compound disclosed in Table 1, Table 2, or Table 3, is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the compound as described by Formula (I), or a compound disclosed in Table 1, Table 2, or Table 3, or pharmaceutically acceptable salt, solvate, tautomer, or regioisomer thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

The dose of the composition comprising at least one compound as described herein differs depending upon the subject or patient's (e.g., human) condition. In some embodiments, such factors include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### Methods of Treatment

One embodiment provides a compound of Formula (I), or a compound disclosed in Table 1, Table 2, or Table 3, or a pharmaceutically acceptable salt, solvate, tautomer, or regioisomer thereof, for use in a method of treatment of the human or animal body.

One embodiment provides a compound of Formula (I), or a compound disclosed in Table 1, Table 2, or Table 3, or a pharmaceutically acceptable salt, solvate, tautomer, or regioisomer thereof, for use in a method of treatment of cancer or neoplastic disease.

One embodiment provides a use of a compound of Formula (I), or a compound disclosed in Table 1, Table 2, or Table 3, or a pharmaceutically acceptable salt, solvate, tautomer, or regioisomer thereof, in the manufacture of a medicament for the treatment of cancer or neoplastic disease.

In some embodiments, described herein is a method of treating cancer in a patient in need thereof comprising administering to the patient a compound of Formula (I), or a pharmaceutically acceptable salt, solvate, tautomer, or regioisomer thereof.

In some embodiments, described herein is a method of treating cancer in a patient in need thereof comprising administering to the patient a compound disclosed in Table 1, Table 2, or Table 3, or a pharmaceutically acceptable salt, solvate, tautomer, or regioisomer thereof.

In some embodiments, also described herein is a method of treating cancer in a patient in need thereof comprising administering to the patient a pharmaceutical composition comprising a compound of Formula (I), or a pharmaceutically acceptable salt, solvate, tautomer, or regioisomer thereof, and a pharmaceutically acceptable excipient.

In some embodiments, also described herein is a method of treating cancer in a patient in need thereof comprising administering to the patient a pharmaceutical composition comprising a compound disclosed in Table 1, Table 2, or Table 3, or a pharmaceutically acceptable salt, solvate, tautomer, or regioisomer thereof, and a pharmaceutically acceptable excipient. In some embodiments, the cancer is selected from chronic and acute myeloid leukemia. In some embodiments, the cancer is selected from chronic lymphocytic leukemia and small lymphocytic lymphoma.

Provided herein is the method wherein the pharmaceutical composition is administered orally. Provided herein is the method wherein the pharmaceutical composition is administered by injection.

One embodiment provides a protein, or an active fragment thereof, modified with a benzenesulfonamide derivative compound as described herein, or a pharmaceutically acceptable salt, solvate, tautomer, or regioisomer thereof, wherein the compound forms a covalent bond with a sulfur atom of a cysteine residue of the protein. In some embodiments, the protein is tubulin. In some embodiments, the protein is BTK. In some embodiments, the protein is EGFR. In some embodiments, the protein is JAK3.

One embodiment provides a method of modifying (e.g., attaching to and/or degrading) a polypeptide with a benzenesulfonamide derivative compound as described herein, or a pharmaceutically acceptable salt, solvate, tautomer, or regioisomer thereof, comprising contacting the polypeptide with the compound to form a covalent bond with a sulfur atom of a cysteine residue of the polypeptide.

One embodiment provides a method of binding a compound to a polypeptide, comprising contacting the polypeptide with a benzenesulfonamide derivative compound as described herein, or a pharmaceutically acceptable salt, solvate, tautomer, or regioisomer thereof. In some embodiments, the polypeptide is tubulin. In some embodiments, the polypeptide is BTK. In some embodiments, the polypeptide is EGFR. In some embodiments, the polypeptide is JAK3.

One embodiment provides a method of disrupting a protein, or an active fragment thereof (e.g. a function thereof), comprising contacting the protein or an active fragment thereof (e.g., polypeptide thereof) with a compound of any one of the preceding claims, or a salt, solvate, tautomer, or regioisomer thereof.

Other embodiments and uses will be apparent to one skilled in the art in light of the present disclosures. The following examples are provided merely as illustrative of various embodiments and shall not be construed to limit the present disclosure in any way.

### EXAMPLES

### I. Chemical Synthesis

In some embodiments, the benzenesulfonamide derivative compounds disclosed herein are synthesized according to the following examples. As used below, and throughout the specification, the following abbreviations, unless otherwise indicated, shall be understood to have the following meanings:
° C. degrees Celsius
δ chemical shift in parts per million downfield from tetramethylsilane
ACN acetonitrile
bs or brs broad singlet
DCM dichloromethane (CH₂Cl₂)
dd doublet of doublets
DMF dimethylformamide
DMSO dimethylsulfoxide
EA or EtOAc ethyl acetate
ESI electrospray ionization
Et ethyl
FA formic acid
g gram(s)
h/hr/hrs hour(s)
HPLC high performance liquid chromatography
Hz hertz
J coupling constant (in NMR spectrometry)
LCMS liquid chromatography mass spectrometry
μ micro
m multiplet (spectral); meter(s); milli
M molar
M⁺ parent molecular ion
Me methyl
MHz megahertz
min minute(s)
mol mole(s); molecular (as in mol wt)
mL milliliter
MS mass spectrometry
nm nanometer(s)
NMR nuclear magnetic resonance
pH potential of hydrogen; a measure of the acidity or basicity of an aqueous solution
PE petroleum ether
PMB para-methoxybenzyl
RT room temperature
s singlet (spectral)
t triplet (spectral)
T temperature
TFA trifluoroacetic acid
THF tetrahydrofuran

Exemplary compounds of the application are synthesized using the methods described herein, or other methods, which are known in the art. Unless otherwise noted, reagents and solvents are obtained from commercial suppliers.

Anhydrous solvents, methanol, acetonitrile, dichloromethane, tetrahydrofuran and dimethylformamide, are purchased from Sigma Aldrich and used directly from Sure-Seal bottles. Reactions are performed under an atmosphere of dry nitrogen in oven-dried glassware and are monitored for completeness by thin-layer chromatography (TLC) using silica gel (visualized by UV light, or developed by treatment with KMnO₄ stain and ninhydrin stain) or by LC/MS. NMR spectra are recorded in Bruker Avance III spectrometer at 23° C., unless otherwise stated, operating at 400 MHz for ¹H NMR and 376 MHz ¹⁹F NMR spectroscopy either in CDCl₃, CD₃OD, CD₃CN or DMSO-d₆. Chemical shifts (d) are reported in parts per million (ppm) after calibration to residual isotopic solvent. Coupling constants (J) are reported in Hz. Mass spectrometry was performed with an Agilent G6110A single quad mass spectrometer with an ESI source associated with an Agilent 1100 HPLC system. Before biological testing, inhibitor purity was evaluated by reversed-phase HPLC (rpHPLC). The following conditions were employed for analysis by rpHPLC:

Method I: Mobile phase is a linear gradient consisting of a changing solvent composition of 10% to 90% ACN in H₂O with 0.1% TFA (v/v) over 7 minutes, followed by 5 minutes of 100% ACN. Method was run on a Waters Atlantis 5 μm C18, 150 mm×4.6 mm column; maintained at a temperature of 30° C.; flow rate of 1.0 mL/min.

Method II: Mobile phase is a linear gradient consisting of a changing solvent composition of 10% to 90% ACN in H₂O with 0.1% Ammonia (v/v) over 7 minutes, followed by 5 minutes of 100% ACN. Method was run on a Waters Atlantis 5 m C18, 150 mm×4.6 mm column; maintained at a temperature of 30° C.; flow rate of 1.0 mL/min.

Method III: Mobile phase is a linear gradient consisting of a changing solvent composition of 15% to 100% ACN in H₂O with 0.1% TFA (v/v) over 15 minutes. Method was run on a Phenomenex Luna 5 μm C18 150 mm×4.6 mm column; column maintained at a temperature of 25° C.; flow rate of 1.0 mL/min.

For reporting HPLC data, percentage purity is given after the retention time for each condition. All biologically evaluated compounds are >95% chemical purity as measured by HPLC.

In some embodiments, compounds of the present disclosure are synthesized using similar protocols based on the general procedures A-J, and Examples 1-10 below.

### General Procedure A′

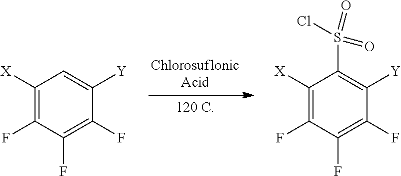

A substituted fluoro-arene (1 eq) was added to a cold solution of chlorosulfonic acid cooled to 0° C. The reaction vessel was outfitted with a water jacketed reflux condenser and subsequently heated to 120° C. using a sand bath for 1-16 hrs. Once starting material was consumed, the reaction was cooled to room temperature then poured slowly over crushed ice. The resulting mixture was partitioned between DCM and 1M HCl and the organic phase separated. The remaining aqueous phase was extracted twice more with DCM. The combined organic phases were washed with brine, dried over sodium sulfate, and concentrated in vacuo to afford the desired arylsulfonylchloride.

### General Procedure B′

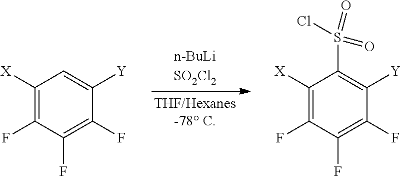

A substituted fluoro-arene (1 eq) was dissolved in anhydrous THE under a positive pressure of argon. The resulting solution was cooled to −78° C. Once at temperature, n-butyllithium (2.5 M in hexane, 1.2 eq) was added dropwise to limit excess evolution of heat. After 30 minutes, a solution of sulfuryl chloride (1.1 eq) in hexanes (0.1 M) was added quickly via syringe. After 1 hour, water was added to quench the reaction and the resulting mixture partitioned between ethyl acetate and cold water. The organic phase was separated, washed with cold water twice, dried over sodium sulfate and concentrated in vacuo to afford the anticipated sulfonylchloride.

### General Procedure C′

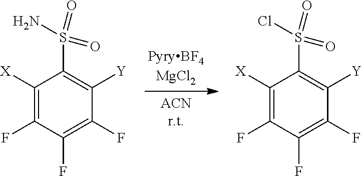

Under an inert atmosphere of argon, an appropriate aryl sulfonamide was added to a suspension of pyrylium tetrafluoroborate (2 eq) and magnesium chloride (2.5 eq) in acetonitrile (0.1 M) stirring at room temperature. The reaction was heated to 75° C. for 6 hours then cooled to room temperature. Once cooled, the mixture was filtered through a short plug of silica and the filtrate concentrated under reduced pressure. The concentrate was separated using flash column chromatography techniques to afford the desired sulfonylchloride.

### General Procedure D′

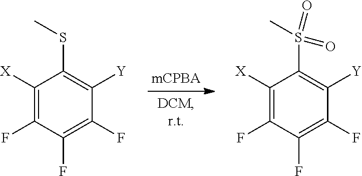

To a solution of thioether (1 eq) in DCM (0.1M-0.3M) at room temperature was added 3-Chloroperoxybenzoic acid (4 eq, 77% purity). Reaction progress was monitored by TLC. Once the starting material was consumed, the reaction was quenched with a 1M aqueous solution of sodium hydroxide. The organic phase was separated and the remaining aqueous extracted twice with dichloromethane. The combined organic phases were washed with brine, dried over anhydrous sodium sulfate and concentrated under vacuum. The crude material was separated using flash column chromatography techniques to afford the desired methylsulfone.

### Synthesis of Sulfonamides: General Procedure E′

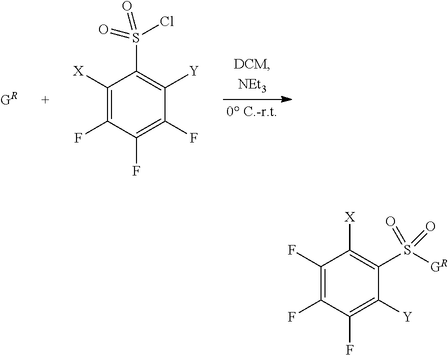

An appropriate sulfonylchloride (0.9-1.2 eq) was incubated with its corresponding pyrazololopyrimidine (1 eq) in anhydrous DCM (0.1 M-0.25 M) under an atmosphere of argon. The resulting mixture was cooled to 0° C. and stirred for 15 minutes. Neat triethylamine (3-5 eq) was slowly added to the mixture and it was stirred at 0° C. for a further 3-16 hrs. The reaction quenched with 0.1M HCl (aq) and vigorously stirred for 10-15 min, after which the organic layer was separated. The aqueous layer was extracted with DCM one further time. The combined organic layers were dried over sodium sulfate, filtered, and evaporated. The crude material was purified by either normal-phase flash column chromatography on silica gel or reverse-phase chromatography.

### Synthesis of Direct-Linked Sulfones: General Procedure F′

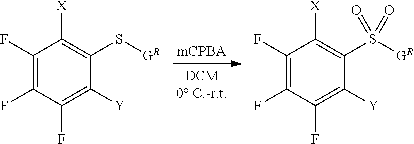

Methods to oxidize analogous thioethers to the corresponding sulfone are known in the art (WO2019/141694). The G linked sulfone can be prepared from the corresponding thioether in the presence of 3-Chloroperoxybenzoic acid (mCPBA, 4 eq.) in DCM under inert conditions (argon or nitrogen). The reaction can be worked up with water, brine and DCM, and the desired sulfone isolated using normal-phase flash column chromatography on silica gel or reverse-phase chromatography.

### Synthesis of Direct-Linked Sulfoximines: General Procedure G′

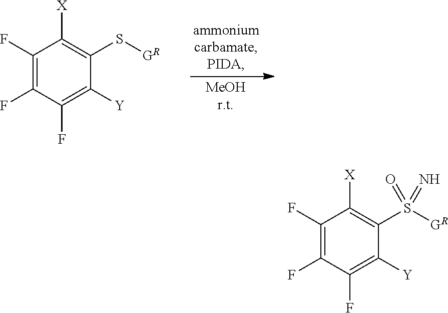

Methods to oxidize analogous thioethers to the corresponding sulfoximine are known in the art (Chem. Comm., 2017, 12, p. 2064-2067). The G linked sulfoximine can be prepared from the corresponding thioether in the presence of ammonium carbamate (1.5 eq.), iodobenzenediacetate (PIDA, 2.1 eq.) in methanol at room temperature. The reaction can be worked up with water, brine and DCM, and the desired sulfoximine isolated using normal-phase flash column chromatography on silica gel or reverse-phase chromatography.

### Synthesis of Sulfonimidamides: General Procedure H′

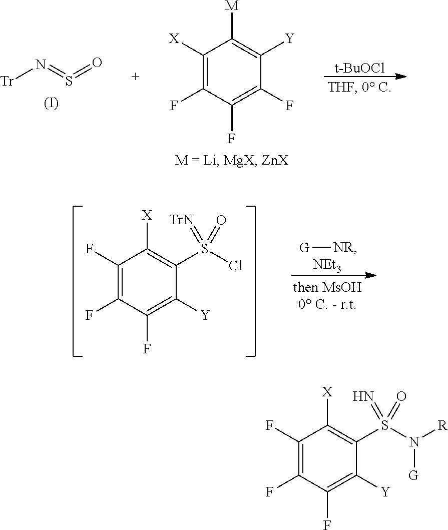

The starting material, compound (I), can be prepared according to previously reported procedures (Angew. Chem. Int. Ed. 2017, 56, 14937). An oven-dried flask charged with (I) (1.0 equiv.) and THF (0.1 M) is cooled to 0° C. Then the corresponding organometallic reagent (1.0 equiv.) can be added dropwise and stirred at 0° C. for 5 min. Next, in a dark fume hood, tert-butyl hypochlorite (1.05 equiv.) is added and the reaction mixture is allowed to stir for 15 min, followed by the addition of triethylamine (1.0 equiv.) and the corresponding ligand (G or GNR) (1.0-1.2 equiv.). The reaction mixture is left stirring at room temperature for 16 h. Finally, methanesulfonic acid (5.0 equiv.) is added, and the reaction stirred vigorously for 15 min at room temperature. The reaction is quenched by diluting it with DCM and the addition of a saturated aqueous solution of sodium bicarbonate. The two layers are partitioned and the aqueous layer is extracted with DCM (×3). Combined organic layers are dried over magnesium sulfate (MgSO₄), filtered and concentrated in vacuo. Crude samples can be purified by either normal-phase flash column chromatography on silica gel or reverse-phase chromatography.

### Synthesis of Para-Direct-Linked Sulfones: General Procedure I′

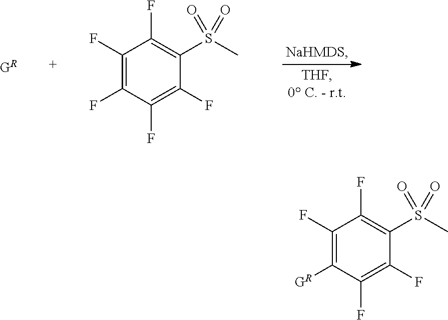

Procedures detailing the addition of a nucleophile to the 4-position of the pentafluorobenzene methyl sulfone are known (J. Chem. Soc., Perkin Trans. 2000, 4265-4278). In one embodiment, G-NHR can be deprotonated in THE using sodium hexamethyldisilazane to prepare the corresponding sodium amide. The sodium amide can then be added to a cold solution (0° C.) of 1,2,3,4,5-pentafluoro-6-(methylsulfonyl)benzene in THF to prepare the anticipated para-substituted tetrafluorobenzene sulfone. The reaction can be worked up with water, brine and EtOAc, and the desired product isolated using normal-phase flash column chromatography on silica gel or reverse-phase chromatography.

### Synthesis of Ortho-Direct-Linked Sulfones: General Procedure J′

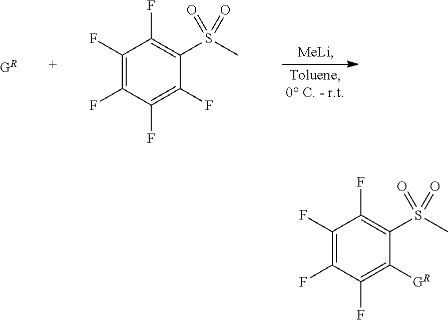

Procedures detailing the addition of a nucleophile to the 2-position of the pentafluorobenzene methyl sulfone/sulfoxide are known (Zhurnal Organicheskoi Khimii, 1980, 5, 1029-1034). In one embodiment, G-NHR can be deprotonated in THE using a strong base, such as methyl lithium, to prepare the corresponding lithium amide. The lithium amide can then be added to a cold solution (0° C.) of 1,2,3,4,5-pentafluoro-6-(methylsulfonyl)benzene in toluene to prepare the anticipated ortho-substituted tetrafluorobenzene sulfone. The reaction can be worked up with water, brine and EtOAc, and the desired product isolated using normal-phase flash column chromatography on silica gel or reverse-phase chromatography.

### Example 1: N-((3S,6S)-1-((2-(difluoromethoxy)-3,4,5,6-tetrafluorophenyl)sulfonyl)-6-methylpiperidin-3-yl)-7H-pyrrolo[2,3-]pyrimidin-4-amine (Compound 7ae Table 1)

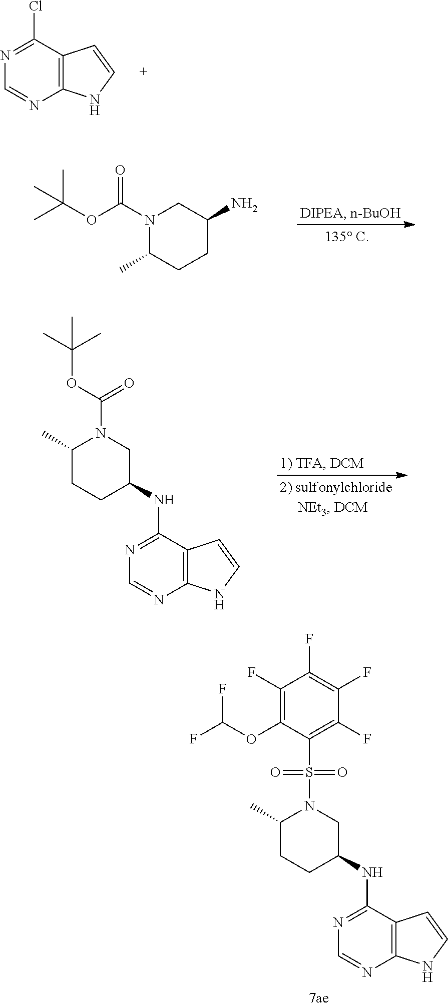 [SMILES: Clc1ncnc2[nH]ccc12 ; C[C@H]1CC[C@H](N)CN1C(=O)OC(C)(C)C ; C[C@H]1CC[C@H](Nc2ncnc3[nH]ccc23)CN1C(=O)OC(C)(C)C ; C[C@H]1CC[C@H](Nc2ncnc3[nH]ccc23)CN1S(=O)(=O)c1c(F)c(F)c(F)c(F)c1OC(F)F]

Compound 7ae, as an example for analogous routes to similar compounds, is synthesized by S_{N}Ar displacement of commercially available chloride followed by Boc-deprotection and sulfonamide formation (detailed in General Procedure E′) with the previously described sulfonyl chlorides to give final product 7ae.

### Example 2: (R)—N-(7-chloro-1-(1-methylazepan-3-yl)-1H-benzo[d]imidazol-2-yl)-2-(difluoromethyl)-3,4,5,6-tetrafluorobenzenesulfonamide and (R)-7-chloro-1-(1-((2-(difluoromethoxy)-3,4,5,6-tetrafluorophenyl)sulfonyl)azepan-3-yl)-1H-benzo[d]imidazol-2-amine (Compound 26a and 28a, Table 2)

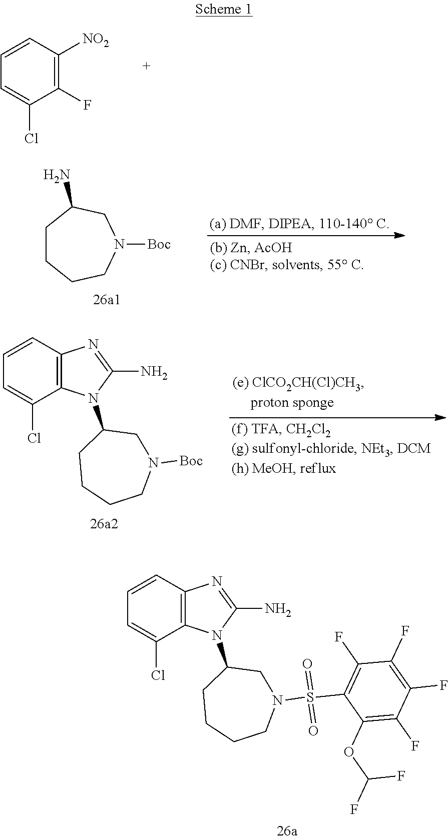 [SMILES: O=[N+]([O-])c1cccc(Cl)c1F ; CC(C)(C)OC(=O)N1CCCC[C@@H](N)C1 ; CC(C)(C)OC(=O)N1CCCC[C@@H](n2c(N)nc3cccc(Cl)c32)C1 ; Nc1nc2cccc(Cl)c2n1[C@@H]1CCCCN(S(=O)(=O)c2c(F)c(F)c(F)c(F)c2OC(F)F)C1]

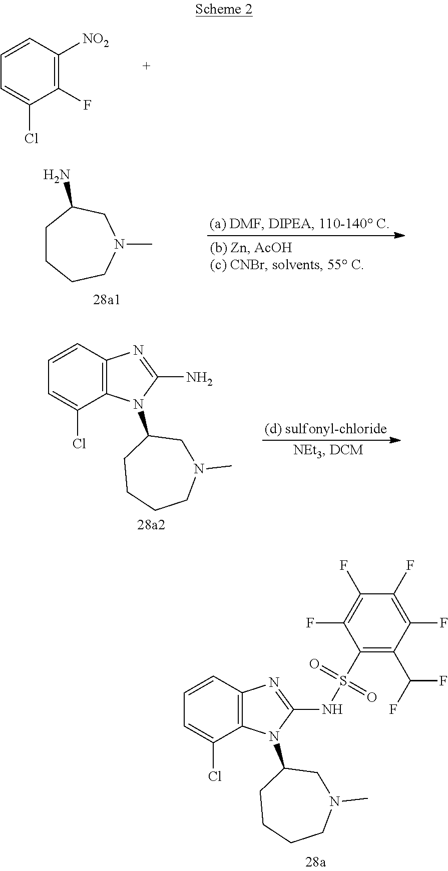

Compound 26a2 (Scheme 1) or 28a2 (Scheme 2), for example, can be prepared as described in Journal of Medicinal Chemistry 2016 59 (14), 6671-6689 by treatment of 1-chloro-2-fluoro-3-nitrobenzene with 26a1 or 28a1 or appropriately protected diamines, followed by nitro reduction and cyclization with cyanogen bromide to afford 2-aminobenzimidazole intermediates 26a2 and 28a2. Reaction of 28a2 with previously prepared sulfonyl chlorides (General Procedure E) will prepare final compound 28a. Similarly, a protection of amine 26a2 as the 1-chloro-ethylcarbamate, followed by Boc-deprotection, coupling with the appropriate sulfonyl chloride, using General Procedure G′, and final deprotection of the carbamate furnishes compound 26a.

### Example 3: (S)-3-(4-phenoxyphenyl)-1-(1-((2,3,4,5-tetrafluoro-6-(fluoromethyl)phenyl)sulfonyl)piperidin-3-yl)-1H-pyrazolo[3,4-d]pyrimidin-4-amine (Compound 45a)

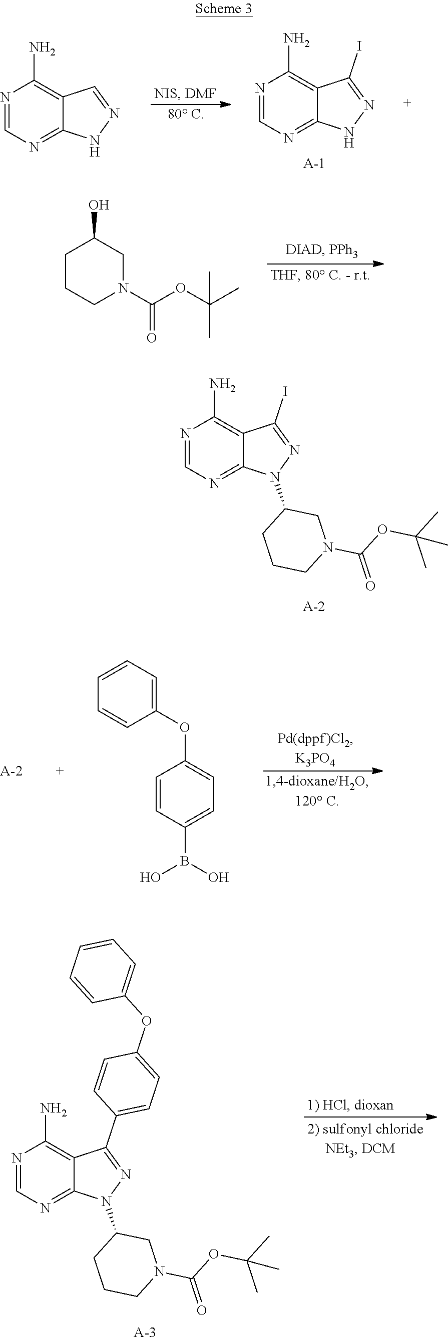

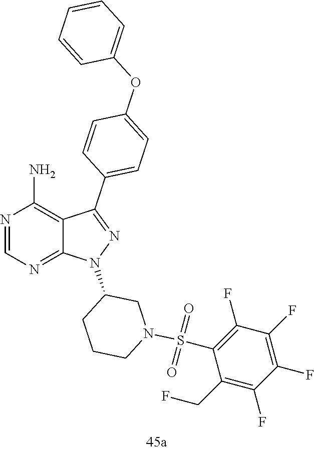 [SMILES: Nc1ncnc2c1c(-c1ccc(Oc3ccccc3)cc1)nn2[C@H]1CCCN(S(=O)(=O)c2c(F)c(F)c(F)c(F)c2CF)C1]

Compound 45a and related examples can be prepared according to the route in Scheme 3 starting from commercially available 1H-Pyrazolo[3,4-d]pyrimidin-4-amine. Iodination using NIS, followed by Mitsunobu functionalization with cyclic and acyclic alcohols provides the appropriate intermediate, example A-2. Suzuki coupling followed by Boc-deprotection and sulfonamide formation according to General Procedure G′ affords the product example 45a.

### Example 4: N-(3-(4-amino-3-(4-phenoxyphenyl)-1H-pyrazolo[3,4-d]pyrimidin-1-yl)phenyl)-2,3,4,5-tetrafluoro-6-(trifluoromethoxy)benzenesulfonamide (Compound 47a)

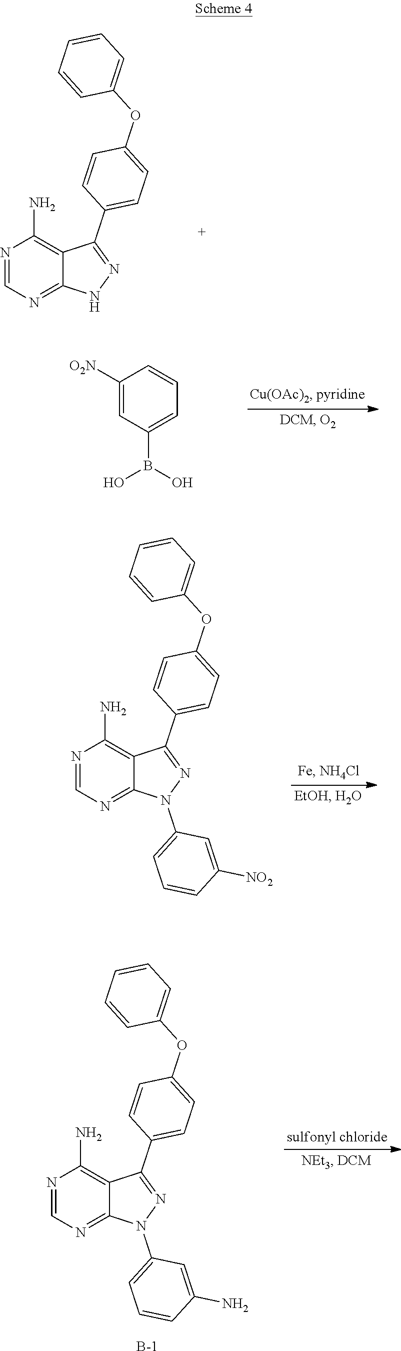

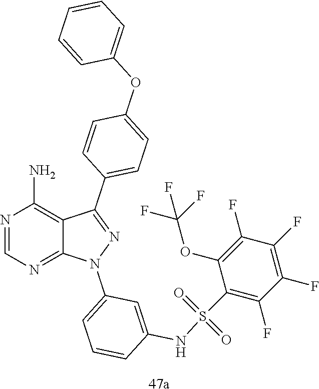

Compound 47a and related examples can be prepared via Scheme 4 starting from commercially available 3-(4-phenoxyphenyl)-1H-pyrazolo[3,4-d]pyrimidin-4-amine. Copper mediated cross-coupling with (3-nitrophenyl)boronic acid, followed by reduction of the installed nitro-group affords intermediate B-1 which can be further derivatized. Sulfonamide formation using General Procedure G affords the final products 47a.

### Example 5: 5-fluoro-N⁴-(4-(2-methoxyethoxy)phenyl)-N²-(3-((2,3,4,5-tetrafluoro-6-(methylsulfonyl)phenyl)amino)phenyl)pyrimidine-2,4-diamine (Compound 75a)

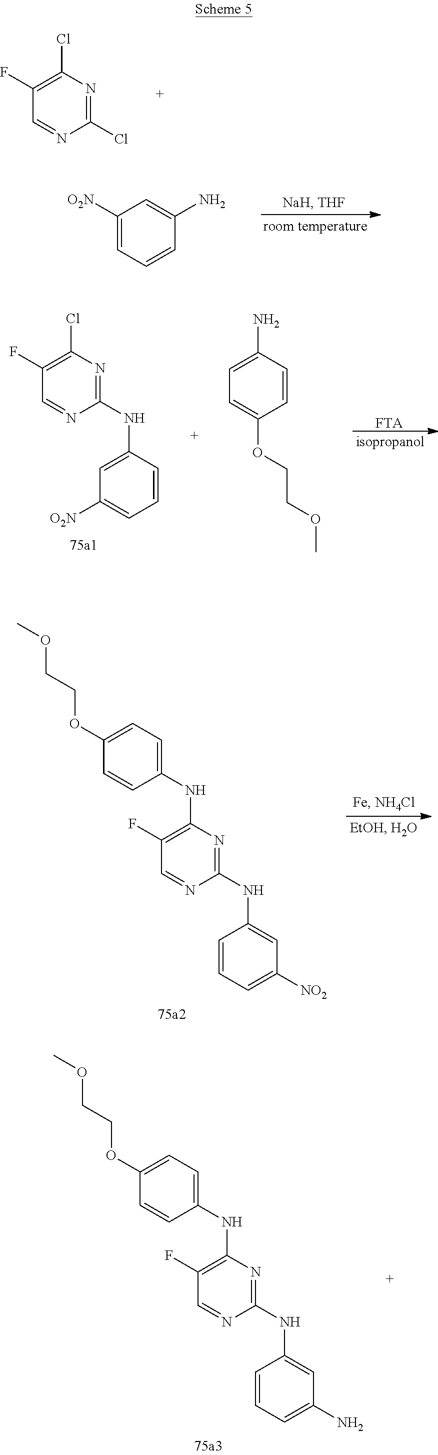

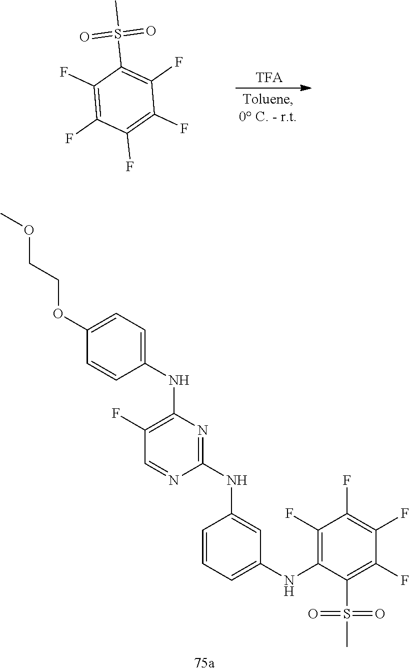 [SMILES: CS(=O)(=O)c1c(F)c(F)c(F)c(F)c1F ; COCCOc1ccc(Nc2nc(Nc3cccc(Nc4c(F)c(F)c(F)c(F)c4S(C)(=O)=O)c3)ncc2F)cc1]

Compound 75a and related examples can be synthesized from commercially available 2,4-dichloro-5-fluoropyrimidine using Scheme 5 and analogous routes known to those in the art. SNAr substitution using the appropriate amine or aniline affords intermediate 75a1. A second SNAr substitution at higher temperature, followed by nitro reduction using iron and ammonium chloride affords the intermediate 75a3. Direct-linked sulfone product 75a can be formed through direct substitution of a methyl sulfone, according to General Procedure I′, using LDA as base.

### Example 6: N-(2-((2-(dimethylamino)ethyl)(methyl)amino)-4-methoxy-5-((4-(1-methyl-1H-indol-3-yl)pyrimidin-2-yl)amino)phenyl)-2,3,4,5-tetrafluoro-6-methoxybenzenesulfonamide (Compounds 85ac, Table 1)

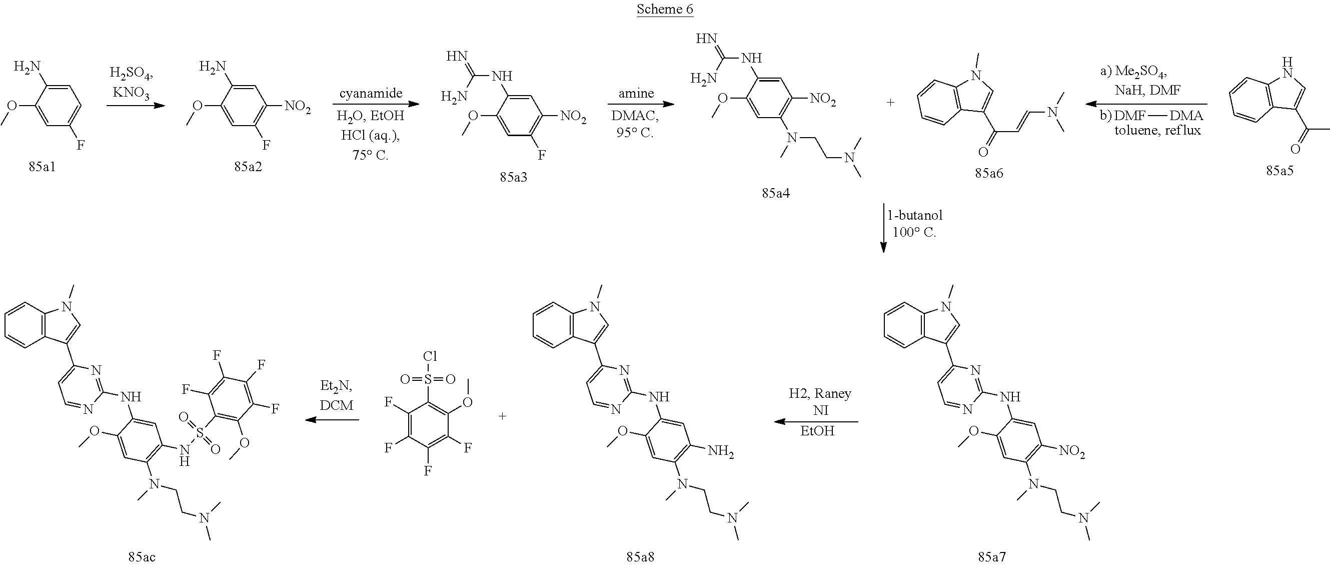

Compound 85ac can be prepared from commercially available 4-fluoro-2-methoxyaniline using similar conditions to those in Journal of Heterocyclic Chemistry, 54(5), 2898-2901. 85a1 was nitrated using H₂SO₄/KNO₃ conditions, followed by guanidinylation with cyanamide and substitution by N,N,N-trimethylethane-1,2-diamine to give the key intermediate 85a4, which is used directly in the next step. The other intermediate 85a6 was prepared by methylation of 1-(1H-indol-3-yl)ethan-1-one and condensation with N,N-dimethylformamide dimethyl acetal. Heating compounds 85a6 and 85a4 in 1-butanol at 100° C. followed by catalytic hydrogenation using H₂/Raney Ni afford 85a8, which can be reacted with previously prepared sulfonylchlorides to afford 85ac, according to General Procedure G′.

### Example 7: N-(4-((3-chloro-4-(pyridin-2-ylmethoxy)phenyl)amino)-3-cyano-7-ethoxyquinolin-6-yl)-2-(difluoromethyl)-3,4,5,6-tetrafluorobenzenesulfonimidamide (Compound 21c)

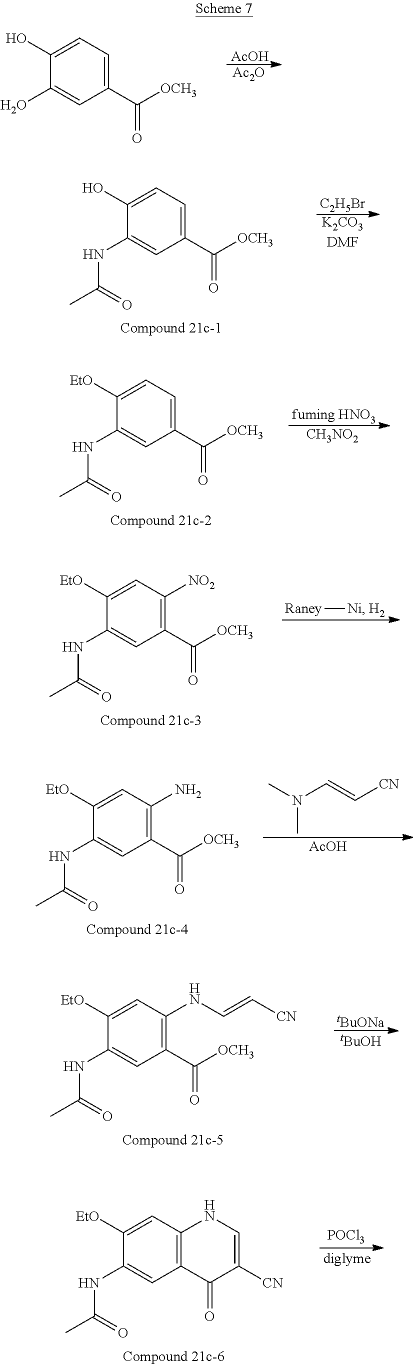

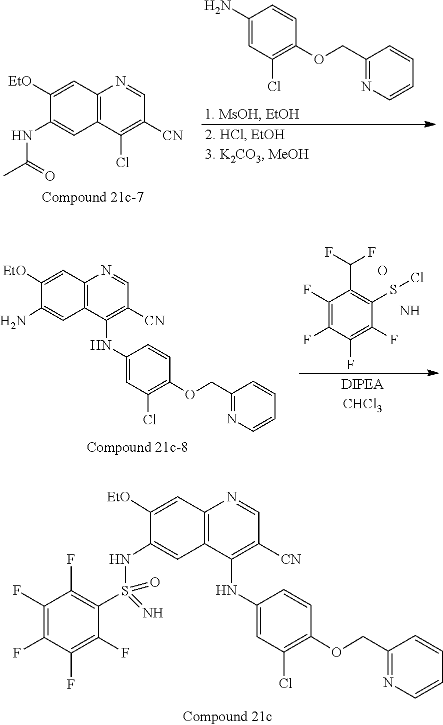

Compound 21c-7 can be prepared from commercially available methyl 3-amino-4-hydroxybenzoate as described in Org. Process Res. Dev. 2012, 16, 12, 1970-1973. Compound 21c-8 can be prepared from Compound 21c-7 as described in WO2010/151710, 2010, whereby a mixture of Compound 21c-7, commercially available 3-chloro-4-(pyridin-2-ylmethoxy)aniline and methanesulfonic acid in ethanol is refluxed for 6 hours, which is sequentially added with HCl and lastly, the isolated product is treated with aqueous K₂CO₃ in MeOH to yield the desired Compound 21c-8. The Compound 21c can be prepared from sulfonylation of Compound 21c-8 with 2-(difluoromethyl)-3,4,5,6-tetrafluorobenzenesulfonimidoyl chloride (prepared as described in General Procedure H′) by using General Procedure H′.

### Example 8: 2,3,4,5-tetrafluoro-N-(7-fluoro-4-(o-tolylamino)imidazo[1,5-a]quinoxalin-8-yl)-N,6-dimethylbenzenesulfonamide (Compound 49b)

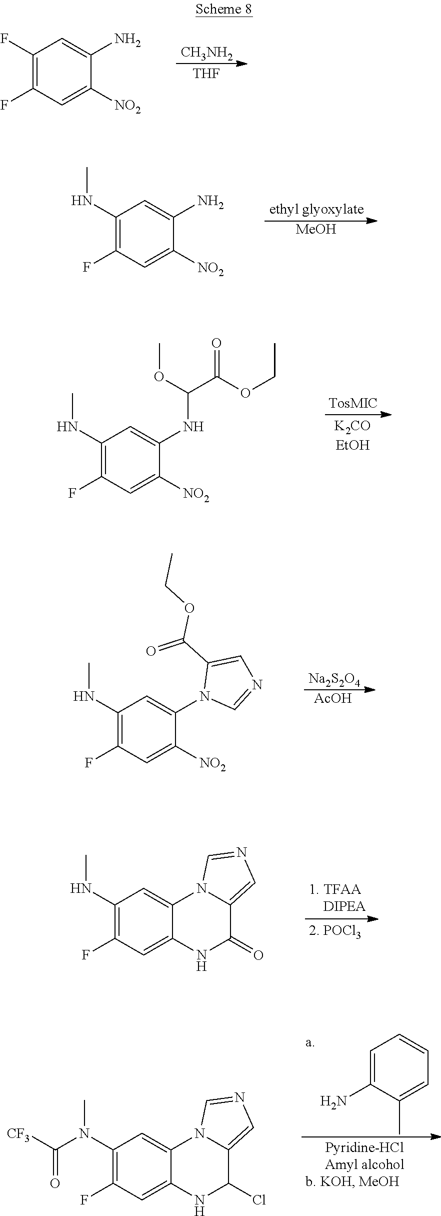

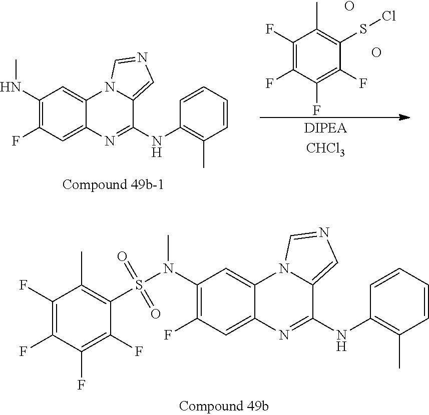

Compound 49b-1 can be prepared from commercially available 4,5-difluoro-2-nitroaniline as described in Bioorg. Med. Chem. Lett. 21 (2011) 6258-6263. The Compound 49b can be prepared from sulfonylation of Compound 49b-1 with 2,3,4,5-tetrafluoro-6-methylbenzenesulfonyl chloride (prepared as described in General Procedure B′) by using General Procedure E′.

### Example 9: 2,3,4,5-tetrafluoro-6-methoxy-N-(6-((3-phenoxybenzyl)amino)pyridin-2-yl)benzenesulfonamide (Compound 69a)

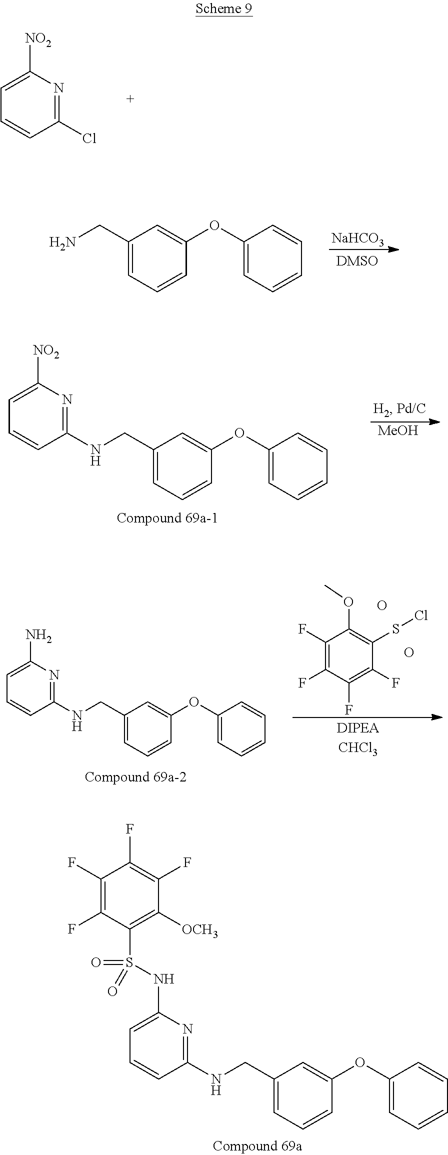

Compound 69a-1 can be prepared from commercially available 2-chloro-6-nitropyridine and (3-phenoxyphenyl)methanamine, which are added with sodium bicarbonate in DMSO and stirred at 80° C. for 4 hours to yield Compound 69a-1 according to the procedure adapted from WO2018/71794, 2018. Compound 69a-2 can be prepared from Compound 69a-1 by reductive hydrogenation with palladium on charcoal in methanol in hydrogen atmosphere using a procedure analogous to the one known in the art. Lastly, Compound 69a can be prepared from sulfonylation of Compound 69a-2 with 2,3,4,5-tetrafluoro-6-methoxybenzenesulfonyl chloride (prepared as described in General Procedure B′) by using General Procedure E′.

### Example 10: N-(3-phenoxybenzyl)-6-((2,3,4,5-tetrafluoro-6-methylphenyl)sulfonyl)pyridin-2-amine (Compound 69a)

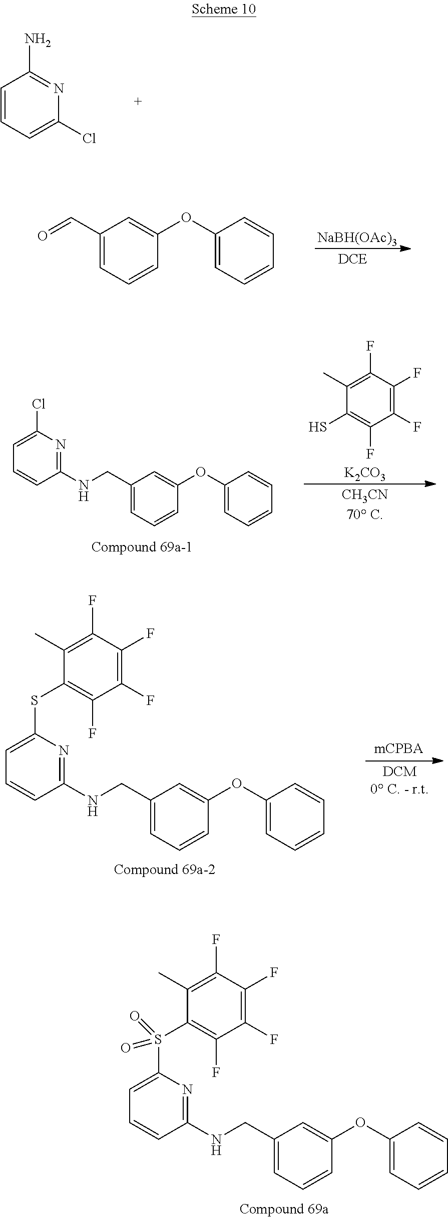

Compound 69a-1 can be prepared from reductive amination of commercially available 3-phenoxybenzaldehyde with 6-chloropyridin-2-amine using sodium triacetoxyborohydride in DCE according to the procedure adapted from J. Med. Chem. 2010, 53, 24, 8556-8568. Compound 69a-2 can be prepared from Compound 69a-1 by nucleophilic aromatic substitution with 2,3,4,5-tetrafluoro-6-methylbenzenethiol in the presence of K₂CO₃ in acetonitrile using a procedure analogous to WO2012/101239, 2012. Lastly, Compound 69a can be prepared from oxidation of Compound 69a-2 by mCPBA using a General Procedure F′.

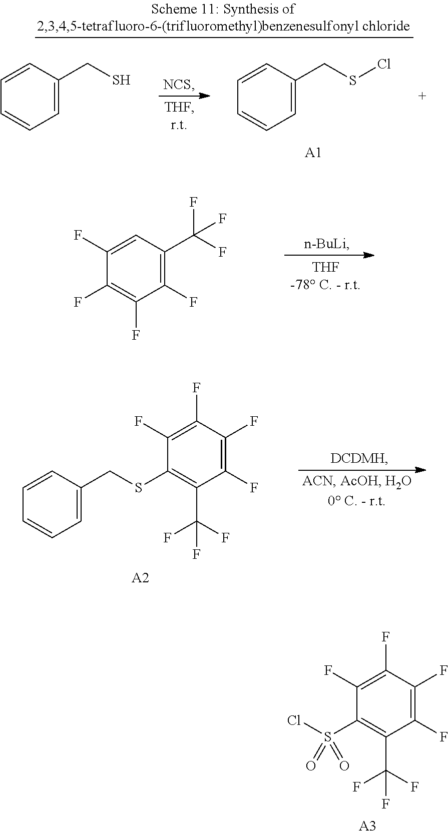

### Synthesis of A1

A dry 25 mL rbf was equipped with a stir bar, sealed with a rubber septum, and flushed with nitrogen for 5 min. After flushing, a solution of phenylmethanethiol (300 mg, 2.42 mmol, 283.55 μL) in THF (5.64 mL) was introduced into the flask. While stirring @ r.t., neat 1-chloropyrrolidine-2,5-dione (354.79 mg, 2.66 mmol, 215.02 μL) was added in one portion to prepare a pale yellow mixture. After 1 hour, the reaction became a dark yellow solution. The solution was used in the next reaction without any further manipulation/purification.

### Synthesis of A2

An oven-dried 25 mL rbf was equipped with a stir bar, capped with a rubber septum, and flushed with dry argon for 10 min. @ r.t. To the flask was added 1,2,3,4-tetrafluoro-5-(trifluoromethyl)benzene (479.76 mg, 2.2 mmol) and THE (10 mL) to prepare a colourless solution. The reaction was cooled to −78° C. before nBuLi (2.5 M in hexanes, 968.00 μL) was added to prepare the corresponding aryl-lithium species. After 20 min, the aryl lithium species (a faint purple colour) was added to a cold (0° C.) solution of benzylsulfinyl chloride (383.93 mg, 2.42 mmol) in THE (6 mL) via cannula. Extra caution was taken to ensure that the organolithium was added directly to the benzylsulfenyl chloride solution. After addition was complete, the r×n was warmed slowly to r.t. over 1 hours. After 2 hours, the reaction was quenched with a 1M HCl and the organic layer separated. The aqueous phase was extracted 2× with EtOAc and the combined organic extracts washed with brine, dried over anhydrous sodium sulfate, and concentrated under vacuum to afford the product (650 mg, 1.9 mmol, 87% yield) as a pale yellow oil. The crude material was used in the next reaction without any further purification.

### Synthesis of A3: 2,3,4,5-tetrafluoro-6-(trifluoromethyl)benzenesulfonyl chloride

Neat 1,3-dichloro-5,5-dimethyl-imidazolidine-2,4-dione (752.74 mg, 3.82 mmol, 501.82 μL) was added to an ice cold solution of 1-benzylsulfanyl-2,3,4,5-tetrafluoro-6-(trifluoromethyl)benzene (650 mg, 1.91 mmol) in CH₃CN/AcOH/H₂O (2 mL/0.075 mL/0.05 mL). The resulting pale yellow mixture was stirred @ 0° C. for 4 hours, then warmed to r.t. overnight. After the overnight period, the r×n was partitioned between DCM and a saturated aqueous solution of NaHCO₃. The organic layer was removed and the remaining aqueous phase extracted 2× with DCM. The organic extracts were combined, washed with brine, dried over anhydrous sodium sulfate, and concentrated in vacuo to afford 2,3,4,5-tetrafluoro-6-(trifluoromethyl)benzenesulfonyl chloride as a beige semi-solid. The crude material was used without any further purification ¹⁹F NMR (376 MHz, CDCl₃) δ −50.67 (d, J=37.7 Hz), −122.70 (ddd, J=23.5, 14.6, 8.8 Hz), −129.90 (ddt, J=37.7, 20.4, 9.6 Hz), −138.15 (td, J=20.6, 13.9 Hz), −142.22 (ddd, J=22.9, 19.8, 10.7 Hz).

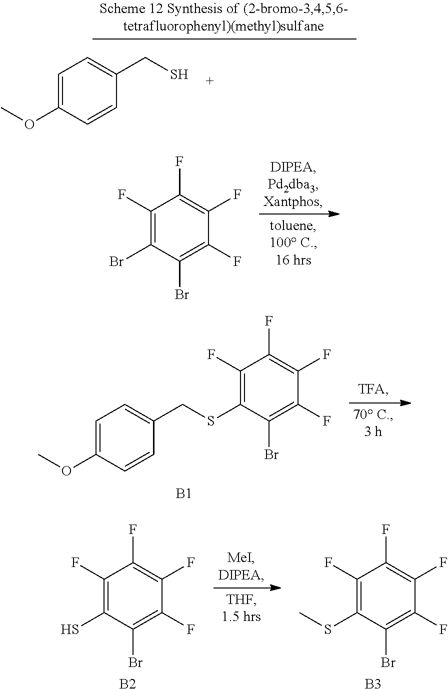

### Synthesis of B1

A solution of (4-methoxyphenyl)methanethiol (16 g, 51.94 mmol), 1,2-dibromo-3,4,5,6-tetrafluorobenzene (8 g, 51.94 mmol), and DIPEA (13.40 mL, 103.00 mmol), in toluene (150 mL) was purged with N₂ for 15 minutes. Once purged, Pd₂dba₃ (1.28 g, 1.40 mmol) and Xantphos (1.23 g, 2.00 mmol) were added at room temperature. The resulting mixture was heated to 100° C. overnight. After 16 hours, the mixture was diluted with water (100 mL) and extracted with EtOAc (2×200 mL). The combined organic phases were dried over anhydrous Na₂SO₄, filtered, and concentrated under reduced pressure. The crude material was purified by flash column chromatography (0.2% EtOAc in hexane) to afford title compound as white solid (8.0 g, 20.99 mmol, 40% yield). ¹H NMR (400 MHz, DMSO-d6) δ 7.12 (d, J=8.8 Hz, 2H), 6.83 (d, J=8.8 Hz, 2H), 4.11 (s, 2H), 3.71 (s, 3H).

### Synthesis of B2

(2-bromo-3,4,5,6-tetrafluorophenyl)(4-methoxybenzyl)sulfane (8.7 g, 22.83 mmol) was added to ice-cold (0° C.) TFA (87 mL). The reaction was gradually warmed to room temperature, then heated to 70° C. After 3 hours, reaction mixture was concentrated under reduced pressure and co-distilled with DCM (5×100 mL) to afford title compound as brown sticky oil (8 g, 30.79 mmol). The obtained material was used in next step without purification.

### Synthesis of B3: (2-bromo-3,4,5,6-tetrafluorophenyl)(methyl)sulfane

To a stirred solution of 2-bromo-3,4,5,6-tetrafluorobenzenethiol (8.0 g, 30.78 mmol) in THF (1.5 mL) @ 0° C. was added DIPEA (16 mL, 92.37 mmol), followed by addition of MeI (2.8 mL, 46.17 mmol). The reaction was permitted to warm to room temperature. After 1.5 hours, mixture was concentrated via fractional distillation to remove THE. The crude material was purified by flash column chromatography (100% hexanes) to afford title compound as colourless liquid (4.5 g, 16.36 mmol, 53% yield). ¹H NMR (400 MHz, CDCl₃) δ 2.51 (s, 3H). ¹⁹F NMR (376 MHz, CDCl₃) δ −125.99-−126.08 (m, 1F), −127.66-−127.75 (m, 1F), −152.66-−152.78 (m, 1F), −154.53-−154.65 (m, 1F).

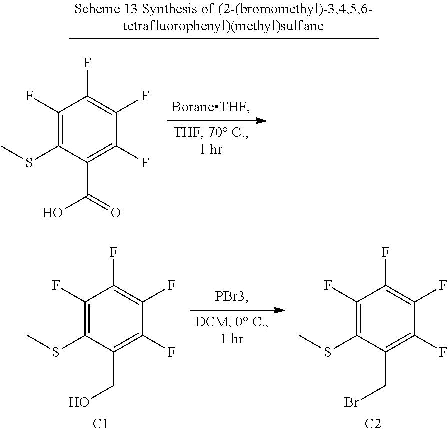

### Synthesis of C1

To a stirred solution of 2,3,4,5-tetrafluoro-6-(methylthio)benzoic acid (1.0 g, 4.16 mmol) in THF (8 mL) at 0° C. was added BH₃-THF (8.3 ml, 8.32 mmol). The reaction was heated to 70° C. for 1 h. Once complete, the reaction was cooled to 0° C. Once cold, the reaction was quenched via dropwise addition of methanol. The mixture was concentrated under reduced pressure and the obtained residue diluted with water (35 mL) then extracted with EtOAc (2×30 mL). The combined organic phases were dried over anhydrous Na₂SO₄, filtered and concentrated under reduced pressure to afford title compound as a yellow liquid (0.33 g, 1.45 mmol, 35% yield).

The obtained material was used without purification.

¹H NMR (400 MHz, DMSO-d₆) δ 5.39 (t, J=5.2, 1H), 4.71-4.69 (m, 2H), 2.42 (s, 3H). ¹⁹F NMR (376 MHz, DMSO-d6) δ −131.05-−131.14 (m, 1F), −142.17-−142.25 (m, 1F), −156.17-−156.39 (m, 2F).

### Synthesis of C2: (2-(bromomethyl)-3,4,5,6-tetrafluorophenyl)(methyl)sulfane

To a stirred solution of (2,3,4,5-tetrafluoro-6-(methylthio)phenyl)methanol (0.3 g, 1.32 mmol) in DCM (3 mL) at 0° C. was added PBr₃ (0.43 g, 1.59 mmol). The reaction was warmed to room temperature. After 1 hr, the reaction was diluted with water (10 mL) and extracted with EtOAc (3×10 mL). The combined organic phases were dried over anhydrous Na₂SO₄, filtered, and concentrated under reduced pressure to afford title compound as a yellow liquid (0.25 g, 0.86 mmol, 65% yield). The obtained material was used without purification. ¹H NMR (400 MHz, DMSO-d₆) δ 4.78 (d, J=2.4, 2H), 2.51 (s, 3H). ¹⁹F NMR (376 MHz, DMSO-d₆) δ −129.51-−129.62 (m, 1F), −139.04-−139.13 (m, 1F), −153.35-−153.48 (m, 1F), −153.95-−153.07 (m, 1F).

### Synthesis of 2-(N,N-bis(4-methoxybenzyl)sulfamoyl)-3,4,5,6-tetrafluorobenzoic acid

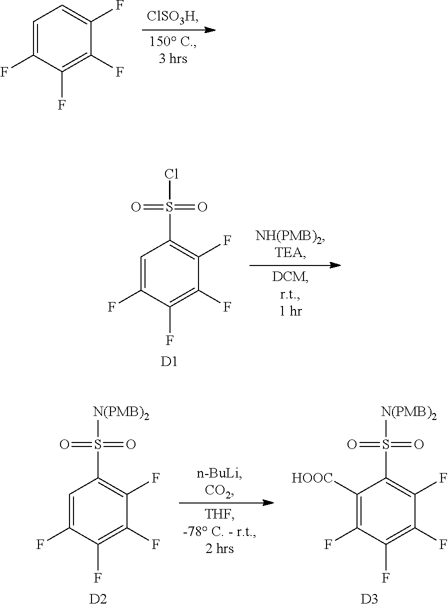

### Synthesis of D1

To a cold solution of chlorosulfonic acid (100 mL, 1499 mmol) was added 1,2,3,4-tetrafluorobenzene (45.0 g, 299.8 mmol) in a drop wise manner at 0° C. The resulting solution was heated to reflux (150° C.) for 3 hrs. After completion of reaction, the reaction mixture was quenched slowly over ice, diluted with 1M HCl (100 mL), then water (500 mL), and extracted with EtOAc (50 mL×3). The combined organic phase was dried over anhydrous Na₂SO₄, filtered, and concentrated under reduced pressure to afford the title compound as light brown oil (70.0 g, 281.5 mmol, 94% yield).

### Synthesis of D2: 2,3,4,5-tetrafluoro-N,N-bis(4-methoxybenzyl)benzenesulfonamide

To a stirred solution of 2,3,4,5-tetrafluorobenzenesulfonyl chloride (49.0 g, 197.11 mmol) in DCM (500 mL) was added TEA (41 mL, 295.60 mmol), followed by addition of Bis(4-methoxybenzyl)amine (50.72 g, 197.11 mmol) while at 0° C. temperature. The reaction was warmed to room temperature. After 1 hr, the reaction was diluted with water (400 mL) and extracted with EtOAc (100 mL×2). The combined organic phases were dried over anhydrous Na₂SO₄, filtered, and concentrated under reduced pressure. The obtained crude was purified by column chromatography (10% EtOAc in hexanes) to afford the title compound as an off white solid (37.0 g, 78.81 mmol, 40% yield). ¹H NMR (400 MHz, DMSO-d₆) δ 7.63-7.58 (m, 1H), 7.06 (d, J=8.4 Hz, 4H), 6.82 (d, J=8.8 Hz, 4H), 4.35 (s, 4H), 3.71 (s, 6H).

### Synthesis of D3: 2-(N,N-bis(4-methoxybenzyl)sulfamoyl)-3,4,5,6-tetrafluorobenzoic acid

A stirred solution of 2,3,4,5-tetrafluoro-N,N-bis(4-methoxybenzyl)benzenesulfonamide (20.0 g, 42.60 mmol) in THF (200 mL) was cooled to −78° C. temperature under N2 atmosphere. To this solution, n-BuLi (25.6 mL, 63.90 mmol, 2.5M in hexane) was added drop wise, followed by addition of TMEDA (9.6 mL, 63.90 mmol). The resulting reaction mixture was stirred at −78° C. temperature. After 1 hr, powdered dry-ice was added to the reaction mixture and stirring maintained for a further 2 hrs at −78° C. The reaction was gradually warmed to room temperature. Once warm, the reaction was quenched with 1M HCl (100 mL), then water (100 mL) and extracted with EtOAc (50 mL×3). The combined organic phase was dried over anhydrous Na₂SO₄, filtered, and concentrated under reduced pressure. The crude material was purified by column chromatography (3% MeOH in DCM) to afford the title compound as white solid (5.0 g, 9.73 mmol, 23% yield). ¹H NMR (400 MHz, DMSO-d₆) δ 14.75 (brs, 1H), 7.02 (d, J=8.4 Hz, 1H), 6.83 (d, J=8.4 Hz, 4H), 4.35 (s, 4H), 3.71 (s, 6H).

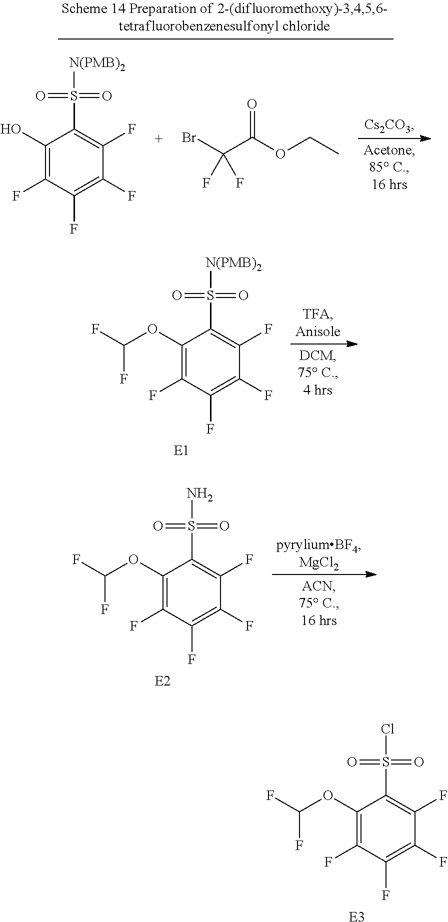

### Synthesis of E1: 2-(difluoromethoxy)-3,4,5,6-tetrafluoro-N,N-bis(4-methoxybenzyl) benzenesulfonamide

To a stirred solution of 2,3,4,5-tetrafluoro-6-hydroxy-N,N-bis(4-methoxybenzyl)benzenesulfonamide (7.0 g, 14.43 mmol) in Acetone (70 mL) was added Cs₂CO₃ (13.68 g, 43.29 mmol) and ethyl 2-bromo-2,2-difluoroacetate (8.78 g, 43.29 mmol). The resulting mixture was heated to 85° C. After 4 hrs, the reaction was cooled to room temperature and concentrated under reduced pressure. The crude material was purified by flash column chromatography (15% EtOAc in hexanes) to afford the title compound as a yellow solid (6.0 g, 11.20 mmol, 77% yield). ¹H NMR (400 MHz, CDCl₃) δ 7.07 (d, J=8.4 Hz, 4H), 6.81 (d, J=8.4 Hz, 4H), 6.69 (t, J=72 Hz, 1H), 4.44 (s, 4H), 3.8 (s, 6H).

### Synthesis of E2: 2-(difluoromethoxy)-3,4,5,6-tetrafluorobenzenesulfonamide

To a stirred solution of 2-(difluoromethoxy)-3,4,5,6-tetrafluoro-N,N-bis(4-methoxybenzyl) benzenesulfonamide (6.5 g, 12.13 mmol) in DCM (65 mL) was added anisole (5.25 g, 48.55 mmol) and the flask purged with N₂ for 10 min. Once flushed, TFA (60.5 mL) was introduced and the reaction heated to 75° C. After 16 hrs, the reaction was cooled to room temperature and concentrated under reduced pressure. The crude material was purified by flash column chromatography (20% EtOAc in hexanes) to afford the title compound as yellow solid (2.7 g, 9.14 mmol, 75% yield). ¹H NMR (400 MHz, DMSO-d₆) δ 8.30 (s, 2H), 7.07 (t, J=72 Hz, 1H), ¹⁹F NMR (400 MHz, DMSO-d₆) δ −81.82-−82.03 (m, 2F), −136.75-−136.85 (m, 1F), −149.04-−149.18 (m, 1F), −150.03-−150.17 (m, 1F), −152.17-−152.25 (m, 1F).

### Synthesis of E3: 2-(difluoromethoxy)-3,4,5,6-tetrafluorobenzenesulfonyl chloride

A suspension of 2-(difluoromethoxy)-3,4,5,6-tetrafluoro-benzenesulfonamide (700 mg, 2.37 mmol), pyrylium tetrafluoroborate (995.46 mg, 5.93 mmol) and magnesium chloride (677.41 mg, 7.11 mmol) in Acetonitrile (23.7 mL) was stirred for 10 minutes under a nitrogen atmosphere. To ensure that the reactants were solubilized, the mixture was sonicated for 5 minutes before being heated to 75° C. After 16 hours, the reaction mixture was cooled to room temperature and filtered through a small plug of silica using EtOAc as the eluent. The filtrate was concentrated down under vacuum and the crude material isolated by flash column chromatography (0-20% EtOAc in Hexanes). The desired compound was isolated as an oily solid (384 mg, 1.2 mmol, 51% yield)¹H NMR (400 MHz, CDCl₃) δ 6.70 (t, J=72 Hz, 1H). ¹⁹F NMR (400 MHz, CDCl₃) δ −82.39-−82.62 (m, 2F), −131.17-−131.28 (m, 1F), −140.23-−140.36 (m, 1F), −145.88-−145.97 (m, 1F), −152.67-−152.80 (m, 1F).

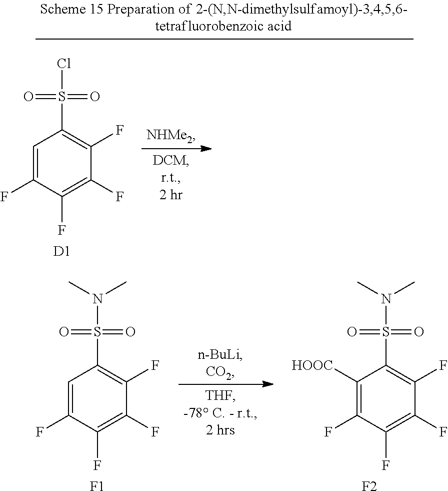

### Synthesis of F1: 2,3,4,5-tetrafluoro-N,N-dimethylbenzenesulfonamide

To a stirred solution of 2,3,4,5-tetrafluorobenzenesulfonyl chloride (15 g, 60.31 mmol) in DCM (150 mL) at 0° C. was added N,N-dimethylamine (2M in THF, 39 mL, 78.44 mmol). The reaction was warmed to room temperature. After 2 hrs, the reaction was concentrated under reduced pressure. The crude material was purified by column chromatography (20% EtOAc in hexanes) to afford the title compound as white solid (9.0 g, 34.99 mmol, 67% yield). ¹H NMR (400 MHz, DMSO-d₆) δ 7.76-7.83 (m, 1H), 2.78 (s, 6H).

### Synthesis of F2: 2-(N,N-dimethylsulfamoyl)-3,4,5,6-tetrafluorobenzoic acid

A solution of 2,3,4,5-tetrafluoro-N,N-dimethylbenzenesulfonamide (9.0 g, 35 mmol) in THF (90 mL) was cooled to −78° C. while under a positive pressure of N₂. To this solution, n-BuLi (2.5M in hexane, 21 mL, 52.52 mmol) was added drop wise, followed by addition of TMEDA (6.10 mL, 52.52 mmol). After 1 h, powdered dry ice was added to the reaction mixture and stirring was maintained for a further hour. The reaction was gradually warmed to room temperature. Once warm, the reaction was quenched with 1M HCl (50 mL), then water (100 mL), and extracted with EtOAc (50 mL×4). The combined organic phase was dried over anhydrous Na₂SO₄, filtered, and concentrated under reduced pressure. The crude material was purified by column chromatography (2% MeOH in DCM) to afford the title compound as white solid (5.0 g, 16.59 mmol, 47% yield). 1H NMR (400 MHz, DMSO-d₆) δ 14.50 (brs, 1H), 2.84 (s, 6H).

### Preparation of (3-(4-amino-3-(4-phenoxyphenyl)-1H-pyrazolo[3,4-d]pyrimidin-1-yl)azetidin-1-yl)(2,3,4,5-tetrafluoro-6-(methylsulfonyl)phenyl)methanone Compound I-27 and (3-(4-amino-3-(4-phenoxyphenyl)-1H-pyrazolo[3,4-d]pyrimidin-1-yl)azetidin-J-yl)(2,3,4,5-tetrafluoro-6-(methylsulfinyl)phenyl)methanone Compound I-37

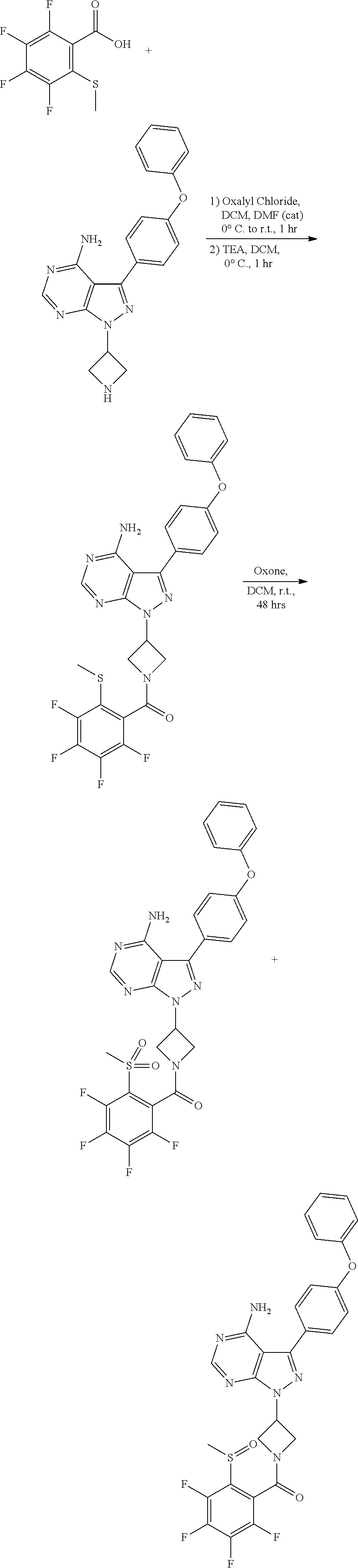 [SMILES: CSc1c(F)c(F)c(F)c(F)c1C(=O)O ; Nc1ncnc2c1c(-c1ccc(Oc3ccccc3)cc1)nn2C1CNC1 ; CSc1c(F)c(F)c(F)c(F)c1C(=O)N1CC(n2nc(-c3ccc(Oc4ccccc4)cc3)c3c(N)ncnc32)C1 ; CS(=O)(=O)c1c(F)c(F)c(F)c(F)c1C(=O)N1CC(n2nc(-c3ccc(Oc4ccccc4)cc3)c3c(N)ncnc32)C1 ; CS(=O)c1c(F)c(F)c(F)c(F)c1C(=O)N1CC(n2nc(-c3ccc(Oc4ccccc4)cc3)c3c(N)ncnc32)C1]

### (3-(4-amino-3-(4-phenoxyphenyl)-1H-pyrazolo[3,4-d]pyrimidin-1-yl)azetidin-1-yl)(2,3,4,5-tetrafluoro-6-(methylthio)phenyl)methanone

To a stirred, ice-cold (0° C.) solution of 2,3,4,5-tetrafluoro-6-(methylsulfonyl)benzoic acid (0.4 g, 1.66 mmol) in DCM (5 mL) was added oxalyl chloride (0.42 g, 3.33 mmol) and DMF (0.1 mL). The reaction mixture was stirred warmed to and maintained at room temperature for 1 hr. After I hr had elapsed, the reaction mixture was concentrated under reduced pressure and stored under a nitrogen atmosphere. The obtained residue was dissolved in THF (5 mL) and added dropwise to a stirring solution of 1-(azetidin-3-yl)-3-(4-phenoxyphenyl)-1H-pyrazolo[3,4-d]pyrimidin-4-amine (0.89 g, 2.42 mmol) in THF (5 mL) and TEA (1.1 mL, 8.33 mmol) cooled to 0° C. The reaction was permitted to warm to room temperature over a 2 hrs. Once the reaction was complete, the mixture was diluted with water (50 mL) and extracted with EtOAc (2×20 mL). The combined organic phases were dried over anhydrous Na₂SO₄, filtered, and concentrated under reduced pressure. The resulting crude was purified by flash column chromatography (50-100% EtOAc in hexane) to afford the title compound as an off-white solid (0.42 g, 0.72 mmol, 43% yield). ¹H NMR (400 MHz, DMSO-d₆) δ 8.26 (s, 1H), 7.70 (d, J=8.4 Hz, 2H), 7.45 (t, J=8.0 Hz, 2H), 7.22-7.13 (m, 5H), 5.84-5.79 (m, 1H), 4.68-4.63 (m, 1H), 4.57-4.53 (m, 1H), 4.43 (d, J=6.4 Hz, 2H), 2.45 (s, 3H). ¹⁹F NMR (376 MHz, DMSO-d₆) δ −130.57-−130.67 (m, 1F), −141.42-−141.51 (m, 1F), −153.26-−153.61 (m, 2F). ESI-MS: measured m/z 581.29 [M+1]⁺. HPLC (Method I): RT=7.21, 99.6%

### (3-(4-amino-3-(4-phenoxyphenyl)-1H-pyrazolo[3,4-d]pyrimidin-1-yl)azetidin-1-yl)(2,3,4,5-tetrafluoro-6-(methylsulfonyl)phenyl)methanone Compound I-27

To the stirred solution of (3-(4-amino-3-(4-phenoxyphenyl)-1H-pyrazolo[3,4-d]pyrimidin-1-yl)azetidin-1-yl)(2,3,4,5-tetrafluoro-6-(methylthio)phenyl)methanone (0.13 g, 0.22 mmol) in DCM (2 mL) was added oxone (0.68 g, 2.2 mmol). The resulting mixture was stirred at room temperature for 48 hrs. After 48 hrs, the reaction mixture was diluted with saturated aqueous solution of NaHCO₃ (30 mL) and extracted with DCM (3×30 mL). The combined organic phases were dried over anhydrous Na₂SO₄, filtered, and concentrated under reduced pressure. The obtained crude was purified by flash column chromatography (0-15% MeOH in DCM) to afford the title compound as an off-white solid (0.03 g, 0.049 mmol, 22% yield). ¹H NMR (400 MHz, DMSO-d₆) δ 8.27 (s, 1H, rotameric peak), 7.72 (d, J=8.8 Hz, 2H), 7.45 (t, J=8.4 Hz, 2H), 7.22-7.13 (m, 5H), 5.75-5.74 (m, 1H), 4.63-4.40 (m, 4H), 3.50 (s, 2H, combined rotameric peak), 3.42 (s, 1H, combined rotameric peak). ¹⁹F NMR (376 MHz, DMSO-d₆) δ −132.10-−132.36 (m, 1F), −139.68-−139.96 (m, 1F), −143.95-−144.29 (m, 1F), −150.94-−151.10 (m, 1F). ESI-MS: measured m/z 613.24 [M+1]⁺. HPLC (Method I) RT=6.53 min., 95.7%.

### (3-(4-amino-3-(4-phenoxyphenyl)-1H-pyrazolo[3,4-d]pyrimidin-1-yl)azetidin-1-yl)(2,3,4,5-tetrafluoro-6-(methylsulfonyl)phenyl)methanone Compound I-37

(3-(4-amino-3-(4-phenoxyphenyl)-1H-pyrazolo[3,4-d]pyrimidin-1-yl)azetidin-1-yl)(2,3,4,5-tetrafluoro-6-(methylsulfinyl)phenyl)methanone was separated from the reaction described for the synthesis of (3-(4-amino-3-(4-phenoxyphenyl)-1H-pyrazolo[3,4-d]pyrimidin-1-yl)azetidin-1-yl)(2,3,4,5-tetrafluoro-6-(methylsulfonyl)phenyl)methanone. The title compound was isolated as a yellow solid (0.015 g, 0.025 mmol, 10% yield). ¹H NMR (400 MHz, DMSO-d₆) δ 8.27 (s, 1H), 7.72 (t, J=8.8 Hz, 2H), 7.47-7.43 (m, 2H), 7.22-7.13 (m, 5H), 5.81-5.69 (m, 1H), 4.63-4.57 (m, 1H), 4.55-4.45 (m, 3H), 3.12 (s, 2H, combined rotameric peaks) 3.07 (s, 1H, combined rotameric peak). ¹⁹F NMR (376 MHz, DMSO-d₆) δ −132.10-−132.36 (m, 1F), −139.69-−139.96 (m, 1F), −143.99-−144.32 (m, 1F), −150.96-−151.11 (m, 1F). ESI-MS: measured m/z 597.24[M+1]⁺. HPLC RT=6.48 min., 95.3%.

### General Procedure A:

### N-(2-(4-amino-3-(4-phenoxyphenyl)-1H-pyrazolo[3,4-d]pyrimidin-1-yl)ethyl)-2-(N,N-dimethylsulfamoyl)-3,4,5,6-tetrafluoro-N-methylbenzamide Compound I-30

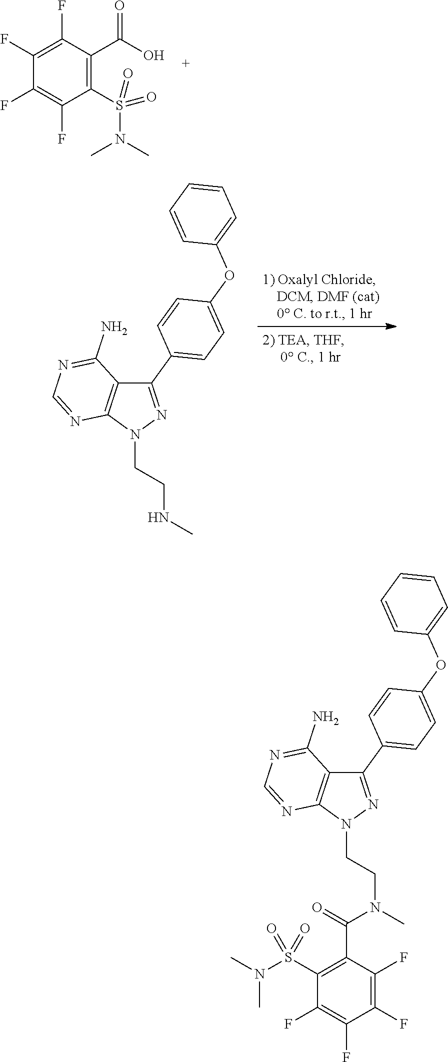 [SMILES: CN(C)S(=O)(=O)c1c(F)c(F)c(F)c(F)c1C(=O)O ; CNCCn1nc(-c2ccc(Oc3ccccc3)cc2)c2c(N)ncnc21 ; CN(CCn1nc(-c2ccc(Oc3ccccc3)cc2)c2c(N)ncnc21)C(=O)c1c(F)c(F)c(F)c(F)c1S(=O)(=O)N(C)C]

N-(2-(4-amino-3-(4-phenoxyphenyl)-1H-pyrazolo[3,4-d]pyrimidin-1-yl)ethyl)-2-(N,N-dimethylsulfamoyl)-3,4,5,6-tetrafluoro-N-methylbenzamide was prepared from 2-(N,N-dimethylsulfamoyl)-3,4,5,6-tetrafluorobenzoic acid (0.4 g, 1.32 mmol) and 1-(2-(methylamino)ethyl)-3-(4-phenoxyphenyl)-1H-pyrazolo[3,4-d]pyrimidin-4-amine (0.62 g, 1.72 mmol) according to the protocol described in general procedure A and isolated as a white solid (0.4 g, 0.62 mmol, 51% yield). ¹H NMR (400 MHz, DMSO-d₆) δ 8.29-8.17 (s, 0.5H, 0.34H, rotameric peaks integrated separately), 7.71 (d, J=8.4 Hz, 1H), 7.65 (d, J=8.4 Hz, 1H), 7.45 (t, J=7.6 Hz, 2H), 7.23-7.13 (m, 5H), 6.67 (s, 1H), 6.62 (s, 1H), 4.68-4.57 (m, 2H), 4.15-4.44 (m, 1H), 3.81-3.87 (m, 1H), 2.88-2.85 (m, 9H). ¹⁹F NMR (376 MHz, DMSO-d₆) δ −130.79-−130.94 (m, 1F), −139.78-−130.86 (m, 0.33F), −140.79-−140.88 (m, 0.67F), −145.98-−146.06 (m, 0.33F), −146.18-−146.32 (m, 0.67F), −152.05-−152.33 (m, 1F). ESI-MS: measured m/z 644.2 [M+1]⁺, HPLC (Method I): RT=6.59 min., 98.2%.

### N-(2-(4-amino-3-(4-phenoxyphenyl)-1H-pyrazolo[3,4-d]pyrimidin-1-yl)ethyl)-2,3,4,5-tetrafluoro-N-methyl-6-sulfamoylbenzamide Compound I-33

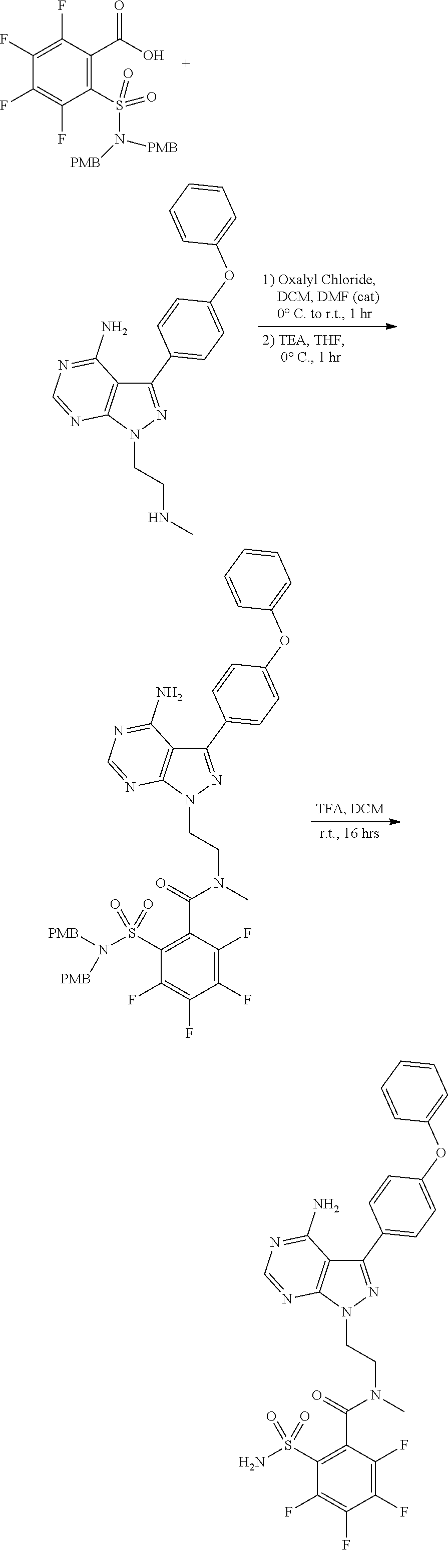

### N-(2-(4-amino-3-(4-phenoxyphenyl)-1H-pyrazolo[3,4-d]pyrimidin-1-yl)ethyl)-2-(N,N-bis(4-methoxybenzyl)sulfamoyl)-3,4,5,6-tetrafluoro-N-methylbenzamide

N-(2-(4-amino-3-(4-phenoxyphenyl)-1H-pyrazolo[3,4-d]pyrimidin-1-yl)ethyl)-2-(N,N-bis(4-methoxybenzyl)sulfamoyl)-3,4,5,6-tetrafluoro-N-methylbenzamide was prepared from 2-(N,N-bis(4-methoxybenzyl)sulfamoyl)-3,4,5,6-tetrafluorobenzoic acid (0.25 g, 0.48 mmol) and 1-(2-(methylamino)ethyl)-3-(4-phenoxyphenyl)-1H-pyrazolo[3,4-d]pyrimidin-4-amine (0.263 g, 0.73 mmol) according to the protocol described in general procedure A and isolated as a yellow solid (0.21 g, 0.24 mmol, 50% yield). ESI-MS: measured m/z 856.3 [M+1]⁺.

### N-(2-(4-amino-3-(4-phenoxyphenyl)-1H-pyrazolo[3,4-d]pyrimidin-1-yl)ethyl)-2,3,4,5-tetrafluoro-N-methyl-6-sulfamoylbenzamide Compound I-33

To a stirred solution of N-(2-(4-amino-3-(4-phenoxyphenyl)-1H-pyrazolo[3,4-d]pyrimidin-1-yl)ethyl)-2-(N,N-bis(4-methoxybenzyl)sulfamoyl)-3,4,5,6-tetrafluoro-N-methylbenzamide (0.21 g, 0.24 mmol) in DCM (2 mL) was added TFA (2 mL). The reaction was stirred at room temperature for 16 hrs. Once complete, the reaction mixture was diluted with saturated aqueous NaHCO₃ (30 mL) and extracted with EtOAc (3×30 mL). The combined organic phases were dried over anhydrous Na₂SO₄, filtered, and concentrated under reduced pressure. The crude material was purified by flash column chromatography (56% EtOAc in hexanes) to afford the title compound as an off-white solid (0.085 g, 0.13 mmol, 56% yield). ¹H NMR (400 MHz, DMSO-d₆) δ 8.27-8.20 (d, 2H), 8.12 (s, 1H), 7.68-7.60 (m, 2H), 7.44 (t, J=7.6 Hz, 2H) 7.21-7.12 (m, 5H), 4.61-4.31 (m, 2H) 4.10-3.60 (m, 2H), 2.81-2.67 (s, 3H, rotameric peaks integrated together). ¹⁹F NMR (376 MHz, DMSO-d₆) δ −134.64-−134.78 (m, 1F), −140.72-−141.61 (m, 1F), −147.71-−147.96 (m, 1F), −153.22-−153.49 (m, 1F). ESI-MS: m/z 616.4 [M+1]⁺, HPLC (Method I): RT=6.10 min., 96%.

### Preparation of N-(2-(4-amino-3-(4-phenoxyphenyl)-1H-pyrazolo[3,4-d]pyrimidin-1-yl)ethyl)-2,3,4,5-tetrafluoro-N-methyl-6-(methylsulfonyl)benzamide Compound I-28

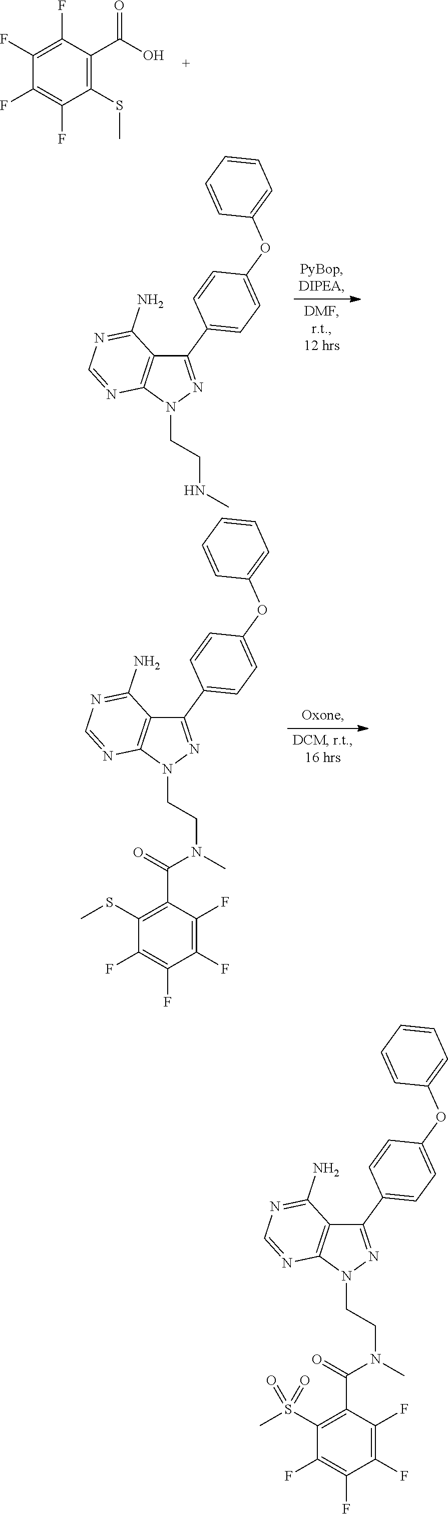

### General Procedure D:

### N-(2-(4-amino-3-(4-phenoxyphenyl)-1H-pyrazolo[3,4-d]pyrimidin-1-yl)ethyl)-2,3,4,5-tetrafluoro-N-methyl-6-(methylthio)benzamide

To a stirred solution of 2,3,4,5-tetrafluoro-6-(methylthio)benzoic acid (0.3 g, 1.25 mmol) and 1-(2-(methylamino)ethyl)-3-(4-phenoxyphenyl)-1H-pyrazolo[3,4-d]pyrimidin-4-amine (0.54 g, 1.5 mmol) in DMF (3 mL) was added PyBop (0.975 g, 1.87 mmol) and reaction mixture was stirred for 5 min at room temperature. DIPEA (0.48 g, 3.75 mmol) was introduced via syringe and the reaction was permitted to stir for a further 12 hrs. After reaction completion, the mixture was diluted with water (50 mL) and extracted with EtOAc (3×30 mL). The combined organic phases were dried over anhydrous Na₂SO₄, filtered, and concentrated under reduced pressure. The resulting crude was purified by flash column chromatography (5% MeOH in DCM) to afford the title compound as an off-white solid (0.450 g, 0.430 mmol, 62% yield). ESI-MS: measured m/z 582.20 [M+1]⁺.

### General Procedure C:

### N-(2-(4-amino-3-(4-phenoxyphenyl)-1H-pyrazolo[3,4-d]pyrimidin-1-yl)ethyl)-2,3,4,5-tetrafluoro-N-methyl-6-(methylsulfonyl)benzamide Compound I-28

To an ice cold (0° C.) solution of N-(2-(4-amino-3-(4-phenoxyphenyl)-1H-pyrazolo[3,4-d]pyrimidin-1-yl)ethyl)-2,3,4,5-tetrafluoro-N-methyl-6-(methylthio)benzamide (0.25 g, 0.42 mmol) in DCM (2.5 mL) was added oxone (0.326 g, 2.14 mmol). The reaction was warmed to room temperature and stirred for 16 hrs. Once the reaction was deemed completed, the mixture was diluted with saturated aqueous Na₂CO₃ (50 mL) and extracted with EtOAc (2×30 mL). The combined organic phases were dried over anhydrous Na₂SO₄, filtered, and concentrated under reduced pressure. The resulting crude was purified by flash column chromatography using reverse phase silica (55% acetonitrile in water) to afford title compound as a brown solid (0.057 g, 0.092 mmol, 22% yield). ¹H NMR (400 MHz, DMSO-d₆) δ 8.27 (s, 1H), 7.67 (d, J=8.4 Hz, 2H), 7.44 (t, J=7.6 Hz, 2H), 7.21-7.12 (m, 5H), 4.60 (t, J=6.4 Hz, 2H), 4.22-4.15 (m, 1H), 3.77-3.72 (m, 1H), 3.39 (s, 3H), 2.88 (s, 3H). ¹⁹F NMR (376 MHz, DMSO-d₆) δ −132.57-−132.69 (m, 1F), 140.79-−140.87 (m, 1F), −144.61-−144.76 (m, 1F), −152.26-−152.39 (m, 1F). ESI-MS: measured m/z 615.4 [M+1]⁺. HPLC (Method I): RT=7.50 min. and 7.61, 10.3% and 82.2%, respectively (anticipated mixture of rotamers).

### 2-(3-(4-amino-3-(4-phenoxyphenyl)-1H-pyrazolo[3,4-d]pyrimidin-1-yl)azetidine-1-carbonyl)-3,4,5,6-tetrafluoro-N,N-dimethylbenzenesulfonamide Compound I-29

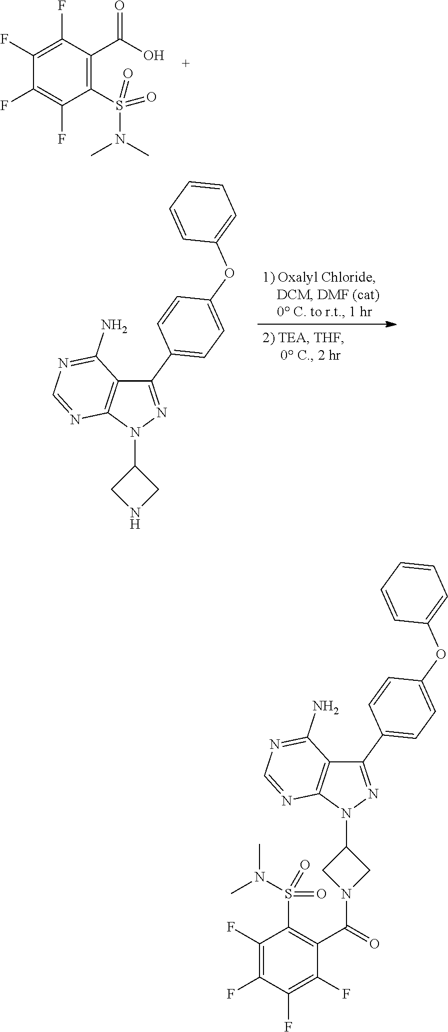 [SMILES: CN(C)S(=O)(=O)c1c(F)c(F)c(F)c(F)c1C(=O)O ; Nc1ncnc2c1c(-c1ccc(Oc3ccccc3)cc1)nn2C1CNC1 ; CN(C)S(=O)(=O)c1c(F)c(F)c(F)c(F)c1C(=O)N1CC(n2nc(-c3ccc(Oc4ccccc4)cc3)c3c(N)ncnc32)C1]

2-(3-(4-amino-3-(4-phenoxyphenyl)-1H-pyrazolo[3,4-d]pyrimidin-1-yl)azetidine-1-carbonyl)-3,4,5,6-tetrafluoro-N,N-dimethylbenzenesulfonamide was prepared from 2-(N,N-dimethylsulfamoyl)-3,4,5,6-tetrafluorobenzoic acid (0.3 g, 1.00 mmol) and 1-(azetidin-3-yl)-3-(4-phenoxyphenyl)-1H-pyrazolo[3,4-d]pyrimidin-4-amine (0.42 g, 1.2 mmol) according to the protocol described in general procedure A and isolated as an off-white solid (0.083 g, 0.12 mmol, 13% yield). ¹H NMR (400 MHz, DMSO-d₆) δ 8.27 (s, 1H), 7.73-7.69 (m, 2H) 7.45 (t, J=8.4 Hz, 2H), 7.22-7.13 (m, 5H), 5.75-5.73 (m, 1H), 4.60-4.52 (m, 2H), 4.44-4.34 (m, 2H), 2.88-2.84 (m, 6H). ¹⁹F NMR (376 MHz, DMSO-d₆) δ −130.29-−130.74 (m, 1F), −139.67-−139.82 (m, 1F), −145.53-−145.93 (m, 1F), −150.89-−151.08 (m, 1F). ESI+MS: measured m/z 642.24 [M+1]⁺. HPLC (Method I): RT=4.28 min., 97.5%.

### Preparation of 2-(3-(4-amino-3-(4-phenoxyphenyl)-1H-pyrazolo[3,4-d]pyrimidin-1-yl)azetidine-1-carbonyl)-3,4,5,6-tetrafluorobenzenesulfonamide Compound I-32

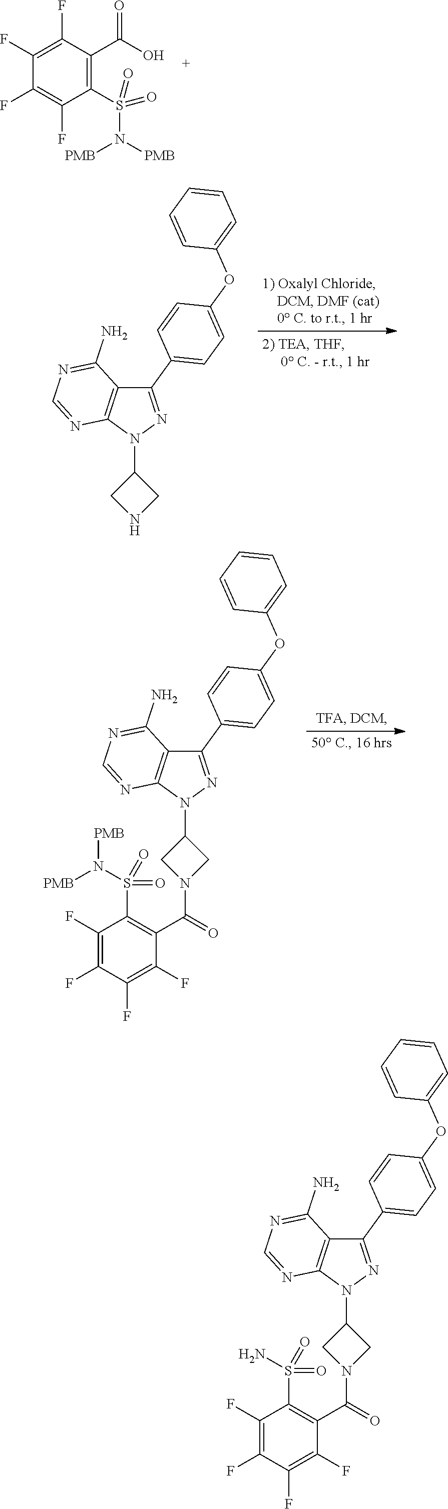

### 2-(3-(4-amino-3-(4-phenoxyphenyl)-1H-pyrazolo[3,4-d]pyrimidin-1-yl)azetidine-1-carbonyl)-3,4,5,6-tetrafluoro-N,N-bis(4-methoxybenzyl)benzenesulfonamide

2-(3-(4-amino-3-(4-phenoxyphenyl)-1H-pyrazolo[3,4-d]pyrimidin-1-yl)azetidine-1-carbonyl)-3,4,5,6-tetrafluoro-N,N-bis(4-methoxybenzyl)benzenesulfonamide was prepared from 2-(N,N-bis(4-methoxybenzyl)sulfamoyl)-3,4,5,6-tetrafluorobenzoic acid (0.25 g, 0.48 mmol) and 1-(azetidin-3-yl)-3-(4-phenoxyphenyl)-1H-pyrazolo[3,4-d]pyrimidin-4-amine (0.20 g, 0.58 mmol) according to the protocol described in general procedure A and isolated as an off-white solid (0.21 g, 0.25 mmol, 72% yield). ESI-MS: measured m/z 854.2 [M+1]⁺.

### General Procedure B:

### 2-(3-(4-amino-3-(4-phenoxyphenyl)-1H-pyrazolo[3,4-d]pyrimidin-1-yl)azetidine-1-carbonyl)-3,4,5,6-tetrafluorobenzenesulfonamide Compound I-32

To a stirred solution of 2-(3-(4-amino-3-(4-phenoxyphenyl)-1H-pyrazolo[3,4-d]pyrimidin-1-yl)azetidine-1-carbonyl)-3,4,5,6-tetrafluoro-N,N-bis(4-methoxybenzyl)benzenesulfonamide (0.2 g, 0.23 mmol) in DCM (2 mL) was added TFA (4 mL) at room temperature. The resulting solution mixture was stirred at 50° C. (oil bath) for 16 hrs. once the reaction was deemed completed, the solution was diluted with a saturated aqueous solution of NaHCO₃ (20 mL) and extracted with EtOAc (2×20 mL). The combined organic phases were dried over anhydrous Na₂SO₄, filtered, and concentrated under reduced pressure. The obtained crude was purified by reverse phase column chromatography (70-80% ACN in water) to afford the title compound as a white solid (0.05 g, 0.08 mmol, 35% yield). ¹H NMR (376 MHz, DMSO-d₆) δ 8.27 (s, 1H), 7.75-7.71 (m, 2H), 7.45 (t, J=7.6 Hz, 2H), 7.20-7.13 (m, 5H), 5.75-5.72 (m, 1H), 4.61-4.51 (m, 2H), 4.43-4.41 (m, 2H). ¹⁹F NMR (376 MHz, DMSO-d₆) δ −134.39-−134.49 (m, 1F), −140.40-−140.72 (m, 1F), −147.55-−147.65 (m, 1F), −152.10-−152.20 (m, 1F). ESI-MS: m/z 614.2 [M+1]⁺. HPLC (Method I) RT=6.32 min., 96.6%.

### (R)-2-(3-(4-amino-3-(4-phenoxyphenyl)-1H-pyrazolo[3,4-d]pyrimidin-1-yl)piperidine-1-carbonyl)-3,4,5,6-tetrafluoro-N,N-dimethylbenzenesulfonamide Compound I-31

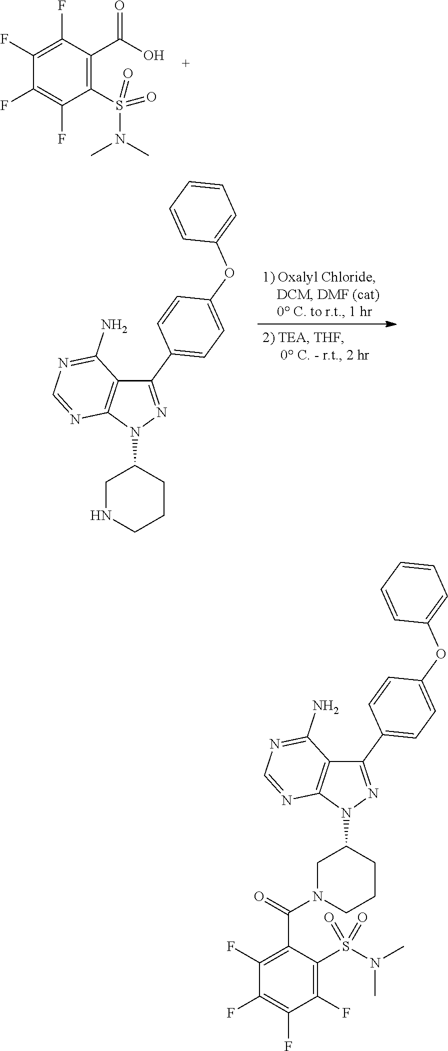 [SMILES: CN(C)S(=O)(=O)c1c(F)c(F)c(F)c(F)c1C(=O)O ; Nc1ncnc2c1c(-c1ccc(Oc3ccccc3)cc1)nn2[C@@H]1CCCNC1 ; CN(C)S(=O)(=O)c1c(F)c(F)c(F)c(F)c1C(=O)N1CCC[C@@H](n2nc(-c3ccc(Oc4ccccc4)cc3)c3c(N)ncnc32)C1]

(R)-2-(3-(4-amino-3-(4-phenoxyphenyl)-1H-pyrazolo[3,4-d]pyrimidin-1-yl)piperidine-1-carbonyl)-3,4,5,6-tetrafluoro-N,N-dimethylbenzenesulfonamide was prepared from 2-(N,N-dimethylsulfamoyl)-3,4,5,6-tetrafluorobenzoic acid (0.3 g, 1.0 mmol) and (R)-3-(4-phenoxyphenyl)-1-(piperidin-3-yl)-1H-pyrazolo[3,4-d]pyrimidin-4-amine (0.46 g, 1.2 mmol) according to the protocol described in general procedure A and isolated as an off-white solid (0.009 g, 0.013 mmol, 1% yield). ¹H NMR (400 MHz, DMSO-d₆) δ 8.82-8.13 (m, 1H), 7.71-7.68 (m, 1H), 7.63-7.59 (m, 1H), 7.47-7.41 (m, 2H), 7.21-7.11 (m, 5H), 4.90-4.50 (m, 2H), 4.65-4.60 (m, 0.5H), 3.90-4.00 (m, 0.5H), 4.00-3.80 (m, 0.5H), 4.80-3.70 (m, 0.5H), 3.50-3.40 (m, 1H), 2.90-2.70 (m, 6H), 2.30-2.30-2.20 (m, 1H), 2.20-2.10 (m, 1H), 2.0-1.90 (m, 1H), 1.80-1.60 (m, 1H). ¹⁹F NMR (376 MHz, DMSO-d₆) −130.66-−131.74 (m, 1F), −140.60-−140.83 (m, 1F), 146.04-−146.24 (m, 1F), −152.36-−152.51 (m, 1F). ESI-MS: measured m/z 670.7 [M+1]⁺. HPLC (Method I): RT=6.94 min. and 7.00 min., 68.4% and 17.0%, respectively (anticipated mixture of rotamers).

### Preparation of (R)-2-(3-(4-amino-3-(4-phenoxyphenyl)-1H-pyrazolo[3,4-d]pyrimidin-1-yl)piperidine-1-carbonyl)-3,4,5,6-tetrafluorobenzenesulfonamide Compound I-35

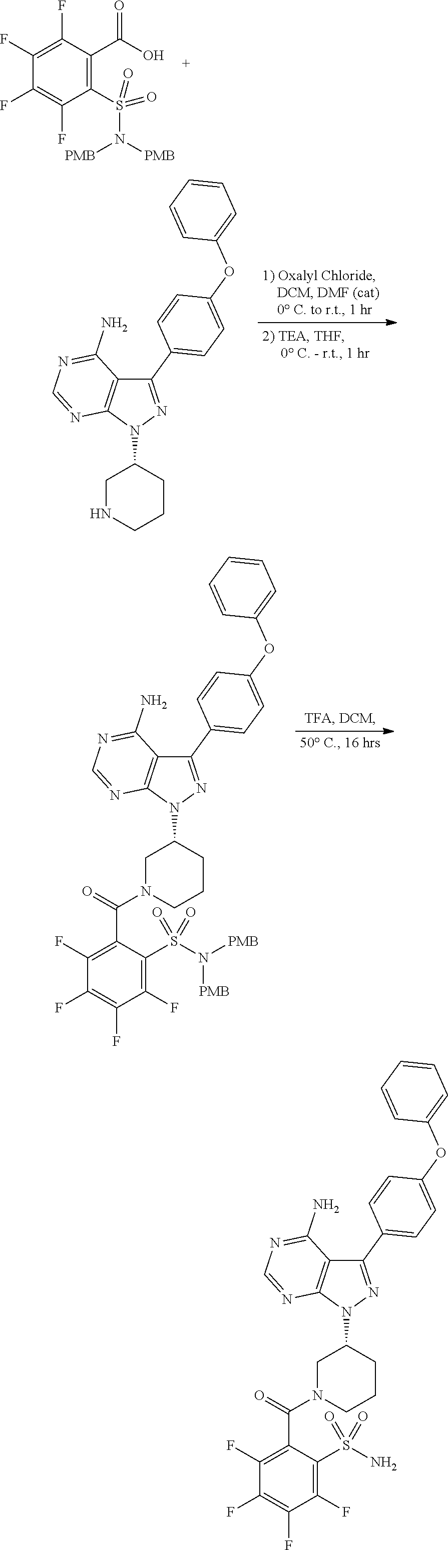

### (R)-2-(3-(4-amino-3-(4-phenoxyphenyl)-1H-pyrazolo[3,4-d]pyrimidin-1-yl) piperidine-1-carbonyl)-3,4,5,6-tetrafluoro-N,N-bis(4-methoxybenzyl)benzenesulfonamide

(R)-2-(3-(4-amino-3-(4-phenoxyphenyl)-1H-pyrazolo[3,4-d]pyrimidin-1-yl) piperidine-1-carbonyl)-3,4,5,6-tetrafluoro-N,N-bis(4-methoxybenzyl)benzenesulfonamide was prepared from 2-(N,N-bis(4-methoxybenzyl)sulfamoyl)-3,4,5,6-tetrafluorobenzoic acid (0.3 g, 0.58 mmol) and (R)-3-(4-phenoxyphenyl)-1-(piperidin-3-yl)-1H-pyrazolo[3,4-d]pyrimidin-4-amine (0.22 g, 0.70 mmol) according to the protocol described in general procedure A and isolated as an off-white solid (0.45 g, 0.51 mmol, 80% yield). ¹⁹F NMR (376 MHz, DMSO-d₆) δ −132.06-−132.77 (m, 1F), −139.73-−140.19 (m, 1F), −145.37-−145.93 (m, 1F), −152.71-−153.00 (m, 1F). ESI-MS: measured m/z 882.6 [M+1f].

### (R)-2-(3-(4-amino-3-(4-phenoxyphenyl)-1H-pyrazolo[3,4-d]pyrimidin-1-yl)piperidine-1-carbonyl)-3,4,5,6-tetrafluorobenzenesulfonamide Compound I-35

(R)-2-(3-(4-amino-3-(4-phenoxyphenyl)-1H-pyrazolo[3,4-d]pyrimidin-1-yl)piperidine-1-carbonyl)-3,4,5,6-tetrafluorobenzenesulfonamide was prepared from (R)-2-(3-(4-amino-3-(4-phenoxyphenyl)-1H-pyrazolo[3,4-d]pyrimidin-1-yl)piperidine-1-carbonyl)-3,4,5,6-tetrafluoro-N,N-bis(4-methoxybenzyl) benzene sulfonamide (0.35 g, 0.39 mmol) according to the protocol described in general procedure B and isolated as a white solid (0.1 g, 0.15 mmol, 46% yield). ¹H NMR (400 MHz, DMSO-d₆) δ 8.27-8.07 (m, 3H), 7.71-7.68 (m, 1H), 7.62-7.60 (m, 1H), 7.46-7.41 (m, 2H), 7.21-7.11 (m, 5H), 4.97-61 (m, 2H), 3.67-3.56 (m, 1H) 3.50-3.41 (m, 1H), 3.22-3.09 (m, 1H), 2.35-2.22 (m, 1H), 2.19-2.09 (m, 1H), 2.01-1.99 (m, 1H), 1.73-1.65 (m, 1H). ¹⁹F NMR (376 MHz, DMSO-d₆) δ −134.59-−135.02 (m, 1F), −141.38-−141.75 (m, 1F), −148.29-−149.00 (m, 1F), −153.56-−153.78 (m, 1F). ESI-MS: m/z 642.4 [M+1]⁺, HPLC (Method I): RT=6.38 min., 6.60 min. and 6.89 min., 28%, 32% and 38.9%, respectively (anticipated mixture of rotamers).

### Preparation of ((R)-3-(4-amino-3-(4-phenoxyphenyl)-1H-pyrazolo[3,4-d]pyrimidin-1-yl)piperidin-1-yl)(2,3,4,5-tetrafluoro-6-(methylsulfinyl)phenyl)methanone Compound I-36

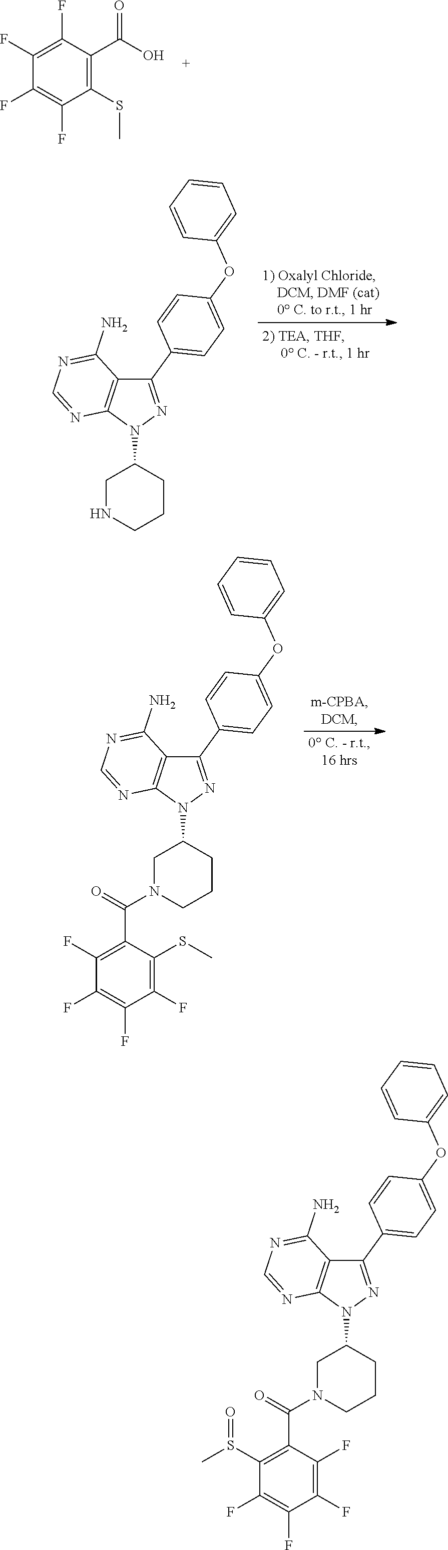 [SMILES: CSc1c(F)c(F)c(F)c(F)c1C(=O)O ; Nc1ncnc2c1c(-c1ccc(Oc3ccccc3)cc1)nn2[C@@H]1CCCNC1 ; CSc1c(F)c(F)c(F)c(F)c1C(=O)N1CCC[C@@H](n2nc(-c3ccc(Oc4ccccc4)cc3)c3c(N)ncnc32)C1 ; CS(=O)c1c(F)c(F)c(F)c(F)c1C(=O)N1CCC[C@@H](n2nc(-c3ccc(Oc4ccccc4)cc3)c3c(N)ncnc32)C1]

### (R)-(3-(4-amino-3-(4-phenoxyphenyl)-1H-pyrazolo[3,4-d]pyrimidin-1-yl)piperidin-1-yl)(2,3,4,5-tetrafluoro-6-(methylthio)phenyl)methanone

(R)-(3-(4-amino-3-(4-phenoxyphenyl)-1H-pyrazolo[3,4-d]pyrimidin-1-yl)piperidin-1-yl)(2,3,4,5-tetrafluoro-6-(methylthio)phenyl)methanone was prepared from 2,3,4,5-tetrafluoro-6-(methylthio)benzoic acid (0.2 g, 0.83 mmol) and (R)-3-(4-phenoxyphenyl)-1-(piperidin-3-yl)-1H-pyrazolo[3,4-d]pyrimidin-4-amine (0.32 g, 0.83 mmol) according to the protocol described in general procedure A and isolated as a colorless oil (0.4 g, 0.65 mmol, 70% yield). ¹H NMR (400 MHz, DMSO-d₆) δ 8.27 (s, 1H), 7.67 (d, J=8.8 Hz, 1H), 7.60-7.57 (m, 1H), 7.45-7.40 (m, 2H), 7.20-7.08 (m, 5H), 4.80-4.73 (m, 1H), 4.65-4.58 (m, 1H), 4.45-4.38 (m, 1H), 4.22-4.15 (m, 1H), 3.48-3.40 (m, 2H), 3.35-3.18 (m, 3H), 2.35-2.22 (m, 3H). ¹⁹F NMR (376 MHz, DMSO-d₆) δ −130.69-−131.58 (m, 1F), −141.41-−142.50 (m, 1F), −153.86-−154.01 (m, 1F), −154.36-−154.47 (m, 1F). ESI-MS: measured m/z 609.2 [M+1]⁺.

### ((R)-3-(4-amino-3-(4-phenoxyphenyl)-1H-pyrazolo[3,4-d]pyrimidin-1-yl)piperidin-1-yl)(2,3,4,5-tetrafluoro-6-(methylsulfinyl)phenyl)methanone Compound I-36

To a stirred, ice-cold (0° C.) solution of (R)-(3-(4-amino-3-(4-phenoxyphenyl)-1H-pyrazolo[3,4-d]pyrimidin-1-yl)piperidin-1-yl)(2,3,4,5-tetrafluoro-6-(methylthio)phenyl)methanone (0.4 g, 0.65 mmol) in DCM (5 mL) was added m-CPBA (0.56 g, 3.28 mmol). The reaction was gradually warmed to room temperature and for 16 hrs. Once deemed complete, the reaction mixture was diluted with a saturated aqueous solution of NaHCO₃ (20 mL) and extracted with EtOAc (2×20 mL). The combined organic phases were dried over anhydrous Na₂SO₄, filtered, and concentrated under reduced pressure. The crude material was purified by reverse phase column chromatography (60-70% ACN in water) to afford the title compound as an off-white solid (0.05 g, 0.08 mmol, 12% yield). ¹H NMR (400 MHz, DMSO-d₆) δ 8.29-8.17 (m, 1H), 7.71-7.59 (m, 2H), 7.46-7.41 (m, 2H), 7.21-7.11 (m, 5H), 4.99-4.85 (m, 1H), 4.74-4.65 (m, 1H), 4.55-4.44 (m, 1H), 3.80-3.65 (m, 1H), 3.52-3.45 (m, 1H), 3.14-3.02 (m, 3H), 2.30-2.10 (m, 2H), 2.20-1.80 (m, 2H). ESI-MS: m/z 625.25 [M+1]⁺, HPLC (Method II): RT=9.12 min. and 9.50 min, 90.2%, 9%, respectively (anticipated mixture of rotamers).

### Preparation of N-(3-(4-amino-3-(4-phenoxyphenyl)-1H-pyrazolo[3,4-d]pyrimidin-1-yl)propyl)-2,3,4,5-tetrafluoro-6-sulfamoylbenzamide Compound I-34

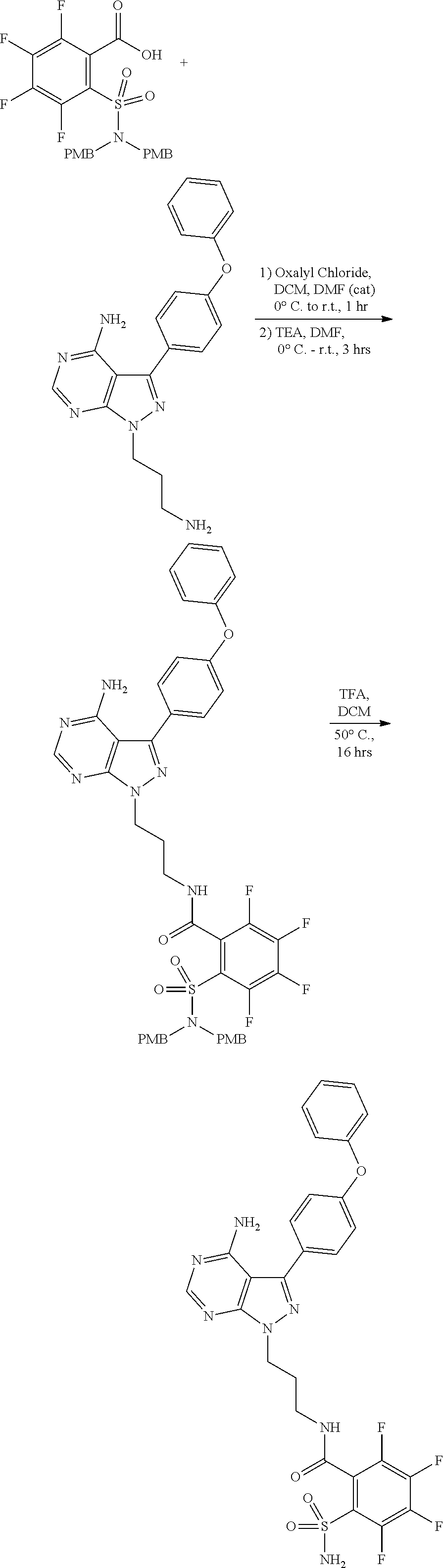

### N-(3-(4-amino-3-(4-phenoxyphenyl)-1H-pyrazolo[3,4-d]pyrimidin-1-yl)propyl)-2-(N,N-bis(4-methoxybenzyl)sulfamoyl)-3,4,5,6-tetrafluorobenzamide

N-(3-(4-amino-3-(4-phenoxyphenyl)-1H-pyrazolo[3,4-d]pyrimidin-1-yl)propyl)-2-(N,N-bis(4-methoxybenzyl)sulfamoyl)-3,4,5,6-tetrafluorobenzamide was prepared from 2-(N,N-bis(4-methoxybenzyl)sulfamoyl)-3,4,5,6-tetrafluorobenzoic acid (0.4 g, 0.77 mmol) and 1-(3-aminopropyl)-3-(4-phenoxyphenyl)-1H-pyrazolo[3,4-d]pyrimidin-4-amine (0.3 g, 0.78 mmol) according to the protocol described in general procedure A and isolated as a white solid (0.33 g, 0.39 mmol, 50% yield). ESI-MS: measured m/z 856.19 [M+1]⁺.

### N-(3-(4-amino-3-(4-phenoxyphenyl)-1H-pyrazolo[3,4-d]pyrimidin-1-yl)propyl)-2,3,4,5-tetrafluoro-6-sulfamoylbenzamide Compound I-34

N-(3-(4-amino-3-(4-phenoxyphenyl)-1H-pyrazolo[3,4-d]pyrimidin-1-yl)propyl)-2,3,4,5-tetrafluoro-6-sulfamoylbenzamide was prepared from N-(3-(4-amino-3-(4-phenoxyphenyl)-1H-pyrazolo[3,4-d]pyrimidin-1-yl)propyl)-2-(N,N-bis(4-methoxybenzyl)sulfamoyl)-3,4,5,6-tetrafluorobenzamide (0.31 g, 0.36 mmol) according to the protocol described in general procedure B and isolated as an off-white solid (0.080 g, 0.13 mmol, 36% yield). ¹H NMR (400 MHz, DMSO-d₆) δ 8.69 (s, 1H), 8.260 (s, 1H), 7.95 (br s, 2H), 7.68 (d, J=8.8 Hz, 2H), 7.443 (t, J=8.4 Hz, 2H), 7.21-7.12 (m, 5H), 4.42 (t, J=7.2 Hz, 2H), 3.28-3.26 (m, 2H), 2.09 (t, J=6.8 Hz, 2H). ¹⁹F NMR (376 MHz, DMSO-d₆) δ −134.70-−134.81 (m, 1F), 141.12-−141.21 (m, 1F), −149.20-149.30 (m, 1F). −153.35-−153.46 (m, 1F). ESI-MS: measured m/z 616.20 [M+1]⁺. HPLC (Method I): RT=6.06 min., 98.6%.

### Preparation of N-(3-(4-amino-3-(4-phenoxyphenyl)-1H-pyrazolo[3,4-d]pyrimidin-1-yl)propyl)-2,3,4,5-tetrafluoro-6-(methylsulfonyl)benzamide Compound I-39

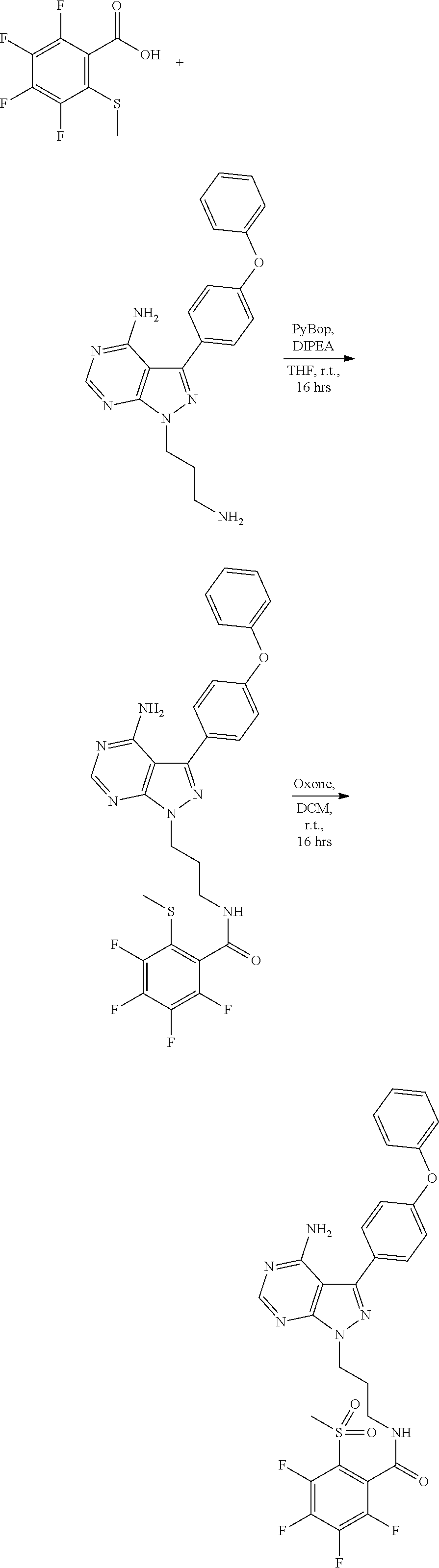 [SMILES: CSc1c(F)c(F)c(F)c(F)c1C(=O)O ; NCCCn1nc(-c2ccc(Oc3ccccc3)cc2)c2c(N)ncnc21 ; CSc1c(F)c(F)c(F)c(F)c1C(=O)NCCCn1nc(-c2ccc(Oc3ccccc3)cc2)c2c(N)ncnc21 ; CS(=O)(=O)c1c(F)c(F)c(F)c(F)c1C(=O)NCCCn1nc(-c2ccc(Oc3ccccc3)cc2)c2c(N)ncnc21]

### N-(3-(4-amino-3-(4-phenoxyphenyl)-1H-pyrazolo[3,4-d]pyrimidin-1-yl)propyl)-2,3,4,5-tetrafluoro-6-(methylthio)benzamide

To a stirred solution of 2,3,4,5-tetrafluoro-6-(methylthio)benzoic acid (0.3 g, 1.24 mmol) in THF (2 mL) was added DIPEA (0.32 g, 2.49 mmol) and PyBOP (0.97 g, 1.87 mmol), and the resulting solution cooled to 0° C. After 5 min., a solution of 1-(3-aminopropyl)-3-(4-phenoxyphenyl)-1H-pyrazolo[3,4-d]pyrimidin-4-amine (0.59 g, 1.49 mmol) was introduced, and the reaction warmed to room temperature. After 16 hrs, the reaction was diluted with water (100 mL) and extracted with EtOAc (2×100 mL). The combined organic phases were dried over anhydrous Na₂SO₄, filtered, and concentrated under reduced pressure. The crude material was purified by flash column chromatography (60% EtOAc in hexane) to afford the title compound (0.5 g, 0.85 mmol, 69% yield). ¹⁹F NMR (376 MHz, DMSO-d₆) δ −131.26-−131.35 (m, 1F), −142.21-−142.30 (m, 1F), −154.42-−154.65 (m, 2F). ESI-MS: measured m/z 583.2 [M+1]⁺.

### N-(3-(4-amino-3-(4-phenoxyphenyl)-1H-pyrazolo[3,4-d]pyrimidin-1-yl)propyl)-2,3,4,5-tetrafluoro-6-(methylsulfonyl)benzamide Compound I-39

N-(3-(4-amino-3-(4-phenoxyphenyl)-1H-pyrazolo[3,4-d]pyrimidin-1-yl)propyl)-2,3,4,5-tetrafluoro-6-(methylsulfonyl)benzamide was prepared from N-(3-(4-amino-3-(4-phenoxyphenyl)-1H-pyrazolo[3,4-d]pyrimidin-1-yl)propyl)-2,3,4,5-tetrafluoro-6-(methylthio)benzamide (0.5 g, 0.85 mmol) according to the protocol described in general procedure C and isolated as an off-white solid (0.26 g, 0.42 mmol, 49% yield). ¹H NMR (400 MHz, DMSO-d₆) δ 8.92 (t, J=5.6 Hz, 1H), 8.26 (s, 1H), 7.68 (d, J=8.8 Hz, 2H), 7.44 (t, J=7.6 Hz, 2H), 7.21-7.12 (m, 5H), 4.42 (t, J=6.8 Hz, 2H), 3.41 (s, 3H), 3.30-3.29 (m, 2H), 2.10 (t, J=7.2 Hz, 2H). ¹⁹F NMR (376 MHz, DMSO-d₆) δ −132.73-−132.85 (m, 1F), −140.20-−140.31 (m, 1F), −145.47-−145.62 (m, 1F), −151.82-−151.94 (m, 1F). ESI-MS: measured m/z 615.3 [M+1]⁺. HPLC (Method I): RT=6.25 min, 99.4%.

### N-(3-(4-amino-3-(4-phenoxyphenyl)-1H-pyrazolo[3,4-d]pyrimidin-1-yl)propyl)-2-(N,N-dimethylsulfamoyl)-3,4,5,6-tetrafluorobenzamide Compound I-41

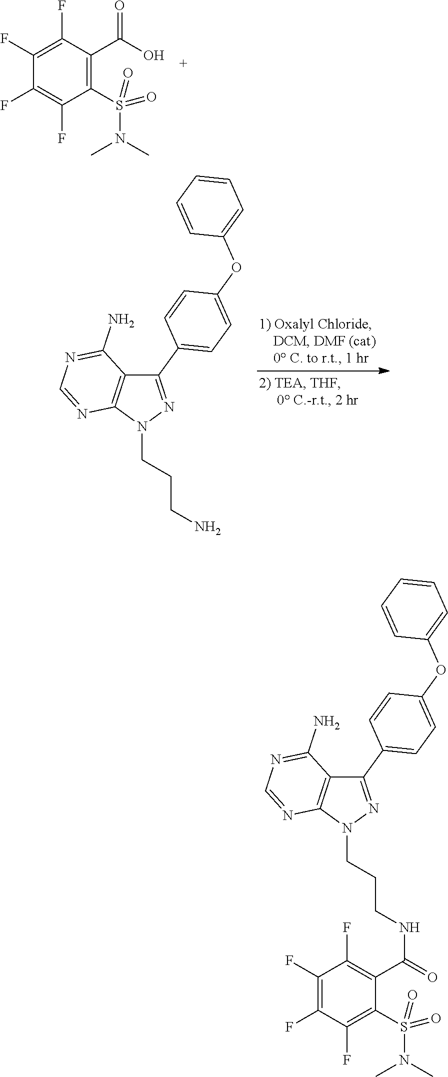 [SMILES: CN(C)S(=O)(=O)c1c(F)c(F)c(F)c(F)c1C(=O)O ; NCCCn1nc(-c2ccc(Oc3ccccc3)cc2)c2c(N)ncnc21 ; CN(C)S(=O)(=O)c1c(F)c(F)c(F)c(F)c1C(=O)NCCCn1nc(-c2ccc(Oc3ccccc3)cc2)c2c(N)ncnc21]

N-(3-(4-amino-3-(4-phenoxyphenyl)-1H-pyrazolo[3,4-d]pyrimidin-1-yl)propyl)-2-(N,N-dimethylsulfamoyl)-3,4,5,6-tetrafluorobenzamide was prepared from 2-(N,N-dimethylsulfamoyl)-3,4,5,6-tetrafluorobenzoic acid (0.3 g, 0.99 mmol) and 1-(3-aminopropyl)-3-(4-phenoxyphenyl)-1H-pyrazolo[3,4-d]pyrimidin-4-amine (0.43 g, 1.2 mmol) according to the protocol described in general procedure A and isolated as an off-white solid (0.11 g, 0.17 mmol, 17% yield). ¹H NMR (400 MHz, DMSO-d₆) δ 8.78 (t, J=5.6 Hz, 1H), 8.23 (s, 1H), 7.68 (d, J=8.8 Hz, 2H), 7.44 (t, J=8.0 Hz, 2H), 7.21-7.12 (m, 5H), 4.42-4.40 (m, 2H), 3.33-3.28 (m, 2H), 2.82 (d, J=1.6 Hz, 6H), 2.08 (m, 2H), ¹⁹F NMR (376 MHz, DMSO-d₆) δ −131.09-−131.21 (m, 1F), −140.46-−140.55 (m, 1F), −147.32-−147.46 (m, 1F), −152.17-−152.28 (m, 1F). ESI-MS: measured m/z 644.3 [M+1]⁺. HPLC (Method I) RT=6.47 min., 99.4%.

### N-(2-(4-amino-3-(4-phenoxyphenyl)-1H-pyrazolo[3,4-d]pyrimidin-1-yl)ethyl)-2-(N,N-dimethylsulfamoyl)-3,4,5,6-tetrafluorobenzamide Compound I-40

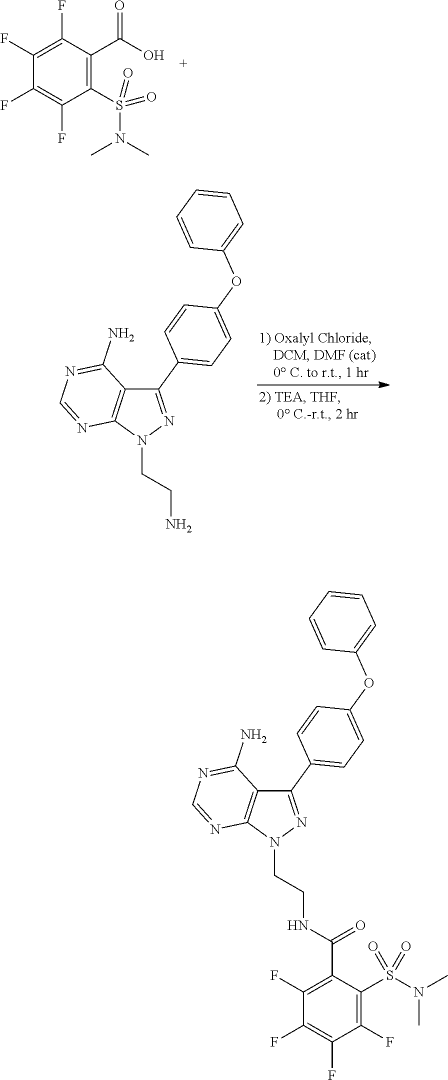 [SMILES: CN(C)S(=O)(=O)c1c(F)c(F)c(F)c(F)c1C(=O)O ; NCCn1nc(-c2ccc(Oc3ccccc3)cc2)c2c(N)ncnc21 ; CN(C)S(=O)(=O)c1c(F)c(F)c(F)c(F)c1C(=O)NCCn1nc(-c2ccc(Oc3ccccc3)cc2)c2c(N)ncnc21]

N-(2-(4-amino-3-(4-phenoxyphenyl)-1H-pyrazolo[3,4-d]pyrimidin-1-yl)ethyl)-2-(N,N-dimethylsulfamoyl)-3,4,5,6-tetrafluorobenzamide was prepared from 2-(N,N-dimethylsulfamoyl)-3,4,5,6-tetrafluorobenzoic acid (0.4 g, 1.32 mmol) and 1-(2-aminoethyl)-3-(4-phenoxyphenyl)-1H-pyrazolo[3,4-d] pyrimidin-4-amine (0.59 g, 1.72 mmol) according to the protocol described in general procedure A and isolated as an off-white solid (0.35 g, 0.55 mmol, 51% yield). ¹H NMR (400 MHz, DMSO-d₆) δ 8.89 (t, J=5.6 Hz, 1H), 8.26 (s, 1H), 7.68 (d, J=8.4 Hz, 2H), 7.44 (t, J=8.4 Hz, 2H), 7.12-7.21 (m, 5H), 4.54-4.42 (m, 2H), 3.76 (brs, 2H), 2.83-2.80 (m, 6H). ¹⁹F NMR (376 MHz, DMSO-d₆) δ −131.07-−131.19 (m, 1F), −140.16-−140.25 (m, 1F), −147.31-−147.45 (m, 1F), −151.94-−152.05 (m, 1F). ESI-MS: measured m/z 630.3 [M+1]⁺. HPLC (Method I) RT=6.38 min., 98.4%.

### Preparation of N-(2-(4-amino-3-(4-phenoxyphenyl)-1H-pyrazolo[3,4-d]pyrimidin-1-yl)ethyl)-2,3,4,5-tetrafluoro-6-(methylsulfonyl)benzamide Compound I-38

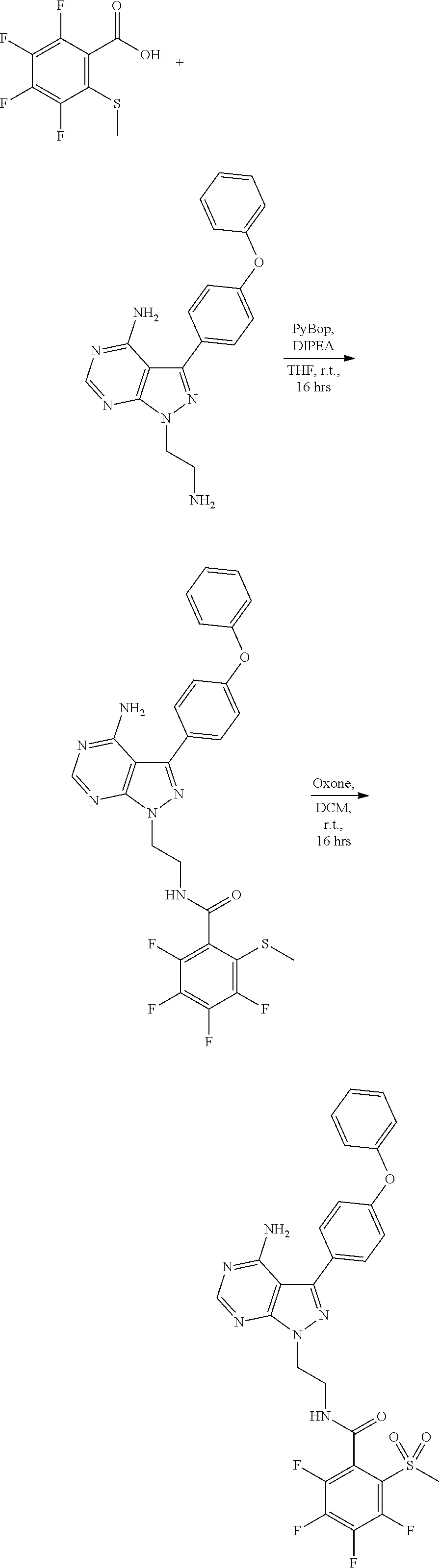 [SMILES: CSc1c(F)c(F)c(F)c(F)c1C(=O)O ; NCCn1nc(-c2ccc(Oc3ccccc3)cc2)c2c(N)ncnc21 ; CSc1c(F)c(F)c(F)c(F)c1C(=O)NCCn1nc(-c2ccc(Oc3ccccc3)cc2)c2c(N)ncnc21 ; CS(=O)(=O)c1c(F)c(F)c(F)c(F)c1C(=O)NCCn1nc(-c2ccc(Oc3ccccc3)cc2)c2c(N)ncnc21]

### N-(2-(4-amino-3-(4-phenoxyphenyl)-1H-pyrazolo[3,4-d]pyrimidin-1-yl)ethyl)-2,3,4,5-tetrafluoro-6-(methylthio)benzamide

N-(2-(4-amino-3-(4-phenoxyphenyl)-1H-pyrazolo[3,4-d]pyrimidin-1-yl)ethyl)-2,3,4,5-tetrafluoro-6-(methylthio)benzamide was prepared from 2,3,4,5-tetrafluoro-6-(methylthio)benzoic acid (0.3 g, 1.24 mmol) and 1-(2-aminoethyl)-3-(4-phenoxyphenyl)-1H-pyrazolo[3,4-d]pyrimidin-4-aminein (0.47 g, 1.37 mmol) according to the protocol described in general procedure D and isolated as an off-white solid (0.5 g, 0.88 mmol, 43% yield). ¹H NMR (400 MHz, DMSO-d₆) δ 8.87 (t, J=5.6 Hz, 1H), 8.25 (s, 1H), 7.68 (d, J=8.8 Hz, 2H), 7.44 (t, J=7.6 Hz, 2H), 7.21-7.12 (m, 5H), 4.52 (t, J=6.0 Hz, 2H) 3.86-3.81 (m, 2H), 2.28 (s, 3H). ¹⁹F NMR (376 MHz, DMSO-d₆) δ −131.33-−131.43 (m, 1F), −141.87-−141.96 (m, 1F), −154.36-−154.79 (m, 2F). ESI-MS: m/z 569.2 [M+1]⁺ HPLC (Method I) RT=6.53 min., 95.6%.

### N-(2-(4-amino-3-(4-phenoxyphenyl)-1H-pyrazolo[3,4-d]pyrimidin-1-yl)ethyl)-2,3,4,5-tetrafluoro-6-(methylsulfonyl)benzamide Compound I-38

N-(2-(4-amino-3-(4-phenoxyphenyl)-1H-pyrazolo[3,4-d]pyrimidin-1-yl)ethyl)-2,3,4,5-tetrafluoro-6-(methylsulfonyl)benzamide was prepared from N-(2-(4-amino-3-(4-phenoxyphenyl)-1H-pyrazolo[3,4-d]pyrimidin-1-yl)ethyl)-2,3,4,5-tetrafluoro-6-(methylthio)benzamide (0.40 g, 0.70 mmol) according to the protocol described in general procedure C and isolated as a yellow solid (0.07 g, 0.116 mmol, 17% yield). ¹H NMR (400 MHz, DMSO-d₆) δ 9.00 (s, 1H), 8.26 (d, J=2.4 Hz, 1H), 7.67 (d, J=8.8 Hz, 2H), 7.43 (t, J=7.6 Hz, 2H), 7.20-7.12 (m, 5H), 4.49 (br s, 2H), 3.76 (br s, 2H), 3.39 (s, 3H). ¹⁹F NMR (376 MHz, DMSO-d₆) δ −132.68-−132.80 (m, 1F), −140.02-−140.11 (m, 1F), −145.48-−145.62 (m, 1F), −151.62-−151.73 (m, 1F). ESI-MS: measured m/z 601.2 [M+1]⁺. HPLC (Method I) RT=6.11 min., 98.3%.

### Preparation of 1-(2-(methyl(2,3,4,5-tetrafluoro-6-(methylsulfonyl)phenyl)amino)ethyl)-3-(4-phenoxyphenyl)-1H-pyrazolo[3,4-d]pyrimidin-4-amine Compound I-43

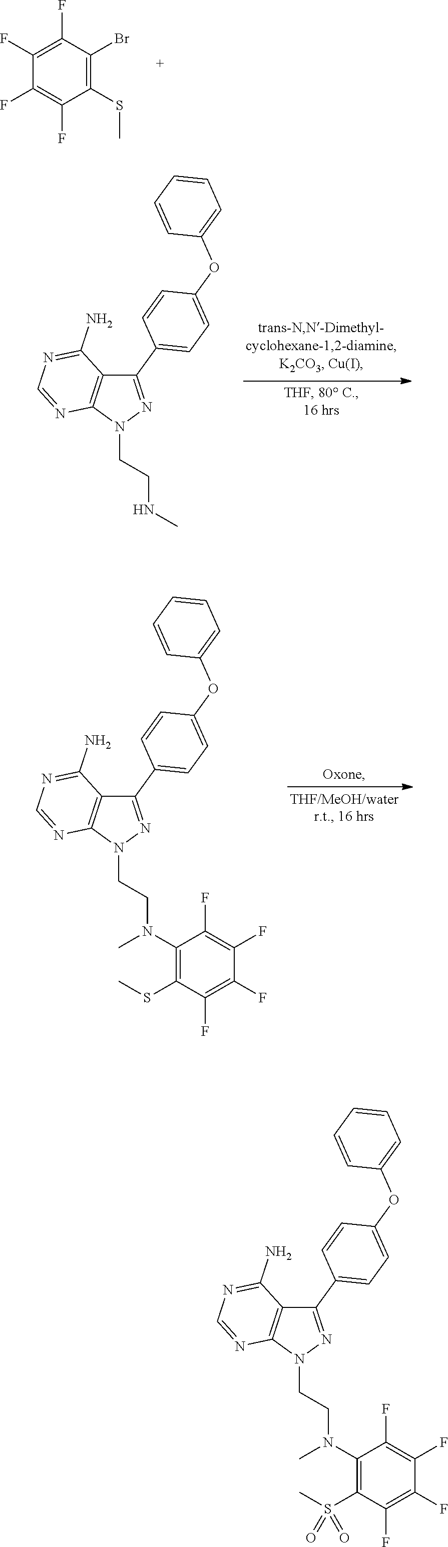 [SMILES: CSc1c(F)c(F)c(F)c(F)c1Br ; CNCCn1nc(-c2ccc(Oc3ccccc3)cc2)c2c(N)ncnc21 ; CSc1c(F)c(F)c(F)c(F)c1N(C)CCn1nc(-c2ccc(Oc3ccccc3)cc2)c2c(N)ncnc21 ; CN(CCn1nc(-c2ccc(Oc3ccccc3)cc2)c2c(N)ncnc21)c1c(F)c(F)c(F)c(F)c1S(C)(=O)=O]

### 1-(2-(methyl(2,3,4,5-tetrafluoro-6-(methylthio)phenyl)amino)ethyl)-3-(4-phenoxyphenyl)-1H-pyrazolo[3,4-d]pyrimidin-4-amine

To a stirred solution of 1-(2-(methylamino)ethyl)-3-(4-phenoxyphenyl)-1H-pyrazolo[3,4-d]pyrimidin-4-amine (0.2 g, 0.55 mmol) in THE (5 mL) was added cesium carbonate (0.54 g, 1.66 mmol) at room temperature. This resulting reaction mixture was purged with N₂ for 10 minutes, followed by addition of (2-bromo-3,4,5,6-tetrafluorophenyl)(methyl)sulfane (0.15 g, 0.55 mmol), CuI (0.021 g, 0.11 mmol) and trans-N,N′-Dimethylcyclohexane-1,2-diamine (0.031 g, 0.22 mmol). The reaction was heated to 80° C. After 16 hrs, the reaction was cooled to ambient temperature and diluted with water (20 mL). The resulting suspension was extracted with EtOAc (2×20 mL) and the combined organic phases were dried over anhydrous Na₂SO₄, filtered, and concentrated under reduced pressure. The crude material was purified by reverse phase column chromatography (70-80% ACN in Water) to afford the title compound as resin (0.16 g, 0.25 mmol, 29% yield). ¹H NMR (400 MHz, DMSO-d₆) δ 8.25-8.20 (brs, 2H), 7.99 (brs, 1H), 7.44-7.40 (m, 2H), 7.19-7.14 (m, 1H), 7.08-6.98 (m, 3H), 4.42 (t, J=6.0 Hz, 2H), 3.04 (m, 2H), 2.34 (s, 3H), 2.31 (s, 3H). ESI-MS: measured m/z 555.2 [M+1]⁺.

### 1-(2-(methyl(2,3,4,5-tetrafluoro-6-(methylsulfonyl)phenyl)amino)ethyl)-3-(4-phenoxyphenyl)-1H-pyrazolo[3,4-d]pyrimidin-4-amine Compound I-43

To a stirred solution of 1-(2-(methyl(2,3,4,5-tetrafluoro-6-(methylthio)phenyl)amino)ethyl)-3-(4-phenoxyphenyl)-1H-pyrazolo[3,4-d]pyrimidin-4-amine (0.04 g, 0.072 mmol) in THF:MeOH:Water (8:1:1, 1.6 mL) at 0° C. was added oxone (0.12 g, 0.39 mmol). The reaction mixture was permitted to warm to room temperature. After 16 hrs, a saturated aqueous solution of NaHCO₃ (20 mL) was added and the mixture extracted with DCM (2×20 mL). The combined organic phases were dried over anhydrous Na₂SO₄, filtered, and concentrated under reduced pressure. The crude material was purified by reverse phase column chromatography (30% Water in MeCN) to isolate an enriched crude which was further purified using Prep-HPLC to afford title compound as a white solid (0.006 g, 0.011 mmol, 5% yield). ¹H NMR (400 MHz, DMSO-d₆) δ 8.42 (d, J=8.4 Hz, 1H), 7.83 (d, J=7.6 Hz, 2H), 7.40 (t, J=7.2 Hz, 2H), 7.14 (t, J=7.2 Hz, 1H), 7.02 (d, J=7.6 Hz, 2H), 6.96 (d, J=8.8 Hz, 2H), 4.36 (t, J=6.0 Hz, 2H), 3.24 (s, 3H), 3.04 (t, J=6.0 Hz, 2H), 2.45 (s, 3H). ¹⁹F NMR (376 MHz, DMSO-d₆) δ −142.55-−142.64 (m, 1F), −144.60-−144.69 (m, 1F), −152.87-−152.95 (m, 1F), −170.75-−170.85 (m, 1F). ESI-MS: measured m/z 587.4 [M+1]⁺. HPLC (Method I): RT=6.35 min., 95.1%.

### Preparation of 3-(4-phenoxyphenyl)-1-(2-((2,3,4,5-tetrafluoro-6-(methylsulfonyl)benzyl)amino) ethyl)-1H-pyrazolo[3,4-d]pyrimidin-4-amine Compound I-44

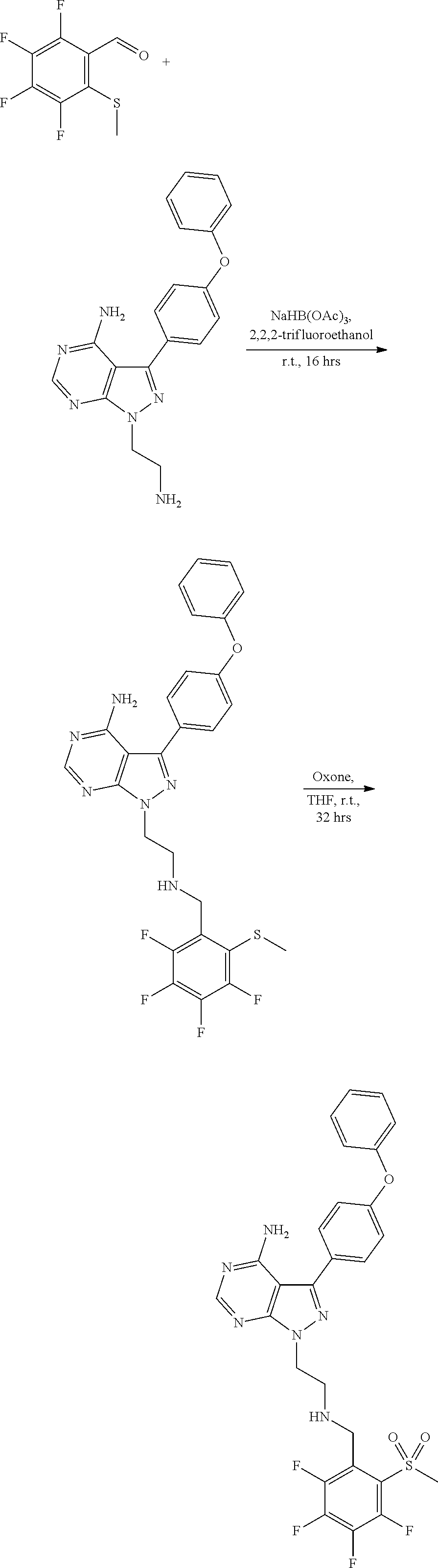 [SMILES: CSc1c(F)c(F)c(F)c(F)c1C=O ; NCCn1nc(-c2ccc(Oc3ccccc3)cc2)c2c(N)ncnc21 ; CSc1c(F)c(F)c(F)c(F)c1CNCCn1nc(-c2ccc(Oc3ccccc3)cc2)c2c(N)ncnc21 ; CS(=O)(=O)c1c(F)c(F)c(F)c(F)c1CNCCn1nc(-c2ccc(Oc3ccccc3)cc2)c2c(N)ncnc21]

### 3-(4-phenoxyphenyl)-1-(2-((2,3,4,5-tetrafluoro-6-(methylthio)benzyl)amino) ethyl)-1H-pyrazolo[3,4-d]pyrimidin-4-amine

To a stirred solution of 2,3,4,5-tetrafluoro-6-(methylthio) benzaldehyde (1.0 g, 4.46 mmol) in 2,2,2 Trifluroethanol (10 mL) was added 1-(2-aminoethyl)-3-(4-phenoxyphenyl)-1H-pyrazolo [3,4-d] pyrimidin-4-amine (1.54 g, 4.46 mmol). The reaction mixture was stirred at room temperature for 1 h and cooled 0° C. Once at temperature, neat sodium cyanoborohydride (2.79 g, 13.38 mmol) was added, and the reaction was permitted to warm to room temperature. After 16 hours, the mixture was concentrated under reduced pressure and the crude material purified by flash column chromatography (60% EtOAc in hexane) to afford the title compound as a white solid (0.27 g, 0.48 mmol, 99% yield). ESI-MS: measured m/z 555.25, [M+1]⁺.

### 3-(4-phenoxyphenyl)-1-(2-((2,3,4,5-tetrafluoro-6-(methylsulfonyl)benzyl)amino) ethyl)-1H-pyrazolo[3,4-d]pyrimidin-4-amine Compound I-44

To a stirred solution of 3-(4-phenoxyphenyl)-1-(2-((2,3,4,5-tetrafluoro-6-(methylthio)benzyl)amino) ethyl)-1H-pyrazolo[3,4-d]pyrimidin-4-amine (0.05 g, 0.09 mmol) in THF (10 mL) was added oxone (0.138 g, 0.45 mmol). The resulting reaction was stirred at room temperature for 32 hrs. Once the reaction was deemed completed, it was diluted with a saturated aqueous solution of NaHCO₃ (100 mL) and extracted with ethyl acetate (2×50 mL). The combined organic phases were dried over anhydrous Na₂SO₄, filtered, and concentrated under reduced pressure. The crude material was purified by reverse phase chromatography (27% acetonitrile in water) to afford the title compound as a white solid (0.015 g, 0.025 mmol, 6% yield). ¹H NMR (400 MHz, DMSO-d₆) δ 8.21 (s, 1H), 7.64 (d, J=8.8 Hz, 2H), 7.44 (t, J=8.4 Hz, 2H), 7.21-7.12 (m, 6H), 4.39 (t, J=6 Hz, 2H), 4.14 (br s, 2H), 3.20 (s, 3H), 3.05-3.10 (m, 2H). ¹⁹F NMR (376 MHz, DMSO-d₆) δ −132.00-−132.07 (m, 1F), −138.35-−138.43 (m, 1F), −146.17-−146.25 (m, 1F), −153.65-−153.77 (m, 1F). ESI-MS: measured m/z 587.25 [M+1]⁺. HPLC (Method I): RT=5.59 min., 96.3%.

### Preparation of N-(2-(4-amino-3-(4-phenoxyphenyl)-1H-pyrazolo[3,4-d]pyrimidin-1-yl)ethyl)-2,3,4,5-tetrafluoro-6-sulfamoylbenzamide Compound I-42

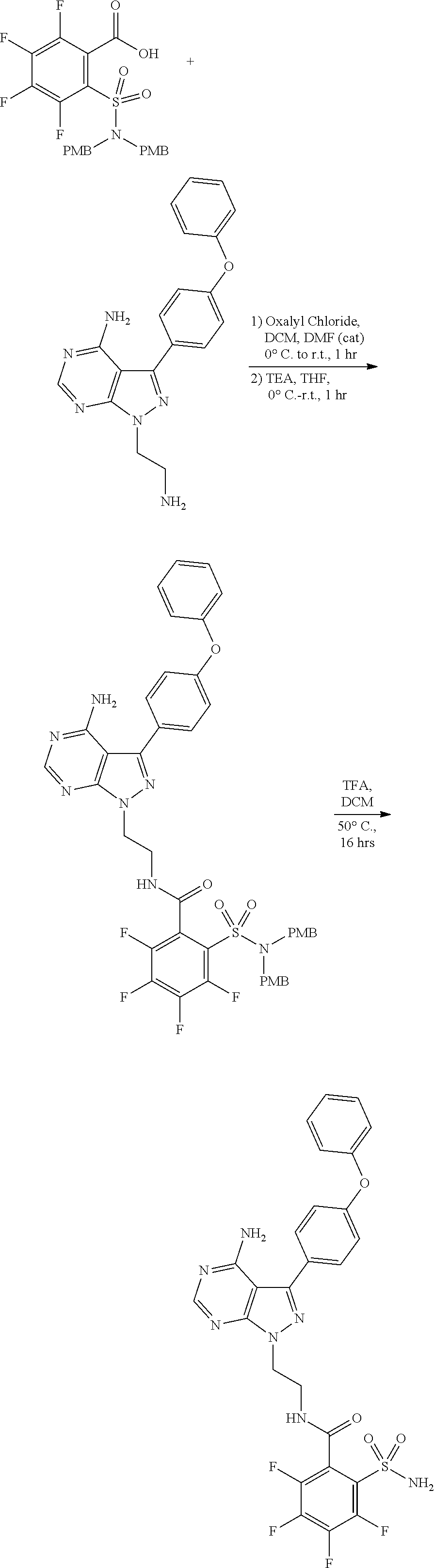

### N-(2-(4-amino-3-(4-phenoxyphenyl)-1H-pyrazolo[3,4-d]pyrimidin-1-yl)ethyl)-2-(N,N-bis(4-methoxybenzyl)sulfamoyl)-3,4,5,6-tetrafluorobenzamide

N-(2-(4-amino-3-(4-phenoxyphenyl)-1H-pyrazolo[3,4-d]pyrimidin-1-yl)ethyl)-2-(N,N-bis(4-methoxybenzyl)sulfamoyl)-3,4,5,6-tetrafluorobenzamide was prepared from 2-(N,N-bis(4-methoxybenzyl)sulfamoyl)-3,4,5,6-tetrafluorobenzoic acid (0.4 g, 0.77 mmol) and 1-(2-aminoethyl)-3-(4-phenoxyphenyl)-1H-pyrazolo[3,4-d]pyrimidin-4-amine (0.35 g, 1.01 mmol) according to the protocol described in general procedure A and isolated as an off-white solid (0.35 g, 0.41 mmol, 53% yield). ESI-MS: measured m/z 843.0 [M+1]⁺.

### N-(2-(4-amino-3-(4-phenoxyphenyl)-1H-pyrazolo[3,4-d]pyrimidin-1-yl)ethyl)-2,3,4,5-tetrafluoro-6-sulfamoylbenzamide Compound I-42

N-(2-(4-amino-3-(4-phenoxyphenyl)-1H-pyrazolo[3,4-d]pyrimidin-1-yl)ethyl)-2,3,4,5-tetrafluoro-6-sulfamoylbenzamide was prepared from N-(2-(4-amino-3-(4-phenoxyphenyl)-1H-pyrazolo[3,4-d]pyrimidin-1-yl)ethyl)-2-(N,N-bis(4-methoxybenzyl)sulfamoyl)-3,4,5,6-tetrafluorobenzamide (0.35 g, 0.41 mmol) according to the protocol described in general procedure B and isolated as a white solid (0.04 g, 0.06 mmol, 35% yield). ¹H NMR (400 MHz, DMSO-d₆) δ 8.27 (s, 1H), 8.26 (s, 1H), 8.08 (brs, 2H) 7.68 (d, J=8.8 Hz, 2H), 7.44 (t, J=8.0 Hz, 3H), 7.21-7.12 (m, 5H), 5.73 (m, 1H), 4.48 (m, 2H), 3.73-3.72 (m, 2H). ¹⁹F NMR (376 MHz, DMSO-d₆) δ −134.62-−134.68 (m, 1F), −140.90-−140.99 (m, 1F), −149.08-−149.17 (m, 1F), −153.02-−153.14 (m, 1F). ESI-MS: m/z 602.3 [M+1]⁺. HPLC (Method I): RT=5.94 min., 98.2%.

### General Procedure E:

### (R)-3-(4-phenoxyphenyl)-1-(1-((2,3,4,5-tetrafluoro-6-(fluoromethoxy)phenyl)sulfonyl) pyrrolidin-3-yl)-1H-pyrazolo[3,4-d]pyrimidin-4-amine Compound I-24

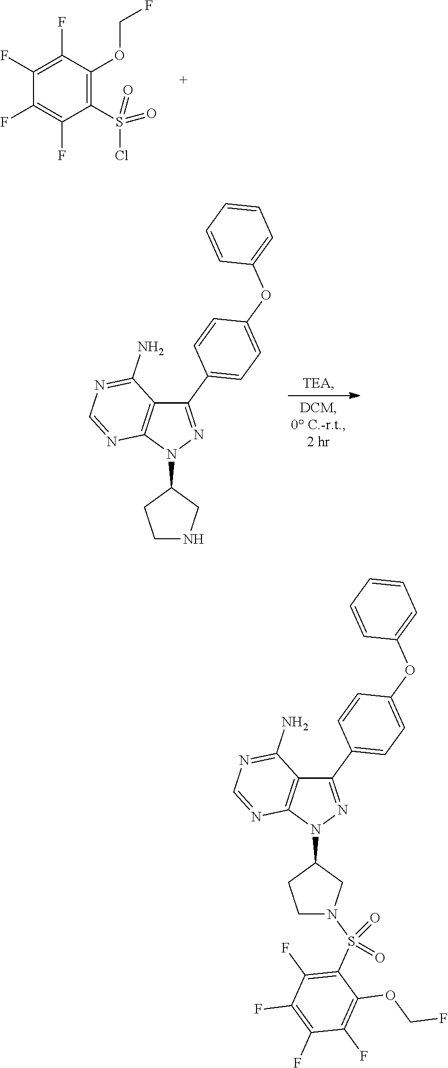 [SMILES: O=S(=O)(Cl)c1c(F)c(F)c(F)c(F)c1OCF ; Nc1ncnc2c1c(-c1ccc(Oc3ccccc3)cc1)nn2[C@@H]1CCNC1 ; Nc1ncnc2c1c(-c1ccc(Oc3ccccc3)cc1)nn2[C@@H]1CCN(S(=O)(=O)c2c(F)c(F)c(F)c(F)c2OCF)C1]

2,3,4,5-tetrafluoro-6-(fluoromethoxy)benzenesulfonyl chloride (382 mg, 1.29 mmol) was added dropwise to a solution of 3-(4-phenoxyphenyl)-1-[(3R)-pyrrolidin-3-yl]pyrazolo[3,4-d]pyrimidin-4-amine (400 mg, 1.07 mmol) in anhydrous dichloromethane (10 mL) at 0° C. under argon and stirred for 15 minutes. Triethylamine (217 mg, 2.15 mmol, 299 uL) was slowly added to the mixture and the reaction permitted to warm to room temperature. After 2 hrs, the reaction was diluted with 0.1M HCl and the organic layer was separated. The aqueous layer was extracted with DCM once more. The combined organic layers were dried over sodium sulfate and evaporated to dryness, providing crude product as a beige foam. The crude material was purified by flash column chromatography (30-50% ethyl acetate/DCM) to afford the title compound as a white foam (54 mg, 0.085 mmol, 8% yield). ¹H NMR (400 MHz, CDCl₃) δ 8.34 (s, 1H), 7.53-7.33 (m, 4H), 7.21 (s, 1H), 7.17-7.05 (m, 4H), 5.74-5.46 (m, 5H), 4.01 (d, J=5.2 Hz, 2H), 3.89 (t, J=8.4 Hz, 1H), 3.75 (ddd, J=9.4, 7.9, 4.2 Hz, 1H), 2.62-2.44 (m, 2H). ¹⁹F NMR (376 MHz, CDCl₃) δ −132.92 (dt, J=24.2, 8.1 Hz), −145.98 (td, J=21.7, 6.9 Hz), −148.65 (td, J=22.0, 9.1 Hz), −149.19-−150.02 (m), −156.36 (dd, J=24.6, 21.2 Hz). ESI-MS: m/z 633.2 [M+1]⁺. HPLC (Method III): RT=5.70 min., 95.2%.

### N-(2-(4-amino-3-(4-phenoxyphenyl)-1H-pyrazolo[3,4-d]pyrimidin-1-yl)ethyl)-2,3,4,5-tetrafluoro-6-(fluoromethoxy)benzenesulfonamide Compound I-25

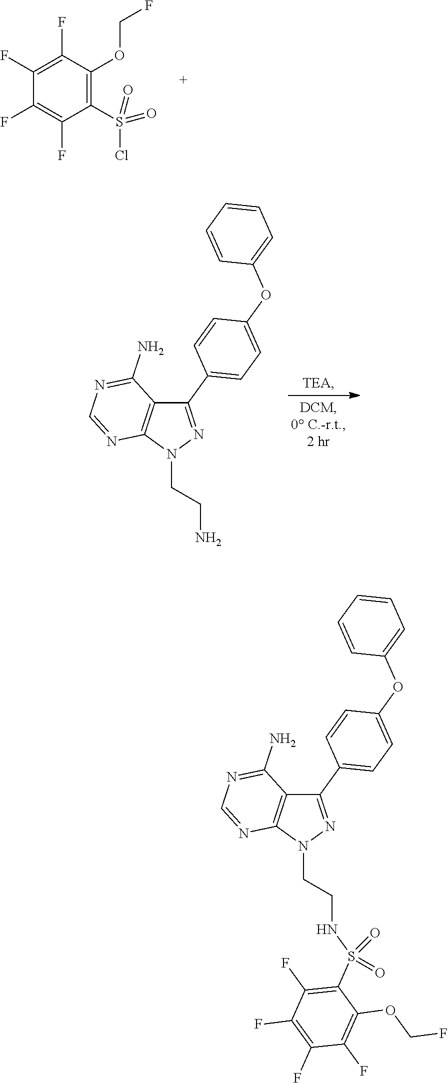 [SMILES: O=S(=O)(Cl)c1c(F)c(F)c(F)c(F)c1OCF ; NCCn1nc(-c2ccc(Oc3ccccc3)cc2)c2c(N)ncnc21 ; Nc1ncnc2c1c(-c1ccc(Oc3ccccc3)cc1)nn2CCNS(=O)(=O)c1c(F)c(F)c(F)c(F)c1OCF]

N-(2-(4-amino-3-(4-phenoxyphenyl)-1H-pyrazolo[3,4-d]pyrimidin-1-yl)ethyl)-2,3,4,5-tetrafluoro-6-(fluoromethoxy)benzenesulfonamide was prepared from 2,3,4,5-tetrafluoro-6-(fluoromethoxy)benzenesulfonyl chloride (216.92 mg, 731.36 μmol) and 1-(2-aminoethyl)-3-(4-phenoxyphenyl)pyrazolo[3,4-d]pyrimidin-4-amine hydrochloride (0.28 g, 731.36 μmol) according to the protocol described in general procedure E and isolated as a white solid (0.062 g, 102 μmol, 14% yield). ¹H NMR (400 MHz, CDCl₃) δ 8.35 (s, 1H), 7.72-7.57 (m, 2H), 7.54-7.36 (m, 2H), 7.29-7.11 (m, 5H), 7.08 (s, 1H), 5.79 (bs, 2H), 5.61 (d, J=53.4 Hz, 2H), 4.70-4.56 (m, 2H), 3.81 (s, 2H). ¹⁹F NMR (376 MHz, CDCl₃) δ −135.21-−136.26 (m), −146.54-−146.90 (m), −149.04-−149.65 (m), −156.26 (dd, J=23.8, 20.8 Hz). ESI-MS: m/z 607.2 [M+1]⁺. HPLC (Method III): RT=4.62 min., 98%.

### 3-(4-phenoxyphenyl)-1-((2,3,4,5-tetrafluoro-6-methoxyphenyl)sulfonyl)-1H-pyrazolo[3,4-d]pyrimidin-4-amine Compound I-23

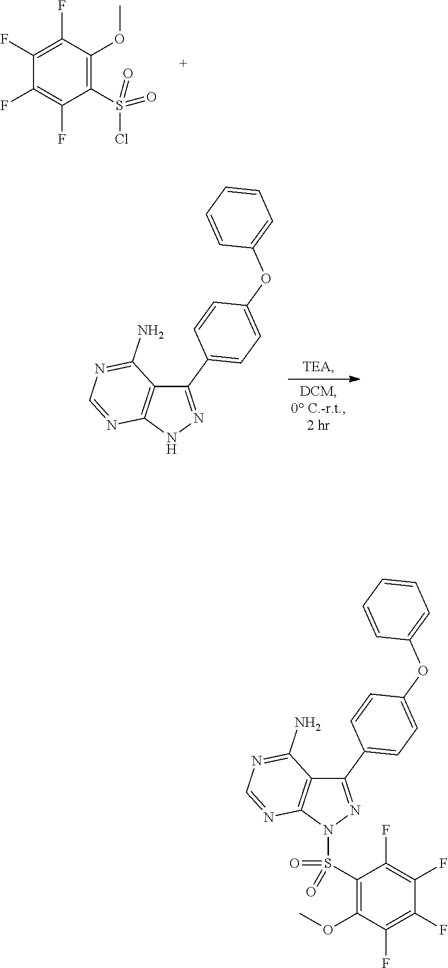 [SMILES: COc1c(F)c(F)c(F)c(F)c1S(=O)(=O)Cl ; Nc1ncnc2[nH]nc(-c3ccc(Oc4ccccc4)cc3)c12 ; COc1c(F)c(F)c(F)c(F)c1S(=O)(=O)n1nc(-c2ccc(Oc3ccccc3)cc2)c2c(N)ncnc21]

3-(4-phenoxyphenyl)-1-((2,3,4,5-tetrafluoro-6-methoxyphenyl)sulfonyl)-1H-pyrazolo[3,4-d]pyrimidin-4-amine was prepared from 2,3,4,5-tetrafluoro-6-methoxybenzenesulfonyl chloride (130 mg, 0.47 mmol) and 3-(4-phenoxyphenyl)-1H-pyrazolo[3,4-d]pyrimidin-4-amine (0.156 g, 0.51 mmol) according to the protocol described in general procedure E and isolated as a white solid (0.004 g, 8 μmol, 2% yield). ¹H NMR (400 MHz, CDCl₃) δ 8.55 (s, 1H), 7.70 (d, J=7.8 Hz, 2H), 7.41 (t, J=8.1 Hz, 2H), 7.23 (t, J=8.1 Hz, 1H), 7.12 (d, J=7.8 Hz, 2H), 7.05 (d, J=8.1 Hz, 2H), 5.80 (bs, 2H), 4.05 (s, 1H). ¹⁹F NMR (376 MHz, CDCl₃) δ −134.04-−134.15 (m, 1F), −143.52-−143.65 (m, 1F), −152.75-−152.83 (m, 1F), −159.47-−159.59 (m, 1F). ESI-MS: m/z 546.17 [M+1]⁺. HPLC (Method III): RT=5.61 min., 95%.

### N-(2-(4-amino-3-(4-phenoxyphenyl)-1H-pyrazolo[3,4-d]pyrimidin-1-yl)ethyl)-2-(difluoromethyl)-3,4,5,6-tetrafluorobenzenesulfonamide Compound I-22

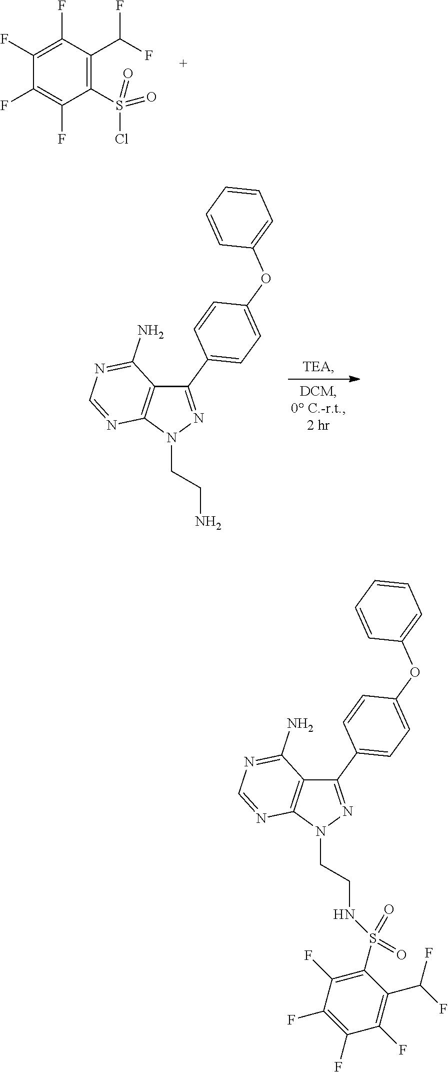 [SMILES: O=S(=O)(Cl)c1c(F)c(F)c(F)c(F)c1C(F)F ; NCCn1nc(-c2ccc(Oc3ccccc3)cc2)c2c(N)ncnc21 ; Nc1ncnc2c1c(-c1ccc(Oc3ccccc3)cc1)nn2CCNS(=O)(=O)c1c(F)c(F)c(F)c(F)c1C(F)F]

N-(2-(4-amino-3-(4-phenoxyphenyl)-1H-pyrazolo[3,4-d]pyrimidin-1-yl)ethyl)-2-(difluoromethyl)-3,4,5,6-tetrafluorobenzenesulfonamide was prepared from 2-(difluoromethyl)-3,4,5,6-tetrafluorobenzenesulfonyl chloride (60 mg, 167 μmol) and 1-(2-aminoethyl)-3-(4-phenoxyphenyl)pyrazolo[3,4-d]pyrimidin-4-amine hydrochloride (0.71 g, 186 μmol) according to the protocol described in general procedure E and isolated as a white solid (0.041 g, 67 μmol, 36% yield). ¹H NMR (400 MHz, CDCl₃) δ 8.27 (d, J=1.5 Hz, 1H), 7.81 (t, J=52.2 Hz, 1H), 7.64-7.53 (m, 2H), 7.47-7.38 (m, 2H), 7.24-7.07 (m, 4H), 5.90 (s, 2H), 4.61 (td, J=4.9, 1.9 Hz, 2H), 3.81 (t, J=5.2 Hz, 2H). ¹⁹F NMR (376 MHz, CDCl₃) δ −112.75 (dd, J=52.3, 27.8 Hz, 2F), −133.47 (dt, J=21.2, 9.9 Hz, 1F), −133.94 (tdt, J=28.7, 19.1, 9.2 Hz, 1F), −145.51 (td, J=20.5, 9.3 Hz, 1F), −147.45 (td, J=21.7, 7.8 Hz, 1F). LC-MS (ESI−) m/z calc'd for [C₂₆H₁₈F₆N₆O₃S]⁻: 607.1. found: 607.1. HPLC purity: 96%

### (R)-1-(1-((2-(difluoromethoxy)-3,4,5,6-tetrafluorophenyl)sulfonyl)pyrrolidin-3-yl)-3-(4-phenoxyphenyl)-1H-pyrazolo[3,4-d]pyrimidin-4-amine Compound I-26

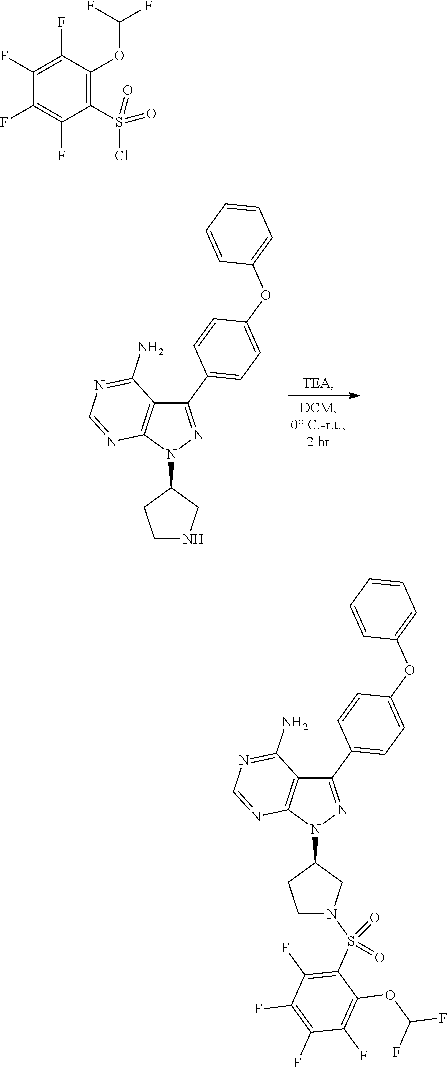 [SMILES: O=S(=O)(Cl)c1c(F)c(F)c(F)c(F)c1OC(F)F ; Nc1ncnc2c1c(-c1ccc(Oc3ccccc3)cc1)nn2[C@@H]1CCNC1 ; Nc1ncnc2c1c(-c1ccc(Oc3ccccc3)cc1)nn2[C@@H]1CCN(S(=O)(=O)c2c(F)c(F)c(F)c(F)c2OC(F)F)C1]

(R)-1-(1-((2-(difluoromethoxy)-3,4,5,6-tetrafluorophenyl)sulfonyl)pyrrolidin-3-yl)-3-(4-phenoxyphenyl)-1H-pyrazolo[3,4-d]pyrimidin-4-amine was prepared from 2-(difluoromethoxy)-3,4,5,6-tetrafluorobenzenesulfonyl chloride (25 mg, 79 μmol) and 3-(4-phenoxyphenyl)-1-[(3R)-pyrrolidin-3-yl]pyrazolo[3,4-d]pyrimidin-4-amine (27 mg, 72 mmol) according to the protocol described in general procedure E and isolated as a white solid (0.028 g, 43 μmol, 60% yield). ¹H NMR (400 MHz, CDCl₃) δ 8.35 (s, 1H), 7.55-7.39 (m, 4H), 7.31-7.23 (m, 1H), 7.23-7.11 (m, 4H), 6.64 (td, J=74.7, 1.0 Hz, 1H), 5.75-5.53 (m, 3H), 4.05 (d, J=5.0 Hz, 2H), 3.94 (q, J=8.5 Hz, 1H), 3.80 (ddd, J=9.4, 7.4, 4.5 Hz, 1H), 2.62-2.46 (m, 2H). ¹⁹F NMR (376 MHz, CDCl₃) δ −83.13 (dd, J=74.8, 13.7 Hz), −132.27 (dt, J=24.4, 8.0 Hz), −145.47 (td, J=21.4, 7.0 Hz), −146.97 (td, J=23.3, 22.8, 13.5 Hz), −154.19 (ddd, J=23.7, 21.0, 2.2 Hz). LC-MS (ESI−) m/z calc'd for [C₂₈H₂₀F₆N₆O₄S]⁺: 651.1. Found: 651.2. LC-MS purity: 97.2%

### 2-(difluoromethyl)-3,4,5,6-tetrafluoro-N-(3-((5-fluoro-2-((4-(2-methoxyethoxy) phenyl)amino)pyrimidin-4-yl)amino)phenyl)benzenesulfonamide Compound I-51

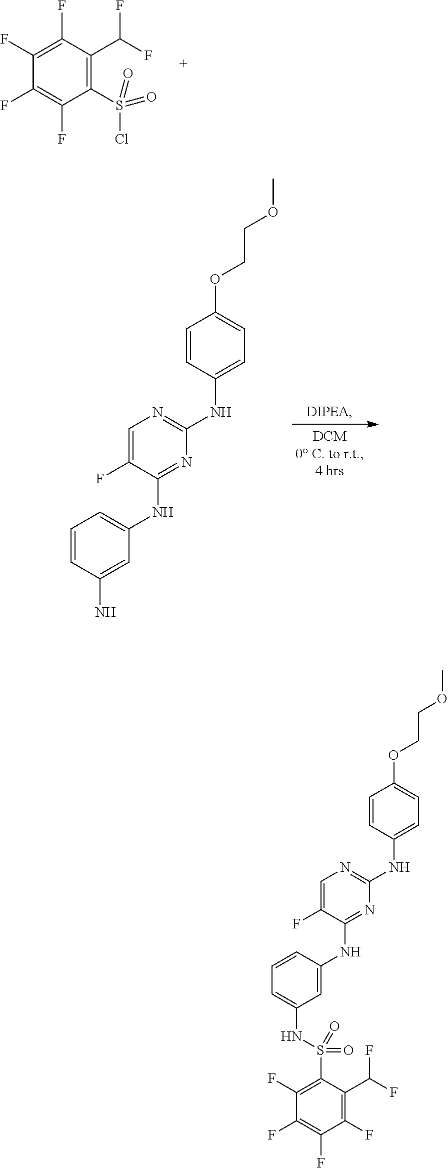 [SMILES: O=S(=O)(Cl)c1c(F)c(F)c(F)c(F)c1C(F)F ; COCCOc1ccc(Nc2ncc(F)c(Nc3cccc(N)c3)n2)cc1 ; COCCOc1ccc(Nc2ncc(F)c(Nc3cccc(NS(=O)(=O)c4c(F)c(F)c(F)c(F)c4C(F)F)c3)n2)cc1]

2-(difluoromethyl)-3,4,5,6-tetrafluoro-N-(3-((5-fluoro-2-((4-(2-methoxyethoxy) phenyl)amino)pyrimidin-4-yl)amino)phenyl)benzenesulfonamide was prepared from 2-(difluoromethyl)-3,4,5,6-tetrafluoro-benzenesulfonyl chloride (50 mg, 0.167 mmol) and N⁴-(3-aminophenyl)-5-fluoro-N²-[4-(2-methoxyethoxy)phenyl]pyrimidine-2,4-diamine (123 mg, 0.334 mmol) according to the protocol described in general procedure E and isolated as a white solid (0.005 g, 8.71 μmol, 5.2% yield). ¹H NMR (400 MHz, CDCl₃) δ 7.98-7.63 (m, 3H), 7.44 (s, 1H), 7.38 (d, J=8.6 Hz, 2H), 7.28-7.20 (m, 2H), 7.00-6.85 (m, 4H), 4.20-4.08 (m, 2H), 3.83-3.75 (m, 2H), 3.49 (s, 3H). ¹⁹F NMR (376 MHz, CDCl₃) δ −112.90 (dd, J=52.2, 27.4 Hz, 2F), −132.29-−132.51 (m, 1F), −132.90-−133.26 (m, 1F), −143.48 (td, J=20.6, 10.2 Hz, 1F), −146.89 (td, J=22.4, 8.1 Hz, 1F), −166.90 (m, 1F). Analytical HPLC purity: 96.0%

### 2-(difluoromethyl)-3,4,5,6-tetrafluoro-N-(3-((5-fluoro-2-((4-(2-methoxyethoxy) phenyl)amino)pyrimidin-4-yl)amino)propyl)benzenesulfonamide Compound I-52

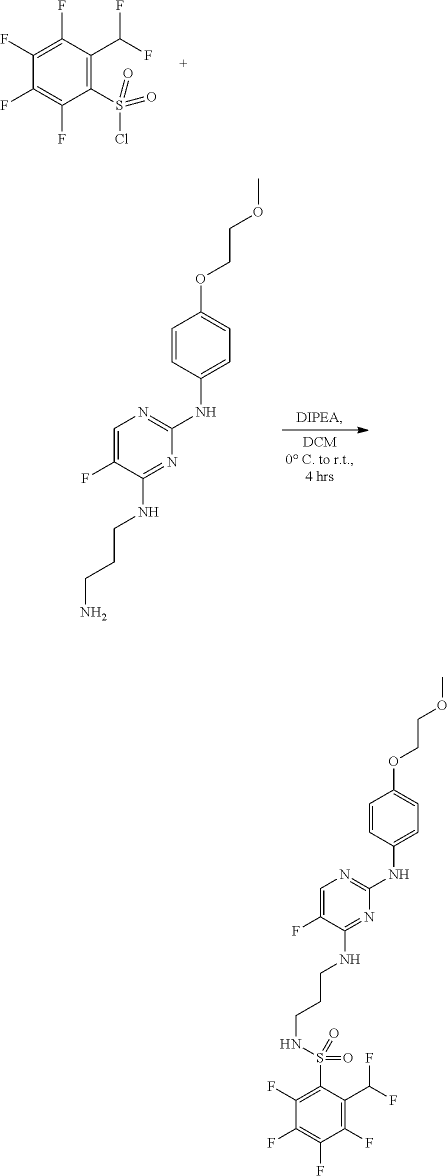 [SMILES: O=S(=O)(Cl)c1c(F)c(F)c(F)c(F)c1C(F)F ; COCCOc1ccc(Nc2ncc(F)c(NCCCN)n2)cc1 ; COCCOc1ccc(Nc2ncc(F)c(NCCCNS(=O)(=O)c3c(F)c(F)c(F)c(F)c3C(F)F)n2)cc1]

A mixture of N⁴-(3-aminopropyl)-5-fluoro-N²-[4-(2-methoxyethoxy)phenyl]pyrimidine-2,4-diamine (60 mg, 178.90 μmol) and DIPEA (46.24 mg, 357.81 μmol, 62.32 uL) in DCM (1.8 mL) was stirred at 0° C. while a solution of 2-(difluoromethyl)-3,4,5,6-tetrafluoro-benzenesulfonyl chloride (48.08 mg, 161.01 mol, 23.84 uL) in DCM (1.8 mL) was added dropwise. The resulting mixture was warmed to room temperature over 4 hours. The reaction was quenched with water and extracted with EtOAc (10 mL×3), washed with brine, and dried over Na₂SO₄. The solvent was removed under reduced pressure, and the residue was separated on a pad of silica eluting with a gradient of EtOAc in Hexanes (30-40%) to obtain the title compound as a beige solid (0.010 g, 16.4 μmol, 9% yield). ¹H NMR (400 MHz, CDCl₃) δ 7.99-7.59 (m, 2H), 7.47-7.41 (m, 2H), 7.24 (s, 1H), 6.94-6.86 (m, 2H), 4.21-4.05 (m, 3H), 3.80-3.73 (m, 2H), 3.66 (q, J=6.3 Hz, 2H), 3.47 (s, 3H), 3.20 (t, J=6.0 Hz, 2H). ¹⁹F NMR (376 MHz, CDCl₃) δ −112.76 (dd, J=52.4, 27.5 Hz, 2F), −133.73 (tdt, J=28.7, 20.2, 9.6 Hz, 1F), −134.45 (dt, J=21.2, 10.2 Hz, 1F), −145.33 (td, J=20.4, 9.2 Hz, 1F), −147.24 (td, J=21.8, 20.6, 7.8 Hz, 1F), −169.11 (1F). Analytical HPLC purity: 97.3%

### 2,3,4,5-tetrafluoro-N-(3-((5-fluoro-2-((4-(2-methoxyethoxy)phenyl)amino)pyrimidin-4-yl)amino)phenyl)-6-(trifluoromethyl)benzenesulfonamide Compound I-53

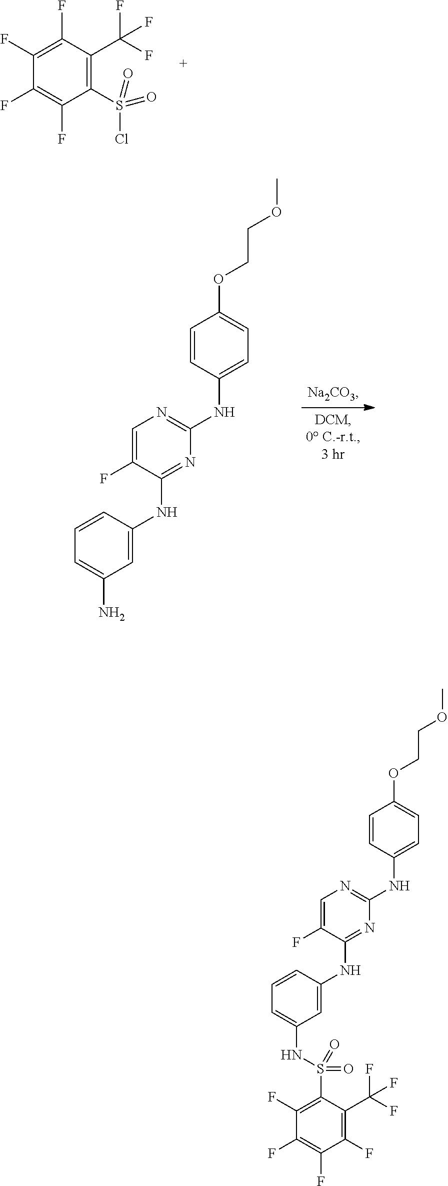 [SMILES: O=S(=O)(Cl)c1c(F)c(F)c(F)c(F)c1C(F)(F)F ; COCCOc1ccc(Nc2ncc(F)c(Nc3cccc(N)c3)n2)cc1 ; COCCOc1ccc(Nc2ncc(F)c(Nc3cccc(NS(=O)(=O)c4c(F)c(F)c(F)c(F)c4C(F)(F)F)c3)n2)cc1]

A mixture of N⁴-(3-aminophenyl)-5-fluoro-N²-[4-(2-methoxyethoxy)phenyl]pyrimidine-2,4-diamine (175.02 mg, 473.81 μmol), DCM (0.1 M) (3.16 mL) and sodium carbonate (33.48 mg, 315.87 μmol, 13.23 uL)) was stirred at 0° C. while a solution of 2,3,4,5-tetrafluoro-6-(trifluoromethyl)benzenesulfonyl chloride (0.1 g, 315.87 μmol, 21.05 uL) in DCM (0.1 M) (3.16 mL) was added dropwise and the resulting mixture allowed to warm to room temperature. After 3 hrs, the reaction was quenched with water and extracted with DCM (×3), washed with brine, and dried over Na₂SO₄. The solvent was removed under reduced pressure, and the residue was separated Biotage Isolera equipped with a reverse phase cartridge (90% to 50% MiliQ water in HPLC-grade acetonitrile) to afford the desired product as light yellow solid (0.014 g, 21.5 μmol, 7% yield). ¹H NMR (400 MHz, CDCl₃) δ 7.78 (s, 3H), 7.33 (d, J=8.4 Hz, 2H), 7.24 (d, J=7.9 Hz, OH), 7.05 (s, 1H), 6.91 (d, J=8.3 Hz, 2H), 4.16 (s, 2H), 3.79 (d, J=4.8 Hz, 2H), 3.48 (s, 3H). ¹⁹F NMR (376 MHz, CDCl₃) δ −51.47 (3F), −129.21 (1F), −130.42 (1F), −143.10 (1F), −144.14 (1F). LC-MS (ESI+) m/z calc'd for [C₂₆H₁₉F₈N₅O₄S]⁺: 648.1. Found: 648.1. Analytical HPLC purity: 97.2%

### 2-(difluoromethoxy)-3,4,5,6-tetrafluoro-N-(3-((5-fluoro-2-((4-(2-methoxyethoxy) phenyl)amino)pyrimidin-4-yl)amino)phenyl)benzenesulfonamide Compound I-54

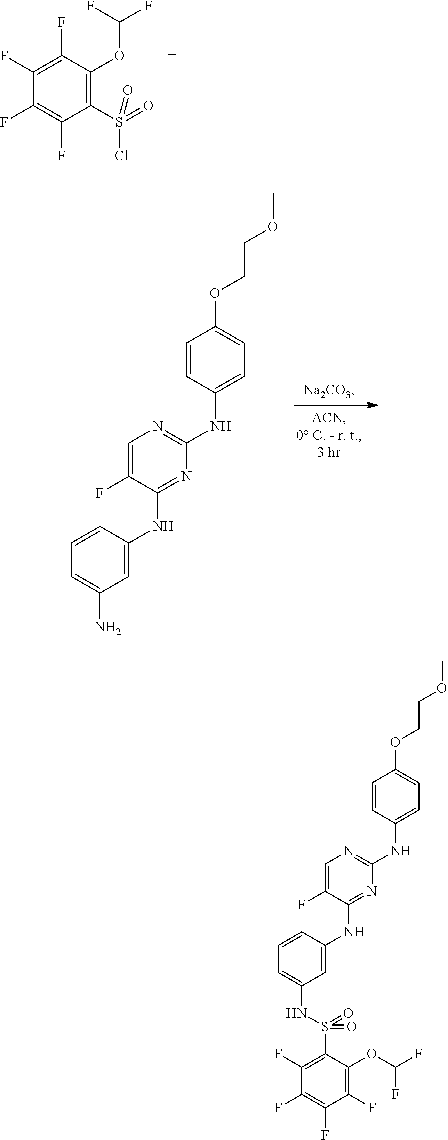 [SMILES: O=S(=O)(Cl)c1c(F)c(F)c(F)c(F)c1OC(F)F ; COCCOc1ccc(Nc2ncc(F)c(Nc3cccc(N)c3)n2)cc1 ; COCCOc1ccc(Nc2ncc(F)c(Nc3cccc(NS(=O)(=O)c4c(F)c(F)c(F)c(F)c4OC(F)F)c3)n2)cc1]

A mixture of N⁴-(3-aminophenyl)-5-fluoro-N²-[4-(2-methoxyethoxy)phenyl]pyrimidine-2,4-diamine (27.96 mg, 75.68 μmol), ACN (0.8 mL) and sodium carbonate (8.02 mg, 75.68 μmol) was stirred at 0° C. while a solution of 2-(difluoromethoxy)-3,4,5,6-tetrafluoro-benzenesulfonyl chloride (0.025 g, 79.47 μmol, 21.05 uL) in CAN (0.8 mL) was added dropwise and the resulting mixture allowed to warm to room temperature. After 3 hrs, the reaction was quenched with water and extracted with DCM (×3), washed with brine, and dried over Na₂SO₄. The solvent was removed under reduced pressure, and the residue was separated on a Biotage Isolera equipped with a normal phase cartridge (30-40% EtOAc in hexanes) to afford the desired product as an oil (0.015 g, 23.2 μmol, 30% yield). ¹H NMR (400 MHz, CDCl₃) δ 7.98 (d, J=3.1 Hz, 1H), 7.87 (s, 1H), 7.45-7.38 (m, 2H), 7.28-7.22 (m, 2H), 6.97-6.84 (m, 5H), 6.79 (d, J=3.1 Hz, 1H), 6.58 (d, J=73.5 Hz, 1H), 4.20-4.13 (m, 2H), 3.82-3.76 (m, 2H), 3.49 (s, 3H). ¹⁹F NMR (376 MHz, CDCl₃) δ −81.85 (dd, J=73.5, 12.8 Hz, 2F), −134.21 (dt, J=24.0, 8.5 Hz, 1F), −144.20 (td, J=21.0, 7.8 Hz, 1F), −146.83-−147.02 (m, 1F), −153.21 (d, J=2.5 Hz, 1F), −167.76 (t, J=3.2 Hz, 1F). LC-MS (ESI−) m/z calc'd for [C₂₆H₂₀F₇N₅O₅S]−: 646.1. Found: 646.1.

### 2-(difluoromethoxy)-3,4,5,6-tetrafluoro-N-(3-((5-fluoro-2-((4-(2-methoxyethoxy) phenyl)amino)pyrimidin-4-yl)amino)propyl)benzenesulfonamide Compound I-55

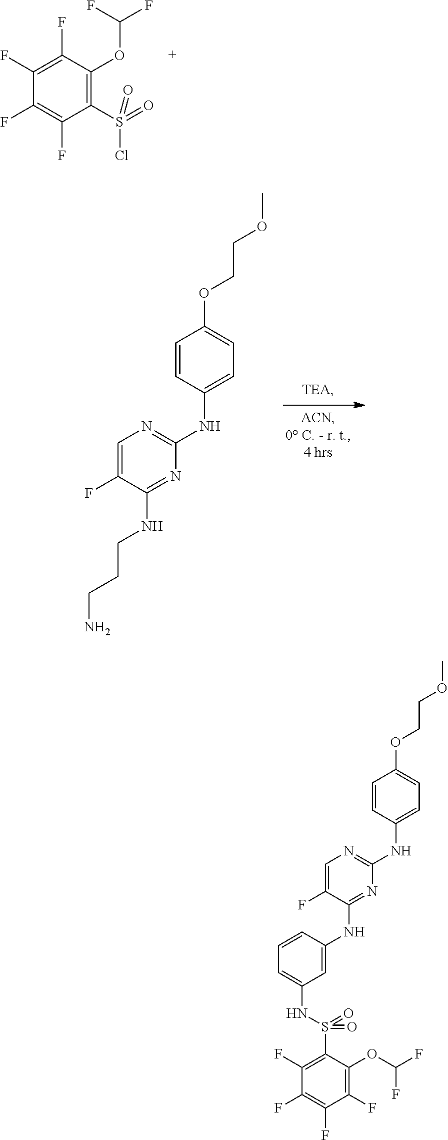 [SMILES: O=S(=O)(Cl)c1c(F)c(F)c(F)c(F)c1OC(F)F ; COCCOc1ccc(Nc2ncc(F)c(NCCCN)n2)cc1 ; COCCOc1ccc(Nc2ncc(F)c(Nc3cccc(NS(=O)(=O)c4c(F)c(F)c(F)c(F)c4OC(F)F)c3)n2)cc1]

A mixture of N⁴-(3-aminopropyl)-5-fluoro-N²-[4-(2-methoxyethoxy)phenyl]pyrimidine-2,4-diamine (31.98 mg, 95.36 μmol), ACN (1 mL) and TEA (29 mg, 286 μmol) was stirred at 0° C. while a solution of 2-(difluoromethoxy)-3,4,5,6-tetrafluoro-benzenesulfonyl chloride (0.025 g, 79.47 μmol, 21.05 uL) in ACN (1 mL) was added dropwise and the resulting mixture allowed to warm to room temperature. After 4 hrs, the reaction was quenched with water and extracted with DCM (×3), washed with brine, and dried over Na₂SO₄. The solvent was removed under reduced pressure, and the residue was separated on a Biotage Isolera equipped with a normal phase cartridge (30-40% EtOAc in hexanes) to afford the desired product (0.02 g, 32.6 μmol, 34% yield). ¹H NMR (400 MHz, CDCl₃) δ 7.80 (d, J=3.0 Hz, 1H), 7.49-7.41 (m, 2H), 7.19 (s, 1H), 6.98-6.87 (m, 3H), 6.82-6.47 (m, 1H), 5.22-5.14 (m, 1H), 4.15-4.08 (m, 3H), 3.80-3.75 (m, 2H), 3.64 (t, J=6.3 Hz, 2H), 3.47 (s, 3H), 3.23 (t, J=6.1 Hz, 2H), 1.84 (p, J=6.1 Hz, 2H). ¹⁹F NMR (376 MHz, CDCl₃) δ −82.37 (dd, J=73.8, 12.7 Hz, 2F), −135.31 (ddd, J=23.8, 9.2, 7.1 Hz, 1F), −146.15 (d, J=7.0 Hz, 1F), −147.07-147.25 (m, 1F), −153.84 (t, J=2.8 Hz, 1F), −169.74 (t, J=2.8 Hz, 1F). LC-MS (ESI+) m/z calc'd for [C₂₃H₂₂F₇N₅O₅S]+: 614.1. Found: 614.2.

### 2,3,4,5-tetrafluoro-N-(3-((5-fluoro-2-((4-(2-methoxyethoxy)phenyl)amino)pyrimidin-4-yl)amino)propyl)-6-(fluoromethyl)benzenesulfonamide Compound I-56

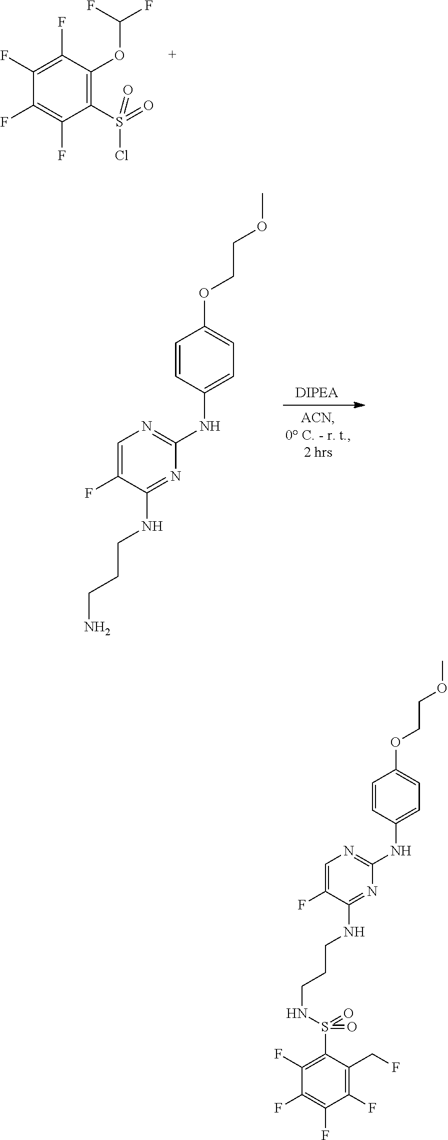 [SMILES: O=S(=O)(Cl)c1c(F)c(F)c(F)c(F)c1OC(F)F ; COCCOc1ccc(Nc2ncc(F)c(NCCCN)n2)cc1 ; COCCOc1ccc(Nc2ncc(F)c(NCCCNS(=O)(=O)c3c(F)c(F)c(F)c(F)c3CF)n2)cc1]

A mixture of N⁴-(3-aminopropyl)-5-fluoro-N²-[4-(2-methoxyethoxy)phenyl]pyrimidine-2,4-diamine (60 mg, 179 μmol), DCM (1 mL) and DIPEA (46 mg, 358 μmol, 62 uL) was stirred at 0° C. while a solution of (fluoromethyl)benzenesulfonyl chloride (45.18 mg, 161.01 μmol, 23.85 uL) in DCM (1 mL) was added dropwise and the resulting mixture allowed to warm to room temperature. After 2 hrs, the reaction was quenched with water and extracted with DCM (×3), washed with brine, and dried over Na₂SO₄. The solvent was removed under reduced pressure, and the residue was separated on a Prep-HPLC equipped with a reverse phase column (90-0% water in ACN) to afford the desired product (0.016 g, 27.6 μmol, 15% yield). ¹H NMR (400 MHz, CDCl₃) δ 7.77 (d, J=3.2 Hz, 1H), 7.49-7.43 (m, 2H), 7.38 (s, 1H), 7.09 (s, 1H), 6.97-6.85 (m, 2H), 5.90 (dd, J=46.2, 3.0 Hz, 2H), 5.36 (s, 1H), 4.16-4.09 (m, 2H), 3.76 (dd, J=5.5, 3.9 Hz, 2H), 3.64 (q, J=6.3 Hz, 2H), 3.47 (s, 3H), 3.22 (s, 2H), 2.67 (s, 1H), 1.84 (p, J=6.2 Hz, 2H). ¹⁹F NMR (376 MHz, CDCl₃) δ −132.30-−132.51 (m, 1F), −137.37 (dddt, J=20.9, 11.6, 5.9, 3.3 Hz, 1F), −146.46 (td, J=20.8, 9.0 Hz, 1F), −149.07 (ddt, J=22.8, 20.0, 6.2 Hz, 1F), −169.18 (1F), −205.51-−205.63 (m, 1F).

### 2,3,4,5-tetrafluoro-N-(3-((5-fluoro-2-((4-(2-methoxyethoxy)phenyl)amino)pyrimidin-4-yl)amino)phenyl)-6-(fluoromethoxy)benzenesulfonamide Compound I-57

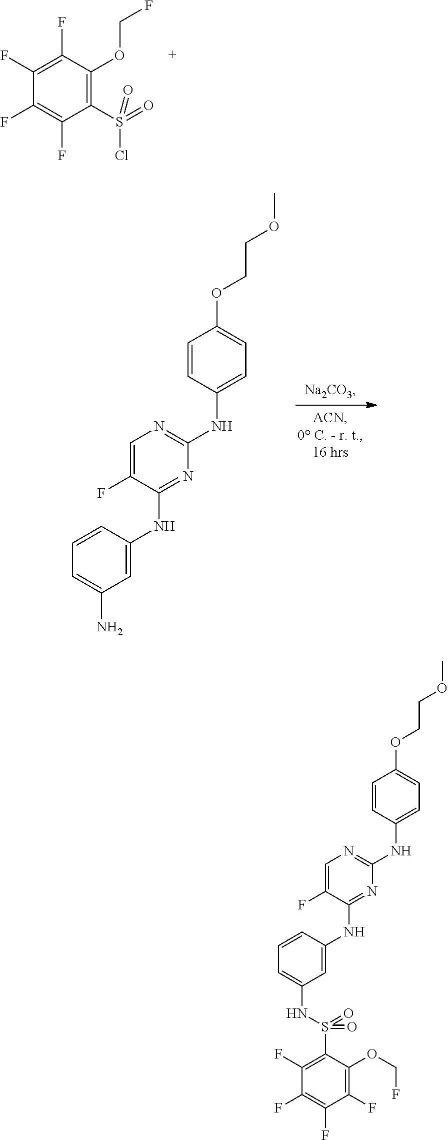 [SMILES: O=S(=O)(Cl)c1c(F)c(F)c(F)c(F)c1OCF ; COCCOc1ccc(Nc2ncc(F)c(Nc3cccc(N)c3)n2)cc1 ; COCCOc1ccc(Nc2ncc(F)c(Nc3cccc(NS(=O)(=O)c4c(F)c(F)c(F)c(F)c4OCF)c3)n2)cc1]

A mixture of N⁴-(3-aminophenyl)-5-fluoro-N²-[4-(2-methoxyethoxy)phenyl]pyrimidine-2,4-diamine (56 mg, 150 μmol), ACN (1.5 mL) and sodium carbonate (16 mg, 150 μmol) was stirred at 0° C. while a solution of 2,3,4,5-tetrafluoro-6-(fluoromethoxy)benzenesulfonyl chloride (46.84 mg, 157.94 μmol, 21.05 uL) in ACN (1.5 mL) was added dropwise and the resulting mixture allowed to warm to room temperature. After 16 hrs, the reaction was quenched with water and extracted with DCM (×3), washed with brine, and dried over Na₂SO₄. The solvent was removed under reduced pressure, and the residue was separated on a Prep-HPLC equipped with a reverse phase column (90-0% water in ACN) to afford the desired product (0.018 g, 28.6 μmol, 19% yield). ¹H NMR (400 MHz, CDCl₃) δ 7.94 (s, 1H), 7.82 (s, 1H), 7.71 (s, 1H), 7.43 (d, J=8.9 Hz, 2H), 7.27-7.21 (m, 2H), 6.94 (dd, J=7.4, 4.9 Hz, 4H), 6.87 (s, 1H), 5.75 (s, 1H), 5.62 (s, 1H), 4.17 (dd, J=5.7, 3.8 Hz, 2H), 3.83-3.76 (m, 2H), 3.49 (s, 3H). ¹⁹F NMR (376 MHz, CDCl₃) δ −134.90 (dt, J=24.2, 8.6 Hz), −144.74 (td, J=21.1, 7.7 Hz), −148.76 (td, J=52.4, 19.7 Hz), −149.39 (td, J=20.3, 9.3 Hz), −155.65-−155.85 (m), −167.39.

### 2,3,4,5-tetrafluoro-N-(3-((5-fluoro-2-((4-(2-methoxyethoxy)phenyl)amino)pyrimidin-4-yl)amino)phenyl)-6-(fluoromethyl)benzenesulfonamide Compound I-58

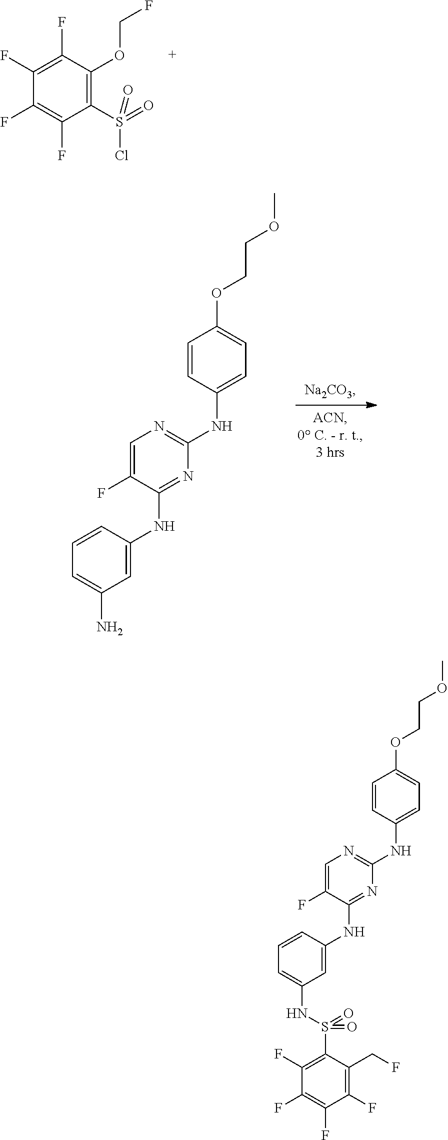 [SMILES: O=S(=O)(Cl)c1c(F)c(F)c(F)c(F)c1OCF ; COCCOc1ccc(Nc2ncc(F)c(Nc3cccc(N)c3)n2)cc1 ; COCCOc1ccc(Nc2ncc(F)c(Nc3cccc(NS(=O)(=O)c4c(F)c(F)c(F)c(F)c4CF)c3)n2)cc1]

A mixture of N⁴-(3-aminophenyl)-5-fluoro-N²-[4-(2-methoxyethoxy)phenyl]pyrimidine-2,4-diamine (60 mg, 164 μmol), THF (1.5 mL) and sodium carbonate (22 mg, 211 μmol) was stirred at 0° C. while a solution of 2,3,4,5-tetrafluoro-6-(fluoromethyl)benzenesulfonyl chloride (59 mg, 211 μmol) in THF (1.5 mL) was added dropwise and the resulting mixture allowed to warm to room temperature. After 3 hrs, the reaction was quenched with water and extracted with DCM (×3), washed with brine, and dried over Na₂SO₄. The solvent was removed under reduced pressure, and the residue was separated on a Prep-HPLC equipped with a reverse phase column (50-0% water in ACN+0.1% FA) to afford the desired product (0.005 g, 9 μmol, 5% yield). ¹H NMR (400 MHz, CDCl₃) δ 7.98 (d, J=3.2 Hz, 1H), 7.83 (s, 1H), 7.42 (d, J=8.8 Hz, 2H), 7.23 (t, J=8.2 Hz, 1H), 6.96 (d, J=8.9 Hz, 1H), 6.94-6.73 (m, 4H), 5.91 (d, J=46.1 Hz, 2H), 4.17 (t, J=4.8 Hz, 2H), 3.83-3.76 (m, 1H), 3.48 (d, J=5.1 Hz, 3H). ¹⁹F NMR (376 MHz, CDCl₃) δ −131.18 (1F), −136.56 (1F) −144.56 (1F), −148.46 (1F), −167.89 (1F), −206.62 (1F).

### 2-(N,N-dimethylsulfamoyl)-3,4,5,6-tetrafluoro-N-(3-((5-fluoro-2-((4-(2-methoxy ethoxy)phenyl)amino)pyrimidin-4-yl)amino)phenyl)benzamide Compound I-59

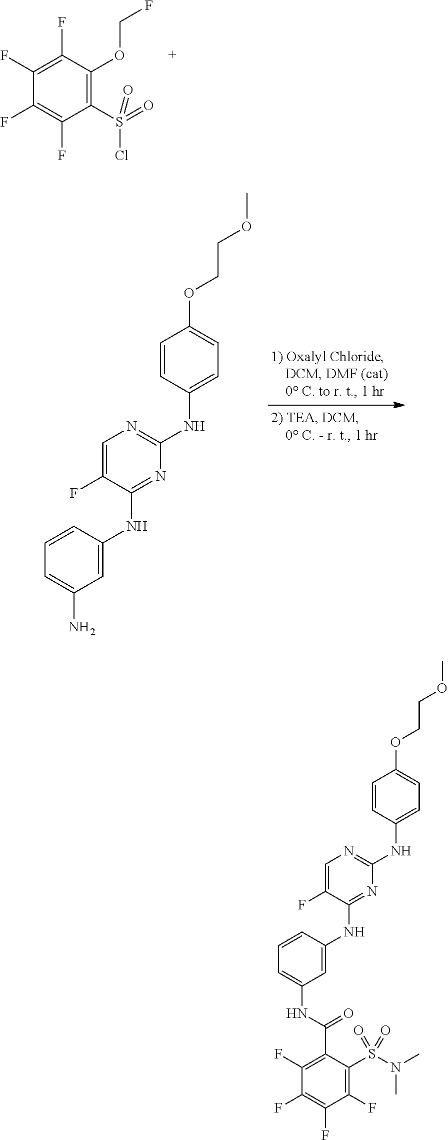 [SMILES: O=S(=O)(Cl)c1c(F)c(F)c(F)c(F)c1OCF ; COCCOc1ccc(Nc2ncc(F)c(Nc3cccc(N)c3)n2)cc1 ; COCCOc1ccc(Nc2ncc(F)c(Nc3cccc(NC(=O)c4c(F)c(F)c(F)c(F)c4S(=O)(=O)N(C)C)c3)n2)cc1]

2-(N,N-dimethylsulfamoyl)-3,4,5,6-tetrafluoro-N-(3-((5-fluoro-2-((4-(2-methoxy ethoxy)phenyl)amino)pyrimidin-4-yl)amino)phenyl)benzamide was prepared from 2-(N,N-dimethylsulfamoyl)-3,4,5,6-tetrafluorobenzoic acid (0.1 g, 0.33 mmol) and N4-(3-aminophenyl)-5-fluoro-N2-(4-(2-methoxyethoxy)phenyl)pyrimidine-2,4-diamine (0.18 g, 0.49 mmol) according to the protocol described in general procedure A and isolated as an off-white solid (0.06 g, 0.092 mmol, 28% yield). ¹H NMR (400 MHz, DMSO-d₆) δ 10.75 (s, 1H), 9.43 (s, 1H), 8.94 (s, 1H), 8.06 (d, J=4 Hz, 1H), 7.80 (s, 1H), 7.77 (d, J=8.0 Hz, 1H), 7.53 (d, J=9.2 Hz, 2H), 7.35-7.27 (m, 2H), 6.80 (d, J=8.8 Hz, 2H), 4.04-4.01 (m, 2H), 3.65-3.63 (m, 2H), 3.31 (s, 3H), 2.86 (s, 6H). ¹⁹F NMR (376 MHz, DMSO-d₆) δ −130.83-−130.95 (m, 1F), −140.38-−140.47 (m, 1F), −146.96-−147.11 (m, 1F), −151.49-−151.60 (m, 1F), −164.76 (1F). ESI-MS: measured m/z 653.2 [M+1]⁺. HPLC (Method I) RT=5.96 min., 98.6%.

### 2-(difluoromethoxy)-3,4,5,6-tetrafluoro-N-((2-((4-(4-methylpiperazin-1-yl)phenyl)amino)pyrimidin-5-yl)methyl)benzenesulfonamide Compound I-60

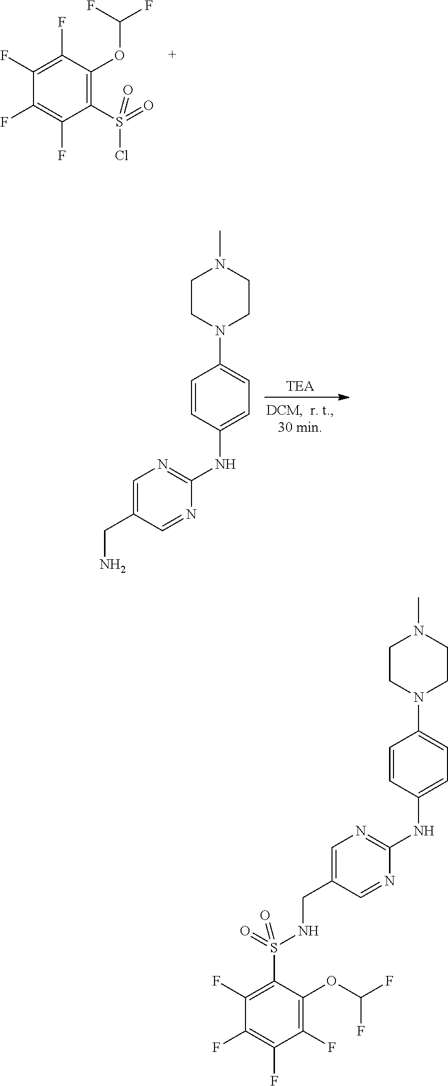 [SMILES: O=S(=O)(Cl)c1c(F)c(F)c(F)c(F)c1OC(F)F ; CN1CCN(c2ccc(Nc3ncc(CN)cn3)cc2)CC1 ; CN1CCN(c2ccc(Nc3ncc(CNS(=O)(=O)c4c(F)c(F)c(F)c(F)c4OC(F)F)cn3)cc2)CC1]

To a stirred solution of 2-(difluoromethoxy)-3,4,5,6-tetrafluorobenzenesulfonyl chloride (0.3 g, 0.95 mmol) in THE (1.5 mL) under an N₂ atmosphere was added 4-(amino methyl)-N-(4-(4-methylpiperazin-1-yl) phenyl) pyrimidin-2-amine (0.28 g, 0.95 mmol) in THE (1.5 mL) and TEA (1.9 mmol, 0.265 mL). After 30 min., the reaction mixture was diluted with water (100 mL) and extracted with EtOAc (2×50 mL). The combined organic phases were dried over anhydrous Na₂SO₄, and concentrated under reduced pressure. The crude material was purified by Prep HPLC to afford title compound as a brown solid (0.009 g, 0.015 mmol, 2% yield). ¹H NMR (400 MHz, DMSO-d6) δ 9.27 (s, 1H), 8.34 (d, J=4.8 Hz, 1H), 8.24 (s, 1H), 7.52 (d, J=9.2 Hz, 2H), 7.25-6.89 (t, J=73.6 Hz, 1H), 6.84 (d, J=9.2 Hz, 2H), 6.71 (d, J=5.2 Hz, 1H), 4.18 (s, 2H), 3.05 (t, J=4.4 Hz, 4H), 2.46 (t, J=5.2 Hz, 4H), 2.22 (s, 3H). ¹⁹F NMR (376 MHz, DMSO-d6) δ −81.01-−83.15 (m, 2F), −135.63-−135.72 (m, 1F), −149.68-−150.20 (m, 1F), −156.20-−156.33 (m, 1F). ESI-MS: measured m/z 577.2, [M+1]. HPLC (Method I): RT=5.52 min., 92.5%

### Preparation of 2,3,4,5-tetrafluoro-N-(3-((5-fluoro-2-((4-(2-methoxyethoxy)phenyl)amino)pyrimidin-4-yl)amino)phenyl)-6-(methylsulfonyl)benzamide Compound I-61 and 2,3,4,5-tetrafluoro-N-(3-((5-fluoro-2-((4-(2-methoxyethoxy)phenyl)amino)pyrimidin-4-yl)amino)phenyl)-6-(methylsulfinyl)benzamide Compound I-62

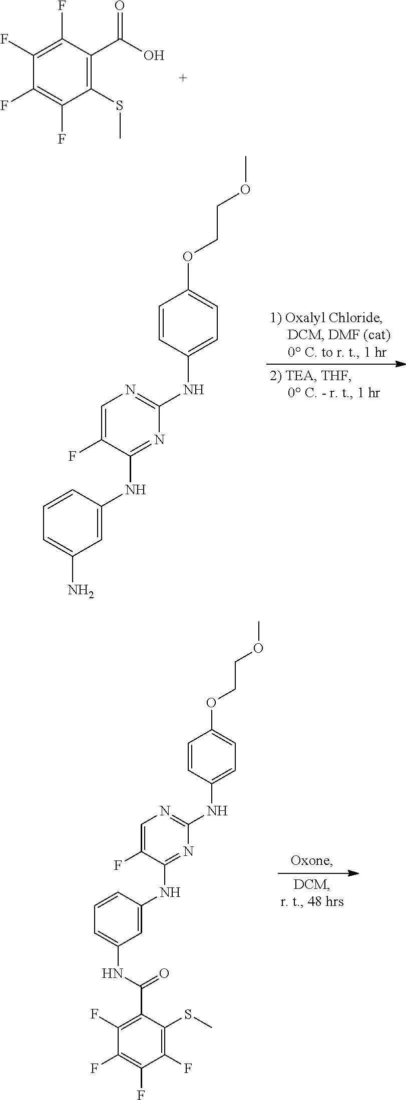 [SMILES: CSc1c(F)c(F)c(F)c(F)c1C(=O)O ; COCCOc1ccc(Nc2ncc(F)c(Nc3cccc(N)c3)n2)cc1 ; COCCOc1ccc(Nc2ncc(F)c(Nc3cccc(NC(=O)c4c(F)c(F)c(F)c(F)c4SC)c3)n2)cc1]

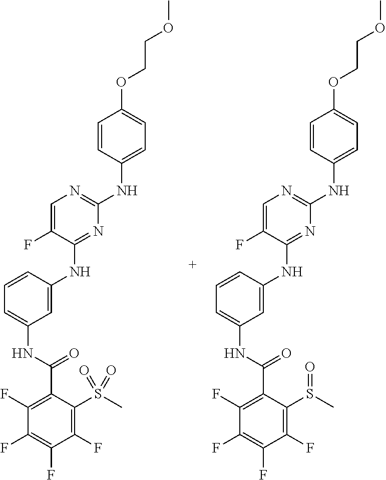 [SMILES: COCCOc1ccc(Nc2ncc(F)c(Nc3cccc(NC(=O)c4c(F)c(F)c(F)c(F)c4S(C)(=O)=O)c3)n2)cc1 ; COCCOc1ccc(Nc2ncc(F)c(Nc3cccc(NC(=O)c4c(F)c(F)c(F)c(F)c4S(C)=O)c3)n2)cc1]

### 2,3,4,5-tetrafluoro-N-(3-((5-fluoro-2-((4-(2-methoxyethoxy)phenyl)amino)pyrimidin-4-yl)amino)phenyl)-6-(methylthio)benzamide

2,3,4,5-tetrafluoro-N-(3-((5-fluoro-2-((4-(2-methoxyethoxy)phenyl)amino)pyrimidin-4-yl)amino)phenyl)-6-(methylthio)benzamide was prepared from 2,3,4,5-tetrafluoro-6-(methylthio)benzoic acid (0.30 g, 1.10 mmol) and N⁴-(3-aminophenyl)-5-fluoro-N²-(4-(2-methoxyethoxy)phenyl)pyrimidine-2,4-diamine (0.48 g, 1.32 mmol) according to the protocol described in general procedure A and isolated as a white solid (0.42 g, 0.71 mmol, 61% yield). ¹H NMR (400 MHz, DMSO-d₆) δ 10.87 (s, 1H), 9.44 (s, 1H), 8.97 (s, 1H), 8.07 (d, J=3.6 Hz, 1H), 7.88 (s, 1H), 7.75 (d, J=6.4 Hz, 1H), 7.53 (d, J=9.2 Hz, 2H), 7.35-7.34 (m, 2H), 6.80 (d, J=9.2 Hz, 2H), 4.03-4.01 (m, 2H), 3.64-3.62 (m, 2H), 3.30 (s, 3H), 2.46 (s, 3H). ¹⁹F NMR (376 MHz, DMSO-d₆) δ −131.04-−131.14 (m, 1F), −141.87-−141.96 (m, 1F), −153.76-−154.34 (m, 2F), −164.84 (1F). ESI-MS: measured m/z 592.2 [M+1]⁺. HPLC (Method I): RT=6.14 min., 95.1%.

### 2,3,4,5-tetrafluoro-N-(3-((5-fluoro-2-((4-(2-methoxyethoxy)phenyl)amino)pyrimidin-4-yl)amino)phenyl)-6-(methylsulfonyl)benzamide Compound I-61

To a stirred solution of 2,3,4,5-tetrafluoro-N-(3-((5-fluoro-2-((4-(2-methoxyethoxy)phenyl)amino)pyrimidin-4-yl)amino)phenyl)-6-(methylthio)benzamide (0.19 g, 0.32 mmol) in DCM (5 mL) at 0° C. was added oxone (1.01 g, 3.28 mmol). The reaction was gradually warmed to room temperature. After 48 hrs, the reaction was diluted with a saturated aqueous solution of NaHCO₃ (50 mL) and extracted with EtOAc (3×40 mL). The combined organic phases were dried over anhydrous Na₂SO₄, filtered, and concentrated under reduced pressure. The crude material was purified by prep TLC product (60% EtOAc in hexane) to afford the title compound as an off-white solid (0.035 g, 0.056 mmol, 11% yield). ¹H NMR (400 MHz, DMSO-d₆) δ 10.89 (s, 1H), 9.43 (s, 1H), 8.96 (s, 1H), 8.07 (d, J=3.6 Hz, 1H), 7.81 (s, 1H), 7.76 (d, J=3.2 Hz, 1H), 7.54 (d, J=8.8 Hz, 2H), 7.34-7.33 (m, 2H), 6.80 (d, J=9.2 Hz, 2H), 4.03-4.01 (m, 2H), 3.64-3.62 (m, 2H), 3.46 (s, 3H), 3.31 (s, 3H). ¹⁹F NMR (376 MHz, DMSO-d₆) δ −132.44-−132.53 (m, 1F), −140.09-−140.18 (m, 1F), −145.19-−145.28 (m, 1F), −151.15-−151.27 (m, 1F), −164.77 (s, 1F). ESI-MS: measured m/z 624.2 [M+1]⁺. HPLC (Method I): RT=5.74 min., 100%.

### 2,3,4,5-tetrafluoro-N-(3-((5-fluoro-2-((4-(2-methoxyethoxy)phenyl)amino)pyrimidin-4-yl)amino)phenyl)-6-(methylsulfinyl)benzamide Compound I-62

The title compound was isolated as a separate entity during the purification of 2,3,4,5-tetrafluoro-N-(3-((5-fluoro-2-((4-(2-methoxyethoxy)phenyl)amino)pyrimidin-4-yl)amino)phenyl)-6-(methylsulfonyl)benzamide. 2,3,4,5-tetrafluoro-N-(3-((5-fluoro-2-((4-(2-methoxyethoxy)phenyl)amino)pyrimidin-4-yl)amino)phenyl)-6-(methylsulfinyl)benzamide was isolated as a pink solid (0.045 g, 0.074 mmol, 15% yield). ¹H NMR (400 MHz, DMSO-d₆) δ 10.98 (s, 1H), 9.49 (s, 1H), 8.97 (s, 1H), 8.07 (d, J=5.2 Hz, 1H), 7.89 (s, 1H), 7.75 (t, J=5.2 Hz, 1H), 7.54 (d, J=8.8 Hz, 2H), 7.34 (d, J=4.4 Hz, 2H), 6.80 (d, J=14 Hz, 2H), 4.03-4.01 (t, J=4.4 Hz, 2H), 3.64-3.62 (m, 2H), 3.30 (s, 3H), 3.13 (s, 3H). ¹⁹F NMR (376 MHz, DMSO-d₆) δ −139.19-−139.30 (m, 1F), −140.28-−140.37 (m, 1F), −149.81-−149.94 (m, 1F). −151.58-−151.70 (m, 1F), 164.80 (s, 1F). ESI-MS: measured m/z 608.3 [M+1]⁺. HPLC (Method I): RT=5.61 min., 100%.

### Preparation of 5-fluoro-N²-(4-(2-methoxyethoxy)phenyl)-N⁴-(3-((2,3,4,5-tetrafluoro-6-(methyl sulfinyl)phenyl)amino)phenyl)pyrimidine-2,4-diamine Compound I-63 and 5-fluoro-N²-(4-(2-methoxyethoxy)phenyl)-N⁴-(3-((2,3,4,5-tetrafluoro-6-(methylsulfonyl)phenyl)amino)phenyl)pyrimidine-2,4-diamine Compound I-64

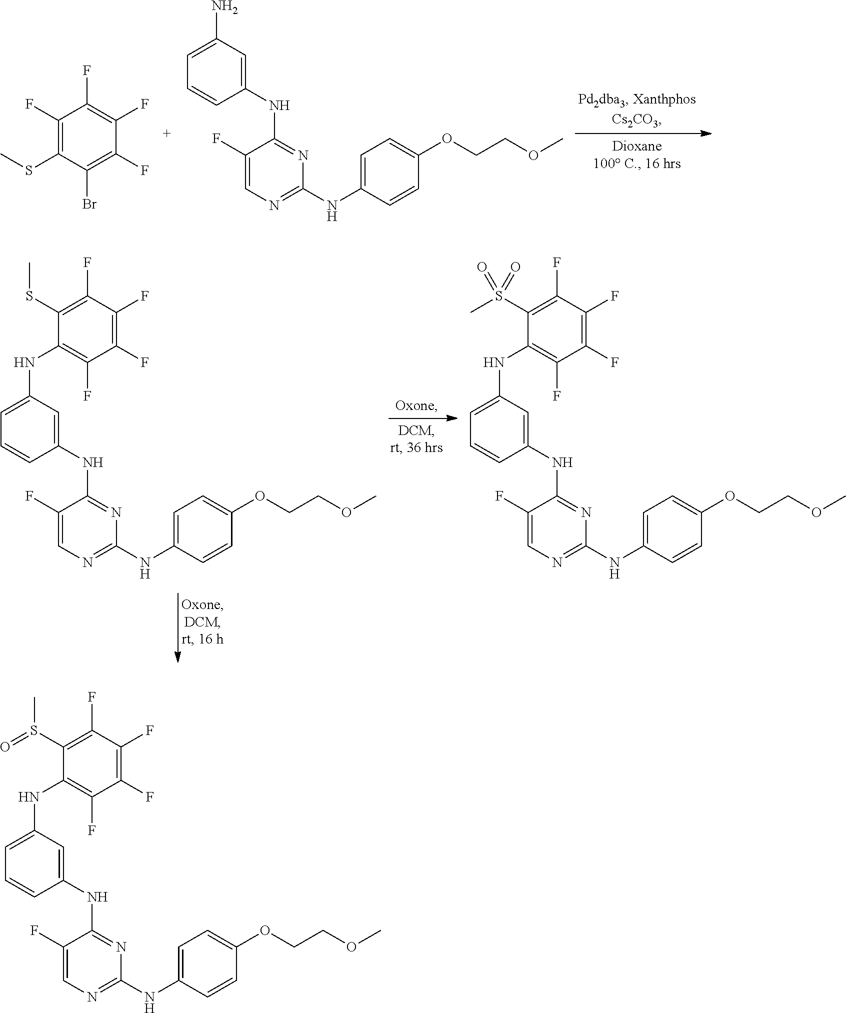

### 5-fluoro-N²-(4-(2-methoxyethoxy)phenyl)-N⁴-(3-((2,3,4,5-tetrafluoro-6-(methylthio)phenyl)amino)phenyl)pyrimidine-2,4-diamine

A reaction vessel containing N⁴-(3-aminophenyl)-5-fluoro-N²-(4-(2-methoxyethoxy)phenyl) pyrimidine-2,4-diamine (0.33 g, 0.90 mmol), 2-bromo-3,4,5,6-tetrafluorophenyl)(methyl) sulfane (0.25 g, 0.90 mmol), Cs₂CO₃ (0.59 g, 1.81 mmol) and 1,4-dioxane (2.5 mL) was purged with N₂ for 15 minutes. While under a positive pressure of inert atmosphere, Pd₂dba₃ (0.08 g, 0.09 mmol) and Xantphos (0.05 g, 0.09 mmol) were added. The resulting mixture was heated to 100° C. and stirred overnight. After 16 hrs, the mixture was diluted with water (30 mL) and extracted with EtOAc (2×30 mL). The combined organic phases were dried over anhydrous Na₂SO₄, filtered, and concentrated under reduced pressure. The crude material was purified by flash column chromatography (38% EtOAc in hexane) to afford the title compound as white solid (0.28 g, 0.49 mmol, 55% yield). ¹H NMR (400 MHz, DMSO-d₆) δ 9.10 (s, 1H), 8.93 (s, 1H), 8.05 (d, J=3.6 Hz, 1H), 7.85 (s, 1H), 7.54 (d, J=8.8 Hz, 2H), 7.47 (d, d, J=8.4 Hz, 1H), 7.13 (t, J=8.0 Hz, 1H), 7.00 (s, 1H), 6.82 (d, J=9.2 Hz, 2H), 6.50 (d, J=7.6 Hz, 1H), 4.01-3.99 (m, 2H), 3.63-3.61 (m, 2H), 3.30 (s, 3H), 2.34 (s, 3H). ¹⁹F NMR (376 MHz, DMSO-d₆) δ −132.12-−132.21 (m, 1F), −144.02-−144.11 (m, 1F), −156.25-−156.37 (m, 1F), −162.88-−163.01 (m, 1F), −164.93 (s, 1F). ESI-MS: measured m/z 564.2 [M+1]⁺.

### 5-fluoro-N²-(4-(2-methoxyethoxy)phenyl)-N⁴-(3-((2,3,4,5-tetrafluoro-6-(methyl sulfinyl)phenyl)amino)phenyl)pyrimidine-2,4-diamine Compound I-63

5-fluoro-N²-(4-(2-methoxyethoxy)phenyl)-N⁴-(3-((2,3,4,5-tetrafluoro-6-(methyl sulfinyl)phenyl)amino)phenyl)pyrimidine-2,4-diamine was prepared from 5-fluoro-N²-(4-(3-methoxypropyl)phenyl)-N⁴-(3-((2,3,4,5-tetrafluoro-6-(methylthio)phenyl)amino)phenyl)pyrimidine-2,4-diamine (0.10 g, 0.17 mmol) according to the protocol described in general procedure C and isolated as a pink solid (0.025 g, 0.043 mmol, 24% yield). ¹H NMR (400 MHz, DMSO-d₆) δ 9.15 (s, 1H), 8.96 (s, 1H), 8.27 (s, 1H), 8.06 (d, J=3.6 Hz, 1H), 7.55-7.52 (m, 3H), 7.18 (t, J=8.4 Hz, 1H), 7.08 (s, 1H), 6.82 (d, J=8.8 Hz, 2H), 6.54 (d, J=8.4 Hz, 1H), 4.00 (t, J=4.8 Hz, 2H), 3.62 (t, J=4.8 Hz, 2H), 3.30 (s, 3H), 3.05 (s, 3H). ¹⁹F NMR (376 MHz, DMSO-d₆) δ −141.46-−141.52 (m, 1F), −143.15-−143.24 (m, 1F), −150.76-−150.87 (m, 1F), −161.78-−161.90 (m, 1F), −164.93 (s, 1F). ESI-MS: measured m/z 580.19 [M+1]⁺. HPLC (Method I): RT=5.94 min., 97.3%.

### 5-fluoro-N²-(4-(2-methoxyethoxy)phenyl)-N⁴-(3-((2,3,4,5-tetrafluoro-6-(methylsulfonyl)phenyl)amino)phenyl)pyrimidine-2,4-diamine Compound I-64

To a stirred solution of 5-fluoro-N²-(4-(3-methoxypropyl)phenyl)-N⁴-(3-((2,3,4,5-tetrafluoro-6-(methylthio)phenyl)amino)phenyl)pyrimidine-2,4-diamine (0.09 g, 0.15 mmol) in THF:MeOH:water (8:1:1 v/v) (1.0 mL) at 0° C. was added Oxone (0.24 g, 0.79 mmol). The resulting mixture was warmed to room temperature. After 16 hrs, another portion of Oxone (0.24 g, 0.79 mmol) was added and the reaction permitted to stir overnight. Once deemed complete, the reaction was diluted with saturated aqueous NaHCO₃ and extracted with EtOAc (2×30 mL). The combined organic phases were dried over anhydrous Na₂SO₄, filtered, and concentrated under reduced pressure. The crude material was purified by flash column chromatography (39% EtOAc in hexanes) to afford the title compound as light pink solid (0.025 g, 0.042 mmol, 26% yield). ¹H NMR (400 MHz, DMSO-d₆) δ 9.16 (s, 1H), 8.96 (s, 1H), 8.06 (d, J=3.6 Hz, 1H), 7.94 (s, 1H), 7.58-7.52 (m, 3H), 7.20-7.16 (m, 2H), 6.82 (d, J=8.8 Hz, 2H), 6.62 (d, J=8.0 Hz, 1H), 4.01-3.99 (m, 2H), 3.63-3.61 (m, 2H), 3.44 (s, 3H), 3.28 (s, 3H). ¹⁹F NMR (376 MHz, DMSO-d₆) δ −135.06-−135.17 (m, 1F), −140.44-−140.52 (m, 1F), −147.53-−147.67 (m, 1F), −162.65-−162.78 (m, 1F), −164.96 (s, 1F). ESI-MS: measured m/z 596.26 [M+1]⁺. HPLC (Method I): RT=6.21 min., 96.6%.

### Preparation of 5-fluoro-N²-(4-(2-methoxyethoxy)phenyl)-N⁴-(3-((2,3,4,5-tetrafluoro-6-(methylsulfonyl)benzyl)oxy)phenyl)pyrimidine-2,4-diamine Compound I-65

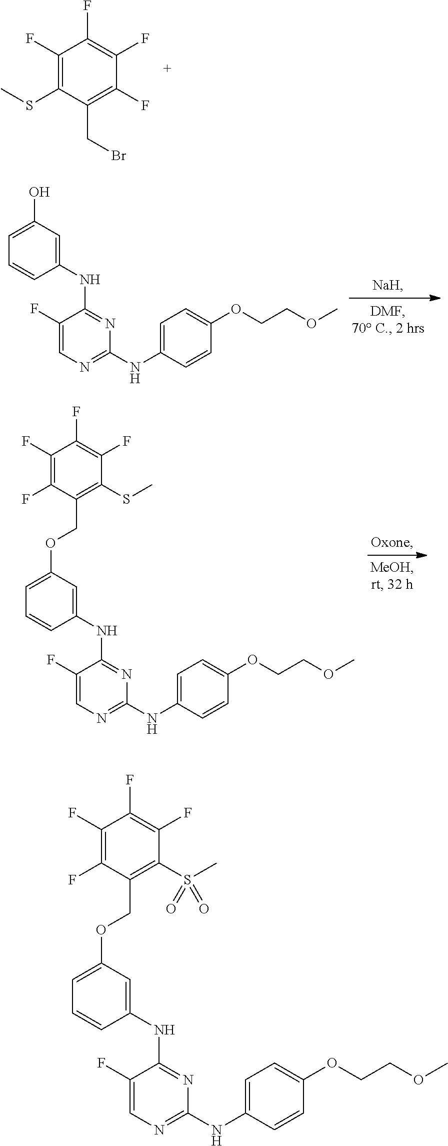 [SMILES: CSc1c(F)c(F)c(F)c(F)c1CBr ; COCCOc1ccc(Nc2ncc(F)c(Nc3cccc(O)c3)n2)cc1 ; COCCOc1ccc(Nc2ncc(F)c(Nc3cccc(OCc4c(F)c(F)c(F)c(F)c4SC)c3)n2)cc1 ; COCCOc1ccc(Nc2ncc(F)c(Nc3cccc(OCc4c(F)c(F)c(F)c(F)c4S(C)(=O)=O)c3)n2)cc1]

### 5-fluoro-N²-(4-(2-methoxyethoxy)phenyl)-N⁴-(3-((2,3,4,5-tetrafluoro-6-(methylthio)benzyl)oxy)phenyl)pyrimidine-2,4-diamine

To a stirred solution of 3-((5-fluoro-2-((4-(2-methoxyethoxy)phenyl)amino)pyrimidin-4-yl)amino)phenol (0.32 g, 0.86 mmol) in DMF (3 mL) at 0° C. was added NaH (0.080 g, 3.29 mmol). The resulting mixture was heated to 70° C. for 1 hour, after which neat (2-(bromomethyl)-3,4,5,6-tetrafluorophenyl)(methyl)sulfane (0.25 g, 0.86 mmol) was added in one portion. The reaction was permitted to continue for a further hour after the addition. Once deemed completed, the reaction mixture was cooled to ambient temperature and diluted with ice cold water (25 mL). The resulting suspension was extracted with EtOAc (2×15 mL) and the combined organic phases were dried over anhydrous Na₂SO₄, filtered, and concentrated under reduced pressure. The crude material was purified by flash column chromatography (43% EtOAc in hexanes) to afford the title compound as a yellow resin (0.11 g, 0.19 mmol, 35% yield). ¹H NMR (400 MHz, DMSO-d₆) δ 9.28 (s, 1H), 9.02 (s, 1H), 8.08 (d, J=4.0 Hz, 1H), 7.52-7.48 (m, 4H), 7.26 (t, 1H), 6.79-6.75 (m, 3H), 5.19 (s, 2H), 3.98-3.96 (m, 2H), 3.62-3.60 (m, 2H), 3.30 (s, 3H), 2.41 (s, 3H). ¹⁹F NMR (376 MHz, DMSO-d₆) δ −130.43-−130.53 (m, 1F), −140.77-−140.87 (m, 1F), −153.89-−154.02 (m, 1F), −155.31-−155.44 (m, 1F), −164.91 (s, 1F). ESI-MS: measured m/z 579.2 [M+1]⁺.

### 5-fluoro-N²-(4-(2-methoxyethoxy)phenyl)-N⁴-(3-((2,3,4,5-tetrafluoro-6-(methylsulfonyl)benzyl)oxy)phenyl)pyrimidine-2,4-diamine Compound I-65

To a stirred solution of 5-fluoro-N²-(4-(2-methoxyethoxy) phenyl)-N⁴-(4-((2,3,4,5-tetrafluoro-6-(methylthio)benzyl)oxy)phenyl)pyrimidine-2,4-diamine (0.1 g, 0.17 mmol) in MeOH (1 mL) was added oxone (0.26 g, 0.86 mmol). After 16 hrs, a second portion of oxone (0.26 g, 0.86 mmol) was added and the reaction stirred overnight. After a further 16 hrs, the mixture was diluted with saturated aqueous NaHCO₃ and extracted with EtOAc (2×20 mL). The combined organic phases were dried over anhydrous Na₂SO₄, filtered, and concentrated under reduced pressure. The resulting crude material was purified by flash column chromatography (45% EtOAc in hexanes) to afford the title compound as a light pink solid (0.027 g, 0.044 mmol, 25% yield). ¹H NMR (400 MHz, DMSO-d₆) δ 9.29 (s, 1H), 9.01 (s, 1H), 8.08 (d, J=3.6 Hz, 1H), 7.56-7.50 (m, 3H), 7.42 (s, 1H), 7.27 (t, J=8.4, 1H), 6.80-6.73 (m, 3H), 5.39 (s, 2H), 3.99 (m, 2H), 3.62 (t, J=4.4 Hz, 2H), 3.44 (s, 3H), 3.30 (s, 3H). ¹⁹F NMR (376 MHz, DMSO-d₆) δ −131.21-−131.33 (m, 1F), −138.98-−139.05 (m, 1F), −146.24-−146.38 (m, 1F), −151.26-−151.37 (m, 1F), −164.91 (s, 1F). ESI-MS: measured m/z 611.2 [M+1]⁺. HPLC (Method I): RT=6.17 min., 95.1%.

### N-(2-((7H-pyrrolo[2,3-d]pyrimidin-4-yl)amino)ethyl)-2-(N,N-dimethylsulfamoyl)-3,4,5,6-tetrafluorobenzamide Compound I-47

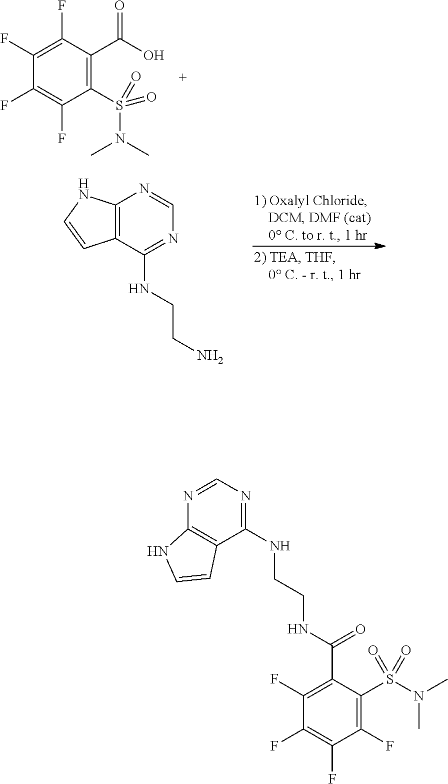

N-(2-((7H-pyrrolo[2,3-d]pyrimidin-4-yl)amino)ethyl)-2-(N,N-dimethylsulfamoyl)-3,4,5,6-tetrafluorobenzamide was prepared from 2-(N,N-dimethylsulfamoyl)-3,4,5,6-tetrafluorobenzoic acid (0.30 g, 0.99 mmol) and N¹-(7H-pyrrolo[2,3-d]pyrimidin-4-yl)ethane-1,2-diamine (0.26 g, 1.49 mmol) according to the protocol described in general procedure A and isolated as an off-white solid (0.25 g, 0.54 mmol, 55% yield). ¹H NMR (400 MHz, DMSO-d₆) δ 11.53 (s, 1H), 8.89 (t, J=5.2 Hz, 1H), 8.11 (s, 1H), 7.35 (s, 1H), 7.09-7.085 (m, 1H), 6.49 (s, 1H), 3.62 (br s, 2H), 3.48 (br s, 2H), 2.84 (s, 6H). ¹⁹F NMR (376 MHz, DMSO-d₆) δ −130.96-−130.08 (m, 1F), −140.33-−140.43 (m, 1F), −147.19-−147.33 (m, 1F), −151.99-−152.10 (m, 1F). ESI-MS: measured m/z 461.3 [M+1]⁺. HPLC (Method I): RT=4.81 min., 99%.

### Preparation of N-(2-((7H-pyrrolo[2,3-d]pyrimidin-4-yl)amino)ethyl)-2,3,4,5-tetrafluoro-6-(methylsulfonyl)benzamide Compound I-45

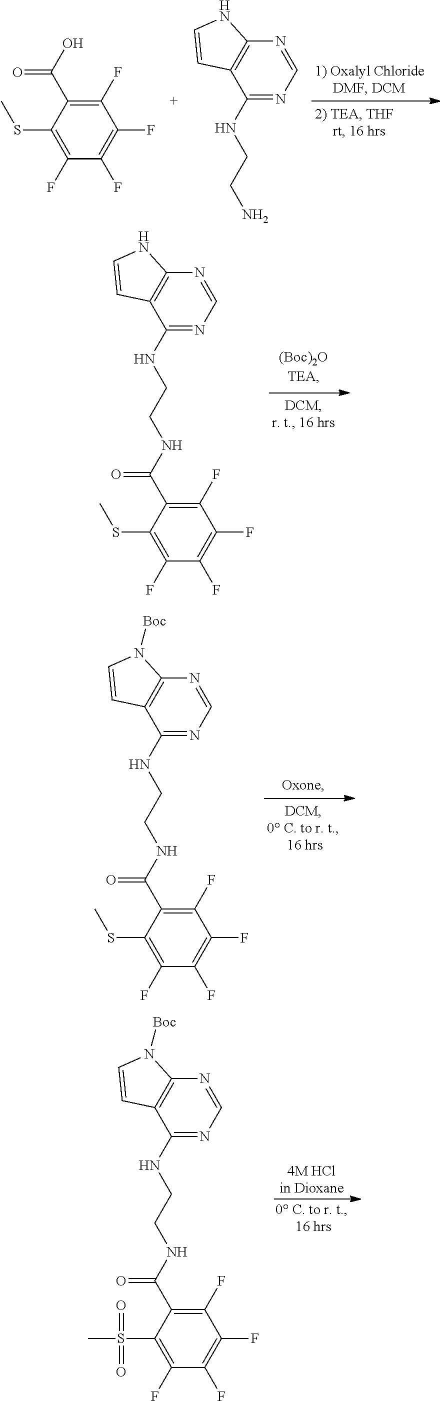

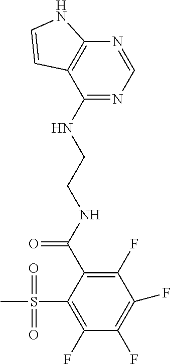 [SMILES: CS(=O)(=O)c1c(F)c(F)c(F)c(F)c1C(=O)NCCNc1ncnc2[nH]ccc12]

### Synthesis of N-(2-((7H-pyrrolo[2,3-d]pyrimidin-4-yl)amino)ethyl)-2,4,5-trifluoro-3-methyl-6-(methylthio)benzamide

Synthesis of N-(2-((7H-pyrrolo[2,3-d]pyrimidin-4-yl)amino)ethyl)-2,4,5-trifluoro-3-methyl-6-(methylthio)benzamide was prepared from 2,3,4,5-tetrafluoro-6-(methylthio)benzoic acid (0.40 g, 1.66 mmol) and N¹-(7H-pyrrolo[2,3-d]pyrimidin-4-yl)ethane-1,2-diamine (0.44 g, 2.40 mmol) according to the protocol described in general procedure A and isolated as a brown solid (0.30 g, 0.75 mmol, 46% yield). ¹H NMR (400 MHz, DMSO-d₆) δ 11.51 (s, 1H), 8.90-8.89 (m, 1H), 8.11 (s, 1H), 7.45 (s, 1H), 7.08 (m, 1H), 6.50 (s, 1H), 3.67-3.62 (m, 2H), 3.54-3.51 (m, 2H), 2.39 (s, 3H). ¹⁹F NMR (376 MHz, DMSO-d₆) δ −131.25-−131.35 (m, 1F), −142.02-−142.12 (m, 1F), −154.40-−154.69 (m, 2F). ESI-MS: measured m/z 400.29 [M+1]⁺.

### tert-butyl 4-((2-(2,3,4,5-tetrafluoro-6-(methylthio)benzamido)ethyl)amino)-7H-pyrrolo[2,3-d]pyrimidine-7-carboxylate

To the stirred solution of N-(2-((7H-pyrrolo[2,3-d]pyrimidin-4-yl)amino)ethyl)-2,4,5-trifluoro-3-methyl-6-(methylthio)benzamide (0.12 g, 0.30 mmol) in DCM (2 mL) at 0° C. was added TEA (0.090 g, 0.9 mmol). The mixture was stirred for 10 min prior to addition of (Boc)₂O (0.065 g, 0.30 mmol). The reaction mixture was warmed to room temperature and stirred overnight. After 16 hrs, the mixture was diluted with water (30 mL) and extracted with EtOAc (2×10 mL). The combined organic phases were dried over anhydrous Na₂SO₄, filtered, and concentrated under reduced pressure. The crude material was purified by normal phase chromatography (0-70% EtOAc in hexane) to afford title compound as a brown sticky solid (0.12 g, 0.24 mmol, 80% yield). ¹H NMR (400 MHz, DMSO-d₆) δ 8.89 (t, J=6.0 Hz, 1H), 8.28 (s, 1H), 7.79 (t, 1H), 7.73 (d, J=4.0 Hz, 1H), 6.53 (d, J=4.0 Hz, 1H), 3.66-3.62 (m, 2H), 3.55-3.50 (m, 2H), 2.39 (s, 3H), 1.59 (s, 9H). ESI-MS: measured m/z 500.27 [M+1]⁺.

### tert-butyl 4-((2-(2,3,4,5-tetrafluoro-6-(methylsulfonyl)benzamido)ethyl)amino)-7H-pyrrolo[2,3-d]pyrimidine-7-carboxylate

tert-butyl 4-((2-(2,3,4,5-tetrafluoro-6-(methylsulfonyl)benzamido)ethyl)amino)-7H-pyrrolo[2,3-d]pyrimidine-7-carboxylate was prepared from tert-butyl 4-((2-(2,3,4,5-tetrafluoro-6-(methylthio) benzamido)ethyl)amino)-7H-pyrrolo[2,3-d]pyrimidine-7-carboxylate (0.20 g, 0.40 mmol) according to the protocol described in general procedure C and isolated as a brown resin (0.13 g, 0.32 mmol, 61% yield). ¹H NMR (400 MHz, DMSO-d₆) δ 9.02 (t, J=5.2 Hz, 1H), 8.29 (s, 1H), 7.69 (t, J=6.0 Hz, 1H), 7.47 (d, J=4.0 Hz, 1H), 6.74 (d, J=4.0 Hz, 1H), 3.64-3.63 (m, 2H), 3.49-3.46 (m, 2H), 3.43 (s, 3H), 1.59 (s, 9H). ¹⁹F NMR (376 MHz, DMSO-d₆) δ −132.64-−132.73 (m, 1F), −140.08-−140.17 (m, 1F), −145.39-−145.51 (m, 1F), −151.65-−151.77 (m, 1F). ESI-MS: measured m/z 532.28 [M+1]⁺.

### N-(2-((7H-pyrrolo[2,3-d]pyrimidin-4-yl)amino)ethyl)-2,3,4,5-tetrafluoro-6-(methylsulfonyl)benzamide Compound I-45

A solution of tert-butyl 4-((2-(2,3,4,5-tetrafluoro-6-(methylsulfonyl)benzamido)ethyl)amino)-7H-pyrrolo[2,3-d]pyrimidine-7-carboxylate (0.12 g, 0.22 mmol) in 4N HCl in Dioxane (2 mL) was stirred at room temperature. After 16 hrs, the reaction mixture was diluted with a saturated solution of NaHCO₃ (30 mL) and extracted with DCM (2×20 mL). The combined organic phases were dried over anhydrous Na₂SO₄, filtered, and concentrated under reduced pressure. The crude material was purified by reverse phase chromatography (0-40% ACN in water) to afford the title compound as a sticky brown solid (0.010 g, 0.023 mmol, 10% yield). ¹H NMR (400 MHz, DMSO-d₆) δ 11.52 (s, 1H), 9.03 (t, J=5.6 Hz, 1H), 8.11 (s, 1H), 7.35 (s, 1H), 7.08 (t, J=3.2 Hz, 1H), 6.50-6.49 (m, 1H), 3.65-3.61 (m, 2H), 3.47-3.44 (m, 2H), 3.42 (s, 3H). ¹⁹F NMR (376 MHz, DMSO-d₆) δ −132.62-−132.72 (m, 1F), −140.07-−140.17 (m, 1F), −145.43-−145.51 (m, 1F), −151.69-−151.81 (m, 1F). ESI-MS: measured m/z 432.25 [M+1]⁺, HPLC (Method I): RT=4.28 min., 90.7%.

### Preparation of 2,3,4,5-tetrafluoro-6-(methylsulfinyl)-N-(7H-pyrrolo[2,3-d]pyrimidin-4-yl)benzamide Compound I-46

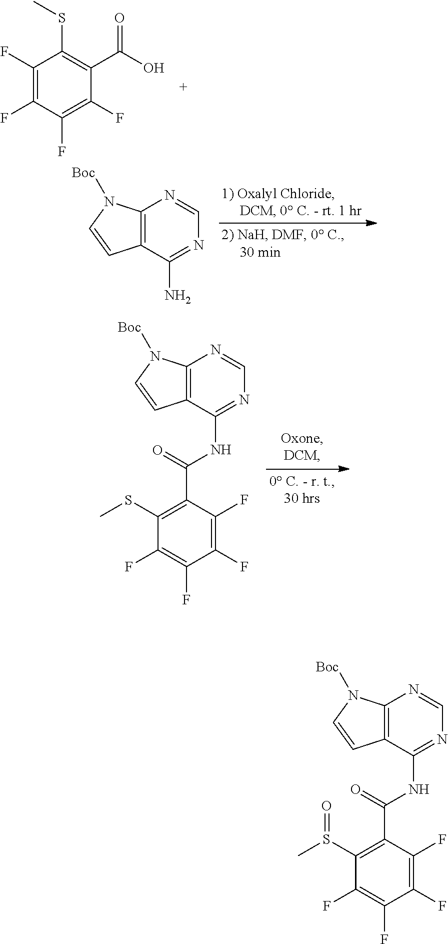

### tert-butyl 4-(2,3,4,5-tetrafluoro-6-(methylthio)benzamido)-7H-pyrrolo[2,3-d]pyrimidine-7-carboxylate

To a stirred solution of 2,3,4,5-tetrafluoro-6-(methylthio)benzoic acid (0.4 g, 1.70 mmol) in DCM (4 mL) were added oxalyl chloride (0.433 g, 3.41 mmol) and DMF (2 drop) at 0° C. The mixture was stirred at room temperature for 1 hr. After completion of reaction, the reaction mixture was concentrated under reduced pressure and stored under a nitrogen atmosphere. The obtained residue was dissolved in DCM (3 mL) and added dropwise to a pre-stirred solution of tert-butyl 4-amino-7H-pyrrolo[2,3-d]pyrimidine-7-carboxylate (0.61 g, 2.56 mmol) in DMF (5 mL) and NaH (60% in mineral oil) (0.13 g, 3.41 mmol) at 0° C. The resulting mixture was stirred for at 0° C. After 30 min., the mixture was diluted with ice-cold water (50 mL) and extracted with EtOAc (3×50 mL). The combined organic phases were dried over anhydrous Na₂SO₄, filtered, and concentrated under reduced pressure. The crude material was purified by flash column chromatography (30% EtOAc in hexanes) to afford the title compound as a yellow solid (0.29 g, 0.63 mmol, 37% yield). ¹H NMR (400 MHz, DMSO-d₆) δ 10.55 (s, 1H), 8.39 (s, 1H), 8.046 (s, 1H), 7.17 (d, J=4 Hz, 1H), 2.27 (s, 3H), 1.51 (s, 9H). ESI-MS: measured m/z 401.2 [M-tBu]⁻.

### tert-butyl 4-(2,3,4,5-tetrafluoro-6-(methylsulfinyl)benzamido)-7H-pyrrolo[2,3-d]pyrimidine-7-carboxylate

To a stirred solution of tert-butyl 4-(2,3,4,5-tetrafluoro-6-(methylthio)benzamido)-7H-pyrrolo[2,3-d]pyrimidine-7-carboxylate (0.25 g, 0.54 mmol) in DCM (3 mL) was added oxone (0.84 g, 2.74 mmol) at 0° C. The resulting reaction mixture was stirred at room temperature for 30 h. After completion of reaction, the mixture was diluted with a saturated aqueous solution of NaHCO₃ (50 mL) and extracted with EtOAc (3×40 mL). The combined organic phases were dried over anhydrous Na₂SO₄, filtered, and concentrated under reduced pressure. The crude material was purified by flash column chromatography (40% EtOAc in hexane) to afford title compound as a yellow solid (0.13 g, 0.27 mmol, 50% yield). ¹H NMR (400 MHz, DMSO-d₆) δ 10.50 (s, 1H), 8.37 (s, 1H), 8.00 (s, 1H), 7.10 (d, J=4 Hz, 1H), 2.61 (s, 3H), 1.51 (s, 9H). ESI-MS: measured m/z 473.2 [M+1]⁺.

### 2,3,4,5-tetrafluoro-6-(methylsulfinyl)-N-(7H-pyrrolo[2,3-d]pyrimidin-4-yl)benzamide Compound I-46

To a stirred solution of tert-butyl 4-(2,3,4,5-tetrafluoro-6-(methylsulfinyl)benzamido)-7H-pyrrolo[2,3-d]pyrimidine-7-carboxylate (0.11 g, 0.23 mmol) in DCM (2 mL) was added 4N HCl in Dioxane (0.5 mL) at 0° C. The resulting solution was stirred at room temperature. After 4 hrs, the reaction was diluted with a saturated aqueous solution of NaHCO₃ (30 mL) and extracted with EtOAc (3×30 mL). The combined organic phases were dried over anhydrous Na₂SO₄, filtered, and concentrated under reduced pressure. The crude material was purified by reverse phase chromatography (33% ACN in water) to afford title compound as a yellow solid (0.040 g, 0.107 mmol, 46% yield). ¹H NMR (400 MHz, DMSO-d₆) δ 7.92 (s, 1H), 7.75 (s, 1H), 7.42 (s, 2H), 6.95 (d, J=4 Hz, 1H), 3.05 (s, 3H). ¹⁹F NMR (376 MHz, DMSO-d₆) δ −137.80-−137.92 (m, 1F), −142.08-−142.20 (m, 1F), −151.05-−151.12 (1F), −151.90-−151.99 (m, 1F). ESI-MS: measured m/z 373.20 [M+1]⁺. HPLC (Method I) RT=4.93 min., 97.3%.

### Preparation of N-(2-((7H-pyrrolo[2,3-d]pyrimidin-4-yl)amino)ethyl)-2-(N,N-bis(4-methoxybenzyl) sulfamoyl)-3,4,5,6-tetrafluorobenzamide Compound I-48

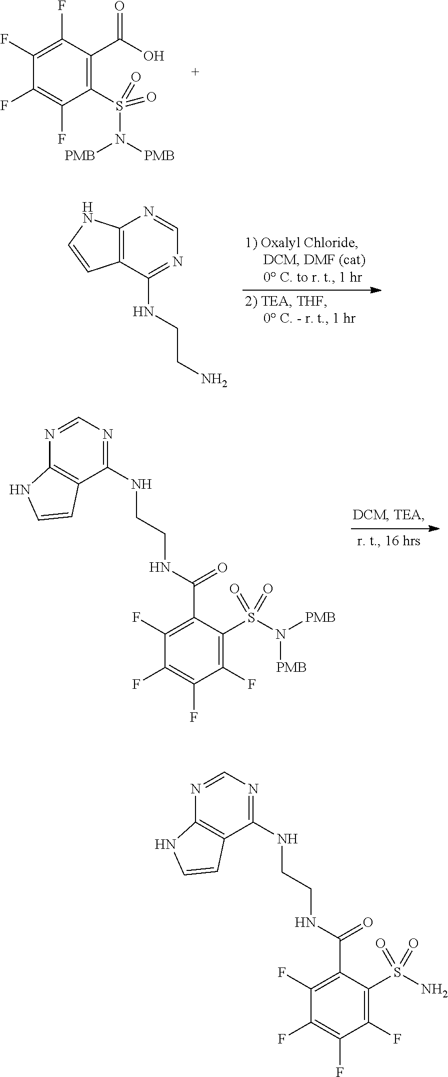

### N-(2-((7H-pyrrolo[2,3-d]pyrimidin-4-yl)amino)ethyl)-2-(N,N-bis(4-methoxybenzyl)sulfamoyl)-3,4,5,6-tetrafluorobenzamide

N-(2-((7H-pyrrolo[2,3-d]pyrimidin-4-yl)amino)ethyl)-2-(N,N-bis(4-methoxybenzyl)sulfamoyl)-3,4,5,6-tetrafluorobenzamide was prepared from 2-(N,N-bis(4-methoxybenzyl)sulfamoyl)-3,4,5,6-tetrafluorobenzoic acid (0.6 g, 1.17 mmol) and N¹-(7H-pyrrolo[2,3-d]pyrimidin-4-yl)ethane-1,2-diamine (0.31 g, 1.75 mmol) according to the protocol described in general procedure A and isolated as a yellow solid (0.55 g, 0.82 mmol, 70% yield). ESI-MS: measured m/z 673.2 [M+1]⁺.

### N-(2-((7H-pyrrolo[2,3-d]pyrimidin-4-yl)amino)ethyl)-2-(N,N-bis(4-methoxybenzyl) sulfamoyl)-3,4,5,6-tetrafluorobenzamide Compound I-48

To stirred solution of N-(2-((7H-pyrrolo[2,3-d]pyrimidin-4-yl)amino)ethyl)-2-(N,N-bis(4-methoxybenzyl)sulfamoyl)-3,4,5,6-tetrafluorobenzamide (0.15 g, 0.22 mmol) in DCM (1.5 mL) was added TFA (1.5 mL). After 16 hrs, the reaction was diluted with saturated aqueous NaHCO₃ (30 mL) and extracted with EtOAc (3×30 mL). The combined organic phases were dried over anhydrous Na₂SO₄, filtered, and concentrated under reduced pressure. The crude material was purified by flash column chromatography (96% EtOAc in hexanes) to afford the title compound as an off-white solid (0.016 g, 0.037 mmol, 16% yield). ¹H NMR (400 MHz, DMSO-d₆) δ 11.51 (s, 1H), 8.80 (s, 1H), 8.11 (s, 2H), 7.30 (s, 1H), 7.08 (s, 1H), 6.50 (s, 1H), 3.64 (d, J=6.4 Hz, 2H), 3.45 (t, J=6.4, 2H). ¹⁹F NMR (376 MHz, DMSO-d₆) δ −134.69-−134.60 (m, 1F), −141.97-−141.06 (m, 1F), −148.96-−149.02 (m, 1F), −153.11-−153.23 (m, 1F). ESI-MS: m/z 433.3 [M+1]⁺, HPLC (Method I): RT=4.05 min., 95.5%.

### Preparation of (4-amino-7H-pyrrolo[2,3-d]pyrimidin-7-yl)(2,3,4,5-tetrafluoro-6-(methylsulfonyl) phenyl)methanone Compound I-49

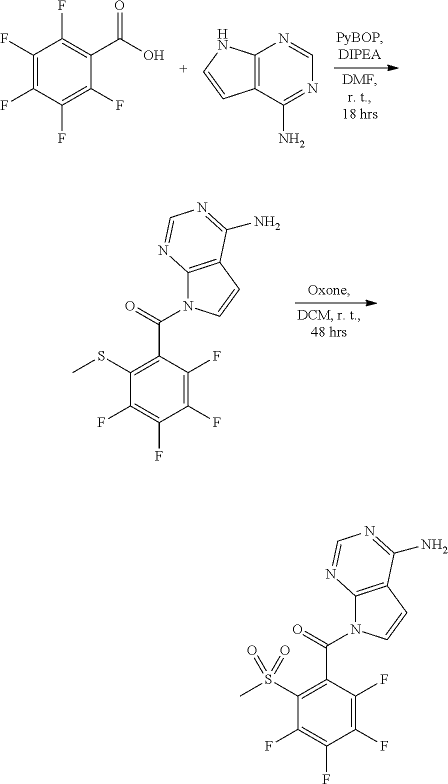 [SMILES: O=C(O)c1c(F)c(F)c(F)c(F)c1F ; Nc1ncnc2[nH]ccc12 ; CSc1c(F)c(F)c(F)c(F)c1C(=O)n1ccc2c(N)ncnc21 ; CS(=O)(=O)c1c(F)c(F)c(F)c(F)c1C(=O)n1ccc2c(N)ncnc21]

### (4-amino-7H-pyrrolo[2,3-d]pyrimidin-7-yl)(2,3,4,5-tetrafluoro-6-(methylthio) phenyl)methanone

(4-amino-7H-pyrrolo[2,3-d]pyrimidin-7-yl)(2,3,4,5-tetrafluoro-6-(methylthio) phenyl)methanone was prepared from 2,3,4,5-tetrafluoro-6-(methylthio)benzoic acid (0.4 g, 1.66 mmol) and 7H-pyrrolo[2,3-d]pyrimidin-4-amine (0.335 g, 2.49 mmol) according to the protocol described in general procedure D and isolated as a green oil (0.30 g, 0.84 mmol, 50% yield). ¹H NMR (400 MHz, DMSO-d₆) δ 7.21 (s, 1H), 7.08 (s, 1H), 6.76 (s, 2H), 6.29 (s, 1H), 2.61 (s, 3H). ESI-MS: measured m/z 357.42 [M+1]⁺.

### (4-amino-7H-pyrrolo[2,3-d]pyrimidin-7-yl)(2,3,4,5-tetrafluoro-6-(methylsulfonyl) phenyl)methanone Compound I-49

(4-amino-7H-pyrrolo[2,3-d]pyrimidin-7-yl)(2,3,4,5-tetrafluoro-6-(methylsulfonyl) phenyl)methanone was prepared from (4-amino-7H-pyrrolo[2,3-d]pyrimidin-7-yl)(2,3,4,5-tetrafluoro-6-(methylthio)phenyl)methanone (0.30 g, 0.84 mmol) according to the protocol described in general procedure C and isolated as a yellow solid (0.15 g, 0.38 mmol, 46% yield). H NMR (400 MHz, DMSO-d₆) δ 7.92 (s, 1H), 7.79 (d, J=4 Hz, 1H), 7.50 (s, 2H), 7.00 (d, J=4 Hz, 1H), 3.29 (s, 3H). ¹⁹F NMR (376 MHz, DMSO-d₆) δ −133.08-−133.16 (m, 1F), −141.65-−141.73 (m, 1F), −145.02-−145.17 (m, 1F). −149.67-−149.79 (m, 1F). ESI-MS: measured m/z 389.28 [M+1]⁺. HPLC (Method I): RT=5.19 min., 100%.

### Preparation of N¹-(7H-pyrrolo[2,3-d] pyrimidin-4-yl)-N2-(2,3,4,5-tetrafluoro-6-(methyl sulfonyl) benzyl) ethane-1,2-diamine Compound I-50

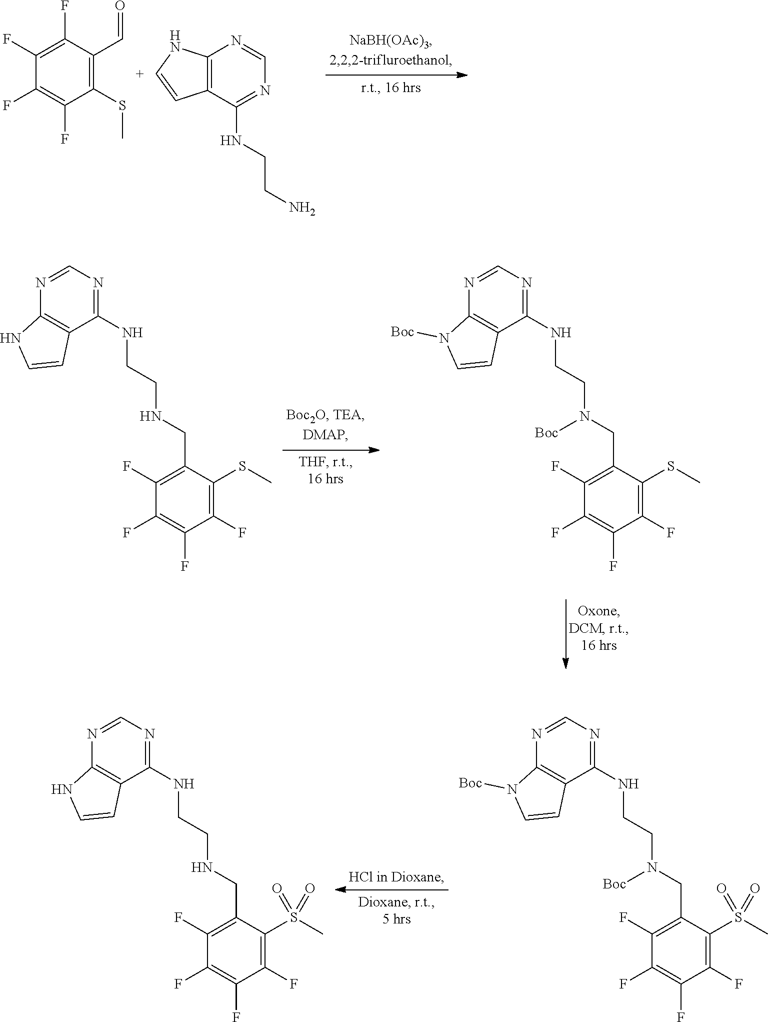

### N¹-(7H-pyrrolo[2,3-d]pyrimidin-4-yl)-N2-(2,3,4,5-tetrafluoro-6-(methylthio)benzyl)ethane-1,2-diamine

To a stirred solution of 2,3,4,5-tetrafluoro-6-(methylthio) benzaldehyde (1.0 g, 4.46 mmol) in 2,2,2-Trifluroethanol (10 mL) was added N¹-(7H-pyrrolo[2,3-d] pyrimidin-4-yl) ethane-1,2-diamine (0.791 g, 4.46 mmol). The reaction mixture was allowed to stir at room temperature for 1 hr, followed by addition of sodium triacetoxyborohydride (2.79 g, 13.39 mmol) at 0° C. The resulting mixture was warmed to room temperature and stirred overnight. After 16 hrs, the reaction was concentrated under reduced pressure. The crude residue was purified by flash column chromatography (10% methanol in DCM) to afford title compound as a white solid (0.32 g, 0.83 mmol, Quantitative). ¹H NMR (400 MHz, DMSO-d₆) δ 11.46 (s, 1H), 8.053 (s, 1H), 7.28 (t, J=5.2 Hz, 1H), 7.04 (t, J=3.2 Hz, 1H), 6.49-6.48 (m, 1H), 3.96 (d, J=2.4 Hz, 2H), 3.53-3.52 (m, 2H), 2.74 (t, J=6.4 Hz, 2H), 2.37 (s, 3H). ¹⁹F NMR (376 MHz, DMSO-d₆) δ −131.14-−131.24 (m, 1F), −141.88-−141.91 (m, 1F), −156.31-−156.44 (m, 1F), −157.08-−157.20 (m, 1F). ESI-MS: measured m/z 386.33 [M+1]⁺.

### tert-butyl 4-((2-((tert-butoxycarbonyl)(2,3,4,5-tetrafluoro-6-(methylthio)benzyl)amino)ethyl)amino)-7H-pyrrolo[2,3-d]pyrimidine-7-carboxylate

To a stirred solution of N¹-(7H-pyrrolo[2,3-d] pyrimidin-4-yl)-N2-(2,3,4,5-tetrafluoro-6-(methylthio) benzyl) ethane-1,2-diamine (0.32 g, 0.83 mmol) in THE (5 mL) was added TEA, (0.633 g, 2.90 mmol), DMAP (0.01 g, 0.083 mmol) and Boc anhydride (0.633 g, 2.90 mmol). After 16 hrs, reaction mixture was diluted with water (100 mL) and extracted with ethyl acetate (2×50 mL). The combined organic phases were dried over anhydrous Na₂SO₄, filtered, and concentrated under reduced pressure. The crude material was purified by flash column chromatography (25% ethyl acetate in hexanes) to afford the title compound as a white solid (0.15 g, 0.25 mmol, 31% yield). ¹H NMR (400 MHz, DMSO-d₆) δ 8.19 (s, 1H), 7.43 (s, 1H), 6.69 (s, 1H), 4.74 (s, 2H), 3.54-5.53 (m, 2H), 2.33 (s, 3H), 1.57 (s, 9H), 1.24 (s, 9H). ESI-MS: measured m/z 586.29 [M+1]⁺.

### tert-butyl 4-((2-((tert-butoxy carbonyl) (2,3,4,5-tetrafluoro-6-(methyl sulfonyl) benzyl) amino) ethyl) amino)-7H-pyrrolo[2,3-d] pyrimidine-7-carboxylate

tert-butyl 4-((2-((tert-butoxy carbonyl) (2,3,4,5-tetrafluoro-6-(methyl sulfonyl) benzyl) amino) ethyl) amino)-7H-pyrrolo[2,3-d] pyrimidine-7-carboxylate was prepared from tert-butyl 4-((2-((tert-butoxy carbonyl) (2,3,4,5-tetrafluoro-6-(methyl lthio) benzyl) amino) ethyl) amino)-7H-pyrrolo[2,3-d] pyrimidine-7-carboxylate (0.15 g, 0.25 mmol) according to the protocol described in general procedure C and isolated as a yellow resin (0.07 g, 0.113 mmol, 44% yield). ESI-MS: measured m/z 618.03 [M+1]⁺.

### N¹-(7H-pyrrolo[2,3-d] pyrimidin-4-yl)-N2-(2,3,4,5-tetrafluoro-6-(methyl sulfonyl) benzyl) ethane-1,2-diamine Compound I-50

To a stirred solution of tert-butyl 4-(2-((tert-butoxy carbonyl) (2,3,4,5-tetrafluoro-6-(methyl sulfonyl) benzyl) amino) ethyl) amino)-7H-pyrrolo[2,3-d] pyrimidine-7-carboxylate (0.07 g, 1.97 mmol) in 1,4 dioxane (0.7 mL) at 0° C. was added HCl in dioxane (4M, 0.7 mL). The reaction was warmed to room temperature. After 5 hrs, the reaction mixture concentrated under reduced pressure. The resulting crude was triturated using n-Pantene to afford title compound as brown solid (0.021 g, 1.97 mmol, 21% yield). ¹H NMR (400 MHz, DMSO-d₆) δ 12.52 (br s, 1H), 9.61 (br s, 2H), 8.35 (s, 1H), 7.39 (s, 1H), 6.96 (s, 1H), 4.66 (s, 2H), 3.95 (br s, 2H), 3.62 (s, 3H), 3.43-3.42 (m, 2H). ¹⁹F NMR (376 MHz, DMSO-d₆) δ −130.79-−130.88 (m, 1F), −133.87-−133.98 (m, 1F), −145.73-−145.85 (m, 1F), −149.43-−149.55 (m, 1F). ESI-MS: measured m/z 418.2 [M+1]⁺. HPLC (Method I): RT=3.07 min, 96.7%

### General Procedure F:

### 2,3,4,5-tetrafluoro-N-(3-fluoro-4-methoxyphenyl)-6-(trifluoromethyl) benzenesulfonamide Compound I-6

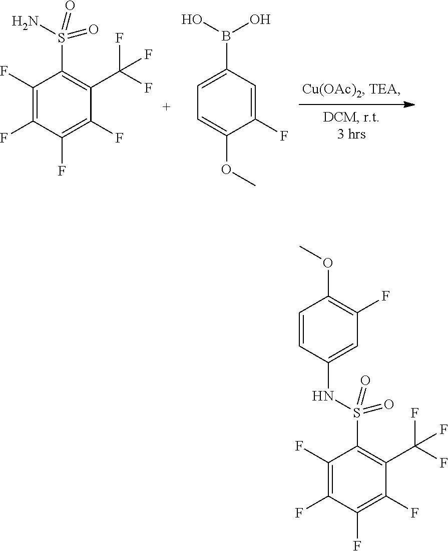

In a 20 mL scintillation vial, 2,3,4,5-tetrafluoro-6-(trifluoromethyl)benzenesulfonamide (0.1 g, 336.53 μmol) was dissolved in DCM (0.1 M) (3.36 mL) under air at r.t. The resulting solution was then added with (3-fluoro-4-methoxy-phenyl)boronic acid (114.38 mg, 673.06 μmol) and oven-dried molecular seives, followed by sequential addition of copper acetate (61.12 mg, 336.53 μmol) and N,N-diethylethanamine (68.11 mg, 673.06 μmol, 93.81 μL). The solution turned from milky heterogeneous blue solution to clear homogeneous blue solution upon addition of TEA. The resulting solution was left to stir until completion. After 3 hours, TLC indicated full consumption of the SM. The mixture was filtered through a pad of Celite and the collected filtrate was concentrated by rotary evaporation under reduced pressure. The mixture was dry-loaded onto a pad of silica equilibrated with 10% EtOAc in Hexanes on Biotage Isolera 25 g cartridge. The mixture was separated by eluting in gradient from 10% EtOAc to 25% EtOAc in Hexanes to afford the desired product as a beige powder (0.064 g, 0.152 mmol, 45% yield). ¹H NMR (400 MHz, CDCl₃) δ 7.03 (dd, J=11.6, 2.4 Hz, 1H), 7.00-6.85 (m, 3H), 3.89 (s, 3H). ¹⁹F NMR (376 MHz, CDCl₃) δ −51.59 (d, J=36.4 Hz, 3F), −129.02 (dt, J=23.9, 10.3 Hz, 1F), −130.39 (qdt, J=36.4, 20.2, 9.8 Hz, 1F), −131.24 (dd, J=11.6, 8.0 Hz, 1F), −142.98 (td, J=20.6, 11.1 Hz, 1F), −144.44 (ddd, J=23.9, 20.1, 10.2 Hz, 1F). ESI+MS: m/z 421.1 [M+1]⁺. HPLC (Method III): RT=8.20 min, 96.5%

### 2,3,4,5-tetrafluoro-N-(3-fluoro-4-methoxyphenyl)-N-methyl-6-(trifluoromethyl) benzenesulfonamide Compound I-7

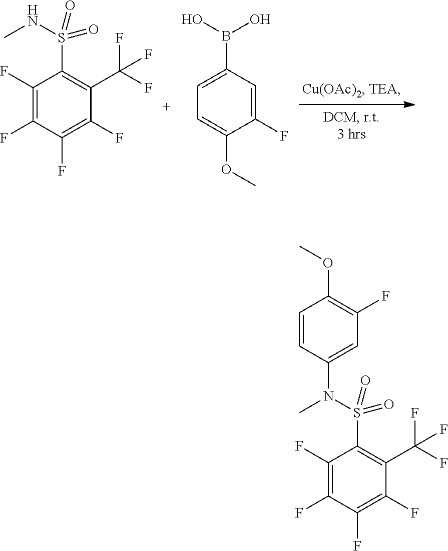

2,3,4,5-tetrafluoro-N-(3-fluoro-4-methoxyphenyl)-N-methyl-6-(trifluoromethyl) benzenesulfonamide was prepared from 2,3,4,5-tetrafluoro-N-methyl-6-(trifluoromethyl)benzenesulfonamide (0.051 g, 0.164 mmol) and (3-fluoro-4-methoxyphenyl)boronic acid (0.056 g, 0.328 mmol) according to the protocol described in general procedure F and isolated as a beige resin (0.016 g, 0.036 mmol, 22% yield). ¹H NMR (400 MHz, CDCl₃) δ 7.09-7.01 (m, 2H), 6.93 (t, J=9.1 Hz, 1H), 3.91 (s, 3H), 3.47 (d, J=2.6 Hz, 3H). ¹⁹F NMR (376 MHz, CDCl₃) δ −51.59 (d, J=37.8 Hz, 3F), −127.98 (dt, J=22.3, 10.1 Hz, 1F), −130.44-−130.90 (m, 1F), −131.58-−131.69 (m, 1F), −144.01 (td, J=20.6, 10.6 Hz, 1F), −145.08 (ddd, J=24.1, 20.1, 9.8 Hz, 1F). ESI+MS: m/z 434.1 [M−1]⁻. HPLC (Method III): RT=8.66 min, 97.2%

### Preparation of 2,3,4,5-tetrafluoro-N-(3-fluoro-4-methoxyphenyl)-6-(methylsulfonyl)benzamide Compound I-8 and 2,3,4,5-tetrafluoro-N-(3-fluoro-4-methoxyphenyl)-6-(methylsulfinyl)benzamide Compound I-10

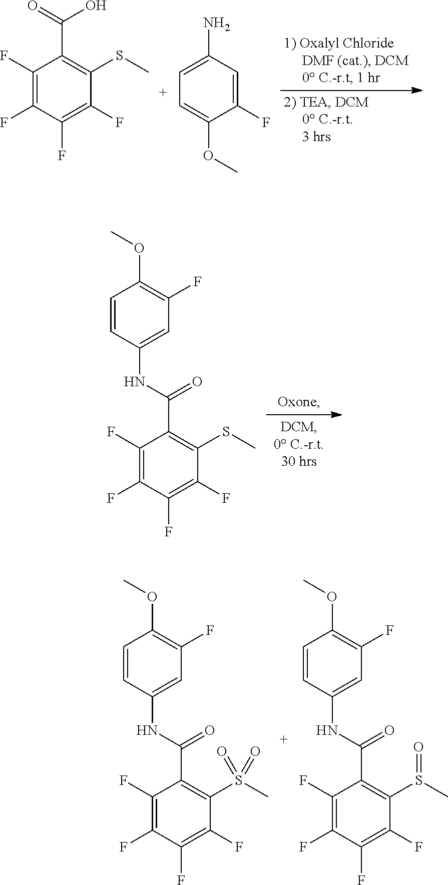

### 2,3,4,5-tetrafluoro-N-(3-fluoro-4-methoxyphenyl)-6-(methylthio)benzamide

2,3,4,5-tetrafluoro-N-(3-fluoro-4-methoxyphenyl)-6-(methylthio)benzamide was prepared from 2,3,4,5-tetrafluoro-6-(methylthio)benzoic acid (0.20 g, 0.83 mmol) and 3-fluoro-4-methoxyaniline (0.11 g, 0.83 mmol) according to the protocol described in general procedure A and isolated as a yellow solid (0.24 g, 0.66 mmol, 80% yield). ¹H NMR (400 MHz, DMSO-d₆) δ 10.82 (s, 1H), 7.62 (dd, J₁=2.4 Hz, J₂=13.2 Hz, 1H), 7.36-7.32 (m, 1H), 7.19 (t, J=9.2 Hz, 1H), 3.83 (s, 3H), 2.50 (s, 3H). ESI-MS: measured m/z 364.3 [M+1]⁺.

### 2,3,4,5-tetrafluoro-N-(3-fluoro-4-methoxyphenyl)-6-(methylsulfonyl)benzamide Compound I-8

2,3,4,5-tetrafluoro-N-(3-fluoro-4-methoxyphenyl)-6-(methylsulfonyl)benzamide was prepared from 2,3,4,5-tetrafluoro-N-(3-fluoro-4-methoxyphenyl)-6-(methylthio)benzamide (0.026 g, 0.07 mmol) according to the protocol described in general procedure C and isolated as a white solid (0.040 g, 0.101 mmol, 28% yield). ¹H NMR (400 MHz, DMSO-d₆) δ 11.09 (s, 1H), 7.56 (dd, J₁=1.6 Hz, J₂=13.2 Hz, 1H), 7.30-7.28 (m, 1H), 7.18 (t, J=9.6 Hz, 1H), 3.83 (s, 3H), 3.47 (s, 3H). ¹⁹F NMR (376 MHz, DMSO-d₆) δ −132.51-−132.63 (m, 1F), −133.76 (s, 1F), −140.26-−140.35 (m, 1F), −145.15-−145.29 (m, 1F), −151.08-−151.20 (m, 1F). ESI-MS: measured m/z 394.30 [M−1]⁻, HPLC (Method I) RT=6.37 min., 97.9%.

### 2,3,4,5-tetrafluoro-N-(3-fluoro-4-methoxyphenyl)-6-(methylsulfinyl)benzamide Compound I-10

2,3,4,5-tetrafluoro-N-(3-fluoro-4-methoxyphenyl)-6-(methylsulfinyl)benzamide was isolated as a white solid (0.085 g, 0.224 mmol, 59% yield). ¹H NMR (400 MHz, DMSO-d₆) δ 11.09 (s, 1H), 7.60 (dd, J₁=2.4 Hz, J2=13.2 Hz, 1H), 7.33-7.30 (m, 1H), 7.19 (t, J=9.2 Hz, 1H), 3.83 (s, 3H), 3.143 (s, 3H). ¹⁹F NMR (376 MHz, DMSO-d₆) δ −133.57 (s, 1F), −139.03-−139.14 (m, 1F), −140.46-−140.55 (m, 1F), −149.79-−149.93 (m, 1F), −151.50-−151.61 (m, 1F). ESI-MS: measured m/z 378.30 [M−1]⁻. HPLC RT=6.21 min., 97.7%.

### 2-(N,N-dimethylsulfamoyl)-3,4,5,6-tetrafluoro-N-(3-fluoro-4-methoxyphenyl) benzamide Compound I-9

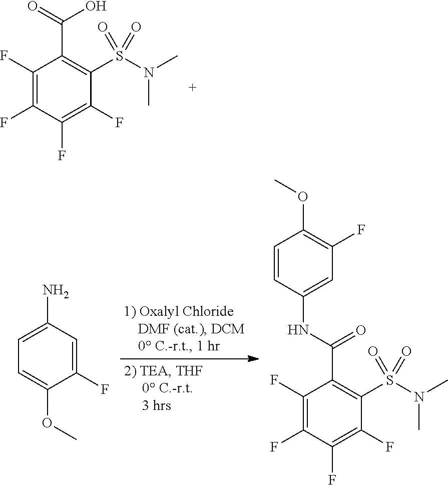

2-(N,N-dimethylsulfamoyl)-3,4,5,6-tetrafluoro-N-(3-fluoro-4-methoxyphenyl) benzamide was prepared from 2-(N,N-dimethylsulfamoyl)-3,4,5,6-tetrafluorobenzoic acid (0.30 g, 1.0 mmol) and 3-fluoro-4-methoxyaniline (0.210 g, 1.5 mmol) according to the protocol described in general procedure A and isolated as a a yellow solid (0.07 g, 0.17 mmol, 18% yield). ¹H NMR (400 MHz, DMSO-d₆) δ 10.79 (s, 1H), 7.54 (dd, J₁=2.4 Hz, J₂=13.2 Hz, 1H), 7.27-7.24 (m, 1H), 7.18 (t, J=9.2 Hz, 1H), 2.85 (s, 6H). ¹⁹F NMR (376 MHz, DMSO-d₆) δ −130.66-−130.78 (m, 1F), 133.65-−133.72 (m, 1F), −140.47-−140.58 (m, 1F), −146.81-−146.96 (m, 1F), −151.13-−151.26 (m, 1F). ESI-MS: measured m/z 425.3 [M+1]⁺. HPLC (Method I): RT=6.78 min., 100%.

### Preparation of (E)-N-(3-fluoro-4-methoxyphenyl)-1-(2,3,4,5-tetrafluoro-6-(methylsulfonyl) phenyl) methanimine Compound I-11

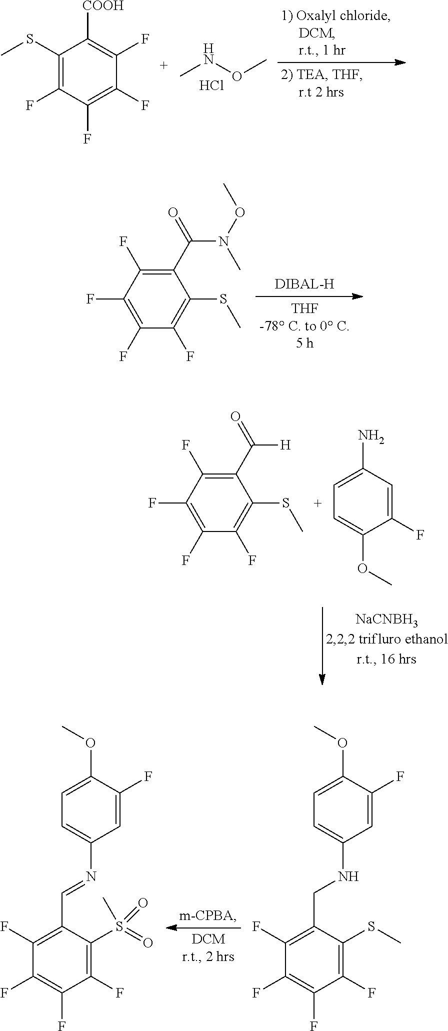

### 2,3,4,5-tetrafluoro-N-methoxy-N-methyl-6-(methylthio) benzamide

2,3,4,5-tetrafluoro-N-methoxy-N-methyl-6-(methylthio) benzamide was prepared from 2,3,4,5-tetrafluoro-6-(methylthio) benzoic acid (1.0 g, 4.16 mmol) and N, O-dimethyl hydroxylamine hydrogen chloride (0.81 g, 8.33 mmol) according to the protocol described in general procedure A and isolated as a yellow gum (0.9 g, 3.17 mmol, 76% yield). ESI-MS: measured m/z 284.2 [M+1]⁺.

### 2,3,4,5-tetrafluoro-6-(methylthio)benzaldehyde

To a stirred solution of 2,3,4,5-tetrafluoro-N-methoxy-N-methyl-6-(methylthio) benzamide (0.8 g, 2.82 mmol) in anhydrous THF (8.0 mL) at −78° C. was added DIBAL-H (1M in toluene, 11.3 mL, 11.3 mmol) under a N₂ atmosphere. The mixture was permitted to warm to 0° C. After 2 hrs, reaction was diluted with aqueous 1N HCl (100 mL) and extracted with EtOAc (2×50 mL). The combined organic phases were dried over anhydrous Na₂SO₄, filtered, and concentrated under reduced pressure to afford title compound as a yellow oil (0.46 g, 2.05 mmol). The obtained material was used without purification. ¹H NMR (400 MHz, CDCl₃) δ 10.49 (s, 1H), 2.55 (s, 3H), ¹⁹F NMR (376 MHz, CDCl₃) δ −130.96-−131.03 (m, 1F), −142.71-−142.81 (m, 1F), −145.34-−145.47 (m, 1F), −154.03-−154.12 (m, 1F).

### 3-fluoro-4-methoxy-N-(2,3,4,5-tetrafluoro-6-(methylthio) benzyl) aniline

To a stirred solution of 2,3,4,5-tetrafluoro-6-(methylthio) benzaldehyde (0.2 g, 0.89 mmol) in 2,2,2 Trifluroethanol (2 mL) was added 3-fluoro-4-methoxyaniline (0.12 g, 0.89 mmol). After 16 hrs, NaCNBH₃ (0.16 g, 2.67 mmol) was added and stirring continued for a further 16 hrs. The reaction was concentrated under reduced pressure and the crude material purified by flash column chromatography (11% EtOAc in hexanes) to afford the title compound as a yellow oil (0.18 g, 0.51 mmol, 58% yield). ¹H NMR (400 MHz, DMSO-d₆) δ 6.94 (t, J=9.6 Hz, 1H), 6.58-6.54 (dd, J₁=2.8 Hz, J₂=14.0 Hz, 1H), 6.44-6.41 (dd, J₁=1.6 Hz, J₂=8.8 Hz, 1H), 5.74 (t, J=5.6, 1H), 4.38-4.36 (m, 2H), 3.70 (s, 3H), 2.43 (s, 3H). ¹⁹F NMR (376 MHz, DMSO-d₆) δ −130.85-−130.94 (m, 1F), −134.02 (s, 1F), −141.04-−141.13 (m, 1F), −156.12-−156.42 (m, 2F). ESI-MS: measured m/z 350.3, [M+1]⁺.

### (E)-N-(3-fluoro-4-methoxyphenyl)-1-(2,3,4,5-tetrafluoro-6-(methylsulfonyl) phenyl) methanimine Compound I-11

To a stirred solution of 3-fluoro-4-methoxy-N-(2,3,4,5-tetrafluoro-6-(methylthio) benzyl) aniline (0.05 g, 0.14 mmol) in DCM (3 mL) was added m-CPBA (0.12 g, 0.71 mmol). After 2 hrs, the reaction mixture was diluted with a saturated aqueous solution of NaHCO₃ (50 mL) and extracted with DCM (2×25 mL). The combined organic phases were dried over anhydrous Na₂SO₄, filtered, and concentrated under reduced pressure. The crude material was purified by flash column chromatography (20% EtOAc in hexanes) to afford the title compound as a yellow solid (0.018 g, 0.051 mmol, 33% yield). ¹H NMR (400 MHz, DMSO-d₆) δ 8.79 (s, 1H), 7.92-7.88 (dd, J₁=2.8 Hz, J₂=12.0 Hz, 1H), 7.82-7.79 (m, 1H), 7.36 (t, J=8.8 Hz, 1H), 3.94 (s, 3H), 3.20 (s, 3H). ¹⁹F NMR (376 MHz, DMSO-d₆) δ −132.74 (s, 1F), −134.83-−134.90 (m, 1F), −139.40-−139.51 (m, 1F), −150.07-−150.62 (m, 2F). ESI-MS: measured m/z 380.3, [M+1]⁺. HPLC RT=6.04 min., 95.8%.

### Scheme --: Preparation of 2,3,4,5-tetrafluoro-N-(3-fluoro-4-methoxyphenyl)-6-(methylsulfonyl)aniline Compound I-12 and 2,3,4,5-tetrafluoro-N-(3-fluoro-4-methoxyphenyl)-6-(methylsulfinyl)aniline Compound I-13

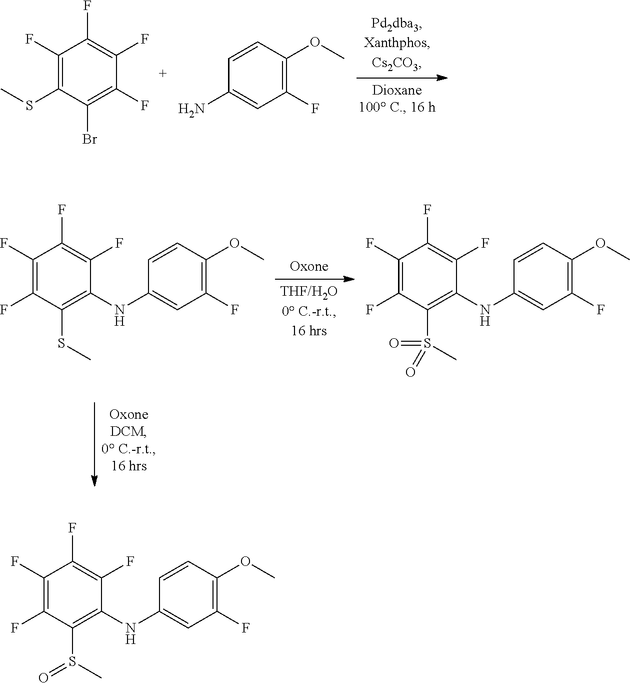

### 2,3,4,5-tetrafluoro-N-(3-fluoro-4-methoxyphenyl)-6-(methylthio)aniline

A solution of 3-fluoro-4-methoxyaniline (0.25 g, 0.90 mmol), (2-bromo-3,4,5,6-tetrafluorophenyl)(methyl)sulfane (0.13 g, 0.90 mmol), and Cs₂CO₃ (0.59 g, 1.81 mmol), in 1,4-dioxane (2.5 mL) was purged with N₂ for 15 minutes. To this reaction mixture was added Pd₂dba₃ (0.08 g, 0.09 mmol) and Xantphos (0.05 g, 0.09 mmol). The resulting mixture was heated to 100° C. as left to stir overnight. After 16 hrs, the mixture was cooled to room temperature and diluted with water (50 mL), then extracted with EtOAc (2×30 mL). The combined organic phases were dried over anhydrous Na₂SO₄, filtered, and concentrated under reduced pressure. The crude material was purified by flash column chromatography (16% EtOAc in hexanes) to afford the title compound as a light yellow solid (0.19 g, 0.56 mmol, 62% yield). ¹H NMR (400 MHz, DMSO-d₆) δ7.84 (s, 1H), 6.99 (t, J=9.6 Hz, 1H), 6.71-6.68 (dd, J₁=1.6 Hz, J₂=13.6 Hz, 1H), 6.56-6.53 (dd, J₁=1.2 Hz, J₂=8.8 Hz, 1H), 3.76 (s, 3H), 2.32 (s, 3H). ¹⁹F NMR (376 MHz, DMSO-d6) δ −132.08-−132.17 (m, 1F), −134.29 (s, 1F), −145.26-−145.35 (m, 1F), −156.13-−156.25 (m, 1F), −163.86-−163.99 (m, 1F). ESI-MS: measured m/z 336.2 [M+1]⁺.

### 2,3,4,5-tetrafluoro-N-(3-fluoro-4-methoxyphenyl)-6-(methylsulfonyl)aniline Compound I-12

To a stirred solution of 2,3,4,5-tetrafluoro-N-(3-fluoro-4-methoxyphenyl)-6-(methylthio)aniline (0.10 g, 0.29 mmol) in THF:Water (0.8 mL:0.2 mL) at 0° C. was added Oxone (0.45 g, 1.49 mmol). The resulting mixture was warmed to room temperature and stirred overnight. After 16 hrs, a second portion of Oxone (0.45 g, 1.49 mmol) was added and the reaction stirred for 16 hrs. Once complete, the reaction was diluted with a saturated aqueous solution of NaHCO₃ and extracted with EtOAc (2×30 mL). The combined organic phases were dried over anhydrous Na₂SO₄, filtered, and concentrated under reduced pressure. The crude material was purified by flash column chromatography (20% EtOAc in hexanes) to afford the title compound as white solid (0.02 g, 0.054 mmol, 18% yield). ¹H NMR (400 MHz, DMSO-d₆) δ 7.95 (s, 1H), 7.04 (t, J=8.0 Hz, 1H), 6.90 (d, J=12.4 Hz, 1H), 6.73 (d, J=6.8 Hz, 1H), 3.78 (s, 3H), 3.42 (s, 3H). ¹⁹F NMR (376 MHz, DMSO-d₆) δ −134.09 (s, 1F), −135.41-−135.52 (m, 1F), −142.01-−142.09 (m, 1F), −147.24-−147.38 (m, 1F), −163.41-−163.53 (m, 1F). ESI-MS: measured m/z 366.4 [M−1]⁻. HPLC RT=7.10 min., 97.5%.

### 2,3,4,5-tetrafluoro-N-(3-fluoro-4-methoxyphenyl)-6-(methylsulfinyl)aniline Compound I-13

2,3,4,5-tetrafluoro-N-(3-fluoro-4-methoxyphenyl)-6-(methylsulfinyl)aniline was prepared from 2,3,4,5-tetrafluoro-N-(3-fluoro-4-methoxyphenyl)-6-(methylthio)aniline (0.08 g, 0.23 mmol) according to the protocol described in general procedure C and isolated an a light pink solid (0.05 g, 0.14 mmol, 59% yield). ¹H NMR (400 MHz, DMSO-d₆) δ 8.23 (s, 1H), 7.05 (t, J=9.2 Hz, 1H), 6.80 (d, J=13.2 Hz, 1H), 6.63 (d, J=8.4 Hz, 1H), 3.78 (s, 3H), 3.04 (s, 3H). ¹⁹F NMR (376 MHz, DMSO-d₆) δ −133.79 (s, 1F), −141.42-−141.53 (m, 1F), −144.13-−144.21 (m, 1F), −150.88-−151.00 (m, 1F), −163.14-−163.26 (m, 1F). ESI-MS: measured m/z 352.3 [M+1]⁺. HPLC RT=8.29 min., 99%.

### Preparation of 1-(benzyloxy)-2,3,4,5-tetrafluoro-6-((3-fluoro-4-methoxybenzyl)sulfonyl)benzene Compound I-16, 2,3,4,5-tetrafluoro-6-((3-fluoro-4-methoxybenzyl)sulfonyl)phenol Compound I-14, 1-(difluoromethoxy)-2,3,4,5-tetrafluoro-6-((3-fluoro-4-methoxybenzyl)sulfonyl) benzene Compound I-15 and 1,2,3,4-tetrafluoro-5-((3-fluoro-4-methoxybenzylsulfonyl)-6-methoxybenzene Compound I-19

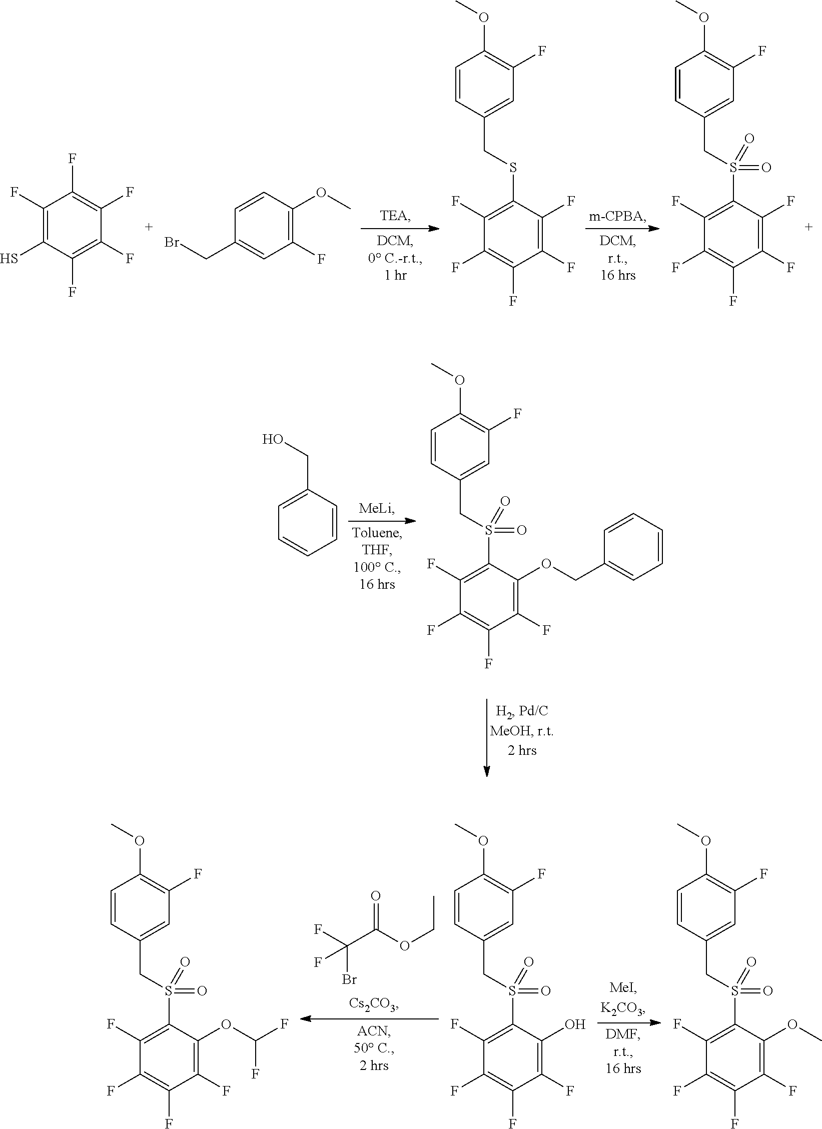

### (3-fluoro-4-methoxybenzyl)(perfluorophenyl)sulfane

To a stirred solution of 2,3,4,5,6-pentafluorobenzenethiol (5 g, 25.0 mmol) in DCM (50 mL) at 0° C. was added TEA (4.37 mL, 32.0 mmol) and 4-(bromomethyl)-2-fluoro-1-methoxybenzene (5.5 g, 25.0 mmol). The reaction was warmed to room temperature and stirred for 1 hr. Once complete, the reaction mixture was diluted with water (200 mL) and extracted with DCM (2×50 mL). The combined organic phases were dried over anhydrous Na₂SO₄, filtered, and concentrated under reduced pressure to afford title compound as a brown solid (8.0 g, 23.66 mmol, 95% yield). ¹H NMR (400 MHz, DMSO-d₆) δ 7.13 (dd, J=2.0, 12.4 Hz, 1H), 7.04 (t, J=8.4 Hz, 1H), 6.94 (d, J=8.0 Hz, 1H), 4.07 (s, 2H), 3.80 (s, 3H). ¹⁹F NMR (376 MHz, DMSO-d₆) δ −132.59-−132.67 (m, 2F), −135.34 (s, 1F), −152.50-150.62 (m, 1F), −161.49-−161.60 (m, 1F).

### 1,2,3,4,5-pentafluoro-6-((3-fluoro-4-methoxybenzyl)sulfonyl)benzene

To a stirred, ice-cold (0° C.) solution of (3-fluoro-4-methoxybenzyl)(perfluorophenyl)sulfane (3.0 g, 8.8 mmol) in DCM (30 mL) was added m-CPBA (7.69 g, 4.4 mmol). The reaction was warmed to room temperature overnight. After 16 hrs, the mixture was diluted with a saturated aqueous solution of NaHCO₃ (250 mL) and extracted with DCM (2×50 mL). The combined organic phases were dried over anhydrous Na₂SO₄, filtered, and concentrated under reduced pressure to afford title compound as an off-white solid. (3.5 g, 9.45 mmol, Quantitative). ¹H NMR (400 MHz, DMSO-d₆) δ 7.22 (dd, J₁=2.4 Hz, J₂=12 Hz, 1H), 7.15 (t, J=8.8 Hz, 1H), 7.08 (d, J=8.4 Hz, 1H), 4.87 (s, 2H), 3.84 (s, 3H).

### 1-(benzyloxy)-2,3,4,5-tetrafluoro-6-((3-fluoro-4-methoxybenzyl)sulfonyl)benzene Compound I-16

A 1.6M MeLi solution in diethyl ether (3.37 mL, 5.4 mmol) was added dropwise to an ice-cold (0° C.) solution of benzyl alcohol (0.35 g, 3.24 mmol) in Toluene:THF (9:1, 30 mL). The resulting reaction mixture was stirred at room temperature for 1 hr, after which it was slowly added to a stirred solution of 1,2,3,4,5-pentafluoro-6-((3-fluoro-4-methoxybenzyl)sulfonyl)benzene (1.0 g, 2.70 mmol) in Toluene:THF (9:1, 30 mL) maintained at 0° C. The reaction was heated to 100° C. overnight. After 16 hrs, the reaction was cooled to room temperature and quenched with 1N HCl solution (50 mL). The resulting biphasic mixture was extracted with EtOAc (2×50 mL) and the combined organic phases were dried over anhydrous Na₂SO₄, filtered, and concentrated under reduced pressure. The crude material was purified by flash column chromatography (0-10% EtOAc in hexanes), followed by trituration using MeOH, to afford the title compound as an off-white solid (0.45 g, 0.98 mmol, 36% yield). ¹H NMR (400 MHz, DMSO-d₆) δ 7.56-7.54 (m, 2H), 7.49-7.43 (m, 3H), 7.11 (t, J=8.8 Hz, 1H), 7.03 (dd, J=12 Hz, 1H), 6.93 (d, J=8.8 Hz, 1H), 5.18 (s, 2H), 4.72 (s, 2H), 3.81 (s, 3H) ¹⁹F NMR (376 MHz, DMSO-d₆) δ −135.31-−135.33 (m, 1F), −138.02-−138.11 (m, 1F), −146.10-−146.24 (m, 1F), −151.31-−151.39 (m, 1F) −160.49-−160.62 (m, 1F). HPLC (Method I): RT=7.81 min., 95.4%.

### 2,3,4,5-tetrafluoro-6-((3-fluoro-4-methoxybenzyl)sulfonyl)phenol Compound I-14

Under an inert atmosphere of nitrogen, 10% wet Pd/C (0.75 g) was added to a solution of 1-(benzyloxy)-2,3,4,5-tetrafluoro-6-((3-fluoro-4-methoxybenzyl)sulfonyl)benzene (0.75 g, 1.63 mmol) in MeOH:THF (1:1, 20 mL). The reaction vessel was flushed with H₂ and the reaction stirred for 2 hrs while under a positive pressure of hydrogen. Once complete, the reaction mixture was filtered through a plug of celite and the filtrate concentrated under reduced pressure. The crude material was purified by reverse phase chromatography (0-30% acetonitrile in water) to afford the title compound as a pink solid (0.2 g, 0.28 mmol, 33% yield). ¹H NMR (400 MHz, DMSO-d₆) δ 7.14-7.10 (m, 2H), 7.01-6.99 (m, 1H), 4.78 (s, 2H), 3.82 (s, 3H). ¹⁹F NMR (376 MHz, CDCl₃) δ −132.85 (s, 1F), −136.93-−137.03 (m, 1F), −143.35-−143.48 (m, 1F), −157.71-−157.79 (m, 1F), −166.24-−166.37 (m, 1F). ESI-MS: measured m/z 367.23 [M−1]⁻. HPLC (Method I): RT=6.70 min., 93%.

### 1-(difluoromethoxy)-2,3,4,5-tetrafluoro-6-((3-fluoro-4-methoxybenzyl)sulfonyl) benzene Compound I-15

To a stirred solution of 2,3,4,5-tetrafluoro-6-((3-fluoro-4-methoxybenzyl)sulfonyl)phenol (0.06 g, 0.16 mmol) in acetonitrile (1 mL) was added ethyl-bromodifluoroacetate (0.099 g, 0.48 mmol) and Cs₂CO₃ (0.15 g, 0.48 mmol) at room temperature. The reaction was heated to 80° C. for 1 hr, after which it was cooled down and diluted with water (20 mL). The resulting biphasic mixture was extracted with EtOAc (2×10 mL) and the combined organic phases were dried over anhydrous Na₂SO₄, filtered, and concentrated under reduced pressure. The crude material was purified by flash column chromatography (0-7% EtOAc in hexanes) to afford title compound as an off-white solid (0.035 g, 0.10 mmol, 36% yield). ¹H NMR (400 MHz, DMSO-d₆) δ 7.20-7.13 (m, 2H), 7.06-6.84 (m, 2H), 4.80 (s, 2H), 3.84 (s, 3H). ¹⁹F NMR (376 MHz, DMSO-d₆) δ −81.89-−81.92 (m, 2F), −134.55-−134.66 (m, 1F), −135.31 (s, 1F), −144.60-−144.74 (m, 1F), −148.99-−149.07 (m, 1F), −155.16-−155.29 (m, 1F). ESI-MS: measured m/z 415.1 [M−3]. HPLC (Method I): RT=7.15 min, 98.3%.

### 1,2,3,4-tetrafluoro-5-((3-fluoro-4-methoxybenzyl)sulfonyl)-6-methoxybenzene Compound I-19

To a stirred solution of 2,3,4,5-tetrafluoro-6-((3-fluoro-4-methoxybenzyl)sulfonyl)phenol (0.08 g, 0.21 mmol) in DMF (1 mL) was added MeI (0.06 g, 0.43 mmol) and K₂CO₃ (0.059 g, 0.43 mmol). After 16 hrs, reaction mixture was diluted with cold water (20 mL) and extracted with EtOAc (2×10 mL). The combined organic phases were dried over anhydrous Na₂SO₄, filtered, and concentrated under reduced pressure. The obtained crude was purified by flash column chromatography (0-30% EtOAc in hexane) to afford the title compound as an off-white solid (0.038 g, 0.10 mmol, 36% yield). ¹H NMR (400 MHz, DMSO-d₆) δ 7.17 (dd, J₁=2.4 Hz, J₂=12 Hz, 1H), 7.15-7.11 (d, J=8.8 Hz, 1H), 7.23 (dd, J₁=0.8 Hz, J₂=8.4 Hz, 1H), 4.77 (s, 2H), 4.01 (s, 3H), 3.83 (s, 3H). ¹⁹F NMR (376 MHz, DMSO-d₆) δ −135.36 (s, 1F), −138.74-−138.85 (m, 1F), −146.15-−146.29 (m, 1F), −152.88-−152.96 (m, 1F), −161.08-−161.21 (m, 1F). ESI-MS: measured m/z 381.54 [M−1]⁻. HPLC (Method I): RT=7.05 min., 99.3%.

### Preparation of 1,2,3,4-tetrafluoro-5-((3-fluoro-4-methoxyphenoxy)methyl)-6-(methylsulfonyl) benzene Compound I-17

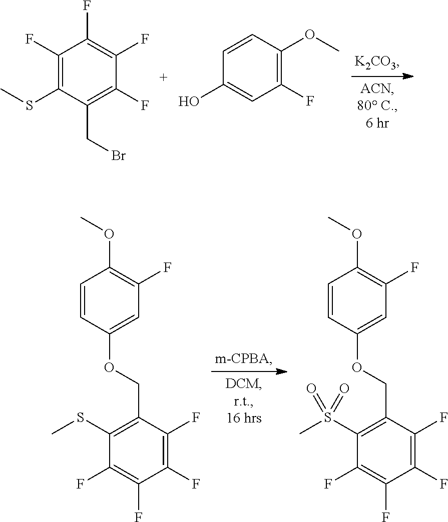

### methyl(2,3,4,5-tetrafluoro-6-((3-fluoro-4-methoxyphenoxy)methyl)phenyl) sulfane

To a stirred solution of (2-(bromomethyl)-3,4,5,6-tetrafluorophenyl)(methyl)sulfane (0.16 g, 1.12 mmol) in acetonitrile (5 mL) was added 3-fluoro-4-methoxyphenol (0.39 g, 1.35 mmol) and K₂CO₃ (0.46 g, 3.38 mmol). The resulting mixture was heated to 80° C. for 6 hrs. Once complete, the reaction was cooled to room temperature and diluted with water (20 mL). The resulting biphasic mixture was extracted with EtOAc (2×20 mL) and the combined organic phases dried over anhydrous Na₂SO₄, filtered, and concentrated under reduced pressure. The crude material was purified by flash column chromatography (0-15% EtOAc in hexanes) to afford title compound as an off-white solid (0.19 g, 0.54 mmol, 40% yield). ¹H NMR (400 MHz, DMSO-d₆) δ 7.11 (t, J=9.2 Hz, 1H), 7.05-7.01 (m, 1H), 6.85-6.82 (m, 1H), 5.20 (d, J=2.8 Hz, 2H), 3.79 (s, 3H), 2.42 (s, 3H). ¹⁹F NMR (376 MHz, DMSO-d₆) δ −130.37-−130.46 (m, 1F), −132.44 (s, 1F), −140.86-−140.96 (m, 1F), −153.66-−153.78 (m, 1F), −155.23-−155.35 (m, 1F).

### 1,2,3,4-tetrafluoro-5-((3-fluoro-4-methoxyphenoxy)methyl)-6-(methylsulfonyl) benzene Compound I-17

To a stirred, ice-cold (0° C.) solution of methyl(2,3,4,5-tetrafluoro-6-((3-fluoro-4-methoxyphenoxy)methyl)phenyl)sulfane (0.18 g, 0.51 mmol) in DCM (2 mL) was added m-CPBA (0.088 g, 0.51 mmol). The reaction mixture was stirred at room temperature for 1 hr, followed by addition of another portion of m-CPBA (0.088 g, 0.51 mmol) at 0° C. The reaction mixture was stirred at room temperature overnight. After 16 hrs, the reaction was diluted with a saturated aqueous solution of NaHCO₃ (20 mL) and extracted with EtOAc (3×20 mL). The combined organic phases were dried over anhydrous Na₂SO₄, filtered, and concentrated under reduced pressure. The crude material was purified by flash column chromatography (0-30% EtOAc in hexanes) to afford the title compound as a brown sticky solid (0.020 g, 0.052 mmol, 10% yield). ¹H NMR (400 MHz, DMSO-d₆) 57.12 (t, J=9.6 Hz, 1H), 7.00 (dd, J₁=3.2 Hz, J₂=13.2 Hz, 1H), 6.83-6.80 (m, 1H), 5.38 (d, J=2.8 Hz, 2H), 3.79 (s, 3H), 3.45 (s, 3H). ¹⁹F NMR (376 MHz, DMSO-d₆) δ −131.14-−131.26 (m, 1F), −132.53 (s, 1F), −139.13-−139.23 (m, 1F), −146.20-−146.32 (m, 1F), −151.06-−151.19 (m, 1F).

### 3-fluoro-4-methoxy-N-(2,3,4,5-tetrafluoro-6-(methyl sulfinyl) benzyl) aniline Compound I-18

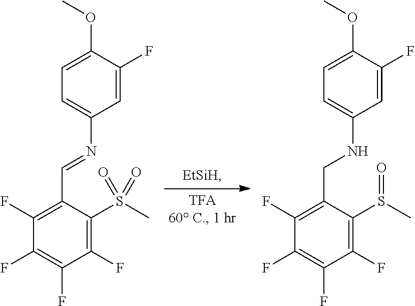 [SMILES: COc1ccc(/N=C/c2c(F)c(F)c(F)c(F)c2S(C)(=O)=O)cc1F ; COc1ccc(NCc2c(F)c(F)c(F)c(F)c2S(C)=O)cc1F]

To a stirred solution of (E)-N-(3-fluoro-4-methoxyphenyl)-1-(2,3,4,5-tetrafluoro-6-(methylsulfonyl) phenyl) methanimine (0.13 g, 0.342 mmol) in TFA (6.5 mL) was added Et₃SiH (0.039 g, 0.342 mmol). The resulting solution heated to 60° C. for 1 hr, after which it was cooled to room temperature and concentrated under reduced vacuum. The crude material was purified by flash column chromatography (37% EtOAc in hexanes) and triturated using 10% diethyl ether in n-pentane to afford the title compound as a brown solid (0.025 g, 0.068 mmol, 20% yield). ¹H NMR (400 MHz, DMSO-d₆) δ 6.96 (t, J=9.2 Hz, 1H), 6.53 (dd, J₁=2.4 Hz, J₂=13.6 Hz, 1H), 6.39-6.36 (m, 1H), 5.93 (t, J=4.8 Hz, 1H), 4.42-4.31 (m, 2H), 3.71 (s, 3H), 3.01 (s, 3H). ¹⁹F NMR (376 MHz, DMSO-d₆) δ −133.82 (s, 1F), −140.31-−140.15 (m, 1F), −141.83-−141.92 (m, 1F), −151.16-−151.23 (m, 1F), −154.37-−154.43 (m, 1F). ESI-MS: measured m/z 366.3 [M+1]⁺. HPLC (HP07_TFRA1): RT=6.49 min., 96.5%

### Preparation of 3-fluoro-4-methoxy-N-methyl-N-(2,3,4,5-tetrafluoro-6-(methylsulfinyl)benzyl)aniline Compound I-20

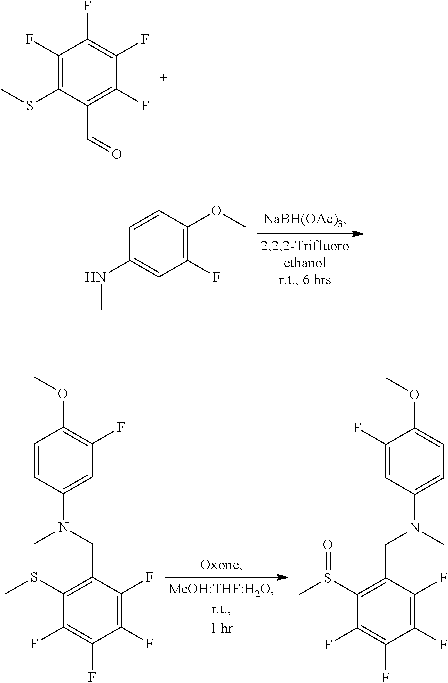

### 3-fluoro-4-methoxy-N-methyl-N-(2,3,4,5-tetrafluoro-6(methylthio)benzyl)aniline

To a stirred solution of 3-fluoro-4-methoxy-N-methylaniline (0.75 g, 4.83 mmol) in 2,2,2-trifluoroethanol (15 mL) was added 2,3,4,5-tetrafluoro-6-(methylthio)benzaldehyde (2.15 g, 9.6 mmol). The resulting mixture was stirred at room temperature for 1 hr, followed by addition of sodium triacetoxyborohydride (3.06 g, 14.51 mmol). After 6 hrs, the reaction was diluted with water (50 mL) and extracted with EtOAc (2×45 mL). The combined organic phases were dried over anhydrous Na₂SO₄, filtered, and concentrated under reduced pressure. The crude material was purified by flash column chromatography (5% EtOAc in Hexanes) to afford the title compound as a yellow oil (0.35 g, 0.96 mmol, 20% yield). ¹H NMR (400 MHz, DMSO-d6) δ 7.02 (t, J=9.2 Hz, 1H), 6.89-6.85 (dd, J₁=2.8 Hz, J₂=14.8 Hz, 1H), 6.69-6.66 (m, 1H), 4.55 (s, 2H), 3.75 (s, 3H), 2.64 (s, 3H), 2.42 (s, 3H). ¹⁹F NMR (376 MHz, DMSO-d₆) −130.56-−130.65 (m, 1F), −133.68 (s, 1F), 140.26-−140.35 (m, 1F), −155.91-−156.09 (m, 2F).

### 3-fluoro-4-methoxy-N-methyl-N-(2,3,4,5-tetrafluoro-6-(methylsulfinyl)benzyl)aniline Compound I-20

To a stirred solution of 3-fluoro-4-methoxy-N-methyl-N-(2,3,4,5-tetrafluoro-6(methylthio)benzyl)aniline (0.1 g, 0.27 mmol) in THF:water:Methanol (8:1:1, 1 mL) was added oxone (0.42 g, 1.3 mmol). After 1 hr, the reaction was diluted with aqueous Na₂CO₃ (30 mL) and extracted with EtOAc (2×20 mL). The combined organic phases were dried over anhydrous Na₂SO₄, filtered, and concentrated under reduced pressure. The crude material was purified by column chromatography (24% EtOAc in hexanes) and the enriched product purified by Prep TLC (50% EtOAc in Hexane). The title compound was isolated as an off-white solid (0.008 g, 0.02 mmol, 8% yield). ¹H NMR (400 MHz, DMSO-d6) δ 7.05 (t, J=9.2 Hz, 1H), 6.93 (d, J=14.8 Hz, 1H), 6.71 (d, J=7.2 Hz, 1H), 4.56 (d, J=13.6 Hz, 1H), 4.31 (d, J=14.0 Hz, 1H), 3.76 (s, 3H), 2.91 (s, 3H), 2.59 (s, 3H). ¹⁹F NMR (376 MHz, DMSO-d₆) −133.49 (s, 1F), −140.30-−140.36 (m, 1F), −141.76-−144.85 (1F), −151.15-−152.27 (m, 1F), −151.24-−152.35 (m, 1F). ESI-MS: measured m/z 380.4 [M+1]⁺. HPLC (Method I): RT=6.77 min., 95.8%.

### (S)—N-(4-((3-chloro-4-fluorophenyl)amino)-7-((tetrahydrofuran-3-yl)oxy)quinazolin-6-yl)-2,3,4,5-tetrafluoro-6-(trifluoromethyl)benzenesulfonamide (I--) Compound I-1

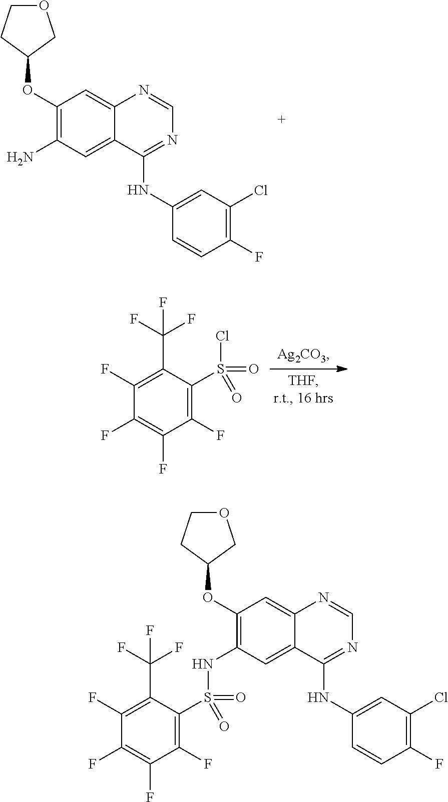 [SMILES: Nc1cc2c(Nc3ccc(F)c(Cl)c3)ncnc2cc1O[C@H]1CCOC1 ; "+" ; O=S(=O)(Cl)c1c(F)c(F)c(F)c(F)c1C(F)(F)F ; O=S(=O)(Nc1cc2c(Nc3ccc(F)c(Cl)c3)ncnc2cc1O[C@H]1CCOC1)c1c(F)c(F)c(F)c(F)c1C(F)(F)F]

To a stirred solution of N⁴-(3-chloro-4-fluoro-phenyl)-7-[(3S)-tetrahydrofuran-3-yl]oxy-quinazoline-4,6-diamine (200 mg, 533.62 μmol) and silver carbonate (294.29 mg, 1.07 mmol) in THF (5 mL) at room temperature was added a solution of 2,3,4,5-tetrafluoro-6-(trifluoromethyl)benzenesulfonyl chloride (168.93 mg, 533.62 μmol) in THF (1 mL). Reaction progress was monitored by TLC. After 16 hrs, the reaction was partitioned between EtOAc and a saturated aqueous solution of ammonium chloride. The organic phase was removed and the remaining aqueous extracted 4× with EtOAc. The combined organic phases were washed with brine, dried over anhydrous sodium sulfate, and concentrated under vacuum. The residue was purified on a biotage isolera equipped with a 60 g C18 column running a solvent gradient of 30% to 100% ACN (0.1% FA) in water (0.1% FA). The product containing fractions were consolidated and concentrated. The product was lyophilized from ACN and water to afford N-[4-(3-chloro-4-fluoro-anilino)-7-[(3S)-tetrahydrofuran-3-yl]oxy-quinazolin-6-yl]-2,3,4,5-tetrafluoro-6-(trifluoromethyl)benzenesulfonamide (10 mg, 14.51 μmol, 2.72% yield) as a tan powder. ¹H NMR (400 MHz, CDCl₃) δ 8.70 (s, 1H), 8.13 (s, 1H), 8.01 (dd, J=6.5, 2.7 Hz, 1H), 7.51 (dt, J=8.9, 3.4 Hz, 1H), 7.24-7.19 (m, 3H), 5.13 (s, 1H), 4.04 (q, J=7.7 Hz, 1H), 3.98 (d, J=2.9 Hz, 2H), 3.96-3.86 (m, 1H), 2.42 (dt, J=14.5, 7.1 Hz, 1H), 2.22-2.11 (m, 1H). ¹⁹F NMR (376 MHz, CDCl₃) δ −50.94 (m, 3F), −119.55 (s, 1F), −127.01-−127.13 (m, 1F), −130.15-−130.48 (m, 1F), −141.83-−141.89 (m, 1F), −143.65-−143.80 (m, 1F). ESI-MS: measured m/z 653.1 [M−H]⁻, Purity by HPLC (procedure Method III): RT=3.27 min (95.7%).

### Preparation of (S)—N-(4-((3-chloro-4-fluorophenyl)amino)-7-((tetrahydrofuran-3-yl)oxy)quinazolin-6-yl)-2,3,4,5-tetrafluoro-6-(methylthio)benzamide (I--) (S)—N-(4-((3-chloro-4-fluorophenyl)amino)-7-((tetrahydrofuran-3-yl)oxy)quinazolin-6-yl)-2,3,4,5-tetrafluoro-6-(methylsulfonyl)benzamide (I--) Compound I-5, and N-(4-((3-chloro-4-fluorophenyl)amino)-7-(((S)-tetrahydrofuran-3-yl)oxy)quinazolin-6-yl)-2,3,4,5-tetrafluoro-6-(methylsulfinyl)benzamide (I--) Compound I-32

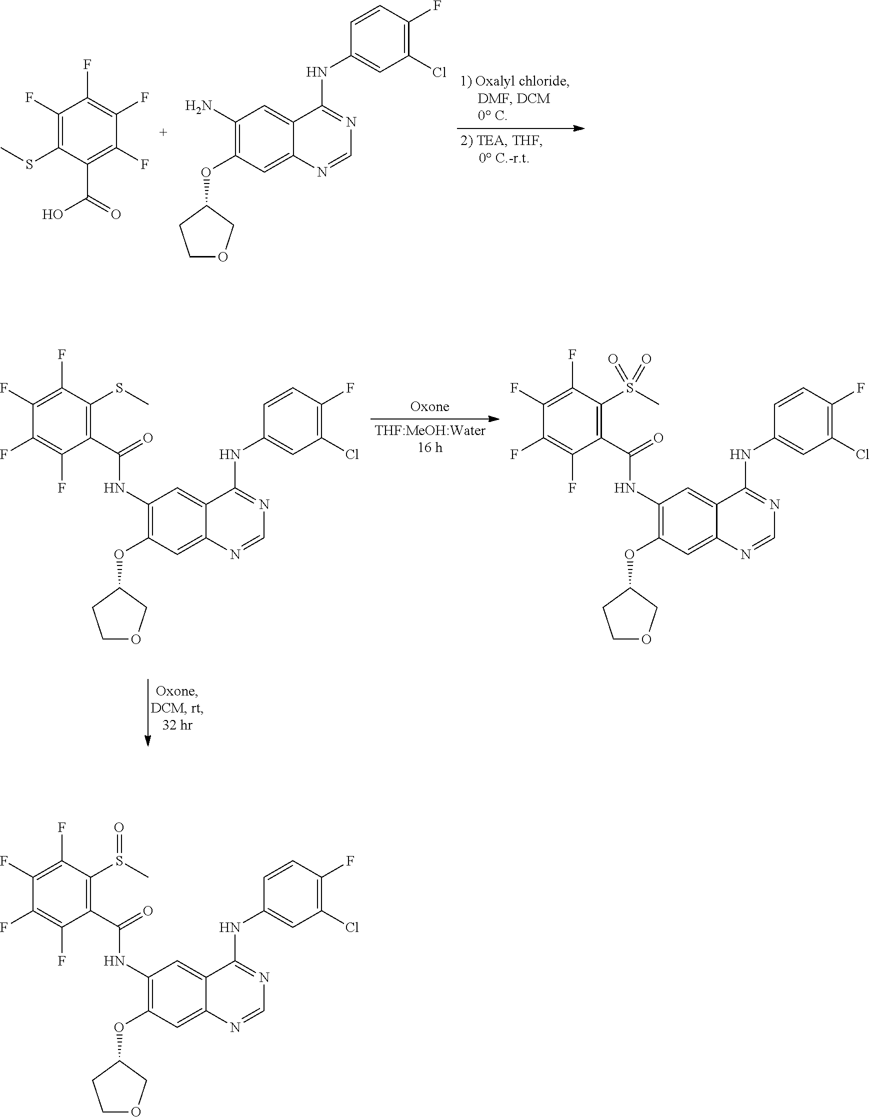

### (S)—N-(4-((3-chloro-4-fluorophenyl)amino)-7-((tetrahydrofuran-3-yl)oxy)quinazolin-6-yl)-2,3,4,5-tetrafluoro-6-(methylthio)benzamide (I--)

To a stirred solution of 2,3,4,5-tetrafluoro-6-(methylthio)benzoic acid (0.52 g, 2.16 mmol) in DCM (5 mL) were added oxalyl chloride (0.55 g, 2.91 mmol) and DMF (1-2 drop) at 0 0° C. The resulting reaction mixture was stirred at room temperature for 30 min. After completion of reaction, the reaction mixture was concentrated under reduced pressure under an atmosphere of N₂. The obtained residue was dissolved in THF (3 mL) and added dropwise to a stirring solution of (S)—N4-(3-chloro-4-fluorophenyl)-7-((tetrahydrofuran-3-yl) oxy) quinazoline-4,6-diamine (0.40 g, 1.08 mmol) in THF (2 mL) and TEA (1.09 g, 10.83 mmol) at 0° C. The resulting reaction mixture was stirred for 1 h at room temperature. After completion of reaction, the mixture was diluted with water (30 mL) extracted with EtOAc (3×30 mL). The combined organic phases were dried over anhydrous Na₂SO₄, filtered, and concentrated under reduced pressure. The resulting crude was purified by reverse phase column chromatography, eluted with 60-70% ACN in water to afford title compound as a brown solid (0.33 g, 0.55 mmol, 51% yield). ¹H NMR (400 MHz, DMSO-d₆) δ 10.30 (s, 1H), 10.02 (s, 1H), 8.89 (s, 1H), 8.56 (s, 1H), 8.10 (d, J=6.4 Hz, 1H), 7.80-7.78 (m, 1H), 7.44 (t, J=9.2 Hz, 1H), 7.30 (s, 1H), 5.33 (brs, 1H), 4.03-3.75 (m, 4H), 2.50 (s, 3H). 2.36-2.30 (m, 1H), 2.15-2.08 (m, 1H). ¹⁹F NMR (376 MHz, DMSO-d₆) δ −122.90 (s, 1F), −131.24-−131.33 (m, 1F), −141.52-−141.61 (M, 1F), −153.98-−154.10 (m, 1F), −154.60-−154.72 (m, 1F). ESI-MS: measured m/z 597.2, [M+H]⁺, Purity by HPLC RT=6.425 min (96.38%).

### (S)—N-(4-((3-chloro-4-fluorophenyl)amino)-7-((tetrahydrofuran-3-yl)oxy)quinazolin-6-yl)-2,3,4,5-tetrafluoro-6-(methylsulfonyl)benzamide (I--) Compound I-5

To the stirred solution of (S)—N-(4-((3-chloro-4-fluorophenyl)amino)-7-((tetrahydrofuran-3-yl)oxy)quinazolin-6-yl)-2,3,4,5-tetrafluoro-6-(methylthio)benzamide (0.04 g, 0.067 mmol) in THF:MeOH:Water (8:1:1, 4 mL) was added Oxone (0.10 g, 0.34 mmol) at 0° C. The resulting reaction mixture was stirred at room temperature for 16 hr. Four separate reactions were performed at this scale and later merged to facilitate the workup procedure. Upon completion of reaction, the reaction mixtures were combined and diluted with a saturated solution of NaHCO₃ (20 mL) and extracted with EtOAc (2×20 mL). The combined organic phases were dried over anhydrous Na₂SO₄, filtered, and concentrated under reduced pressure. The obtained crude was purified by reverse phase column chromatography, eluting with 60-70% ACN in water to afford title compound as a white solid (0.035 g, 0.05 mmol, 13% yield). ¹H NMR (400 MHz, DMSO-d₆) δ 10.31 (s, 1H), 10.01 (s, 1H), 8.93 (s, 1H), 8.54 (s, 1H), 8.07-8.05 (dd, J_{z}=2.8 Hz, J₂=6.8 Hz, 1H), 7.78-7.74 (m, 1H), 7.44 (t, J=9.2 Hz, 1H), 7.30 (s, 1H), 5.33 (brs, 1H), 3.99 (d, J=4.0 Hz, 2H), 3.92-3.86 (m, 1H), 3.79-3.73 (m, 1H), 3.46 (s, 3H), 2.35-2.30 (m, 1H), 2.14-2.09 (m, 1H). ¹⁹F NMR (376 MHz, DMSO-d₆) δ −122.95 (s, 1F), −132.93-−133.05 (m, 1F), −139.90-−139.99 (m, 1F), −145.76-−145.90 (m, 1F), −151.58-−151.69 (m, 1F). ESI-MS: measured m/z 629.2, [M+H]⁺, Purity by HPLC RT=6.053 min (97.29%).

### N-(4-((3-chloro-4-fluorophenyl)amino)-7-(((S)-tetrahydrofuran-3-yl)oxy)quinazolin-6-yl)-2,3,4,5-tetrafluoro-6-(methylsulfinyl)benzamide (I-) Compound I-32

To the stirred solution of (S)—N-(4-((3-chloro-4-fluorophenyl)amino)-7-((tetrahydrofuran-3-yl)oxy)quinazolin-6-yl)-2,3,4,5-tetrafluoro-6-(methylthio)benzamide (0.21 g, 0.35 mmol) in DCM (5 mL) at 0′C was added Oxone (0.16 g, 0.53 mmol). The resulting reaction mixture was warmed to room temperature and permitted to stir for 32 hr. Once complete, the reaction mixture was diluted with a saturated aqueous solution of NaHCO₃ (20 mL) and extracted with EtOAc (2×20 mL). The combined organic phases were dried over anhydrous Na₂SO₄, filtered, and concentrated under reduced pressure. The obtained crude was purified by reverse phase column chromatography, eluting with 50-60% ACN in Water to afford title compound as a white solid (0.06 g, 0.09 mmol, 30% yield). ¹H NMR (400 MHz, DMSO-d₆) δ 10.52 (d, J=5.2 Hz, 1H), 9.98 (s, 1H), 8.81 (d, J=4.4 Hz, 1H), 8.56 (s, 1H), 8.11-8.09 (dd, J₁=2.4 Hz, J₂=6.8 Hz, 1H), 7.80-7.76 (m, 1H), 7.45 (t, J=9.2 Hz, 1H), 7.31 (s, 1H), 5.34 (brs, 1H), 4.01-3.88 (m, 3H), 3.81-3.77 (m, 1H), 3.14 (d, J=1.6 Hz, 3H), 2.35-2.31 (m, 1H), 2.12-2.08 (m, 1H). ¹⁹F NMR (376 MHz, DMSO-d₆) δ −122.83 (s, 1F), −139.93-−140.09 (m, 2F), −149.96-−150.02 (m, 1F), −151.43-−151.54 (m, 1F). ESI-MS: measured m/z 613.2, 615.2 [M+H]⁺, Purity by HPLCRT=5.853 min (95.04%)

### Preparation of (S)—N⁴-(3-chloro-4-fluorophenyl)-N⁶-(2,3,4,5-tetrafluoro-6-(methylthio) phenyl)-7-((tetrahydrofuran-3-yl)oxy)quinazoline-4,6-diamine, (S)—N⁴-(3-chloro-4-fluorophenyl)-N⁶-(2,3,4,5-tetrafluoro-6-(methylsulfonyl)phenyl)-7-((tetrahydrofuran-3-yl)oxy)quinazoline-4,6-diamine (I--) Compound I-2, and N⁴-(3-chloro-4-fluorophenyl)-N⁶-(2,3,4,5-tetrafluoro-6-(methylsulfinyl)phenyl)-7-(((S)-tetrahydrofuran-3-yl)oxy)quinazoline-4,6-diamine (I--) Compound I-4

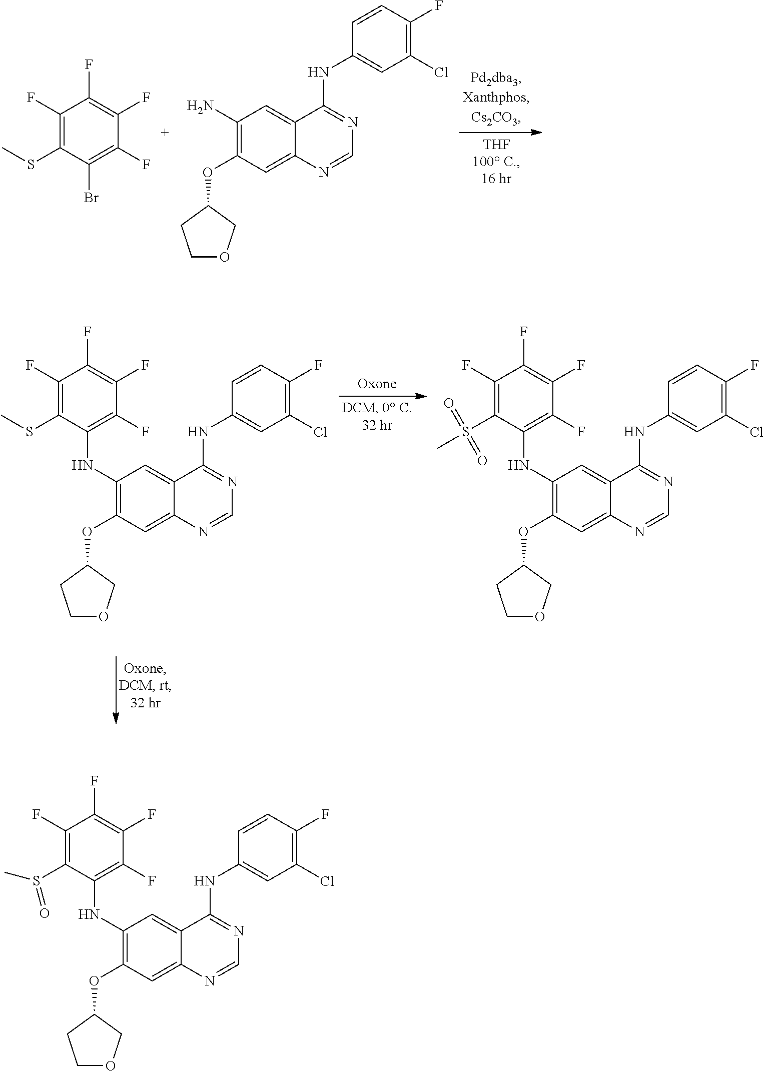

### (S)—N⁴-(3-chloro-4-fluorophenyl)-N⁶-(2,3,4,5-tetrafluoro-6-(methylthio) phenyl)-7-((tetrahydrofuran-3-yl)oxy)quinazoline-4,6-diamine

A stirred solution of (S)—N4-(3-chloro-4-fluorophenyl)-7-((tetrahydrofuran-3-yl)oxy)quinazoline-4,6-diamine (0.4 g, 1.09 mmol), (2-bromo-3,4,5,6-tetrafluorophenyl) (methyl)sulfane (0.3 g, 1.09 mmol), and Cs₂CO₃ (0.71 g, 2.18 mmol), in THF (3 mL) was purged with N₂ for 15 minutes. To this reaction mixture were added Pd₂dba₃ (0.099 g, 0.11 mmol) and xantphos (0.063 g, 0.11 mmol) at rt. The resulting reaction mixture was heated to 100° C. for 16 h using conventional heating methods (oil bath). After completion of reaction, the mixture was diluted with water (50 mL) and extracted with EtOAc (2×30 mL). The combined organic phases were dried over anhydrous Na₂SO₄, filtered, and concentrated under reduced pressure. The resulting crude was purified by flash column chromatography employing a mobile phase comprised of 78% EtOAc in hexane. The title compound was isolated as white solid (0.3 g, 0.52 mmol, 48% yield). 1H NMR (400 MHz, DMSO-d6) δ 9.29 (s, 1H), 8.43 (s, 1H), 8.05-8.03 (dd, J₁=2.8 Hz, J₂=6.8 Hz, 1H), 7.67-7.63 (m, 1H), 7.59 (s, 1H), 7.41 (t, J=9.2 Hz, 1H), 7.28 (d, J=1.6 Hz, 1H), 7.22 (s, 1H), 5.36 (s, 1H), 4.03-3.99 (m, 4H), 2.36 (s, 3H), 2.34-2.26 (m, 1H), 2.16-2.09 (m, 1H). 19F NMR (376 MHz, DMSO-d6) δ −122.95 (s, 1F), −132.29-−132.39 (m, 1F), −144.26-−144.35 (m, 1F), −156.06-−156.18 (m, 1F), −161.87-−162.0 (m, 1F), ESI-MS: measured m/z 569.1 [M+H]⁺.

### (S)—N⁴-(3-chloro-4-fluorophenyl)-N-(2,3,4,5-tetrafluoro-6-(methylsulfonyl)phenyl)-7-((tetrahydrofuran-3-yl)oxy)quinazoline-4,6-diamine (I--) Compound I-2

To stirred solution of (S)—N⁴-(3-chloro-4-fluorophenyl)-N⁶-(2,3,4,5-tetrafluoro-6-(methylthio)phenyl)-7-((tetrahydrofuran-3-yl)oxy)quinazoline-4,6-diamine (0.15 g, 0.26 mmol) in DCM (1.5 mL) at 0° C. was added Oxone (0.40 g, 1.32 mmol). The resulting mixture was stirred at room temperature for 16 h. Another portion of Oxone (0.40 g, 1.32 mmol) was added and stirring continued for a further 16 h. After completion of reaction, the mixture was diluted with saturated aqueous NaHCO₃ and extracted with EtOAc (2×30 mL). The combined organic phases were dried over anhydrous Na₂SO₄, filtered, and concentrated under reduced pressure. The resulting crude was purified by flash column chromatography, employing a mobile phase comprised of 81% EtOAc in hexane to afford the title compound as a white solid (0.025 g, 0.041 mmol, 16% yield). 1H NMR (400 MHz, DMSO-d6) δ 9.41 (s, 1H), 8.49 (s, 1H), 8.18 (s, 1H), 8.08-8.06 (dd, J₁=2.8 Hz, J₂=7.2 Hz, 1H), 7.70-7.66 (m, 1H), 7.59 (d, J=4.4 Hz, 1H), 7.43 (t, J=9.2 Hz, 1H), 7.28 (s, 1H), 5.38 (s, 1H), 4.02-3.98 (m, 1H), 3.90-3.78 (m, 3H), 3.53 (s, 3H), 2.36-2.31 (m, 1H), 2.09-2.06 (m, 1H). 19F NMR (376 MHz, DMSO-d6) δ −122.69 (s, 1F), −135.09-−135.18 (m, 1F), −139.83-−139.91 (m, 1F), −146.75-−146.89 (m, 1F), −163.08-−163.21 (m, 1F). ESI-MS: measured m/z 601.1 [M+H]⁺. HPLC RT=6.02 min., (95.38%).

### N⁴-(3-chloro-4-fluorophenyl)-N⁶-(2,3,4,5-tetrafluoro-6-(methylsulfinyl)phenyl)-7-(((S)-tetrahydrofuran-3-yl)oxy)quinazoline-4,6-diamine (I--) Compound I-4

To a stirred solution of (S)—N⁴-(3-chloro-4-fluorophenyl)-N⁶-(2,3,4,5-tetrafluoro-6-(methylthio)phenyl)-7-((tetrahydrofuran-3-yl)oxy)quinazoline-4,6-diamine (0.10 g, 0.17 mmol) in DCM (1.0 mL) at 0° C. was added Oxone (0.27 g, 0.88 mmol). The resulting mixture was warmed to room temperature and stirred for 16 h. After completion of reaction, the mixture was diluted with saturated aqueous NaHCO₃ and extracted with EtOAc (2×30 mL). The combined organic phases were dried over anhydrous Na₂SO₄, filtered, and concentrated under reduced pressure. The obtained crude was purified by flash column chromatography and eluted with 91% EtOAc in hexane to afford the title compound as a white solid (0.025 g, 0.064 mmol, 24% yield). 1H NMR (400 MHz, DMSO-d6) δ 9.41 (s, 1H), 8.69 (d, J=4 Hz, 1H), 8.47 (s, 1H), 8.07-8.05 (dd, J₁=1.6 Hz, J₂=6.8 Hz, 1H), 7.70-7.66 (m, 1H), 7.59 (t, J=4.0 Hz, 1H), 7.43 (t, J=8.8 Hz, 1H), 7.26 (d, J=2.8 Hz, 1H), 5.36 (s, 1H), 4.02-3.98 (m, 1H), 3.91-3.78 (m, 3H), 3.09 (s, 3H), 2.34-2.31 (m, 1H), 2.09-2.05 (m, 1H). 19F NMR (376 MHz, DMSO-d6) δ −122.77 (s, 1F), −140.30-−140.36 (m, 2F), −150.27-−150.46 (m, 1F), −163.49-−163.68 (m, 1F), ESI-MS: measured m/z 585.1 [M+H]⁺. HPLC RT=5.900 min (95.29%).

### II. Biological Evaluation

### Examples B1: In Vitro Cell Viability Studies

Anti-cancer efficacy of exemplary compounds of this application is assessed in vitro in different cancer cell lines. Cell viability is examined following treatment at various concentration of inhibitor (0.097656-50 μM) using a cell Titer-Blue cell viability assay. 1×104 cells (NHF cells)/well are plated in 96-well assay plates in culture medium. All cells are grown in DMEM, IMDM and RPMI-1640 supplemented with 10% FBS. After 24 hrs, test compounds and vehicle controls are added to appropriate wells so the final volume is 100 μl in each well. The cells are cultured for the desired test exposure period (72 hrs) at 37° C. and 5% CO2. The assay plates are removed from 37° C. incubator and 20 μl/well of CellTiter-Blue® Reagent is added. The plates are incubated using standard cell culture conditions for 1-4 hours and the plates are shaken for 10 seconds and record fluorescence at 560/590 nm.

### Examples B2 Reactivity Profiling With Glutathione

The experiment is started by placing 1 μL of 1 mM stocking solution of the test compound in DMSO in 199 μL of PBS buffer at pH 7.4 with 5 mM GSH to reach a final concentration of 5 μM. The final DMSO concentration is 0.5%. The solution is then incubated at 25° C. at 600 rpm, and is quenched with 600 μL solution of acetonitrile at 0, 30, 60 and 120 minutes. The quenched solution is vortexed for 10 minutes and centrifuged for 40 minutes at 3,220 g. An aliquot of 100 μL of the supernatant is diluted by 100 μL ultra-pure water, and the mixture is used for LC/MS/MS analysis. The data is processed and analyzed using Microsoft Excel.

### Examples B3 Parallel Artificial Membrane Permeability Assay (PAMPA)

The stock solutions of positive controls are prepared in DMSO at the concentration of 10 mM. Testosterone and methotrexate are used as control compounds in this assay. Prepare a stock solution of compounds in DMSO at the concentration of 10 mM, and further dilute with PBS (pH 7.4). The final concentration of the test compound is 10 μM.

Assay Procedures. 1) Prepare a 1.8% solution (w/v) of lecithin in dodecane, and sonicate the mixture to ensure a complete dissolution. 2)Carefully pipette 5 μL of the lecithin/dodecane mixture into each acceptor plate well (top compartment), avoiding pipette tip contact with the membrane. 3) Immediately after the application of the artificial membrane (within 10 minutes), add 300 μL of PBS (pH 7.4) solution to each well of the acceptor plate. Add 300 μL of drug-containing solutions to each well of the donor plate (bottom compartment) in triplicate. 4) Slowly and carefully place the acceptor plate into the donor plate, making sure the underside of the membrane is in contact with the drug-containing solutions in all wells. 5) Replace the plate lid and incubate at 25° C., 60 rpm for 16 hours. 6) After incubation, aliquots of 50 μL from each well of acceptor and donor plate are transferred into a 96-well plate. Add 200 μL of methanol (containing IS: 100 nM Alprazolam, 200 nM Labetalol and 2 μM Ketoprofen) into each well. 7) Cover with plate lid. Vortex at 750 rpm for 100 seconds. Samples are centrifuged at 3,220 g for 20 minutes. Determine the compound concentrations by LC/MS/MS.

### Examples B4 Kinase Assay

A 96-well half-area clear flat-bottom microplate (Corning® #3697) is pre-heated in a plate reader (Cytation 3, BioTek) at 37° C. for 15 minutes prior to the start of each assay. Kinase buffer (80 mM PIPES pH 6.9, 2 mM MgCl₂, 0.5 mM EGTA, 15% glycerol, 1 mM GTP) is prepared from stock solutions and placed on ice. Inhibitors of this application are prepared to 10 M concentrations in buffer (80 mM PIPES pH 6.9, 2 mM MgCl₂, 0.5 mM EGTA, 5% DMSO) from DMSO stock solutions. After the assay plate is pre-warmed, 10 μL of inhibitor or buffer control is added to selected wells. Every assay contained a kinase only negative control for normalization of data, and a known compound positive control. The assay plate is incubated at 37° C. for 3 minutes. During this time, a frozen aliquot of the kinase (10 mg/mL) in buffer (80 mM PIPES pH 6.9, 2 mM MgCl2, 0.5 mM EGTA) is defrosted by placing in a room temperature water bath. Once thawed, 200 μL of the kinase is mixed with 420 μL of the ice-cold kinase buffer (3 mg/mL kinase in 80 mM PIPES, pH 6.9, 2 mM MgCl₂, 0.5 mM EGTA, 1 mM GTP, 10.2% glycerol). To a 96-well plate on ice, aliquots of 100 μL kinase is added to each well. From this plate, 90 μL of the kinase is immediately pipetted into all sample wells of the warmed assay plate using a multi-channel pipette. The assay plate is immediately put in the reader at 37° C. and shook for 5 s with orbital shaking at medium speed. The reader records the absorbance at 340 nm every 15 s for 30 min.

The resulting absorbance curves are normalized by subtracting each data point by the absorbance at time 0. The slope of the initial linear portion (“Vₘₐₓ”) is determined in mOD/min, and normalized to the Vₘₐₓ value of the kinase only control, using the following equation, resulting in comparable % inhibition values:

$$\% inhibition = ( 1 - \frac{{V}_{max ( kinase + inhibitor )}}{{V}_{max ( kinase )}} ) \times 100$$

### Intact Mass Analysis

The covalent modification of the proteins with the compounds were evaluated using intact mass analysis by liquid chromatography-mass spectrometry instrument (LC-MS/MS).

The reaction solution (20 μL) was prepared in 96-well plate and contained the protein (2 μM), the compound (100 μM), HEPES buffer (20 mM, pH 8), 2% DMSO, 2% glycerol, and 150 mM NaCl. The reaction was allowed to proceed for 24 h at 25° C. The reaction solution (1 μL) was injected into the LC/MS/MS without any further sample preparation.

The LC-MS/MS instrument comprises of a Waters G2-XS quadrupole-time of flight (QTof) mass spectrometer and a Waters Acuity I-class Ultra-High Performance Liquid Chromatography (UPLC) system. The I-class UPLC system includes a binary solvent manager (BSM), and a Acquity sample manager (SM). The mobile phase consisted of: A) 0.1% (v/v) formic acid in MilliQ water; B) 0.1% (v/v) formic acid acetonitrile. Gradients were run over 5 min and proceeded as follows: A:B, 85:15, 0.0-0.7 min, 85:15→15:85, 0.7-1.5 min, 10:90, 1.5-4 min, 10:90→85:15, 4-4.5 min, 85:15, 4.5-5 min. The analytical column was a Waters BEH C4 column 1.7 μm (50×1 mm) column with pore sizes of 300 k. The TOF MS data was collected in positive ion mode (m/z of 400-2000 Da) using MassLynx software (Waters).

The spectral deconvolution was performed using UNIFI software (Waters). The added mass of the protein upon covalent modification to cysteine residues were specified. Multiple modification of up to 8 cysteine was allowed. All the adducts with signal intensities of <2% of the base peak were ignored. The % modification was calculated as the adduct signal intensity over the total intensities of the protein peaks and the adducts.

### Peptide Mapping

The site(s) of compounds covalent modification on proteins were identified using a peptide mapping analysis by liquid chromatography-mass spectrometry instrument (LC-MS/MS).

The reaction solution (100 μL) was prepared in a 1.5-mL Eppendorf tube and contained protein (2-10 μM), the compound (10-100 μM), HEPES buffer (20 mM, pH 8), 2% DMSO, 2% glycerol, and 150 mM NaCl. The reaction was allowed to proceed for 5-24 h at 25° C. or 37° C. Thereafter, the reaction was quenched by the addition of 500 μL of cold acetone and incubated at −20° C. for 2 h. Then, the tube was centrifuged for 10 min at 10,000 xg, and the supernatant was discarded. The pellet was washed by adding 200 μL of cold acetone and centrifugation at 10,000×g for 10 min. The pellet was re-dissolved in 50 μL of ammonium bicarbonate solution (ABC, 100 mM, pH 7.9) containing 8 M urea. The tube was centrifuged for 10 min at 10,000×g, and the supernatant was transferred to a new tube. The protein was first reduced by adding 1.25 μL of 200 mM DTT and incubation at 37° C. for 30 min, then alkylated by adding 1.5 μL of 400 mM iodoacetamide incubation at room temperature for another 20 min. Then the solution was diluted 8 times in ammonium bicarbonate. Sequencing-grade trypsin (Promega) was added at an enzyme-to-protein ratio of 1:50, and the tube was incubated overnight at 37° C. After digestion, the solution was acidified by trifluoracetic acid at 0.1%, and tubes were centrifuged at 10,000×g for 10 min. The supernatant was transferred to an autosampler vial, and 2 μL was injected into the LC-MS/MS for peptide mapping analysis.

The LC-MS/MS instrument comprises of a Waters G2-XS quadrupole-time of flight (QTof) mass spectrometer and a Waters Acuity M-class Ultra-High Performance Liquid Chromatography (UPLC) system. The M-class UPLC system includes a micro binary solvent manager (μBSM), a micro sample manager (μSM), and an IonKey (iKey) separation system. The mobile phase consisted of: A) 0.1% (v/v) formic acid in MilliQ water; B) 0.1% (v/v) formic acid acetonitrile. Gradients were run over 20 min and proceeded as follows: A:B, 97:3, 0.0-1 min, 97:3→60:40, 1-12 min, 60:40→15:85, 12-12.5 min, 15:85, 12.5-17 min, 15:85→97:3, 17.5-20 min. The analytical column was a Waters BEH C18 iKey 1.7 μm (50×0.15 mm) column with pore sizes of 150 Å. The TOF MSE data was collected in positive ion mode (m/z of 350-2000 Da) using MassLynx software (Waters).

The peptide mapping analysis was performed using UNIFI software (Waters). Carbamidomethyl (+57 Da) and the compound mass addition upon covalent modification were specified as variable modification to cysteine residues.

### HTRF Assay for Tyrosine Kinases (BTK, BMX, EGFR, FGFR4, JAK3)

### BTK

Kinase activity was monitored using a HTRF® KinEASE-TK kit from Cisbio (62TK0PEC). For BTK, the 1× kinase buffer was supplemented with 10 mM MnCl2, 5 mM MgCl2, 1 uM ATPγS, 1 mM TCEP and 100 fold diluted Supplementary Enzyme Buffer. BTK was purchased from Promega. 0.111 ng/μL BTK (1.42 nM) was preincubated in the absence or presence of inhibitor at room temperature for 3 hours. Reaction with substrate was then initiated by adding biotinylated substrate and ATP and the reaction was allowed to proceed for 45 min. Final concentration of substrate in the reaction mixture was 1 uM and ATP was 28 uM (reported Km value). The reaction was terminated by adding 62.5 nM SA-XL665 and 100-fold diluted europium labelled antibody (Eu-Ab), diluted in 1× detection buffer that contained EDTA. After 60 min of incubation, the fluorescence emission was measured at 620 nm and 665 nm. The ratio of Em 665 nm to Em 620 nm was proportional to the amount of substrate phosphorylated by the kinase.

### BMX

Kinase activity was monitored using a HTRF® KinEASE-TK kit from Cisbio (62TK0PEC). The buffer used was 1× kinase buffer supplemented with 2 mM MnCl2, 5 mM MgCl2, 1 mM TCEP and 100× diluted Supplementary Enzyme Buffer. BMX was purchased from Promega. 0.333 ng/ul BMX (3.03 nM) was preincubated in the absence or presence of inhibitor at room temperature for 3 hours. The reaction with substrate was then initiated by adding biotinylated substrate and ATP. The concentration of substrate in the reaction mixture was 0.5 uM and ATP was 26 uM (reported Km value). Reaction was terminated by adding 31.25 nM SA-XL665 and 100 fold diluted europium labelled antibody (Eu-Ab), diluted in 1× detection buffer that contained EDTA. After 60 min of incubation, the fluorescence emission was measured at 620 nm and 665 nm. The ratio of Em 665 nm to Em 620 nm was proportional to the amount of substrate phosphorylated by the kinase.

### EGFR

Kinase activity was monitored using a HTRF® KinEASE-TK kit from Cisbio (62TK0PEC). The buffer used was 1× kinase buffer supplemented with 2 mM MnCl2, 5 mM MgCl2, 1 mM TCEP and 100× diluted Supplementary Enzyme Buffer. EGFR was purchased from Promega. 0.041 ng/ul EGFR (0.46 nM) was preincubated in the absence or presence of inhibitor at room temperature for 3 hours. Reaction with substrate was then initiated by adding biotinylated substrate and ATP. The concentration of substrate in the reaction mixture was 0.5 uM and ATP was 1.57 uM (reported Km value). The reaction was terminated by adding 31.25 nM SA-XL665 and 100 fold diluted europium labelled antibody (Eu-Ab), diluted in 1× detection buffer that contained EDTA. After 60 min of incubation, the fluorescence emission was measured at 620 nm and 665 nm. The ratio of Em 665 nm to Em 620 nm was proportional to the amount of substrate phosphorylated by the kinase.

### FGFR4

Kinase activity was monitored using a HTRF® KinEASE-TK kit from Cisbio (62TK0PEC). The buffer used was 1× kinase buffer supplemented with 2 mM MnCl2, 5 mM MgCl2, 1 mM TCEP and 100× diluted Supplementary Enzyme Buffer. FGFR-4 was purchased from Promega. 0.333 ng/ul FGFR4 (5.12 nM) was preincubated in the absence or presence of inhibitor at room temperature for 3 hours. Reaction with substrate was then initiated by adding biotinylated substrate and ATP. The concentration of substrate in the reaction mixture was 0.5 uM and ATP was 113 uM (reported Km value). The reaction was terminated by adding 31.25 nM SA-XL665 and 100 fold diluted europium labelled antibody (Eu-Ab), diluted in 1× detection buffer that contained EDTA. After 60 min of incubation, the fluorescence emission was measured at 620 nm and 665 nm. The ratio of Em 665 nm to Em 620 nm was proportional to the amount of substrate phosphorylated by the kinase.

### JAK3

Kinase activity was monitored using a HTRF® KinEASE-TK kit from Cisbio (62TK0PEC). The buffer used was 1× kinase buffer supplemented with 2 mM MnCl2, 5 mM MgCl2, 1 mM TCEP and 100× diluted Supplementary Enzyme Buffer. JAK-3 was purchased from Promega. 0.0133 ng/ul JAK3 (0.21 nM) was preincubated in the absence or presence of inhibitor at room temperature for 3 hours. Reaction with substrate was then initiated by adding biotinylated substrate and ATP. The concentration of substrate in the reaction mixture was 0.5 uM and ATP was 1.434 uM (reported Km value). The reaction was terminated by adding 31.25 nM SA-XL665 and 100 fold diluted europium labelled antibody (Eu-Ab), diluted in 1× detection buffer that contained EDTA. After 60 min of incubation, the fluorescence emission was measured at 620 nm and 665 nm. The ratio of Em 665 nm to Em 620 nm was proportional to the amount of substrate phosphorylated by the kinase.

### Inhibitor Treatment

For initial screening, enzymes were pre incubated with compounds at 2 different concentrations (1 and 10 uM final) and 0.5% DMSO. The top 13 compounds together with known inhibitor Ibrutinib were further selected for dose response analysis where compounds were tested from final concentrations ranging from 24 μM to 25 uM.

### Time-Dependent Inhibition

### BTK

For the time-dependent inhibition experiments, the activity of BTK kinases was monitored using a HTRF® KinEASE-TK kit from Cisbio (62TK0PEC) as described above. Six compounds showing higher potency during dose response analysis were further evaluated for their change in inhibitory potency over time. Ibrutinib was used as a positive control. The selected compounds were pre incubated with the kinase at 11 different concentrations, ranging from 24 μM to 25 uM final compound concentrations. The pre incubation times were 0, 1, 3, 5, 10, 15, 30 and 45 min. After the preincubation, reaction was initiated by adding biotinylated substrate and ATP and the reaction was allowed to proceed for 45 min. The concentration of substrate in the reaction mixture was 1 uM and ATP was 28 uM (reported Km value). The reaction was terminated by adding 62.5 nM SA-XL665 and 100fold diluted europium labelled antibody (Eu-Ab), diluted in 1× detection buffer that contained EDTA. After 60 min of incubation, the fluorescence emission was measured at 620 nm and 665 nm. The ratio of Em 665 nm to Em 620 nm was proportional to the amount of substrate phosphorylated by the kinase. Product formation vs pre-incubation time data were fitted to a one phase exponential decay equation using GraphPad Prism. The same product formation vs inhibitor concentration data were also fitted to a log(antagonist) vs. response—Variable slope equation in GraphPad Prism to provide IC₅₀ values at different pre incubation times.

### EGFR

For the time-dependent inhibition experiments, the activity of EGFR kinase was monitored using a HTRF® KinEASE-TK kit from Cisbio (62TK0PEC) as described above. Six compounds showing higher potency during dose response analysis against EGFR, were further evaluated for their change in inhibitory potency over time. Ibrutinib was used as a positive control. The selected compounds were pre incubated with the kinase at 11 different concentrations, ranging from 24 μM to 25 uM final compound concentrations. The pre incubation times were 0, 2, 5, 10, 15, 30, 45 and 60 min. After the preincubation, the reaction was initiated by adding biotinylated substrate and ATP and the reaction was allowed to proceed for 45 min. The concentration of substrate in the reaction mixture was 0.5 uM and ATP was 1.57 uM (reported Km value). The reaction was terminated by adding 31.25 nM SA-XL665 and 100 fold diluted europium labelled antibody (Eu-Ab), diluted in 1× detection buffer that contained EDTA. After 60 min of incubation, the fluorescence emission was measured at 620 nm and 665 nm. The ratio of Em 665 nm to Em 620 nm was proportional to the amount of substrate phosphorylated by the kinase. Product formation vs pre-incubation time data were fitted to a one phase exponential decay equation using GraphPad Prism. The same product formation vs inhibitor concentration data were also fitted to a log(antagonist) vs. response—Variable slope equation in GraphPad Prism to provide IC₅₀ values at different pre incubation times.

Cell viability assays (Ramos RA1, A549, K562, MIA PaCa-2)

### Ramos RA1

Ramos RA 1 cells (ATCC) were cultured in RPMI-1640 media (Wisent) supplemented with 10% heat-inactivated FBS and 1% penicillin/streptomycin. Cells were seeded in 96-well plates at 57,000 cells/well and incubated at 37° C., 5% CO₂ for 16 hours. Serially-diluted compounds or DMSO alone were added to cells and incubated at 37° C., 5% CO₂ for 24 hours. Cell viability was measured using the CellTiter-Glo® Luminescent Cell Viability Assay (Promega) according to the manufacturer's protocol. The luminescence signal of each treated well was normalized to the DMSO control well and the medium-only background was subtracted. Cell viability curves and IC₅₀ values were visualized using Prism (GraphPad).

### A549

A549 cells (ATCC) were cultured in Dulbecco's Modified Eagle's Medium (Wisent) supplemented with 10% FBS and 1% penicillin/streptomycin. Cells were seeded in 96-well plates at 17,500 cells/well and incubated at 37° C., 5% CO2 for 16 hours. Serially-diluted compounds or DMSO alone were added to cells and incubated at 37° C., 5% CO2 for 24 hours. Cell viability was measured using the CellTiter-Glo® Luminescent Cell Viability Assay (Promega) according to the manufacturer's protocol. The luminescence signal of each treated well was normalized to the DMSO control well and the medium-only background was subtracted. Cell viability curves and IC50 values were visualized using Prism (GraphPad).

### K562

K562 cells (ATCC) were cultured in Iscove's Modified Dulbecco's Medium (IMDM) (Wisent) supplemented with 10% FBS and 1% penicillin/streptomycin. Cells were seeded in 96-well plates at 57,000 cells/well and incubated at 37° C., 5% CO₂ for 16 hours. Serially-diluted compounds or DMSO alone were added to cells and incubated at 37° C., 5% CO₂ for 24 hours. Cell viability was measured using the CellTiter-Glo® Luminescent Cell Viability Assay (Promega) according to the manufacturer's protocol. The luminescence signal of each treated well was normalized to the DMSO control well and the medium-only background was subtracted. Cell viability curves and IC₅₀ values were visualized using Prism (GraphPad).

### Cell Lysis and Immunoassays

### Ramos RA1 Cell Lysis and Immunoassay to Detect BTK and Phospho-BTK

Ramos RA 1 cells (ATCC) were cultured in RPMI-1640 media (Wisent) supplemented with 10% heat-inactivated FBS and 1% penicillin/streptomycin. Cells were seeded in 6-well plates at 1.71×10⁶ cells/well and incubated at 37° C., 5% CO2 for 16 hours. Cells were treated with compound at the indicated concentrations or DMSO alone and incubated at 37° C., 5% CO2 for 24 hours. Cell suspensions were centrifugated at 1,400-1,600 RPM for 5 minutes at room temperature and supernatants were discarded. Cell pellets were resuspended with 1× RIPA buffer (Millipore) and incubated on ice for 15 minutes. Protein lysates were extracted by centrifugating at 20,000 RPM for 20 minutes at 4° C. Proteins were separated and total BTK protein levels were quantified by Simple Western Immunoassay (ProteinSimple) using the 12-230 kDa Jess Separation Module according to the manufacturer's protocol, using a protein concentration of 0.25 μg/μl and antibodies targeting BTK (clone #D3H5, Cell Signalling) and β-Actin (clone #AC-15, Santa Cruz) diluted to 1:400 and 1:10, respectively, where the latter was used as a loading control. An equal ratio of rabbit and mouse HRP-conjugated secondary antibodies was used for detection. BTK protein levels were quantified relative to S-Actin loading levels and subsequently normalized to the DMSO control. Phosphorylated BTK levels were quantified using the Protein Normalization Assay Module for Jess (ProteinSimple) according to the manufacturer's protocol, using a protein concentration of 1.5 μg/μl and an antibody targeting pBTK (Y223) (Cell Signalling) diluted to 1:50. Phosphorylated BTK levels were quantified relative to the Protein Normalization Reagent and subsequently normalized to the DMSO control.

### A549 Cell Lysis and Immunoassay to Detect Total EGFR and Phospho-EGFR

A549 cells (ATCC) were cultured in Dulbecco's Modified Eagle's Medium (DMEM) (Wisent) supplemented with 10% FBS and 1% penicillin/streptomycin. Cells were seeded in 6-well plates at 530,000 cells/well and incubated at 37° C., 5% CO2 for 16 hours. Cells were starved in DMEM containing 1% FBS for 4 hours before they were treated with the indicated concentrations of compound or DMSO alone and incubated at 37° C., 5% CO2 for 24 hours. Prior to cell harvest and lysis, 50 ng/ml human epidermal growth factor (hEGF) (Sigma) was added to the cells and incubated at 37° C., 5% CO2 for 10 minutes. Conditioned media was discarded, and adherent cells were washed with PBS before they were scraped on ice. Cell suspensions were centrifugated at 2,000 RPM for 5 minutes at 4° C. and supernatants were discarded. Cell pellets were resuspended with 1× RIPA buffer (Millipore) and incubated on ice for 15 minutes. Protein lysates were extracted by centrifugating at 20,000 RPM for 20 minutes at 4° C. Proteins were separated and total EGFR protein levels were quantified by Simple Western Immunoassay (ProteinSimple) using the Protein Normalization Assay Module for Jess according to the manufacturer's protocol, using a protein concentration of 0.125 μg/μl and an antibody targeting EGFR (clone #EP38Y, Abcam) diluted to 1:400. Phosphorylated EGFR protein levels were quantified with the same assay using a protein concentration of 1 μg/μl and an antibody targeting pEGFR (Y1068) (Cell Signalling) diluted to 1:25. EGFR and phosphorylated EGFR levels were normalized to the Jess Protein Normalization Reagent loading control and subsequently to the DMSO control.

### K562 Cell Lysis and Immunoassay to Detect β-Tubulin

K562 cells (ATCC) were cultured in Iscove's Modified Dulbecco's Medium (IMDM) (Wisent) supplemented with 10% FBS and 1% penicillin/streptomycin. Cells were seeded in 6-well plates at 1.71×10⁶ cells/well and incubated at 37° C., 5% CO2 for 16 hours. Cells were treated with compounds at the indicated concentrations or DMSO alone and incubated at 37° C., 5% CO2 for 24 hours. Cell suspensions were centrifugated at 1,400 RPM for 5 minutes at room temperature and supernatants were discarded. Cell pellets were resuspended with 1× RIPA buffer (Millipore) and incubated on ice for 15 minutes. Protein lysates were extracted by centrifugating at 20,000 RPM for 20 minutes at 4° C. Proteins were separated and total β-Tubulin levels were quantified by Simple Western Immunoassay (ProteinSimple) using the Protein Normalization Assay Module for Jess (ProteinSimple) according to the manufacturer's protocol, using a protein concentration of 0.05 μg/μl and an antibody targeting β-Tubulin (Abcam) diluted to 1:800. β-Tubulin levels were quantified relative to the Protein Normalization Reagent and subsequently normalized to the DMSO control.

### Time-Dependent Inhibition

### BTK

For the time-dependent inhibition experiments, the activity of BTK kinases was monitored using a HTRF® KinEASE-TK kit from Cisbio (62TK0PEC) as described above. Six compounds showing higher potency during dose response analysis were further evaluated for their change in inhibitory potency over time. Ibrutinib was used as a positive control. The selected compounds were pre incubated with the kinase at 11 different concentrations, ranging from 24 μM to 25 uM final compound concentrations. The pre incubation times were 0, 1, 3, 5, 10, 15, 30 and 45 min. After the preincubation, reaction was initiated by adding biotinylated substrate and ATP and the reaction was allowed to proceed for 45 min. The concentration of substrate in the reaction mixture was 1 uM and ATP was 28 uM (reported Km value). The reaction was terminated by adding 62.5 nM SA-XL665 and 100fold diluted europium labelled antibody (Eu-Ab), diluted in 1× detection buffer that contained EDTA. After 60 min of incubation, the fluorescence emission was measured at 620 nm and 665 nm. The ratio of Em 665 nm to Em 620 nm was proportional to the amount of substrate phosphorylated by the kinase. Product formation vs pre-incubation time data were fitted to a one phase exponential decay equation using GraphPad Prism. The same product formation vs inhibitor concentration data were also fitted to a log(antagonist) vs. response—Variable slope equation in GraphPad Prism to provide IC₅₀ values at different pre incubation times.

### EGFR

For the time-dependent inhibition experiments, the activity of EGFR kinase was monitored using a HTRF® KinEASE-TK kit from Cisbio (62TK0PEC) as described above. Six compounds showing higher potency during dose response analysis against EGFR, were further evaluated for their change in inhibitory potency over time. Ibrutinib was used as a positive control. The selected compounds were pre incubated with the kinase at 11 different concentrations, ranging from 24 μM to 25 uM final compound concentrations. The pre incubation times were 0, 2, 5, 10, 15, 30, 45 and 60 min. After the preincubation, the reaction was initiated by adding biotinylated substrate and ATP and the reaction was allowed to proceed for 45 min. The concentration of substrate in the reaction mixture was 0.5 uM and ATP was 1.57 uM (reported Km value). The reaction was terminated by adding 31.25 nM SA-XL665 and 100 fold diluted europium labelled antibody (Eu-Ab), diluted in 1× detection buffer that contained EDTA. After 60 min of incubation, the fluorescence emission was measured at 620 nm and 665 nm. The ratio of Em 665 nm to Em 620 nm was proportional to the amount of substrate phosphorylated by the kinase. Product formation vs pre-incubation time data were fitted to a one phase exponential decay equation using GraphPad Prism. The same product formation vs inhibitor concentration data were also fitted to a log(antagonist) vs. response—Variable slope equation in GraphPad Prism to provide IC₅₀ values at different pre incubation times.

### Jump Dilution Experiment

### BTK

Six compounds, along with ibrutinib as a control compound (used at 10 times their IC₅₀ concentrations) were pre incubated with BTK (at 142 nM or 100-fold the normal assay concentration) for 1.5 h at room temperature. Sample containing only DMSO vehicle was used as a positive (full activity) control while sample with no enzyme was used as a negative (zero activity) control. After the preincubation, the mixture was diluted 100-fold into a reaction mixture containing 1 uM biotinylated substrate and 28 uM ATP and the reaction was allowed to proceed for various time points (2, 5, 10, 15, 20, 30, 45 and 60 min), at which the reaction was terminated by adding 1 ul of 0.5 M EDTA. 62.5 nM SA-XL665 and 100-fold diluted europium labelled antibody (Eu-Ab), diluted in 1× detection buffer, was added as detection mixture. After 60 min of incubation, the fluorescence emission was measured at 620 nm and 665 nm. The ratio of Em 665 nm to Em 620 nm was proportional to the amount of substrate phosphorylated by the kinase.

### EGFR

Six compounds (used at 10 times their IC₅₀ concentrations) were pre incubated with EGFR (at 46 nM or 100-fold the normal assay concentration) for 1.5 h at room temperature. Sample containing only DMSO vehicle was used as a positive (full activity) control while sample with no enzyme was used as a negative (zero activity) control. After the preincubation, the mixture was diluted 100-fold into a reaction mixture containing 0.5 uM biotinylated substrate and 1.57 uM ATP and the reaction was allowed to proceed for various time points (2, 5, 10, 15, 30, 40, 45 and 60 min), at which the reaction was terminated by adding 1 ul of 0.5 M EDTA. 31.25 nM SA-XL665 and 100-fold diluted europium labelled antibody (Eu-Ab), diluted in 1× detection buffer, was added as detection mixture. After 60 min of incubation, the fluorescence emission was measured at 620 nm and 665 nm. The ratio of Em 665 nm to Em 620 nm was proportional to the amount of substrate phosphorylated by the kinase.

In some instances, Table 9-Table 24 demonstrate the (e.g., binding) activity of a compound provided herein.

In some instances, Table 9 demonstrates the extent to which a compound binds to EGFR. In some instances, in vitro binding to EGFR shows the extent to which a compound binds to EGFR. In some instances, Table 9 shows the extent to which a compound binds to EGFR and inhibits phosphorylation of a peptide substrate. In some instances, a compound demonstrates strong binding to EGFR when the remaining EGFR activity is low. In some instances, in vitro EGFR inhibition is shown in Table 9.

TABLE 9

| | % EGFR Remaining | % EGFR Remaining |
|---|---|---|
| Compound | activity vs DMSO | activity vs DMSO |
| Number | (1 μM, 3 h) | (10 μM, 3 h) |
| I-51 | A | A |
| I-52 | A | B |
| I-53 | B | C |
| I-54 | A | B |
| I-55 | A | A |
| I-56 | A | A |
| I-57 | A | A |
| I-58 | A | A |
| I-22 | A | C |
| I-23 | B | C |
| I-24 | A | A |
| I-25 | A | A |
| I-26 | A | B |
| I-1 | C | C |
| I-27 | C | C |
| I-28 | C | C |
| I-45 | A | C |
| I-29 | C | C |
| I-30 | A | B |
| I-31 | A | A |
| I-39 | C | C |
| I-34 | C | C |
| I-41 | C | C |
| I-38 | C | C |
| I-40 | C | C |
| I-42 | B | C |
| I-35 | A | A |
| I-36 | A | A |
| I-60 | A | A |
| I-32 | C | C |
| I-33 | A | C |
| I-37 | C | C |
| I-46 | A | A |
| I-48 | A | A |
| I-47 | A | A |
| I-59 | A | A |
| I-49 | A | A |
| I-61 | A | C |
| I-2 | C | C |
| I-62 | A | B |
| I-3 | C | C |
| I-63 | A | A |
| I-4 | C | C |
| I-64 | A | A |
| I-65 | A | A |
| I-5 | C | C |
| I-50 | A | B |
| I-43 | A | B |
| I-44 | A | C |

A: >90%;
B: 50-90%;
C: <50%

In some instances, Table 10 demonstrates the extent to which a compound binds to EGFR. In some instances, in vitro EFGR inhibition shows the extent to which a compound binds to EGFR. In some instances, Table 10 shows the extent to which a compound binds to EGFR and inhibits phosphorylation of a peptide substrate across a dose response. In some instances, in vitro EGFR inhibition is shown in Table 10.

TABLE 10

| Compound | EGFR Inhibition IC50 |
|---|---|
| Number | (nM) |
| I-1 | a |
| I-27 | a |
| I-39 | a |
| I-38 | b |
| I-37 | a |
| I-2 | a |
| I-3 | a |
| I-4 | a |
| I-5 | a |

a: <1000 nM;
b: >1000 nM

In some instances, Table 11 demonstrates the in cellulo EFGR inhibition of a compound provided herein. In some instances, the in cellulo EGFR inhibition demonstrates the extent to which a compound binds to EGFR in cells (e.g., and inhibits autophosphorylation of EGFR). In some instances, Table 11 shows in cellulo pEGFR inhibition in A549 cells.

TABLE 11

| | | pEGFR inhibition | Compound |
|---|---|---|---|
| Compound | pEGFR inhibition | (A549, compound | concentration |
| Number | (A549, 1 μM, 24 h) | concentration, 24 h) | (μM) |
| I-39 | X | X | 5 |
| I-34 | X | n.d. | 10 |
| I-41 | X | n.d. | 10 |
| I-38 | X | n.d. | 10 |
| I-40 | X | X | 5 |
| I-42 | X | X | 10 |
| I-35 | n.d. | n.d. | 10 |
| I-36 | X | X | 10 |
| I-32 | X | X | 10 |
| I-33 | X | X | 10 |
| I-37 | X | X | 10 |
| I-46 | X | X | 10 |
| I-48 | X | X | 10 |
| I-47 | X | n.d. | 10 |
| I-59 | X | X | 10 |
| I-61 | X | n.d. | 10 |
| I-2 | X | X | 10 |
| I-62 | X | n.d. | 10 |
| I-3 | X | X | 10 |
| I-63 | X | X | 10 |
| I-4 | X | X | 10 |
| I-64 | X | n.d. | 10 |
| I-65 | X | X | 10 |
| I-5 | X | X | 10 |
| I-50 | X | X | 10 |

n.d.: not determined
X: EGFR bound and autophosphoration of EGFR inhibited in cells

In some instances, Table 12 demonstrates the in cellulo EFGR degradation using a compound provided herein. In some instances, the in cellulo EGFR degradation demonstrates the extent to which a compound binds to EGFR in cells (e.g., and causes destabilization and degradation of EGFR). In some instances, Table 12 shows in cellulo EGFR degradation in A549 cells.

TABLE 12

| | | EGFR degradation | Compound |
|---|---|---|---|
| Compound | EGFR degradation | (A549, compound | concentration |
| Number | (A549, 1 μM, 24 h) | concentration, 24 h) | (1 uM) |
| I-39 | Y | Y | 5 |
| I-34 | Y | Y | 10 |
| I-41 | Y | Y | 5 |
| I-38 | Y | Y | 10 |
| I-40 | n.d. | Y | 5 |
| I-42 | Y | Y | 10 |
| I-35 | n.d. | n.d. | 10 |
| I-36 | Y | Y | 10 |
| I-32 | Y | Y | 10 |
| I-33 | Y | Y | 10 |
| I-37 | Y | Y | 10 |
| I-46 | Y | Y | 10 |
| I-48 | Y | Y | 10 |
| I-47 | Y | Y | 10 |
| I-59 | Y | Y | 5 |
| I-61 | Y | Y | 10 |
| I-2 | Y | Y | 10 |
| I-62 | Y | Y | 10 |
| I-3 | n.d. | Y | 10 |
| I-63 | Y | Y | 10 |
| I-4 | Y | Y | 10 |
| I-64 | Y | Y | 10 |
| I-65 | Y | Y | 10 |
| I-5 | Y | Y | 10 |
| I-50 | Y | n.d. | 10 |

n.d.: not determined
Y: EGFR bound in cells and EGFR degradation occurred

In some instances, Table 13 demonstrates the extent to which a compound binds to BTK. In some instances, in vitro binding to BTK shows the extent to which a compound binds to BTK. In some instances, Table 13 shows the extent to which a compound binds to BTK and inhibits phosphorylation of a peptide substrate. In some instances, a compound demonstrates strong binding to BTK when the remaining BTK activity is low. In some instances, in vitro BTK inhibition is shown in Table 13.

TABLE 13

| | % BTK Remaining | % BTK Remaining |
|---|---|---|
| Compound | activity vs DMSO | activity vs DMSO |
| Number | (1 μM, 3 h) | (10 μM, 3 h) |
| I-51 | B | C |
| I-52 | C | C |
| I-53 | C | C |
| I-54 | C | C |
| I-55 | C | C |
| I-56 | C | C |
| I-57 | C | C |
| I-58 | C | C |
| I-22 | C | C |
| I-23 | C | C |
| I-24 | C | C |
| I-25 | C | C |
| I-26 | C | C |
| I-1 | B | C |
| I-27 | C | C |
| I-28 | C | C |
| I-45 | C | C |
| I-29 | C | C |
| I-30 | C | C |
| I-31 | C | C |
| I-39 | C | C |
| I-34 | C | C |
| I-41 | C | C |
| I-38 | C | C |
| I-40 | C | C |
| I-42 | C | B |
| I-35 | C | C |
| I-36 | C | C |
| I-60 | C | C |
| I-32 | C | C |
| I-33 | C | C |
| I-37 | C | C |
| I-46 | B | C |
| I-48 | C | C |
| I-47 | C | C |
| I-59 | C | C |
| I-49 | B | C |
| I-61 | C | C |
| I-2 | A | B |
| I-62 | C | C |
| I-3 | B | B |
| I-63 | C | B |
| I-4 | A | C |
| I-64 | C | C |
| I-65 | C | C |
| I-5 | C | C |
| I-50 | C | C |
| I-43 | A | A |
| I-44 | C | C |

A: >90%;
B: 50-90%;
C: <50%

In some instances, Table 14 demonstrates the extent to which a compound binds to BTK. In some instances, in vitro BTK inhibition shows the extent to which a compound binds to BTK. In some instances, Table 14 shows the extent to which a compound binds to BTK and inhibits phosphorylation of a peptide substrate across a dose response. In some instances, in vitro BTK inhibition is shown in Table 14.

TABLE 14

| Compound | BTK Inhibition IC50 |
|---|---|
| Number | (nM) |
| I-38 | a |
| I-33 | a |
| I-32 | a |
| I-42 | a |
| I-36 | a |
| I-37 | a |
| I-34 | a |
| I-40 | a |
| I-62 | a |
| I-50 | a |
| I-53 | a |

a: <1000 nM;
b: >1000 nM

In some instances, Table 15 demonstrates the in cellulo BTK inhibition of a compound provided herein. In some instances, the in cellulo BTK inhibition demonstrates the extent to which a compound binds to BTK in cells (e.g., and inhibits autophosphorylation of BTK). In some instances, Table 15 shows in cellulo phosphoEGFR inhibition in RAMOS cells.

TABLE 15

| Compound | pBTK inhibition | Compound concentration |
|---|---|---|
| Number | (RAMOS, 24 h) | (μM) |
| I-37 | X | 1 |
| I-26 | X | 1 |
| I-35 | X | 1 |
| I-22 | X | 1 |
| I-32 | X | 1 |
| I-33 | X | 1 |
| I-34 | X | 1 |
| I-42 | X | 1 |
| I-24 | X | 1 |
| I-39 | X | 1 |
| I-54 | X | 5 |
| I-41 | X | 1 |
| I-36 | X | 1 |
| I-63 | X | 1 |
| I-3 | X | 1 |
| I-23 | X | 1 |
| I-60 | X | 1 |
| I-53 | X | 2.5 |
| I-38 | X | 1 |
| I-46 | X | 1 |
| I-25 | X | 1 |
| I-62 | X | 1 |
| I-40 | X | 1 |
| I-65 | X | 1 |
| I-61 | X | 1 |
| I-52 | X | 1 |
| I-64 | X | 1 |
| I-50 | X | 1 |
| I-5 | X | 1 |
| I-59 | X | 1 |
| I-47 | n.d. | 1 |
| I-55 | n.d. | 2.5 |
| I-48 | n.d. | 1 |
| I-2 | n.d. | 1 |
| I-49 | n.d. | 1 |
| I-4 | n.d. | 1 |
| I-51 | X | 10 |

n.d.: not determined
X: BTK bound and autophosphoration of BTK inhibited in cells

In some instances, Table 16 demonstrates the in cellulo BTK degradation using a compound provided herein. In some instances, the in cellulo BTK degradation demonstrates the extent to which a compound binds to BTK in cells (e.g., and causes destabilization and degradation of BTK). In some instances, Table 16 shows in cellulo BTK degradation in RAMOS cells.

TABLE 16

| Compound | % BTK degradation | % BTK degradation |
|---|---|---|
| Number | (RAMOS, 1 mM, 24 h) | (RAMOS, 10 uM, 24 h) |
| I-38 | Y | n.d. |
| I-33 | Y | n.d. |
| I-32 | Y | n.d. |
| I-42 | Y | n.d. |
| I-36 | Y | n.d. |
| I-35 | Y | n.d. |
| I-59 | Y | n.d. |
| I-49 | Y | n.d. |
| I-60 | Y | n.d. |
| I-39 | Y | n.d. |
| I-37 | Y | n.d. |
| I-48 | Y | n.d. |
| I-34 | Y | n.d. |
| I-5 | Y | n.d. |
| I-46 | Y | n.d. |
| I-64 | Y | n.d. |
| I-65 | Y | n.d. |
| I-47 | Y | n.d. |
| I-3 | Y | n.d. |
| I-4 | Y | Y |
| I-40 | Y | n.d. |
| I-63 | Y | Y |
| I-2 | Y | n.d. |
| I-62 | Y | n.d. |
| I-50 | Y | n.d. |
| I-41 | Y | n.d. |
| I-61 | n.d. | n.d. |

n.d.: not determined
Y: EGFR bound in cells and EGFR degradation occurred

In some instances, Table 17 demonstrates the extent to which a compound binds to BMX. In some instances, in vitro binding to BMX shows the extent to which a compound binds to BMX. In some instances, Table 17 shows the extent to which a compound binds to BMX and inhibits phosphorylation of a peptide substrate. In some instances, a compound demonstrates strong binding to BMX when the remaining BMX activity is low. In some instances, in vitro BMX inhibition is shown in Table 17.

TABLE 17

| Compound | BMX - 1 uM | BMX - 10 uM |
|---|---|---|
| Number | % remaining | % remaining |
| I-26 | C | C |
| I-56 | C | C |
| I-25 | C | C |
| I-41 | C | C |
| I-65 | C | C |
| I-47 | C | C |
| I-48 | C | C |
| I-50 | C | C |
| I-42 | C | C |
| I-28 | C | C |
| I-34 | C | C |
| I-51 | C | C |
| I-52 | C | C |
| I-53 | C | C |
| I-54 | C | C |
| I-55 | C | C |
| I-57 | C | C |
| I-58 | C | C |
| I-22 | C | C |
| I-23 | C | C |
| I-24 | C | C |
| I-1 | B | B |
| I-27 | C | C |
| I-45 | C | C |
| I-29 | C | C |
| I-30 | B | C |
| I-31 | C | C |
| I-39 | C | C |
| I-40 | C | C |
| I-35 | C | C |
| I-36 | C | C |
| I-60 | C | C |
| I-32 | C | C |
| I-33 | C | C |
| I-46 | B | B |
| I-59 | C | C |
| I-49 | B | C |
| I-61 | C | C |
| I-2 | B | A |
| I-62 | C | C |
| I-3 | B | A |
| I-63 | C | C |
| I-4 | A | A |
| I-64 | C | C |
| I-5 | B | C |
| I-43 | B | A |
| I-44 | C | C |
| I-38 | C | C |
| I-37 | C | C |

A: >90%;
B: 50-90%;
C: <50%

In some instances, Table 18 demonstrates the extent to which a compound binds to BMX. In some instances, in vitro BMX inhibition shows the extent to which a compound binds to BMX. In some instances, Table 18 shows the extent to which a compound binds to BMX and inhibits phosphorylation of a peptide substrate across a dose response. In some instances, in vitro BMX inhibition is shown in Table 18.

TABLE 18

| Compound | BMX IC50 |
|---|---|
| Number | (nM) |
| I-26 | a |
| I-56 | a |
| I-25 | a |
| I-41 | a |
| I-65 | a |
| I-47 | a |
| I-48 | a |
| I-50 | a |
| I-42 | a |
| I-28 | a |
| I-34 | a |
| I-38 | a |
| I-37 | a |

a: <1000 nM;
b: >1000 nM

In some instances, Table 19 demonstrates the extent to which a compound binds to JAK3. In some instances, in vitro binding to JAK3 shows the extent to which a compound binds to JAK3. In some instances, Table 19 shows the extent to which a compound binds to JAK3 and inhibits phosphorylation of a peptide substrate. In some instances, a compound demonstrates strong binding to JAK3 when the remaining JAK3 activity is low. In some instances, in vitro JAK3 inhibition is shown in Table 19.

TABLE 19

| | % JAK3 Remaining | % JAK3 Remaining |
|---|---|---|
| Compound | activity vs DMSO | activity vs DMSO |
| Number | (1 μM, 3 h) | (10 μM, 3 h) |
| I-51 | A | B |
| I-52 | C | C |
| I-53 | C | C |
| I-54 | C | C |
| I-55 | C | C |
| I-56 | C | C |
| I-57 | A | C |
| I-58 | A | C |
| I-22 | A | B |
| I-23 | A | B |
| I-24 | A | B |
| I-25 | A | B |
| I-26 | B | B |
| I-1 | B | C |
| I-27 | C | C |
| I-28 | A | C |
| I-45 | C | C |
| I-29 | C | C |
| I-30 | A | B |
| I-31 | A | A |
| I-39 | B | C |
| I-34 | B | C |
| I-41 | A | A |
| I-38 | A | C |
| I-40 | A | B |
| I-42 | A | B |
| I-35 | A | A |
| I-36 | A | B |
| I-60 | B | B |
| I-32 | C | C |
| I-33 | A | B |
| I-37 | C | C |
| I-46 | A | A |
| I-48 | B | C |
| I-47 | C | C |
| I-59 | B | B |
| I-49 | A | A |
| I-61 | C | C |
| I-2 | B | C |
| I-62 | A | C |
| I-3 | A | A |
| I-63 | A | B |
| I-4 | A | A |
| I-64 | B | C |
| I-65 | B | C |
| I-5 | B | C |
| I-50 | C | C |
| I-43 | A | A |
| I-44 | A | C |

A: >90%;
B: 50-90%;
C: <50%

In some instances, Table 20 demonstrates the extent to which a compound binds to JAK3. In some instances, in vitro JAK3 inhibition shows the extent to which a compound binds to JAK3. In some instances, Table 20 shows the extent to which a compound binds to JAK3 and inhibits phosphorylation of a peptide substrate across a dose response. In some instances, in vitro JAK3 inhibition is shown in Table 20.

TABLE 20

| Compound Number | JAK3 IC50 |
|---|---|
| I-53 | a |
| I-47 | a |
| I-52 | a |
| I-55 | a |
| I-61 | a |
| I-32 | a |
| I-27 | a |
| I-54 | b |
| I-50 | b |
| I-37 | b |

a: <1000 nM;
b: >1000 nM

In some instances, Table 21 demonstrates the extent to which a compound binds to FGFR4. In some instances, in vitro binding to FGFR4 shows the extent to which a compound binds to FGFR4. In some instances, Table 21 shows the extent to which a compound binds to FGFR4 and inhibits phosphorylation of a peptide substrate. In some instances, a compound demonstrates strong binding to FGFR4 when the remaining FGFR4 activity is low. In some instances, in vitro FGFR4 inhibition is shown in Table 21.

TABLE 21

| | % FGFR4 Remaining | % FGFR4 Remaining |
|---|---|---|
| Compound | activity vs DMSO | activity vs DMSO |
| Number | (1 μM, 3 h) | (10 μM, 3 h) |
| I-51 | A | C |
| I-52 | A | C |
| I-53 | C | C |
| I-54 | C | C |
| I-55 | B | C |
| I-56 | B | C |
| I-57 | C | C |
| I-58 | C | C |
| I-22 | A | C |
| I-23 | A | A |
| I-24 | C | C |
| I-25 | B | C |
| I-26 | C | C |
| I-1 | A | A |
| I-27 | B | C |
| I-28 | C | C |
| I-45 | A | C |
| I-29 | B | A |
| I-30 | C | C |
| I-31 | B | A |
| I-39 | C | C |
| I-34 | C | C |
| I-41 | B | C |
| I-38 | C | C |
| I-40 | B | C |
| I-42 | B | B |
| I-35 | B | C |
| I-36 | A | C |
| I-60 | A | A |
| I-32 | B | C |
| I-33 | B | C |
| I-37 | A | A |
| I-46 | B | A |
| I-48 | A | B |
| I-47 | B | B |
| I-59 | C | C |
| I-49 | B | C |
| I-61 | C | C |
| I-2 | A | B |
| I-62 | C | C |
| I-3 | A | A |
| I-63 | A | A |
| I-4 | A | A |
| I-64 | C | C |
| I-65 | B | B |
| I-5 | A | A |
| I-50 | A | B |
| I-43 | A | A |
| I-44 | A | C |

A: >90%;
B: 50-90%;
C: <50%

In some instances, Table 22 demonstrates the extent to which a compound binds to FGFR4. In some instances, in vitro FGFR4 inhibition shows the extent to which a compound binds to FGFR4. In some instances, Table 22 shows the extent to which a compound binds to FGFR4 and inhibits phosphorylation of a peptide substrate across a dose response. In some instances, in vitro FGFR4 inhibition is shown in Table 22.

TABLE 22

| Compound | FGFR4 IC50 |
|---|---|
| Number | (nM) |
| I-61 | a |
| I-54 | a |
| I-57 | a |
| I-28 | a |
| I-58 | a |
| I-62 | a |
| I-34 | a |
| I-59 | a |
| I-53 | a |
| I-30 | a |
| I-26 | a |
| I-24 | b |
| I-39 | a |

a: <1000 nM;
b: >1000 nM

In some instances, Table 23 demonstrates the extent to which a compound binds to RIPK2. In some instances, in vitro binding to RIPK2 shows the extent to which a compound binds to RIPK2. In some instances, Table 23 shows the extent to which a compound binds to RIPK2 and inhibits phosphorylation of a peptide substrate. In some instances, a compound demonstrates strong binding to RIPK2 when the remaining RIPK2 activity is high. In some instances, in vitro RIPK2 inhibition is shown in Table 23.

TABLE 23

| | % RIPK2 Remaining | % RIPK2 Remaining |
|---|---|---|
| Compound | activity vs DMSO | activity vs DMSO |
| Number | (1 μM, 3 h) | (10 μM, 3 h) |
| I-27 | B | C |
| I-28 | B | C |
| I-45 | A | B |
| I-29 | B | B |
| I-30 | B | C |
| I-31 | B | C |
| I-39 | C | C |
| I-34 | C | C |
| I-41 | C | C |
| I-38 | C | C |
| I-40 | C | C |
| I-42 | C | C |
| I-35 | C | C |
| I-36 | C | C |
| I-60 | A | A |
| I-32 | C | C |
| I-33 | B | C |
| I-37 | B | C |
| I-46 | A | B |
| I-48 | A | A |
| I-47 | A | A |
| I-59 | A | A |
| I-49 | A | A |

C: >90%;
B: 50-90%;
A: <50%

In some instances, Table 24 demonstrates the in cellulo tubulin degradation using a compound provided herein. In some instances, the in cellulo tubulin degradation demonstrates the extent to which a compound binds to tubulin (e.g., β-tubulin) in cells (e.g., and causes destabilization and degradation of tubulin). In some instances, Table 24 shows in cellulo tubulin degradation in K562 cells.

TABLE 24

| | Beta-tubulin - | Beta-tubulin | |
|---|---|---|---|
| Compound | K562 | K562, compound | Compound |
| Number | 24 hrs (1 μM) | concentration, 24 hrs | concentration |
| I-6 | Y | Y | 10 |
| I-7 | Y | Y | 0.5 |
| I-8 | n.d. | n.d. | 10 |
| I-9 | n.d. | Y | 10 |
| I-10 | n.d. | n.d. | 10 |
| I-11 | n.d. | Y | 0.5 |
| I-12 | n.d. | n.d. | 10 |
| I-13 | n.d. | n.d. | 10 |
| I-14 | n.d. | n.d. | 10 |
| I-15 | n.d. | Y | 10 |
| I-16 | n.d. | Y | 10 |
| I-17 | n.d. | Y | 5 |
| I-19 | n.d. | Y | 5 |

n.d.: not determined
Y: β-tubulin bound in cells and tubulin degradation occurred

### II. Preparation of Pharmaceutical Dosage Forms

### Example P1: Solution for Injection

The active ingredient is a compound of Table 1, Table 2, or Table 3, or a pharmaceutically acceptable salt thereof. A solution for intraperitoneal administration is prepared by mixing 1-1000 mg of active ingredient with 10-50 mL of a solvent mix made up by 25% dimethylacetamide, 50% propylene glycol and 25% Tween 80. Filter through millipore sterilizing filter and then distribute in 1 mL amber glass ampoules, performing all the operations under sterile conditions and under nitrogen atmosphere. 1 mL of such solution is mixed with 100 or 200 mL of sterile 5% glucose solution before using intraperitoneally.

The examples and embodiments described herein are for illustrative purposes only and various modifications or changes suggested to persons skilled in the art are to be included within the spirit and purview of this application and scope of the appended claims.

What is claimed is:

1. A compound of Formula (I), or a salt, solvate, tautomer, or regioisomer thereof: 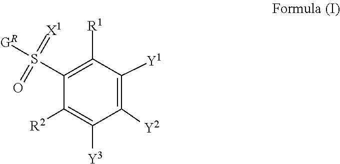
wherein,
G^{R} is alkyl, haloalkyl, heteroalkyl, —N(R⁵)₂, or G;
G is or comprises a protein-binding ligand, is or comprises (e.g., unsaturated) carbocycle, is or comprises (e.g., unsaturated) heterocycle, or is -L²-G¹, wherein L² is a linker (e.g., —O— or —NR⁵—), and G¹ is hydrogen or an organic residue (e.g., is or comprises a protein-binding ligand, is or comprises (e.g., unsaturated) carbocycle, or is or comprises (e.g., unsaturated) heterocycle);
X¹ is absent, O or NR;
each Y¹, Y², and Y³ is independently hydrogen, halo, alkyl, haloalkyl (e.g., with at least two Y being halo or haloalkyl, such as fluoroalkyl, e.g., at least one Y (e.g., Y²) being halo) (e.g., Y¹, Y², Y³ all being F), or G;
R is hydrogen or R⁷;
R¹ is R⁷;
R² is hydrogen, halogen, or R⁷;
each R³ is independently hydrogen, -L¹R⁴, —C(═O)L¹R⁴, —C(═O)OL¹R⁴, or —C(═O)NR⁴L¹R⁴, wherein each L¹ is independently alkyl, heteroalkyl, aryl, or heteroaryl; and each R⁴ is independently hydrogen, cycloalkyl, heterocycloalkyl, aryl, or heteroaryl;
each R⁵ is independently hydrogen, —CN, —C(═O)R⁶, —C(═O)OR⁶, —C(═O)NR³R⁶, alkyl, heteroalkyl, aryl, or heteroaryl;
each R⁶ is independently hydrogen, alkyl, or heteroalkyl;
each R⁷ is independently G, —CN, —OR³, —S(═O)ₓR³, —S(═O)(═NR³)R³, —S(═O)₂N(R³)₂, —OS(═O)₂R³, —N(R³)₂, —NR³C(═O)R³, —NR³C(═O)N(R³)₂, —NR³C(═NR³)N(R³)₂, —C(═O)R³, —OC(═O)R³, —C(═O)OR³, —OC(═O)OR³, —OC(═O)N(R³)₂, —C(═O)N(R³)₂, alkyl, heteroalkyl, cycloalkyl, heterocycloalkyl, aryl, or heteroaryl; and
x is 0, 1, or 2;
provided that when X¹ is O, G^{R} is G, G is L²G¹ and L² is amino or —NR⁵, then Y¹, Y², and Y³ are not all F.

2. The compound of claim 1, wherein the compound comprises only one G.

3. The compound according to claim 1 or 2, or a salt, solvate, tautomer, or regioisomer thereof, wherein when —S(═O)(═X¹)G^{R} is —S(═O)(═X¹)G, X¹ is O, then G is not:
(R)-3-(4-phenoxyphenyl)-1-(1λ²-piperidin-3-yl)-1H-pyrazolo[3,4-d]pyrimidin-4-amine;
1-(2-(λ²-azaneyl)ethyl)-3-(4-phenoxyphenyl)-1H-pyrazolo[3,4-d]pyrimidin-4-amine;
(R)-3-(4-phenoxyphenyl)-1-(1λ²-pyrrolidin-3-yl)-1H-pyrazolo[3,4-d]pyrimidin-4-amine;
4-(λ²-azaneyl)-7H-pyrrolo[2,3-d]pyrimidine;
N4-(3-(λ²-azaneyl)phenyl)-5-fluoro-N2-(4-(2-methoxyethoxy)phenyl)pyrimidine-2,4-diamine;
4-(λ²-azaneyl)-5-fluoro-N-(4-(2-methoxyethoxy)phenyl)pyrimidin-2-amine; or
3-(4-phenoxyphenyl)-1λ²-pyrazolo[5,4-d]pyrimidin-4-amine.

4. The compound according to claim 1 or 2, or a salt, solvate, tautomer, or regioisomer thereof, wherein when —S(═O)(═X¹)G^{R} is —S(═O)(═X¹)N(R⁵)G, X¹ is O, then one or more of G and R⁵ is not or does not comprise:
substituted or unsubstituted phenyl; substituted or unsubstituted benzyl; 1-naphthyl; pyridin-3-yl; pyridin-4-yl; 2-fluoropyridin-4-yl; or 2,6-difluoropyridin-3-yl.

5. The compound of any one of claims 1-4, or a salt, solvate, tautomer, or regioisomer thereof, wherein G is -L²-G¹, wherein L² is a linker, and G is an organic residue (e.g., is or comprises a protein-binding ligand, is or comprises (e.g., unsaturated) carbocycle, or is or comprises (e.g., unsaturated) heterocycle).

6. The compound of claim 5, or a salt, solvate, tautomer, or regioisomer thereof, wherein L² is a substituted or unsubstituted unsaturated alkylene (e.g., alkenylene or alkynylene), substituted or unsubstituted arylene, or substituted or unsubstituted heteroarylene, and G is an organic residue (e.g., is or comprises a protein-binding ligand).

7. The compound of claim 5, or a salt, solvate, tautomer, or regioisomer thereof, wherein L² is a bond, —O—, —NR⁸—, —N(R⁸)₂⁺—, —S—, —S(═O)—, —S(═O)₂—, —CH═CH—, ═CH—, —C≡C—, —C(═O)—, —C(═O)O—, —OC(═O)—, —OC(═O)O—, —C(═O)NR⁸—, —NR⁸C(═O)—, —OC(═O)NR⁸—, —NR⁸C(═O)O—, —NR⁸C(═O)NR⁸—, —NR⁸S(═O)₂—, —NR⁸S(═O)(═NR⁸)—, —S(═O)₂NR⁸—, —S(═O)(═NR⁸)NR⁸—, —C(═O)NR⁸S(═O)₂—, —S(═O)₂NR⁸C(═O)—, substituted or unsubstituted C₁-C₄ alkylene, substituted or unsubstituted C₁-C₈ heteroalkylene, —(C₁-C₄ alkylene)-O—, —O—(C₁-C₄ alkylene)-, —(C₁-C₄ alkylene)-NR⁸—, —NR⁸—(C₁-C₄ alkylene)-, —(C₁-C₄ alkylene)-N(R⁸)₂⁺—, or —N(R⁸)₂⁺—(C₁-C₄ alkylene)-; each R⁸ is independently hydrogen, substituted or unsubstituted C₁-C₄ alkyl, substituted or unsubstituted C₁-C₄ haloalkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and G¹ is an organic residue (e.g., is or comprises a protein-binding ligand).

8. The compound of any one of claims 1-4, or a salt, solvate, tautomer, or regioisomer thereof, wherein G is substituted or unsubstituted unsaturated carbocycle or substituted or unsubstituted unsaturated heterocycle, wherein G and R⁵ on a single N, if present, are optionally taken together to form a substituted or unsubstituted N-containing heterocycloalkyl.

9. The compound of any one of claims 1-8, or a salt, solvate, tautomer, or regioisomer thereof, wherein G comprises one or more cyclic ring systems selected from substituted or unsubstituted unsaturated carbocycles and substituted or unsubstituted unsaturated heterocycles.

10. The compound of any one of claims 1-9, or a salt, solvate, tautomer, or regioisomer thereof, wherein G comprises two or more cyclic ring systems selected from substituted or unsubstituted unsaturated carbocycles and substituted or unsubstituted unsaturated heterocycles.

11. The compound of any one of claims 5-7, or a salt, solvate, tautomer, or regioisomer thereof, wherein G¹ comprises one or more cyclic ring systems selected from substituted or unsubstituted carbocycles and substituted or unsubstituted heterocycles.

12. The compound of any one of claims 5-7 or 11, or a salt, solvate, tautomer, or regioisomer thereof, wherein G¹ comprises two or more cyclic ring systems selected from substituted or unsubstituted carbocycles and substituted or unsubstituted heterocycles.

13. The compound of claim 10 or 12, or a salt, solvate, tautomer, or regioisomer thereof, wherein the two or more cyclic ring systems are connected via a bond.

14. The compound of claim 10 or 12, or a salt, solvate, tautomer, or regioisomer thereof, wherein the two or more cyclic ring systems are connected via one or more linker and/or bond.

15. The compound of claim 14, or a salt, solvate, tautomer, or regioisomer thereof, wherein the linker is —O—, —NR⁸—, —N(R⁸)₂⁺—, —S—, —S(═O)—, —S(═O)₂—, —CH═CH—, ═CH—, —C≡C—, —C(═O)—, —C(═O)O—, —OC(═O)—, —OC(═O)O—, —C(═O)NR⁸—, —NR⁸C(═O)—, —OC(═O)NR⁸—, —NR⁸C(═O)O—, —NR⁸C(═O)NR⁸—, —NR⁸S(═O)₂—, —S(═O)₂NR⁸—, —C(═O)NR⁸S(═O)₂—, —S(═O)₂NR⁸C(═O)—, substituted or unsubstituted C₁-C₄ alkylene, substituted or unsubstituted C₁-C₈ heteroalkylene, —(C₁-C₄ alkylene)-O—, —O—(C₁-C₄ alkylene)-, —(C₁-C₄ alkylene)-NR⁸—, —NR⁸—(C₁-C₄ alkylene)-, —(C₁-C₄ alkylene)-N(R⁸)₂⁺—, or —N(R⁸)₂⁺—(C₁-C₄ alkylene)-; and each R⁸ is independently hydrogen, substituted or unsubstituted C₁-C₄ alkyl, substituted or unsubstituted C₁-C₄ haloalkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl.

16. The compound of any one of claims 9-15, or a salt, solvate, tautomer, or regioisomer thereof, wherein the cyclic ring system comprises substituted or unsubstituted monocyclic aryl or substituted or unsubstituted monocyclic heteroaryl.

17. The compound of any one of claims 9-15, or a salt, solvate, tautomer, or regioisomer thereof, wherein the cyclic ring system comprises substituted or unsubstituted bicyclic aryl or substituted or unsubstituted bicyclic heteroaryl.

18. The compound of any one of claims 1-4, or a salt, solvate, tautomer, or regioisomer thereof, wherein G or G¹ is or comprises a protein-binding ligand selected from a BTK, EGFR (e.g., EGFR T790M), JAK3, RIPK2, or tubulin binding ligand.

19. The compound of any one of claims 1-4, or a salt, solvate, tautomer, or regioisomer thereof, wherein G or G¹ is or comprises a protein-binding ligand selected from:

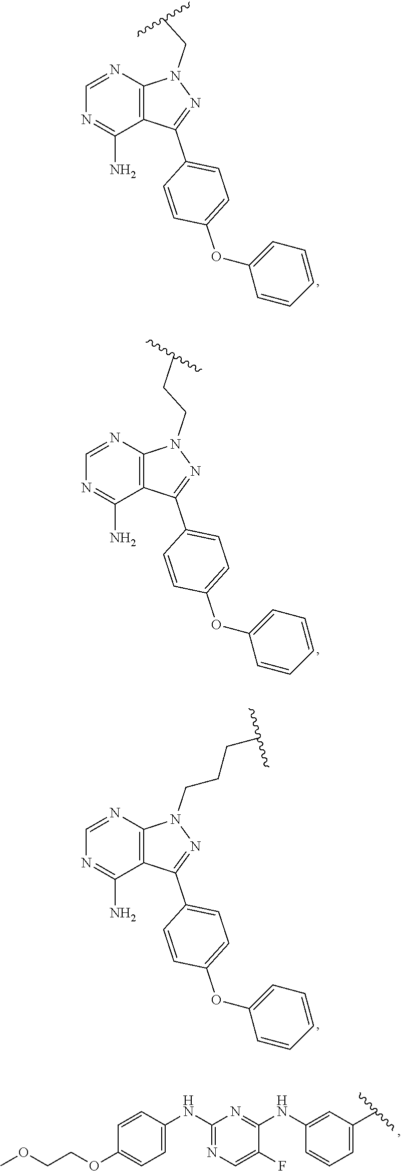 [SMILES: *Cn1nc(-c2ccc(Oc3ccccc3)cc2)c2c(N)ncnc21 ; *CCn1nc(-c2ccc(Oc3ccccc3)cc2)c2c(N)ncnc21 ; *CCCn1nc(-c2ccc(Oc3ccccc3)cc2)c2c(N)ncnc21 ; *c1cccc(Nc2nc(Nc3ccc(OCCOC)cc3)ncc2F)c1]

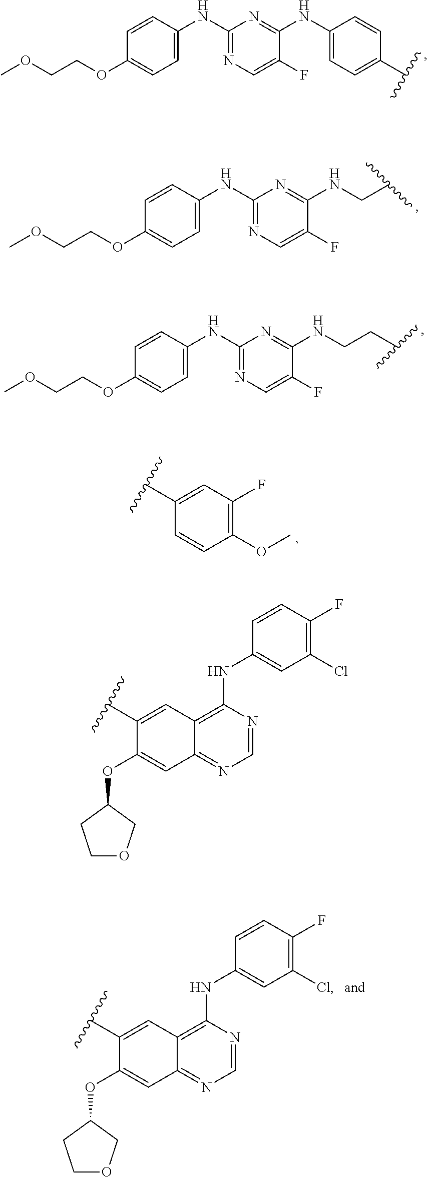

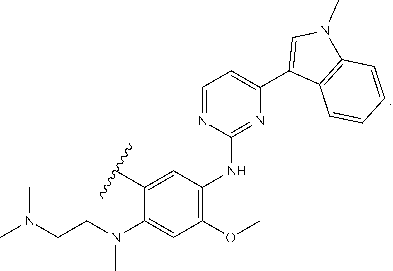 [SMILES: *c1cc(Nc2nccc(-c3cn(C)c4ccccc34)n2)c(OC)cc1N(C)CCN(C)C]

20. The compound of any one of claims 1-4, or a salt, solvate, tautomer, or regioisomer thereof, wherein G or G is or comprises a protein-binding ligand selected from:

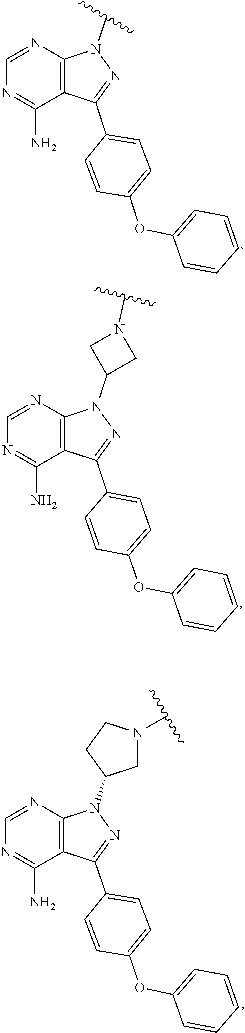 [SMILES: *n1nc(-c2ccc(Oc3ccccc3)cc2)c2c(N)ncnc21 ; *N1CC(n2nc(-c3ccc(Oc4ccccc4)cc3)c3c(N)ncnc32)C1 ; *N1CC[C@@H](n2nc(-c3ccc(Oc4ccccc4)cc3)c3c(N)ncnc32)C1]

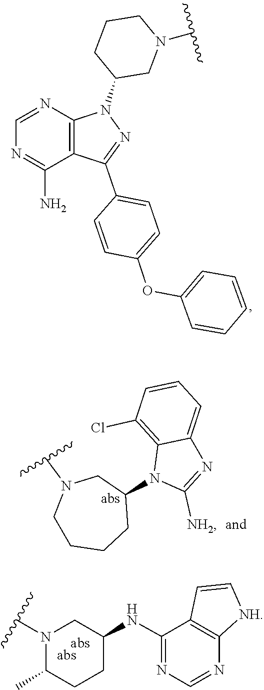 [SMILES: *N1CCC[C@@H](n2nc(-c3ccc(Oc4ccccc4)cc3)c3c(N)ncnc32)C1 ; *N1CCCC[C@H](n2c(N)nc3cccc(Cl)c32)C1 ; *N1C[C@@H](Nc2ncnc3[nH]ccc23)CC[C@@H]1C]

21. The compound of any one of claims 1-4, or a salt, solvate, tautomer, or regioisomer thereof, wherein G or G is or comprises a protein-binding ligand that is:

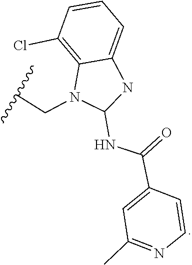

22. The compound of any one of claims 1-4, or a salt, solvate, tautomer, or regioisomer thereof, wherein G or G is or comprises a protein-binding ligand that is:

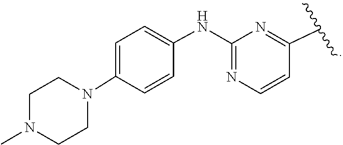 [SMILES: *c1ccnc(Nc2ccc(N3CCN(C)CC3)cc2)n1]

23. The compound of any one of claims 1-4, or a salt, solvate, tautomer, or regioisomer thereof, wherein G or G is or comprises a protein-binding ligand that is:

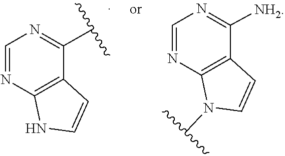 [SMILES: *c1ncnc2[nH]ccc12 ; "." ; *n1ccc2c(N)ncnc21]

24. The compound of any one of claims 1-18, or a salt, solvate, tautomer, or regioisomer thereof, wherein each R⁵ is independently hydrogen, —CN, —CH₃, —CH₂CH₃, —CH₂NH₂, —CH₂NHCH₃, —CH₂N(CH₃)₂, —CH₂F, —CHF₂, —CF₃, cyclopropyl, cyclobutyl, or cyclopentyl.

25. The compound of any one of claims 1-18, or a salt, solvate, tautomer, or regioisomer thereof, wherein each R⁵ is independently hydrogen, —CN, —CH₃, —CF₃, or cyclopropyl.

26. The compound of any one of claims 1-18, or a salt, solvate, tautomer, or regioisomer thereof, wherein each R⁵ is hydrogen.

27. The compound of any one of claims 1-26, or a salt, solvate, tautomer, or regioisomer thereof, wherein each R⁸ is independently hydrogen, substituted or unsubstituted C₁-C₄ alkyl, or substituted or unsubstituted C₁-C₄ heteroalkyl.

28. The compound of any one of claims 1-26, or a salt, solvate, tautomer, or regioisomer thereof, wherein each R⁸ is independently hydrogen, —OCH₂F, —OCHF₂, —OCF₃, —OCH₂CH₂F, —OCH₂CHF₂, —OCH₂CF₃, —NHCF₃, or —NHCH₂CF₃.

29. The compound of any one of claims 1-26, or a salt, solvate, tautomer, or regioisomer thereof, wherein each R⁸ is independently hydrogen, —OCH₃, —OCH₂CH₃, —OCH₂F, —OCHF₂, —OCF₃, —OCH₂CH₂F, —OCH₂CHF₂, —OCH₂CF₃, cyclopropyloxy, or cyclobutyloxy.

30. The compound of any one of claims 1-26, or a salt, solvate, tautomer, or regioisomer thereof, wherein each R⁸ is independently hydrogen, —CH₃, or —OCH₃.

31. The compound of any one of claims 1-30, or a salt, solvate, tautomer, or regioisomer thereof, wherein X¹ is O, NH, or N(substituted or unsubstituted alkyl).

32. The compound of any one of claims 1-30, or a salt, solvate, tautomer, or regioisomer thereof, wherein X¹ is O, NH, or N(alkyl).

33. The compound of any one of claims 1-30, or a salt, solvate, tautomer, or regioisomer thereof, wherein X¹ is O, NH, or N(CH₃).

34. The compound of any one of claims 1-30, or a salt, solvate, tautomer, or regioisomer thereof, wherein X¹ is O.

35. The compound of any one of claims 1-30, or a salt, solvate, tautomer, or regioisomer thereof, wherein X¹ is NH or N(CH₃).

36. The compound of any one of claims 1-35, or a salt, solvate, tautomer, or regioisomer thereof, wherein each Y¹, Y², and Y³ is independently halo or alkyl.

37. The compound of claim 36, or a salt, solvate, tautomer, or regioisomer thereof, wherein Y² is fluoro.

38. The compound of any one of the preceding claims, wherein R² is fluoro.

39. The compound of any one of the preceding claims, wherein Y¹ and Y³ are fluoro.

40. The compound of any one of the preceding claims, wherein R², Y¹, and Y³ are fluoro.

41. The compound of any one of the preceding claims, wherein R², Y¹, and Y³ are fluoro and G^{R} is G.

42. The compound of any one of the preceding claims, wherein R², Y¹, and Y³ are fluoro, R¹ is R⁷ (e.g., sulfone (e.g., —SO₂CH₃), sulfoxide (e.g., —S(═O)CH₃), sulfonamide (e.g., —SO₂NH₂ or —SO₂N(CH₃)₂), —OR³ (e.g., R³ being hydrogen, substituted or unsubstituted alkyl (e.g., haloalkyl), or substituted or unsubstituted aryl (e.g., phenyl)), or substituted or unsubstituted alkyl (e.g., haloalkyl)), and G^{R} is G.

43. The compound of any one of the preceding claims, wherein R², Y¹, Y², and Y³ are fluoro and R¹ is G.

44. The compound of any one of the preceding claims, wherein X is absent or O; R², Y¹, Y², and Y³ are fluoro; G^{R} is —NH₂, —N(CH₃)₂, or substituted or unsubstituted alkyl; and R¹ is G.

45. The compound of any one of claims 1-34 or 36-40, wherein 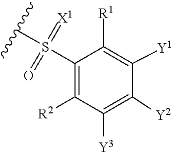
is selected from

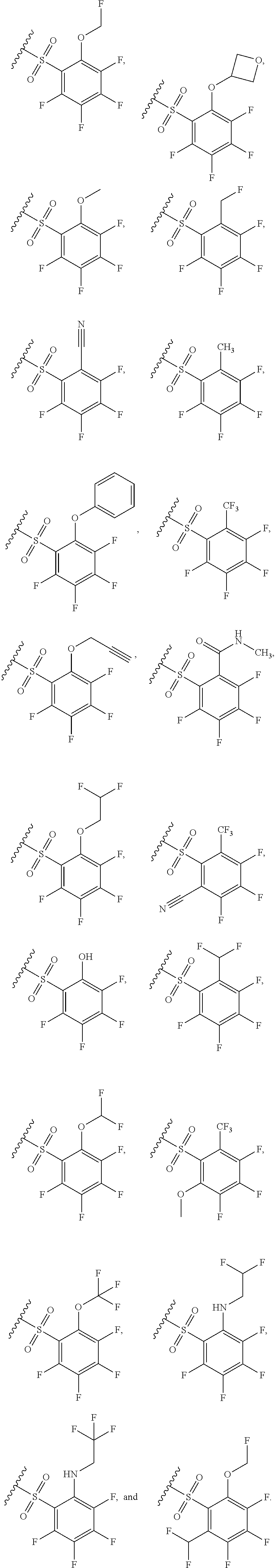

46. The compound of any one of claims 1-34 or 36-40, wherein 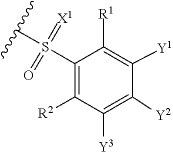
is selected from

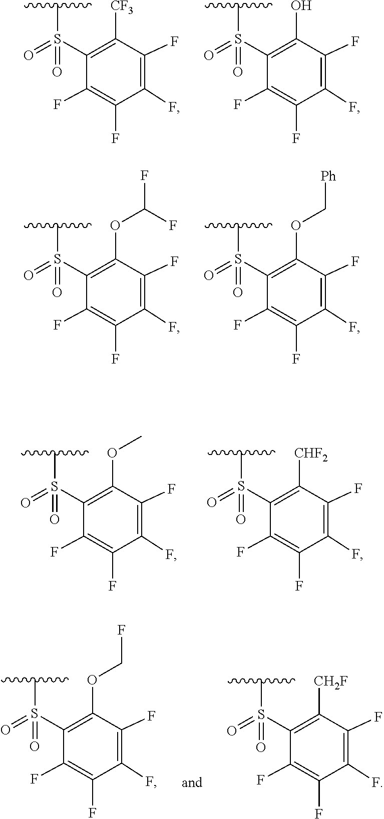

47. The compound of any one of claims 1-34 or 36-40, wherein 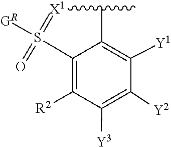
is selected from

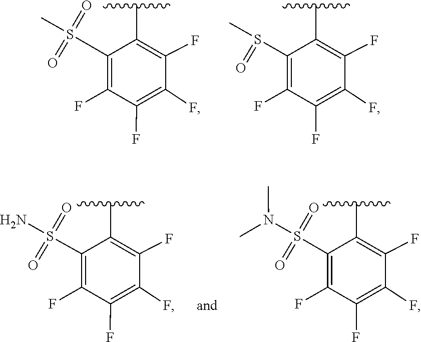

48. A compound or a salt, solvate, tautomer, or regioisomer thereof, wherein the compound is a compound from Table 1, Table 2, or Table 3.

49. A compound having a structure represented by Formula (I-A):
D1-L-D2 Formula (I-A)
wherein:
D1 is a radical of a protein-binding ligand;
D2 is a warhead radical (e.g., an aromatic (e.g., substituted phenyl) warhead radical); and
L is a linker,
or a pharmaceutically acceptable salt or solvate thereof.

50. The compound of claim 49, wherein D2 covalently modifies a target protein (e.g., tubulin (e.g., β-tubulin), Janus kinase 3 (JAK3), epidermal growth factor receptor (EGFR), Bruton's tyrosine kinase (BTK), Fibroblast Growth Factor Receptor 4 (FGFR4), receptor-interacting serine/threonine-protein kinase 2 (RIPK2), or cytoplasmic tyrosine-protein kinase (BMX)).

51. The compound according to claim 49 or 50, wherein D2 binds to, disrupts, and/or modifies a target protein (e.g., tubulin, JAK3, EGFR, BTK, FGFR4, RIPK2, or BMX).

52. The compound of any one of claims 49-51, wherein D2 comprises one or more warhead group, each warhead group being independently selected from the group consisting of substituted or unsubstituted sulfonamide (e.g., unsubstituted sulfonamide or sulfonamide substituted with alkyl (e.g., methyl)), sulfone, sulfoxide, substituted or unsubstituted amino (e.g., a secondary amine (e.g., —NH— or —NCH₃—) or a tertiary amine (e.g., >N—)), or substituted aryl (e.g., aryl substituted with one or more substituent, each substituent being independently selected from sulfone, sulfoxide, sulfonamide, halogen (e.g., fluoro), hydroxy, substituted or unsubstituted alkoxy (e.g., unsubstituted alkoxy (e.g., methoxy), alkoxy substituted with halogen (e.g., fluoro) (e.g., —OCH₂F, —OCHF₂, or —OCF₃), or alkoxy substituted with substituted or unsubstituted aryl (e.g., phenyl)), substituted or unsubstituted alkyl (e.g., alkyl substituted with halogen (e.g., fluoro) (e.g., —CH₂F, —CHF₂, or —CF₃)))).

53. The compound of any one of claims 49-52, wherein D2 comprises an aryl substituted with one or more substituent, each substituent being independently selected from halogen (e.g., fluoro), hydroxy, substituted or unsubstituted alkoxy (e.g., unsubstituted alkoxy (e.g., methoxy), alkoxy substituted with halogen (e.g., fluoro) (e.g., —OCH₂F, —OCHF₂, or —OCF₃), or alkoxy substituted with substituted or unsubstituted aryl (e.g., phenyl)), substituted or unsubstituted alkyl (e.g., alkyl substituted with halogen (e.g., fluoro) (e.g., —CH₂F, —CHF₂, or —CF₃))).

54. The compound of any one of claims 49-53, wherein D2 comprises a sulfone, a sulfoxide, or a sulfonamide.

55. The compound of any one of claims 49-54, wherein D2 comprises a sulfone and an aryl substituted with one or more substituent, each substituent being independently selected from halogen (e.g., fluoro), hydroxy, substituted or unsubstituted alkoxy (e.g., unsubstituted alkoxy (e.g., methoxy), alkoxy substituted with halogen (e.g., fluoro) (e.g., —OCH₂F, —OCHF₂, or —OCF₃), or alkoxy substituted with substituted or unsubstituted aryl (e.g., phenyl)), substituted or unsubstituted alkyl (e.g., alkyl substituted with halogen (e.g., fluoro) (e.g., —CH₂F, —CHF₂, or —CF₃))).

56. The compound of any one of claims 49-55, wherein D2 comprises a sulfoxide and an aryl substituted with one or more substituent, each substituent being independently selected from halogen (e.g., fluoro), hydroxy, substituted or unsubstituted alkoxy (e.g., unsubstituted alkoxy (e.g., methoxy), alkoxy substituted with halogen (e.g., fluoro) (e.g., —OCH₂F, —OCHF₂, or —OCF₃), or alkoxy substituted with substituted or unsubstituted aryl (e.g., phenyl)), substituted or unsubstituted alkyl (e.g., alkyl substituted with halogen (e.g., fluoro) (e.g., —CH_{2F}, —CHF₂, or —CF₃))).

57. The compound of any one of claims 49-56, wherein D2 comprises a sulfonamide and an aryl substituted with one or more substituent, each substituent being independently selected from halogen (e.g., fluoro), hydroxy, substituted or unsubstituted alkoxy (e.g., unsubstituted alkoxy (e.g., methoxy), alkoxy substituted with halogen (e.g., fluoro) (e.g., —OCH₂F, —OCHF₂, or —OCF₃), or alkoxy substituted with substituted or unsubstituted aryl (e.g., phenyl)), substituted or unsubstituted alkyl (e.g., alkyl substituted with halogen (e.g., fluoro) (e.g., —CH₂F, —CHF₂, or —CF₃))).

58. The compound of any one of claims 49-57, wherein D2 is or comprises an aryl substituted with halogen (e.g., fluoro).

59. The compound of any one of claims 49-58, wherein D2 is or comprises an aryl substituted with halogen (e.g., fluoro) and alkyl substituted with halogen (e.g., fluoro) (e.g., —CH₂F, —CHF₂, or —CF₃).

60. The compound of any one of claims 49-59, wherein D2 is or comprises an aryl substituted with halogen (e.g., fluoro) and hydroxy.

61. The compound of any one of claims 49-60, wherein D2 is or comprises an aryl substituted with halogen (e.g., fluoro) and unsubstituted alkoxy (e.g., methoxy).

62. The compound of any one of claims 49-61, wherein D2 is or comprises an aryl substituted with halogen (e.g., fluoro) and alkoxy substituted with substituted or unsubstituted an aryl (e.g., phenyl).

63. The compound of any one of claims 49-62, wherein D2 is or comprises an aryl substituted with halogen (e.g., fluoro) and alkoxy substituted with halogen (e.g., fluoro) (e.g., —OCH₂F, —OCHF₂, or —OCF₃).

64. The compound of any one of claims 49-63, wherein D2 is or comprises an aryl substituted with halogen (e.g., fluoro) and sulfone.

65. The compound of any one of claims 49-64, wherein D2 is or comprises an aryl substituted with halogen (e.g., fluoro) and sulfoxide.

66. The compound of any one of claims 49-65, wherein D2 is or comprises an aryl substituted with halogen (e.g., fluoro) and unsubstituted sulfonamide.

67. The compound of any one of claims 49-66, wherein D2 is or comprises an aryl substituted with halogen (e.g., fluoro) and substituted sulfonamide (e.g., sulfonamide substituted with alkyl (e.g., methyl)).

68. The compound of any one of claims 49-63, wherein D2 comprises a sulfone.

69. The compound of any one of claims 49-63, wherein D2 comprises a sulfonamide.

70. The compound of any one of claims 49-63, wherein D2 comprises a sulfoxide.

71. The compound of any one of claims 49-70, wherein the linker is a non-releasable linker (e.g., the linker does not decompose (e.g., hydrolyze) or release the warhead radical (or a free form thereof), the radical of the protein-binding ligand (or a free form thereof), or any other portion of the compound (e.g., a radical of any Formula provided herein) (or a free form thereof)).

72. The compound of any one of claims 49-71, wherein the linker comprises one or more linker group, each linker group being independently selected from the group consisting of —O—, (substituted or unsubstituted) amino, substituted or unsubstituted (e.g., acyclic (e.g., straight or branched) or cyclic) alkyl(ene), substituted or unsubstituted (e.g., acyclic (e.g., straight or branched) or cyclic) heteroalkyl(ene), and substituted or unsubstituted alkoxy.

73. The compound of any one of claims 49-72, wherein the linker comprises one or more linker group, each linker group being independently selected from the group consisting of (substituted or unsubstituted) amino and substituted or unsubstituted (e.g., acyclic (e.g., straight or branched) or cyclic) heteroalkyl(ene).

74. The compound of any one of claims 49-73, wherein the linker is —O—, (substituted or unsubstituted) amino or substituted or unsubstituted (e.g., acyclic (e.g., straight or branched) or cyclic) heteroalkyl(ene).

75. The compound of any one of claims 49-74, wherein L is a bond, substituted or unsubstituted alkylene (e.g., C(═O), methylene, ethylene, or alkyl substituted with oxo and/or heterocyclyl (e.g., azetidinyl, pyrrolidinyl, or piperidinyl)), substituted or unsubstituted heteroalkylene (e.g., —N═CH—, —CH₂NH—, —CH₂NCH₃—, —CH₂CH₂NH—, —NHCH₂CH₂NH—, —CH₂CH₂NCH₃—, —CH₂CH₂NHCH₂—, —NHCH₂CH₂NHCH₂—, —NHCH₂CH₂CH₂NH—, —CH₂CH₂CH₂NH—, or heteroalkyl substituted with oxo (e.g., —C(═O)NH—, —CH₂CH₂N(CH₃)C(═O)—, —CH₂CH₂NHC(═O)—, —NHCH₂CH₂N(CH₃)C(═O)—, —NHCH₂CH₂NHC(═O)—, —CH₂CH₂CH₂N(CH₃)C(═O)—, or —CH₂CH₂CH₂NHC(═O)—)), substituted or unsubstituted alkoxy (e.g., methoxy), substituted or unsubstituted piperidinyl, substituted or unsubstituted pyrrolidinyl, substituted or unsubstituted azetidinyl, or substituted or unsubstituted amino (e.g., —NH—, amino substituted with substituted or unsubstituted aryl (e.g., —NH-phenyl-, aryl substituted with amino (e.g., —NH-phenyl-NH—) or aryl substituted with alkoxy (e.g., —NH-phenyl-OCH₂—))).

76. The compound of any one of claims 49-75, wherein L is a bond.

77. The compound of any one of claims 49-76, wherein D1 has a structure represented in Table 2 or Table 3 (e.g., and L is a bond).

78. A compound selected from Table 4, Table 5, Table 6, Table 7, or Table 8.

79. A pharmaceutically acceptable composition comprising a compound of any one of the preceding claims, or a salt, solvate, tautomer, or regioisomer thereof, and one or more of pharmaceutically acceptable excipients.

80. A protein modified with a compound of any one of the preceding claims, or a salt, solvate, tautomer, or regioisomer thereof, wherein the compound forms a covalent bond with a sulfur atom of a cysteine residue of the protein.

81. A method of modifying (e.g., attaching to and/or degrading) a polypeptide with a compound, comprising contacting the polypeptide with a compound of any one of the preceding claims, or a salt, solvate, tautomer, or regioisomer thereof, to form a covalent bond with a sulfur atom of a cysteine residue of the polypeptide.

82. A method of binding a compound to a polypeptide, comprising contacting the polypeptide with a compound of any one of the preceding claims, or a salt, solvate, tautomer, or regioisomer thereof.

83. A method of disrupting a polypeptide (e.g. the function thereof), comprising contacting the polypeptide with a compound of any one of the preceding claims, or a salt, solvate, tautomer, or regioisomer thereof.
